(19) United States
(12) Patent Application Publication
(10) Pub. No.: US 2026/0035403 A1
(43) Pub. Date: Feb. 5, 2026

(54) NUCLEOSIDE LINE-1 INHIBITORS

(71) Applicant: TRANSPOSON THERAPEUTICS, INC., San Diego, CA (US)

(72) Inventors: Eckard WEBER, Los Angeles, CA (US); Michael G. CORDINGLEY, Rosemère (CA); Andrew James BURNIE, Montreal (CA); William BROWN, Montreal (CA); Chandra Mohan DARAPANENI, Montreal (CA); Marco PALADINO, Montreal (CA); Malay DOSHI, Ottawa (CA); Jigneshkumar Jashbhai PATEL, Brampton (CA)

(73) Assignee: TRANSPOSON THERAPEUTICS, INC., San Diego, CA (US)

(21) Appl. No.: 18/851,877

(22) PCT Filed: Mar. 30, 2023
(86) PCT No.: PCT/US2023/016921

§ 371 Date: Sep. 27, 2024

Related U.S. Application Data
(60) Provisional application No. 63/325,442, filed on Mar. 30, 2022.

(51) Int. Cl. C07H 19/173; A61K 31/7064; A61K 31/7068; A61K 31/7072; A61K 31/7076; A61K 31/708; C07H 19/073

(52) U.S. Cl. CPC C07H 19/173; A61K 31/7064; A61K 31/7068; A61K 31/7072; A61K 31/7076; A61K 31/708; C07H 19/073

(57) ABSTRACT

The present disclosure provides LINE-1 inhibitors and methods of treating or preventing a disease, disorder, or condition in a subject in need thereof, the methods comprising administering to the subject a therapeutically effective amount of a LINE-1 inhibitor.

### BACKGROUND

### Field

The present disclosure provides LINE-1 inhibitors and methods of treating or preventing a disease, disorder, or condition caused by LINE-1 retrotransposition in a subject by administering a LINE-1 inhibitor, or a pharmaceutical composition thereof, or a tautomer thereof, to the subject.

### Background

Long INterspersed Element-1 (LINE-1 or L1) retrotransposons form the only autonomously active family of transposable elements in humans. They are expressed and mobile in the germline, in embryonic stem cells, and in the early embryo, but are silenced in most somatic tissues. LINE-1 plays an important role in individual genome variations through insertional mutagenesis and sequence transduction, which occasionally lead to genetic diseases and disorders. For example, retrotransposable element activity is increased in many neurological disorders. See, e.g., Terry and Devine, Front. Genet., 8 Jan. 2020, https://doi.org/10.3389/fgene.2019.01244 and WO 2020/154656.

The LINE-1 element codes for two proteins, ORF1p and ORF2p, which are essential for its mobility. ORF1p is an RNA-binding protein with nucleic acid chaperone activity. ORF2p possesses endonuclease and reverse transcriptase activities. These proteins and the LINE-1 RNA assemble into a ribonucleoprotein particle (LINE-1 RNP)—the core of the retrotransposition machinery. The LINE-1 RNP mediates the synthesis of new LINE-1 copies upon cleavage of the target DNA and reverse transcription of the LINE-1 RNA at the target site. The LINE-1 element takes benefit of cellular host factors to complete its life cycle, however several cellular pathways also limit the cellular accumulation of LINE-1 RNPs and their deleterious activities. See, e.g., Pizarro and Cristofari (2016) Front. Cell Dev. Biol. 4:14. doi: 10.3389/fcell.2016.00014. There exists a need in the art for LINE-1 inhibitors to treat or prevent diseases, disorders, or conditions caused by LINE-1 retrotransposition in a subject.

### BRIEF SUMMARY

In one aspect, the present disclosure provides compounds of Table 1, see below, and the pharmaceutically acceptable salts or solvates thereof, and/or tautomers thereof; compounds of Table 2, see below, and the pharmaceutically acceptable salts or solvates thereof, and/or tautomers thereof; and compounds of Table 3, see below, and the pharmaceutically acceptable salts or solvates thereof, and/or tautomers thereof. The compounds of Tables 1-3, and the pharmaceutically acceptable salts or solvates thereof, and/or tautomers thereof, are collectively referred to as “Compounds of the Disclosure” or individually referred to as a “Compound of the Disclosure.”

In another aspect, the present disclosure provides methods of treating or preventing a disease, disorder, or condition in a subject in need thereof, the method comprising administering to the subject a therapeutically effective amount of a Compound of the Disclosure. Exemplary diseases, disorders, or conditions include, but are not limited, to neurodegenerative diseases, autoimmune diseases, and age-associated diseases.

In another aspect, the present disclosure provides methods of treating or preventing a disease, disorder, or condition caused by a pathophysiological retrotransposon-associated process in a subject in need thereof, the method comprising administering to the subject a therapeutically effective amount of a Compound of the Disclosure.

In another aspect, the present disclosure provides methods of treating or preventing a symptom of a disease, disorder, or condition caused by a pathophysiological retrotransposon-associated process in a subject in need thereof, the method comprising administering to the subject a therapeutically effective amount of a Compound of the Disclosure, or a pharmaceutical composition thereof.

In another aspect, the present disclosure provides methods of inhibiting a LINE-1 retrotransposition event, e.g., a somatic LINE-1 insertion, that causes a disease, disorder, or condition, in a subject in need thereof, the method comprising administering to the subject a therapeutically effective amount of a Compound of the Disclosure to the subject, or a pharmaceutical composition thereof.

In another aspect, the present disclosure provides methods of treating a disease, condition, or disorder in a subject in need thereof, the method comprising (a) determining whether an overexpression of a biomarker, e.g., retrotransposon RNA, retrotransposon reverse transcriptase, or retrotransposon DNA, e.g., LINE-1 RNA, ORF1p, or ORF2p, is present or absent in a biological sample taken from the subject; and (b) administering a therapeutically effective amount of a compound of a Compound of the Disclosure, or a pharmaceutical composition thereof, to the subject if an overexpression of the biomarker is present in the biological sample.

In another aspect, the present disclosure provides methods of identifying whether a subject having a disease, condition, or disorder is a candidate for treatment with a Compound of the Disclosure, or a pharmaceutical composition thereof, the method comprising (a) determining whether an overexpression of a biomarker, e.g., retrotransposon RNA, retrotransposon reverse transcriptase, or retrotransposon DNA, e.g., LINE-1 RNA, ORF1p, or ORF2p, is present or absent in a biological sample taken from the subject; and (b) identifying the subject as being a candidate for treatment if an overexpression of the biomarker is present; or (c) identifying the subject as not being a candidate for treatment if an overexpression of the biomarker is absent.

In another aspect, the present disclosure provides methods of predicting treatment outcome in a subject having a disease, condition, or disorder, the method comprising determining whether an overexpression of a biomarker, e.g., retrotransposon RNA, retrotransposon reverse transcriptase, or retrotransposon DNA, e.g., LINE-1 RNA, ORF1p, or ORF2p, is present or absent in a biological sample taken from the subject, wherein (a) the presence of an overexpression of the biomarker in the biological sample indicates that administering a Compound of the Disclosure, or a pharmaceutical composition thereof, to the subject will likely cause a favorable therapeutic response; and (b) the absence of an overexpression of the biomarker in the biological sample indicates that administering a Compound of the Disclosure, or a pharmaceutical composition thereof, to the subject will likely cause an unfavorable therapeutic response.

In another aspect, the present disclosure provides methods, comprising administering a therapeutically effective amount of a Compound of the Disclosure, or a pharmaceutical composition thereof, to a subject in need thereof, wherein (a) the subject has a disease, disorder, or condition; and (b) the disease, disorder, or condition is characterized as having an overexpression of a biomarker, e.g., retrotransposon RNA, retrotransposon reverse transcriptase, or retrotransposon DNA, e.g., LINE-1 RNA, ORF1p, or ORF2p.

In another aspect, the present disclosure provides a kit comprising a Compound of the Disclosure, or a pharmaceutical composition thereof, and instructions for administering the compound or composition to a subject having a disease, condition, or disorder caused by a pathophysiological retrotransposon-associated process.

In another aspect, the present disclosure provides a Compound of the Disclosure, or a pharmaceutical composition thereof, for use in treating or preventing a disease, disorder, or condition.

In another aspect, the present disclosure provides a Compound of the Disclosure, or a pharmaceutical composition thereof, for use in treating or preventing a disease, disorder, or condition caused by a pathophysiological retrotransposon-associated process.

In another aspect, the present disclosure provides a Compound of the Disclosure, or a pharmaceutical composition thereof, for use in treating or preventing a symptom of a disease, disorder, or condition caused by a pathophysiological retrotransposon-associated process.

In another aspect, the present disclosure provides the use of Compound of the Disclosure, or a pharmaceutical composition thereof, for the manufacture of a medicament for treating or preventing a disease, disorder, or condition.

In another aspect, the present disclosure provides the use of Compound of the Disclosure, or a pharmaceutical composition thereof, for the manufacture of a medicament for treating or preventing a disease, disorder, or condition caused by a pathophysiological retrotransposon-associated process.

In another aspect, the present disclosure provides the use of Compound of the Disclosure, or a pharmaceutical composition thereof, for the manufacture of a medicament for treating or preventing a symptom of a disease, disorder, or condition.

In another aspect, the present disclosure provides the use of Compound of the Disclosure, or a pharmaceutical composition thereof, for the manufacture of a medicament for treating or preventing a symptom of a disease, disorder, or condition caused by a pathophysiological retrotransposon-associated process.

### DETAILED DESCRIPTION

### I. Compounds of the Disclosure

In one embodiment, Compounds of the Disclosure are compounds of Table 1, or a pharmaceutically acceptable salt or solvate thereof, or a tautomer thereof.

TABLE 1

| Cpd. | | |
|---|---|---|
| No. | Structure | Chemical Name |
| 1 | 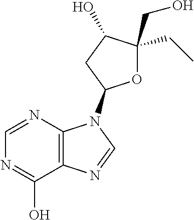 [SMILES: CC[C@]1(CO)O[C@@H](n2cnc3c(O)ncnc32)C[C@@H]1O] | (9-((2R,4S,5R)-5-ethyl-4-hydroxy-5- (hydroxymethyl)tetrahydrofuran-2- yl)-9H-purin-6-ol |
| 2 | 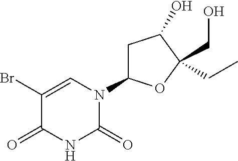 [SMILES: CC[C@]1(CO)O[C@@H](n2cc(Br)c(=O)[nH]c2=O)C[C@@H]1O] | 5-bromo-1-((2R,4S,5R)-5-ethyl-4- hydroxy-5- (hydroxymethyl)tetrahydrofuran-2- yl)-4-hydroxypyrimidin-2(1H)-one |
| 3 | 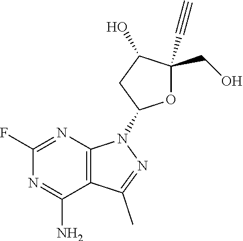 [SMILES: C#C[C@]1(CO)O[C@H](n2nc(C)c3c(N)nc(F)nc32)C[C@@H]1O] | (2R,3S,5S)-5-(4-amino-6-fluoro-3- methyl-1H-pyrazolo[3,4-d]pyrimidin- 1-yl)-2-ethynyl-2- (hydroxymethyl)tetrahydrofuran-3-ol |
| 4 | 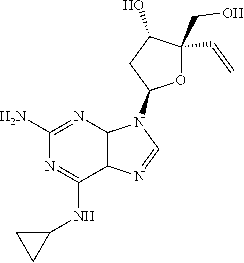 [SMILES: C=C[C@]1(CO)O[C@@H](N2C=NC3C(NC4CC4)=NC(N)=NC32)C[C@@H]1O] | (2R,3S,5R)-5-(2-amino-6- (cyclopropylamino)-9H-purin-9-yl)-2- (hydroxymethyl)-2- vinyltetrahydrofuran-3-ol |
| 5 | 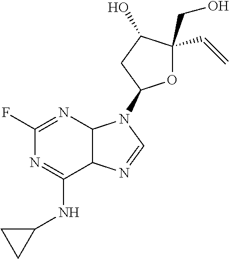 [SMILES: C=C[C@]1(CO)O[C@@H](N2C=NC3C(NC4CC4)=NC(F)=NC32)C[C@@H]1O] | (2R,3S,5R)-5-(6-(cyclopropylamino)- 2-fluoro-9H-purin-9-yl)-2- (hydroxymethyl)-2- vinyltetrahydrofuran-3-ol |
| 6 | 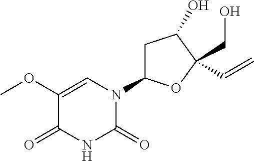 [SMILES: C=C[C@]1(CO)O[C@@H](n2cc(OC)c(=O)[nH]c2=O)C[C@@H]1O] | 1-((2R,4S,5R)-4-hydroxy-5- (hydroxymethyl)-5- vinyltetrahydrofuran-2-yl)-5- methoxypyrimidine-2,4(1H,3H)-dione |
| 7 | 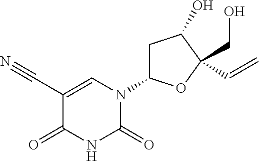 [SMILES: C=C[C@]1(CO)O[C@H](n2cc(C#N)c(=O)[nH]c2=O)C[C@@H]1O] | 1-((2S,4S,5R)-4-hydroxy-5- (hydroxymethyl)-5- vinyltetrahydrofuran-2-yl)-2,4-dioxo- 1,2,3,4-tetrahydropyrimidine-5- carbonitrile |
| 8 | 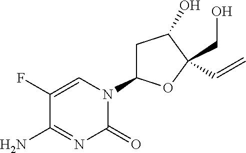 [SMILES: C=C[C@]1(CO)O[C@@H](n2cc(F)c(N)nc2=O)C[C@@H]1O] | 4-amino-5-fluoro-1-((2R,4S,5R)-4- hydroxy-5-(hydroxymethyl)-5- vinyltetrahydrofuran-2-yl)pyrimidin- 2(1H)-one |
| 9 | 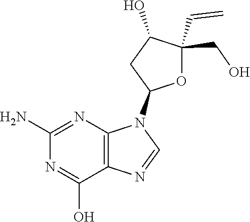 [SMILES: C=C[C@]1(CO)O[C@@H](n2cnc3c(O)nc(N)nc32)C[C@@H]1O] | 2-amino-9-((2R,4S,5R)-4-hydroxy-5- (hydroxymethyl)-5- vinyltetrahydrofuran-2-yl)-1H-purin- 6(9H)-one |
| 10 | 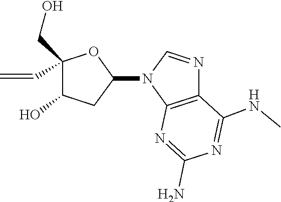 [SMILES: C=C[C@]1(CO)O[C@@H](n2cnc3c(NC)nc(N)nc32)C[C@@H]1O] | (2R,3S,5R)-5-(2-amino-6- (methylamino)-9H-purin-9-yl)-2- (hydroxymethyl)-2- vinyltetrahydrofuran-3-ol |
| 11 | 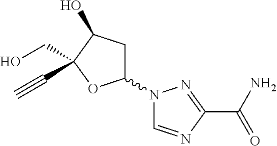 [SMILES: C#C[C@]1(CO)OC(n2cnc(C(N)=O)n2)C[C@@H]1O] | 1-((4S,5R)-5-ethynyl-4-hydroxy-5- (hydroxymethyl)tetrahydrofuran-2- yl)-1H-1,2,4-triazole-3-carboxamide |
| 12 | 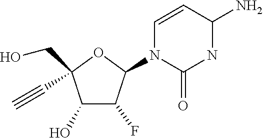 [SMILES: C#C[C@]1(CO)O[C@@H](N2C=CC(N)NC2=O)[C@H](F)[C@@H]1O] | 4-amino-1-((2R,3R,4R,5R)-5-cthynyl- 3-fluoro-4-hydroxy-5- (hydroxymethyl)tetrahydrofuran-2- yl)pyrimidin-2(1H)-one |
| 13 | 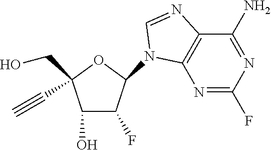 [SMILES: C#C[C@]1(CO)O[C@@H](n2cnc3c(N)nc(F)nc32)[C@H](F)[C@@H]1O] | (2R,3R,4R,5R)-5-(6-amino-2-fluoro- 9H-purin-9-yl)-2-ethynyl-4-fluoro-2- (hydroxymethyl)tetrahydrofuran-3-ol |
| 14 | 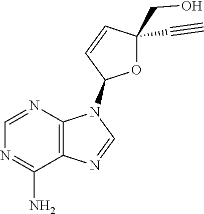 [SMILES: C#C[C@@]1(CO)C=C[C@H](n2cnc3c(N)ncnc32)O1] | ((2R,5R)-5-(6-amino-9H-purin-9-yl)- 2-ethynyl-2,5-dihydrofuran-2- yl)methanol |
| 15 | 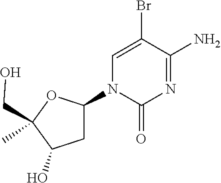 [SMILES: C[C@]1(CO)O[C@@H](n2cc(Br)c(N)nc2=O)C[C@@H]1O] | 4-amino-5-bromo-1-((2R,4S,5R)-4- hydroxy-5-(hydroxymethyl)-5- methyltetrahydrofuran-2- yl)pyrimidin-2(1H)-one |
| 16 | 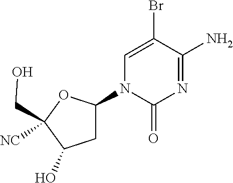 [SMILES: N#C[C@]1(CO)O[C@@H](n2cc(Br)c(N)nc2=O)C[C@@H]1O] | (2R,3S,5R)-5-(4-amino-5-bromo-2- oxopyrimidin-1(2H)-yl)-3-hydroxy-2- (hydroxymethyl)tetrahydrofuran-2- carbonitrile |
| 17 | 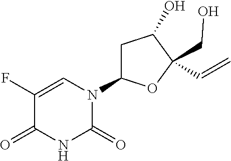 [SMILES: C=C[C@]1(CO)O[C@@H](n2cc(F)c(=O)[nH]c2=O)C[C@@H]1O] | 5-fluoro-1-((2R,4S,5R)-4-hydroxy-5- (hydroxymethyl)-5- vinyltetrahydrofuran-2-yl)pyrimidine- 2,4(1H,3H)-dione |
| 18 | 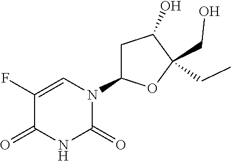 [SMILES: CC[C@]1(CO)O[C@@H](n2cc(F)c(=O)[nH]c2=O)C[C@@H]1O] | 1-((2R,4S,5R)-5-ethyl-4-hydroxy-5- (hydroxymethyl)tetrahydrofuran-2- yl)-5-fluoropyrimidine-2,4(1H,3H)- dione |
| 19 | 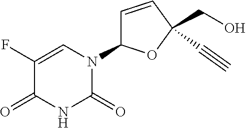 [SMILES: C#C[C@@]1(CO)C=C[C@H](n2cc(F)c(=O)[nH]c2=O)O1] | 1-((2R,5R)-5-ethynyl-5- (hydroxymethyl)-2,5-dihydrofuran-2- yl)-5-fluoropyrimidine-2,4(1H,3H)- dione |
| 20 | 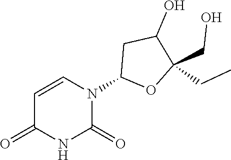 [SMILES: CC[C@]1(CO)O[C@H](n2ccc(=O)[nH]c2=O)CC1O] | 1-((2S,5R)-5-ethyl-4-hydroxy-5- (hydroxymethyl)tetrahydrofuran-2- yl)pyrimidine-2,4(1H,3H)-dione |
| 81 | 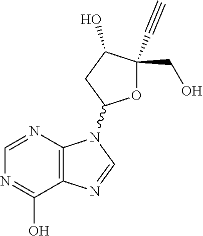 [SMILES: C#C[C@]1(CO)OC(n2cnc3c(O)ncnc32)C[C@@H]1O] | 9-((4S,5R)-5-ethynyl-4-hydroxy-5- (hydroxymethyl)tetrahydrofuran-2- yl)-9H-purin-6-ol |
| 100 | 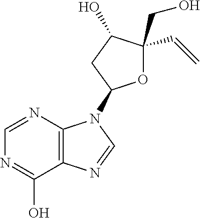 [SMILES: C=C[C@]1(CO)O[C@@H](n2cnc3c(O)ncnc32)C[C@@H]1O] | 9-((2R,4S,5R)-4-hydroxy-5- (hydroxymethyl)-5- vinyltetrahydrofuran-2-yl)-9H-purin- 6-01 |
| 101 | 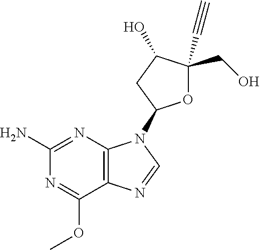 [SMILES: C#C[C@]1(CO)O[C@@H](n2cnc3c(OC)nc(N)nc32)C[C@@H]1O] | (2R,3S,5R)-5-(2-amino-6-methoxy- 9H-purin-9-yl)-2-ethynyl-2- (hydroxymethyl)tetrahydrofuran-3-ol |
| 112 | 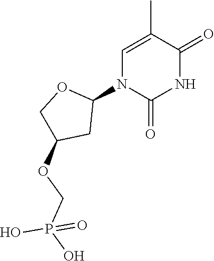 [SMILES: Cc1cn([C@H]2C[C@@H](OCP(=O)(O)O)CO2)c(=O)[nH]c1=O] | ((((3R,5R)-5-(5-methyl-2,4-dioxo-3,4- dihydropyrimidin-1(2H)- yl)tetrahydrofuran-3- yl)oxy)methyl)phosphonic acid |
| 113 | 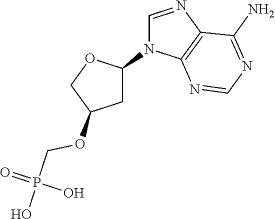 [SMILES: Nc1ncnc2c1ncn2[C@H]1C[C@@H](OCP(=O)(O)O)CO1] | ((((3R,5R)-5-(6-amino-9H-purin-9- yl)tetrahydrofuran-3- yl)oxy)methyl)phosphonic acid |
| 116 | 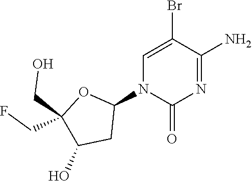 [SMILES: Nc1nc(=O)n([C@H]2C[C@H](O)[C@](CO)(CF)O2)cc1Br] | 4-amino-5-bromo-1-((2R,4S,SR)-5- (fluoromethyl)-4-hydroxy-5- (hydroxymethyl)tetrahydrofuran-2- yl)pyrimidin-2(1H)-one |
| 117 | 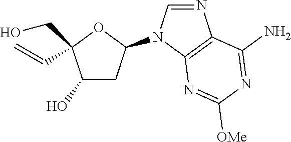 [SMILES: C=C[C@]1(CO)O[C@@H](n2cnc3c(N)nc(OC)nc32)C[C@@H]1O] | (2R,3S,5R)-5-(6-amino-2-methoxy- 9H-purin-9-yl)-2-(hydroxymethyl)-2- vinyltetrahydrofuran-3-ol |
| 120 | 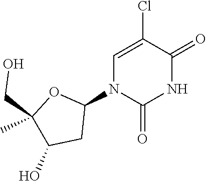 [SMILES: C[C@]1(CO)O[C@@H](n2cc(Cl)c(=O)[nH]c2=O)C[C@@H]1O] | 5-chloro-1-((2R,4S,5R)-4-hydroxy-5- (hydroxymethyl)-5- methyltetrahydrofuran-2- yl)pyrimidine-2,4(1H,3H)-dione |
| 122 | 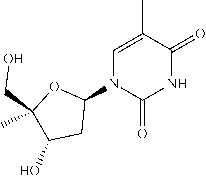 [SMILES: Cc1cn([C@H]2C[C@H](O)[C@@](C)(CO)O2)c(=O)[nH]c1=O] | 1-((2R,4S,5R)-4-hydroxy-5- (hydroxymethyl)-5- methyltetrabydrofuran-2-yl)-5- methylpyrimidine-2,4(1H,3H)-dione |
| 129 | 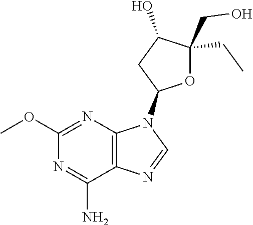 [SMILES: CC[C@]1(CO)O[C@@H](n2cnc3c(N)nc(OC)nc32)C[C@@H]1O] | (2R,3S,5R)-5-(6-amino-2-methoxy- 9H-purin-9-yl)-2-ethyl-2- (hydroxymethyl)tetrahydrofuran-3-ol |
| 131 | 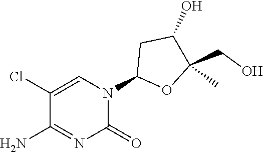 [SMILES: C[C@]1(CO)O[C@@H](n2cc(Cl)c(N)nc2=O)C[C@@H]1O] | 4-amino-5-chloro-1-((2R,4S,5R)-4- hydroxy-5-(hydroxymethyl)-5- methyltetrahydrofuran-2- yl)pyrimidin-2(1H)-one |
| 139 | 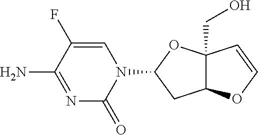 [SMILES: Nc1nc(=O)n([C@H]2C[C@@H]3OC=C[C@]3(CO)O2)cc1F] | 4-amino-5-fluoro-1-((2R,3aS,6aR)-6a- (hydroxymethyl)-2,3,3a,6a- tetrahydrofuro[3,2-b]furan-2- yl)pyrimidin-2(1H)-one |
| 140 | 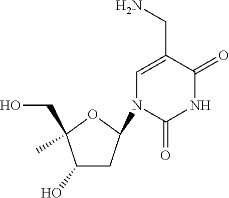 [SMILES: C[C@]1(CO)O[C@@H](n2cc(CN)c(=O)[nH]c2=O)C[C@@H]1O] | 5-(aminomethyl)-1-((2R,4S,5R)-4- hydroxy-5-(hydroxymethyl)-5- methyltetrahydrofuran-2- yl)pyrimidine-2,4(1H,3H)-dione |
| 161 | 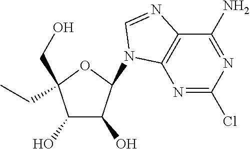 [SMILES: CC[C@]1(CO)O[C@@H](n2cnc3c(N)nc(Cl)nc32)[C@@H](O)[C@@H]1O] | (2R,3S,4S,5R)-5-(6-amino-2-fluoro- 9H-purin-9-yl)-2-ethyl-2- (hydroxymethyl)tetrahydrofuran-3,4- diol |
| 219 | 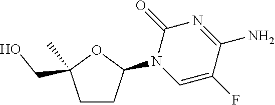 [SMILES: C[C@@]1(CO)CC[C@H](n2cc(F)c(N)nc2=O)O1] | 4-amino-5-fluoro-1-((2R,5S)-5- (hydroxymethyl)-5- methyltetrahydrofuran-2- yl)pyrimidin-2(1H)-one |
| 167 | 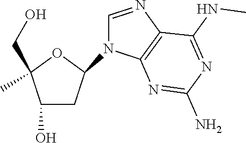 | (2R,3S,5R)-5-(2-amino-6- (methylamino)-9H-purin-9-yl)-2- (hydroxymethyl)~2- methyltetrahydrofuran-3-ol |
| 188 | 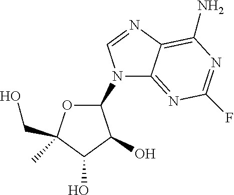 [SMILES: C[C@]1(CO)O[C@@H](n2cnc3c(N)nc(F)nc32)[C@@H](O)[C@@H]1O] | (2R,3S,4S,5R)-5-(6-amino-2-fluoro- 9H-purin-9-yl)-2-(hydroxymethyl)-2- methyltetrahydrofuran-3,4-diol |
| 189 | 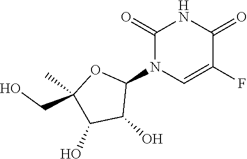 [SMILES: C[C@]1(CO)O[C@@H](n2cc(F)c(=O)[nH]c2=O)[C@H](O)[C@@H]1O] | 1-((2R,3R,4S,5R)-3,4-dihydroxy-5- (hydroxymethyl)-5- methyltetrahydrofuran-2-yl)-5- fluoropyrimidine-2,4(1H,3H)-dione |

In another embodiment, Compounds of the Disclosure are compounds of Table 2, or a pharmaceutically acceptable salt or solvate thereof, or a tautomer thereof.

TABLE 2

| Cpd. | | |
|---|---|---|
| No. | Structure | Chemical Name |
| 21 | 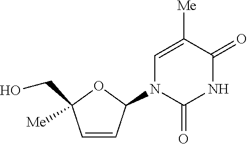 [SMILES: Cc1cn([C@H]2C=C[C@@](C)(CO)O2)c(=O)[nH]c1=O] | 1-((2R,5S)-5-(hydroxymethyl)-5-methyl- 2,5-dihydrofuran-2-yl)-5-methylpyrimidine- 2,4(1H,3H)-dione |
| 22 | 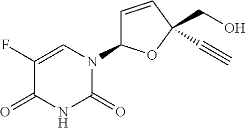 [SMILES: C#C[C@@]1(CO)C=C[C@H](n2cc(F)c(=O)[nH]c2=O)O1] | 1-((5R)-5-ethynyl-5-(hydroxymethyl)-2,5- dihydrofuran-2-yl)-5-fluoropyrimidine- 2,4(1H,3H)-dione |
| 23 | 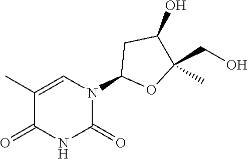 [SMILES: Cc1cn([C@H]2C[C@@H](O)[C@@](C)(CO)O2)c(=O)[nH]c1=O] | 1-((2R,4R,5R)-4-hydroxy-5- (hydroxymethyl)-5-methyltetrahydrofuran- 2-yl)-5-methylpyrimidine-2,4(1H,3H)-dione |
| 24 | 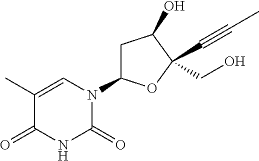 [SMILES: CC#C[C@@]1(CO)O[C@@H](n2cc(C)c(=O)[nH]c2=O)C[C@H]1O] | 1-((2R,4R,5S)-4-hydroxy-5- (hydroxymethyl)-5-(prop-1-yn-1- yl)tetrahydrofuran-2-yl)-5- methylpyrimidine-2,4(1H,3H)-dione |
| 25 | 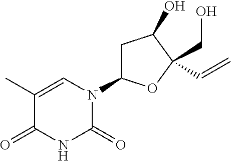 [SMILES: C=C[C@]1(CO)O[C@@H](n2cc(C)c(=O)[nH]c2=O)C[C@H]1O] | 1-((2R,4R,5R)-4-hydroxy-5- (hydroxymethyl)-5-vinyltetrahydrofuran-2- yl)-5-methylpyrimidine-2,4(1H,3H)-dione |
| 26 | 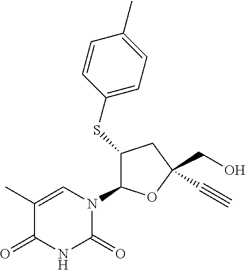 [SMILES: C#C[C@@]1(CO)C[C@@H](Sc2ccc(C)cc2)[C@H](n2cc(C)c(=O)[nH]c2=O)O1] | 1-((2R,3R,5R)-5-ethynyl-5- (hydroxymethyl)-3-(p- tolylthio)tetrahydrofuran-2-yl)-5- methylpyrimidine-2,4(1H,3H)-dione |
| 27 | 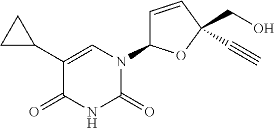 [SMILES: C#C[C@@]1(CO)C=C[C@H](n2cc(C3CC3)c(=O)[nH]c2=O)O1] | 5-cyclopropyl-1-((2R,5R)-5-ethynyl-5- (hydroxymethyl)-2,5-dihydrofuran-2- yl)pyrimidine-2,4(1H,3H)-dione |
| 28 | 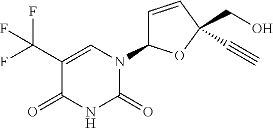 [SMILES: C#C[C@@]1(CO)C=C[C@H](n2cc(C(F)(F)F)c(=O)[nH]c2=O)O1] | 1-((2R,5R)-5-ethynyl-5-(hydroxymethyl)- 2,5-dihydrofuran-2-yl)-5- (trifluoromethyl)pyrimidine-2,4(1H,3H)- dione |
| 29 | 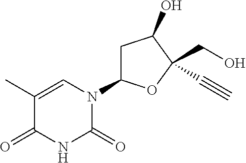 [SMILES: C#C[C@]1(CO)O[C@@H](n2cc(C)c(=O)[nH]c2=O)C[C@H]1O] | 1-((2R,4R,5R)-5-ethynyl-4-hydroxy-5- (hydroxymethyl)tetrahydrofuran-2-yl)-5- methylpyrimidine-2,4(1H,3H)-dione |
| 30 | 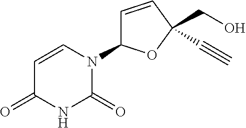 [SMILES: C#C[C@@]1(CO)C=C[C@H](n2ccc(=O)[nH]c2=O)O1] | 1-((2R,5R)-5-ethynyl-5-(hydroxymethyl)- 2,5-dihydrofuran-2-yl)pyrimidine- 2,4(1H,3H)-dione |
| 31 | 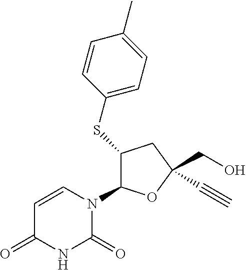 [SMILES: C#C[C@@]1(CO)C[C@@H](Sc2ccc(C)cc2)[C@H](n2ccc(=O)[nH]c2=O)O1] | 1-((2R,3R,5R)-5-ethynyl-5- (hydroxymethyl)-3-(p- tolylthio)tetrahydrofuran-2-yl)pyrimidine- 2,4(1H,3H)-dione |
| 32 | 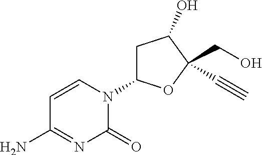 [SMILES: C#C[C@]1(CO)O[C@H](n2ccc(N)nc2=O)C[C@@H]1O] | 4-amino-1-((2S,4S,5R)-5-ethynyl-4- hydroxy-5-(hydroxymethyl)tetrahydrofuran- 2-yl)pyrimidin-2(1H)-one |
| 33 | 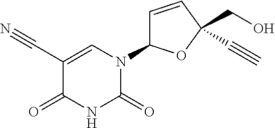 [SMILES: C#C[C@@]1(CO)C=C[C@H](n2cc(C#N)c(=O)[nH]c2=O)O1] | 1-((2R,5R)-5-ethynyl-5-(hydroxymethyl)- 2,5-dihydrofuran-2-yl)-2,4-dioxo-1,2,3,4- tetrahydropyrimidine-5-carbonitrile |
| 34 | 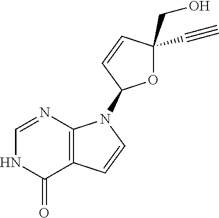 [SMILES: C#C[C@@]1(CO)C=C[C@H](n2ccc3c(=O)[nH]cnc32)O1] | 7-((2R,5R)-5-ethynyl-5-(hydroxymethyl)- 2,5-dihydrofuran-2-yl)-3,7-dihydro-4H- pyrrolo[2,3-d]pyrimidin-4-one |
| 35 | 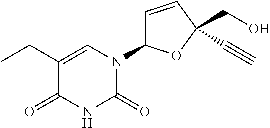 [SMILES: C#C[C@@]1(CO)C=C[C@H](n2cc(CC)c(=O)[nH]c2=O)O1] | 5-ethyl-1-((2R,5R)-5-ethynyl-5- (hydroxymethyl)-2,5-dihydrofuran-2- yl)pyrimidine-2,4(1H,3H)-dione |
| 36 | 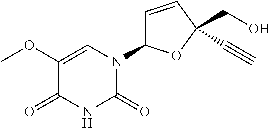 [SMILES: C#C[C@@]1(CO)C=C[C@H](n2cc(OC)c(=O)[nH]c2=O)O1] | 1-((2R,5R)-5-ethynyl-5-(hydroxymethyl)- 2,5-dihydrofuran-2-yl)-5- methoxypyrimidine-2,4(1H,3H)-dione |
| 37 | 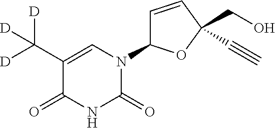 [SMILES: [2H]C([2H])([2H])c1cn([C@H]2C=C[C@@](C#C)(CO)O2)c(=O)[nH]c1=O] | 1-((2R,5R)-5-ethynyl-5-(hydroxymethyl)- 2,5-dihydrofuran-2-yl)-5-(methyl- d3)pyrimidine-2,4(1H,3H)-dione |
| 38 | 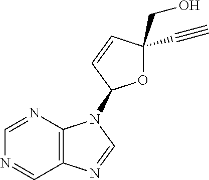 [SMILES: C#C[C@@]1(CO)C=C[C@H](n2cnc3cncnc32)O1] | ((2R,5R)-2-ethynyl-5-(9H-purin-9-yl)-2,5- dihydrofuran-2-yl)methanol |
| 39 | 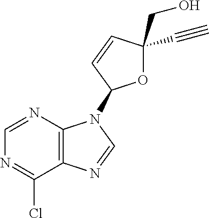 [SMILES: C#C[C@@]1(CO)C=C[C@H](n2cnc3c(Cl)ncnc32)O1] | ((2R,5R)-5-(6-chloro-9H-purin-9-yl)-2- ethynyl-2,5-dihydrofuran-2-yl)methanol |
| 40 | 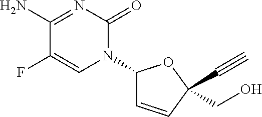 [SMILES: C#C[C@@]1(CO)C=C[C@H](n2cc(F)c(N)nc2=O)O1] | 4-amino-1-((2R,5R)-5-ethynyl-5- (hydroxymethyl)-2,5-dihydrofuran-2-yl)-5- fluoropyrimidin-2(1H)-one |
| 41 | 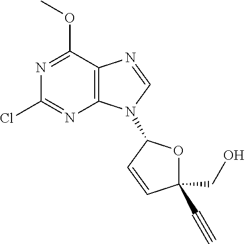 [SMILES: C#C[C@@]1(CO)C=C[C@H](n2cnc3c(OC)nc(Cl)nc32)O1] | ((2R,5R)-5-(2-chloro-6-methoxy-9H-purin- 9-yl)-2-ethynyl-2,5-dihydrofuran-2- yl)methanol |
| 42 | 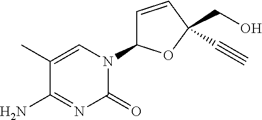 [SMILES: C#C[C@@]1(CO)C=C[C@H](n2cc(C)c(N)nc2=O)O1] | 4-amino-1-((2R,5R)-5-ethynyl-5- (hydroxymethyl)-2,5-dihydrofuran-2-yl)-5- methylpyrimidin-2(1H)-one |
| 43 | 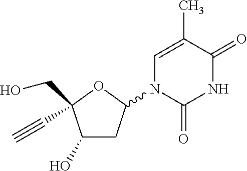 [SMILES: C#C[C@]1(CO)OC(n2cc(C)c(=O)[nH]c2=O)C[C@@H]1O] | 1-((4S,5R)-5-ethynyl-4-hydroxy-5- (hydroxymethyl)tetrahydrofuran-2-yl)-5- methylpyrimidine-2,4(1H,3H)-dione |
| 44 | 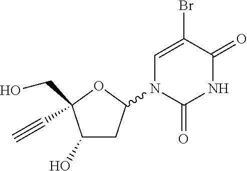 [SMILES: C#C[C@]1(CO)OC(n2cc(Br)c(=O)[nH]c2=O)C[C@@H]1O] | 5-bromo-1-((4S,5R)-5-ethynyl-4-hydroxy-5- (hydroxymethyl)tetrahydrofuran-2- yl)pyrimidine-2,4(1H,3H)-dione |
| 45 | 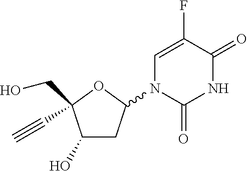 [SMILES: C#C[C@]1(CO)OC(n2cc(F)c(=O)[nH]c2=O)C[C@@H]1O] | 1-((4S,5R)-5-ethynyl-4-hydroxy-5- (hydroxymethyl)tetrahydrofuran-2-yl)-5- fluoropyrimidine-2,4(1H,3H)-dione |
| 46 | 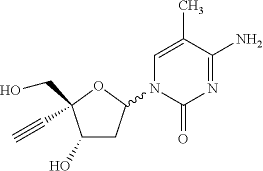 [SMILES: C#C[C@]1(CO)OC(n2cc(C)c(N)nc2=O)C[C@@H]1O] | 4-amino-1-((4S,5R)-5-ethynyl-4-hydroxy-5- (hydroxymethyl)tetrahydrofuran-2-yl)-5- methylpyrimidin-2(1H)-one |
| 47 | 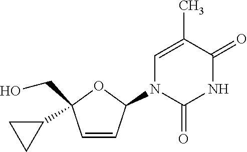 [SMILES: Cc1cn([C@H]2C=C[C@](CO)(C3CC3)O2)c(=O)[nH]c1=O] | 1-((2R,5S)-5-cyclopropyl-5- (hydroxymethyl)-2,5-dihydrofuran-2-yl)-5- methylpyrimidine-2,4(1H,3H)-dione |
| 48 | 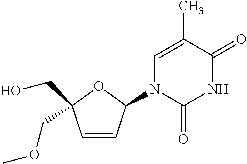 [SMILES: COC[C@@]1(CO)C=C[C@H](n2cc(C)c(=O)[nH]c2=O)O1] | 1-((2R,5S)-5-(hydroxymethyl)-5- (methoxymethyl)-2,5-dihydrofuran-2-yl)-5- methylpyrimidine-2,4(1H,3H)-dione |
| 49 | 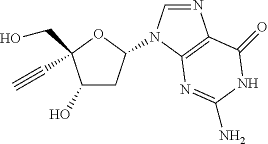 [SMILES: C#C[C@]1(CO)O[C@H](n2cnc3c(=O)[nH]c(N)nc32)C[C@@H]1O] | 2-amino-9-((2S,48,5R)-5-ethynyl-4- hydroxy-5-(hydroxymethyl)tetrahydrofuran- 2-yl)-1,9-dihydro-6H-purin-6-one |
| 50 | 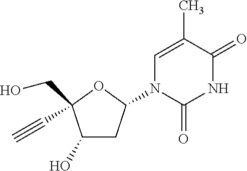 [SMILES: C#C[C@]1(CO)O[C@H](n2cc(C)c(=O)[nH]c2=O)C[C@@H]1O] | 1-((2S,4S,5R)-5-ethynyl-4-hydroxy-5- (hydroxymethyl)tetrahydrofuran-2-yl)-5- methylpyrimidine-2,4(1H,3H)-dione |
| 51 | 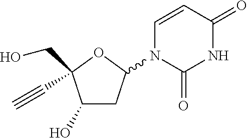 [SMILES: C#C[C@]1(CO)OC(n2ccc(=O)[nH]c2=O)C[C@@H]1O] | 1-((4S,5R)-5-ethynyl-4-hydroxy-5- (hydroxymethyl)tetrahydrofuran-2- yl)pyrimidine-2,4(1H,3H)-dione |
| 52 | 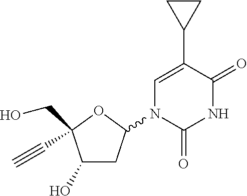 [SMILES: C#C[C@]1(CO)OC(n2cc(C3CC3)c(=O)[nH]c2=O)C[C@@H]1O] | 5-cyclopropyl-1-((4S,5R)-5-ethynyl-4- hydroxy-5-(hydroxymethyl)tetrahydrofuran- 2-yl)pyrimidine-2,4(1H,3H)-dione |
| 53 | 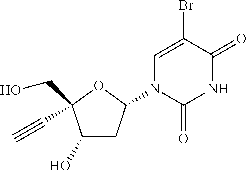 [SMILES: C#C[C@]1(CO)O[C@H](n2cc(Br)c(=O)[nH]c2=O)C[C@@H]1O] | 5-bromo-1-((2S,4S,SR)-5-ethynyl-4- hydroxy-5-(hydroxymethyl)tetrahydrofuran- 2-yl)pyrimidine-2,4(1H,3H)-dione |
| 55 | 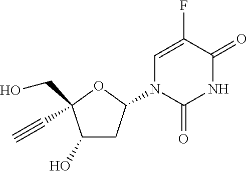 [SMILES: C#C[C@]1(CO)O[C@H](n2cc(F)c(=O)[nH]c2=O)C[C@@H]1O] | 1-((2S,4S,5R)-5-ethynyl-4-hydroxy-5- (hydroxymethyl)tetrahydrofuran-2-yl)-5- fluoropyrimidine-2,4(1H,3H)-dione |
| 55 | 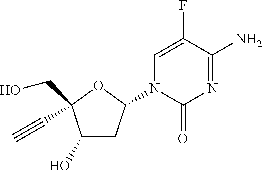 [SMILES: C#C[C@]1(CO)O[C@H](n2cc(F)c(N)nc2=O)C[C@@H]1O] | 4-amino-1-((2S,4S,5R)-5-ethynyl-4- hydroxy-5-(hydroxymethyl)tetrahydrofuran- 2-yl)-5-fluoropyrimidin-2(1H)-one |
| 56 | 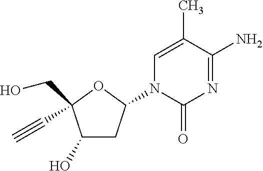 [SMILES: C#C[C@]1(CO)O[C@H](n2cc(C)c(N)nc2=O)C[C@@H]1O] | 4-amino-1-((2S,4S,5R)-5-ethynyl-4- hydroxy-5-(hydroxymethyl)tetrahydrofuran- 2-yl)-5-methylpyrimidin-2(1H)-one |
| 57 | 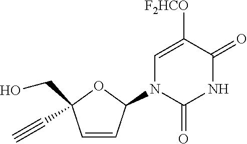 [SMILES: C#C[C@@]1(CO)C=C[C@H](n2cc(OC(F)F)c(=O)[nH]c2=O)O1] | 5-(difluoromethoxy)-1-((2R,5R)-5-ethynyl- 5-(hydroxymethyl)-2,5-dihydrofuran-2- yl)pyrimidine-2,4(1H,3H)-dione |
| 58 | 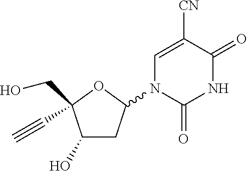 [SMILES: C#C[C@]1(CO)OC(n2cc(C#N)c(=O)[nH]c2=O)C[C@@H]1O] | 1-((4S,5R)-5-ethynyl-4-hydroxy-5- (hydroxymethyl)tetrahydrofuran-2-yl)-2,4- dioxo-1,2,3,4-tetrahydropyrimidine-5- carbonitrile |
| 59 | 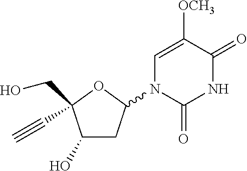 [SMILES: C#C[C@]1(CO)OC(n2cc(OC)c(=O)[nH]c2=O)C[C@@H]1O] | 1-((4S,5R)-5-ethynyl-4-hydroxy-5- (hydroxymethyl)tetrahydrofuran-2-yl)-5- methoxypyrimidine-2,4(1H,3H)-dione |
| 60 | 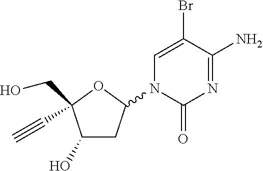 [SMILES: C#C[C@]1(CO)OC(n2cc(Br)c(N)nc2=O)C[C@@H]1O] | 4-amino-5-bromo-1-((4S,5R)-5-ethynyl-4- hydroxy-5-(hydroxymethyl)tetrahydrofuran- 2-yl)pyrimidin-2(1H)-one |
| 61 | 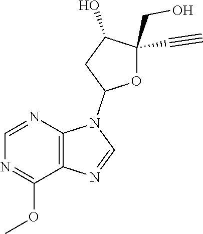 [SMILES: C#C[C@]1(CO)OC(n2cnc3c(OC)ncnc32)C[C@@H]1O] | (2R,3S)-2-ethynyl-2-(hydroxymethyl)-5-(6- methoxy-9H-purin-9-yl)tetrahydrofuran-3-ol |
| 62 | 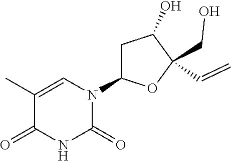 [SMILES: C=C[C@]1(CO)O[C@@H](n2cc(C)c(=O)[nH]c2=O)C[C@@H]1O] | 1-((2R,4S,SR)-4-hydroxy-5- (hydroxymethyl)-5-vinyltetrahydrofuran-2- yl)-5-methylpyrimidine-2,4(1H,3H)-dione |
| 63 | 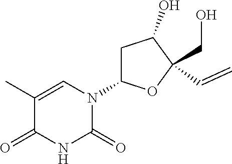 [SMILES: C=C[C@]1(CO)O[C@H](n2cc(C)c(=O)[nH]c2=O)C[C@@H]1O] | 1-((2S,4S,5R)-4-hydroxy-5- (hydroxymethyl)-5-vinyltetrahydrofuran-2- yl)-5-methylpyrimidine-2,4(1H,3H)-dione |
| 64 | 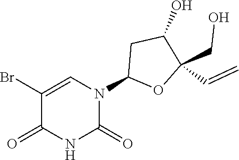 [SMILES: C=C[C@]1(CO)O[C@@H](n2cc(Br)c(=O)[nH]c2=O)C[C@@H]1O] | 5-bromo-1-((2R,4S,5R)-4-hydroxy-5- (hydroxymethyl)-5-vinyltetrahydrofuran-2- yl)pyrimidine-2,4(1H,3H)-dione |
| 65 | 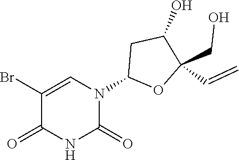 [SMILES: C=C[C@]1(CO)O[C@H](n2cc(Br)c(=O)[nH]c2=O)C[C@@H]1O] | 5-bromo-1-((2S,4S,5R)-4-hydroxy-5- (hydroxymethyl)-5-vinyltetrahydrofuran-2- yl)pyrimidine-2,4(1H,3H)-dione |
| 66 | 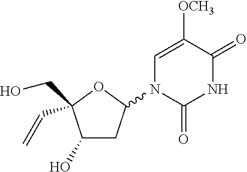 [SMILES: C=C[C@]1(CO)OC(n2cc(OC)c(=O)[nH]c2=O)C[C@@H]1O] | 1-((4S,5R)-4-hydroxy-5-(hydroxymethyl)-5- vinyltetrahydrofuran-2-yl)-5- methoxypyrimidine-2,4(1H,3H)-dione |
| 67 | 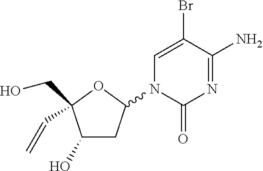 [SMILES: C=C[C@]1(CO)OC(n2cc(Br)c(N)nc2=O)C[C@@H]1O] | 4-amino-5-bromo-1-((4S,5R)-4-hydroxy-5- (hydroxymethyl)-5-vinyltetrahydrofuran-2- yl)pyrimidin-2(1H)-one |
| 68 | 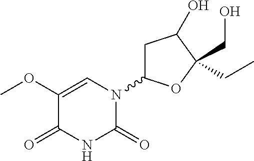 [SMILES: CC[C@]1(CO)OC(n2cc(OC)c(=O)[nH]c2=O)CC1O] | 1-((5R)-5-ethyl-4-hydroxy-5- (hydroxymethyl)tetrahydrofuran-2-yl)-5- methoxypyrimidine-2,4(1H,3H)-dione |
| 69 | 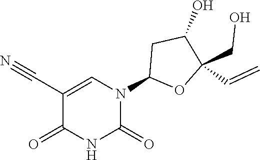 [SMILES: C=C[C@]1(CO)O[C@@H](n2cc(C#N)c(=O)[nH]c2=O)C[C@@H]1O] | 1-((2R,4S,5R)-4-hydroxy-5- (hydroxymethyl)-5-vinyltetrahydrofuran-2- yl)-2,4-dioxo-1,2,3,4-tetrahydropyrimidine- 5-carbonitrile |
| 70 | 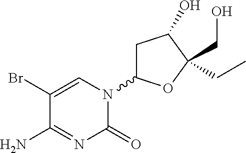 [SMILES: CC[C@]1(CO)OC(n2cc(Br)c(N)nc2=O)C[C@@H]1O] | 4-amino-5-bromo-1-((4S,SR)-5-ethyl-4- hydroxy-5-(hydroxymethyl)tetrahydrofuran- 2-yl)pyrimidin-2(1H)-one |
| 71 | 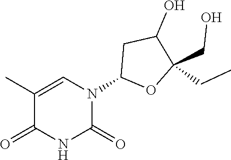 [SMILES: CC[C@]1(CO)O[C@H](n2cc(C)c(=O)[nH]c2=O)CC1O] | 1-((2S,5R)-5-ethyl-4-hydroxy-5- (hydroxymethyl)tetrahydrofuran-2-yl)-5- methylpyrimidine-2,4(1H,3H)-dione |
| 72 | 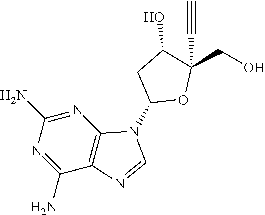 [SMILES: C#C[C@]1(CO)O[C@H](n2cnc3c(N)nc(N)nc32)C[C@@H]1O] | (2R,3S,5S)-5-(2,6-diamino-9H-purin-9-yl)- 2-ethynyl-2- (hydroxymethyl)tetrahydrofuran-3-ol |
| 73 | 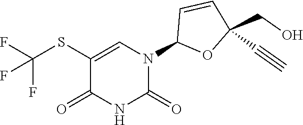 [SMILES: C#C[C@@]1(CO)C=C[C@H](n2cc(SC(F)(F)F)c(=O)[nH]c2=O)O1] | 1-((2R,5R)-5-ethynyl-5-(hydroxymethyl)- 2,5-dihydrofuran-2-yl)-5- ((trifluoromethyl)thio)pyrimidine- 2,4(1H,3H)-dione |
| 74 | 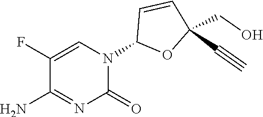 [SMILES: C#C[C@]1(CO)C=C[C@@H](n2cc(F)c(N)nc2=O)O1] | 4-amino-1-((2S,5S)-5-ethynyl-5- (hydroxymethyl)-2,5-dihydrofuran-2-yl)-5- fluoropyrimidin-2(1H)-one |
| 75 | 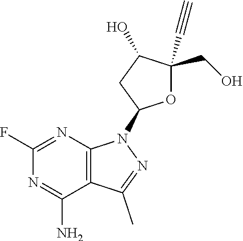 [SMILES: C#C[C@]1(CO)O[C@@H](n2nc(C)c3c(N)nc(F)nc32)C[C@@H]1O] | (2R,3S,5R)-5-(4-amino-6-fluoro-3-methyl- 1H-pyrazolo[3,4-d]pyrimidin-1-yl)-2- ethynyl-2-(hydroxymethyl)tetrahydrofuran- 3-ol |
| 76 | 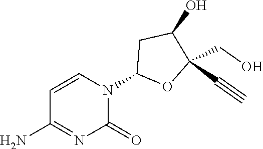 [SMILES: C#C[C@@]1(CO)O[C@H](n2ccc(N)nc2=O)C[C@H]1O] | 4-amino-1-((2S,4R,5S)-5-ethynyl-4- hydroxy-5-(hydroxymethyl)tetrahydrofuran- 2-yl)pyrimidin-2(1H)-one |
| 77 | 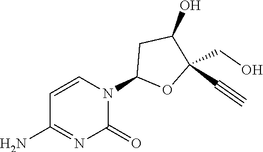 [SMILES: C#C[C@@]1(CO)O[C@@H](n2ccc(N)nc2=O)C[C@H]1O] | 4-amino-1-((2R,4R,5S)-5-ethynyl-4- hydroxy-5-(hydroxymethyl)tetrahydrofuran- 2-yl)pyrimidin-2(1H)-one |
| 78 | 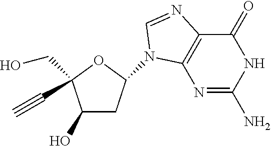 [SMILES: C#C[C@@]1(CO)O[C@H](n2cnc3c(=O)[nH]c(N)nc32)C[C@H]1O] | 2-amino-9-((2S,4R,5S)-5-ethynyl-4- hydroxy-5-(hydroxymethyl)tetrahydrofuran- 2-yl)-1,9-dihydro-6H-purin-6-one |
| 79 | 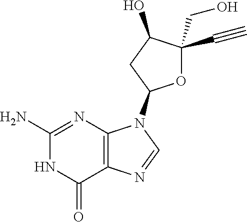 [SMILES: C#C[C@@]1(CO)O[C@@H](n2cnc3c(=O)[nH]c(N)nc32)C[C@H]1O] | 2-amino-9-((2R,4R,5S)-5-ethynyl-4- hydroxy-5-(hydroxymethyl)tetrahydrofuran- 2-yl)-1,9-dihydro-6H-purin-6-one |
| 80 | 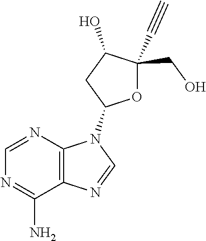 [SMILES: C#C[C@]1(CO)O[C@H](n2cnc3c(N)ncnc32)C[C@@H]1O] | (2R,3S,5S)-5-(6-amino-9H-purin-9-yl)-2- ethynyl-2-(hydroxymethyl)tetrahydrofuran- 3-ol |
| 82 | 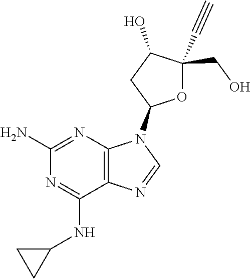 [SMILES: C#C[C@]1(CO)O[C@@H](n2cnc3c(NC4CC4)nc(N)nc32)C[C@@H]1O] | (2R,3S,5R)-5-(2-amino-6- (cyclopropylamino)-9H-purin-9-yl)-2- ethynyl-2-(hydroxymethyl)tetrahydrofuran- 3-ol |
| 83 | 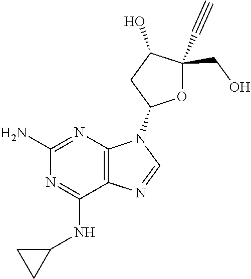 [SMILES: C#C[C@]1(CO)O[C@H](n2cnc3c(NC4CC4)nc(N)nc32)C[C@@H]1O] | (2R,3S,5S)-5-(2-amino-6- (cyclopropylamino)-9H-purin-9-yl)-2- ethynyl-2-(hydroxymethyl)tetrahydrofuran- 3-ol |
| 84 | 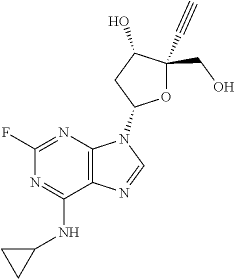 [SMILES: C#C[C@]1(CO)O[C@H](n2cnc3c(NC4CC4)nc(F)nc32)C[C@@H]1O] | (2R,3S,5S)-5-(6-(cyclopropylamino)-2- fluoro-9H-purin-9-yl)-2-ethynyl-2- (hydroxymethyl)tetrahydrofuran-3-ol |
| 85 | 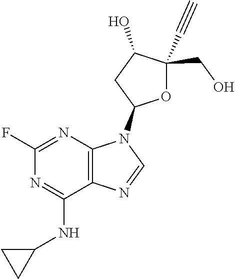 [SMILES: C#C[C@]1(CO)O[C@@H](n2cnc3c(NC4CC4)nc(F)nc32)C[C@@H]1O] | (2R,3S,5R)-5-(6-(cyclopropylamino)-2- fluoro-9H-purin-9-yl)-2-ethynyl-2- (hydroxymethyl)tetrahydrofuran-3-ol |
| 86 | 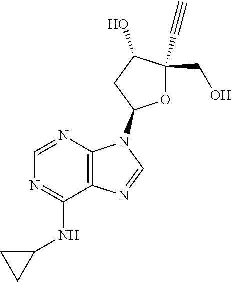 [SMILES: C#C[C@]1(CO)O[C@@H](n2cnc3c(NC4CC4)ncnc32)C[C@@H]1O] | (2R,3S,5R)-5-(6-(cyclopropylamino)-9H- purin-9-yl)-2-ethynyl-2- (hydroxymethyl)tetrahydrofuran-3-ol |
| 87 | 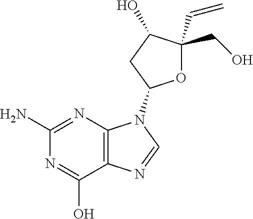 [SMILES: C=C[C@]1(CO)O[C@H](n2cnc3c(O)nc(N)nc32)C[C@@H]1O] | 2-amino-9-((2S,4S,5R)-4-hydroxy-5- (hydroxymethyl)-5-vinyltetrahydrofuran-2- yl)-9H-purin-6-ol |
| 88 | 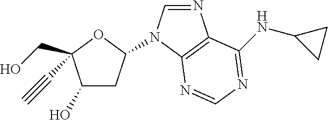 [SMILES: C#C[C@]1(CO)O[C@H](n2cnc3c(NC4CC4)ncnc32)C[C@@H]1O] | (2R,3S,5S)-5-(6-(cyclopropylamino)-9H- purin-9-yl)-2-ethynyl-2- (hydroxymethyl)tetrahydrofuran-3-ol |
| 89 | 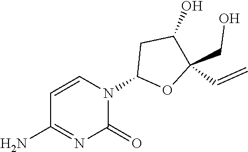 [SMILES: C=C[C@]1(CO)O[C@H](n2ccc(N)nc2=O)C[C@@H]1O] | 4-amino-1-((2S,4S,5R)-4-hydroxy-5- (hydroxymethyl)-5-vinyltetrahydrofuran-2- yl)pyrimidin-2(1H)-one |
| 90 | 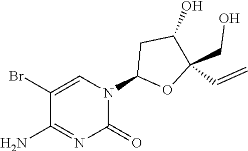 [SMILES: C=C[C@]1(CO)O[C@@H](n2cc(Br)c(N)nc2=O)C[C@@H]1O] | 4-amino-5-bromo-1-((2R,48,5R)-4- hydroxy-5-(hydroxymethyl)-5- vinyltetrahydrofuran-2-yl)pyrimidin-2(1H)- one |
| 91 | 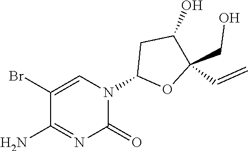 [SMILES: C=C[C@]1(CO)O[C@H](n2cc(Br)c(N)nc2=O)C[C@@H]1O] | 4-amino-5-bromo-1-((2S,4S,5R)-4-hydroxy- 5-(hydroxymethyl)-5-vinyltetrahydrofuran- 2-yl)pyrimidin-2(1H)-one |
| 92 | 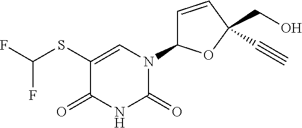 [SMILES: C#C[C@@]1(CO)C=C[C@H](n2cc(SC(F)F)c(=O)[nH]c2=O)O1] | 5-((difluoromethyl)thio)-1-((2R,5R)-5- ethynyl-5-(hydroxymethyl)-2,5- dihydrofuran-2-yl)pyrimidine-2,4(1H,3H)- dione |
| 93 | 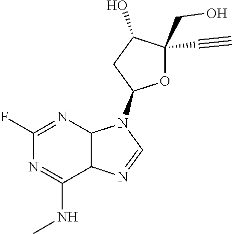 [SMILES: C#C[C@]1(CO)O[C@@H](N2C=NC3C(NC)=NC(F)=NC32)C[C@@H]1O] | (2R,3S,5R)-2-ethynyl-5-(2-fluoro-6- (methylamino)-4,5-dihydro-9H-purin-9-yl)- 2-(hydroxymethyl)tetrahydrofuran-3-ol |
| 94 | 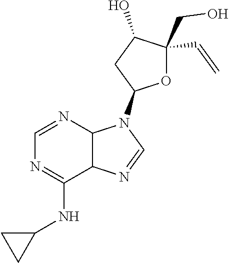 [SMILES: C=C[C@]1(CO)O[C@@H](N2C=NC3C(NC4CC4)=NC=NC32)C[C@@H]1O] | (2R,3S,5R)-5-(6-(cyclopropylamino)-4,5- dihydro-9H-purin-9-yl)-2-(hydroxymethyl)- 2-vinyltetrahydrofuran-3-ol |
| 95 | 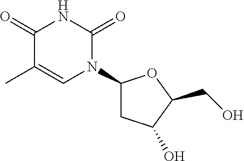 [SMILES: Cc1cn([C@@H]2C[C@@H](O)[C@H](CO)O2)c(=O)[nH]c1=O] | 1-((2S,4R,5S)-4-hydroxy-5- (hydroxymethyl)tetrahydrofuran-2-yl)-5- methylpyrimidine-2,4(1H,3H)-dione |
| 96 | 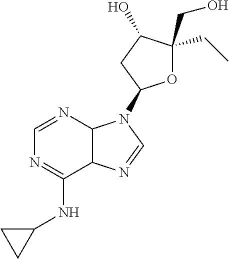 [SMILES: CC[C@]1(CO)O[C@@H](N2C=NC3C(NC4CC4)=NC=NC32)C[C@@H]1O] | (2R,3S,5R)-5-(6-(cyclopropylamino)-4,5- dihydro-9H-purin-9-yl)-2-ethyl-2- (hydroxymethyl)tetrahydrofuran-3-ol |
| 97 | 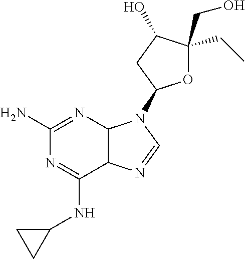 [SMILES: CC[C@]1(CO)O[C@@H](N2C=NC3C(NC4CC4)=NC(N)=NC32)C[C@@H]1O] | (2R,3S,5R)-5-(2-amino-6- (cyclopropylamino)-4,5-dihydro-9H-purin- 9-yl)-2-ethyl-2- (hydroxymethyl)tetrahydrofuran-3-ol |
| 98 | 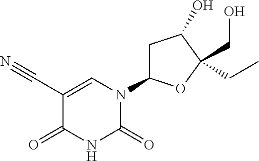 [SMILES: CC[C@]1(CO)O[C@@H](n2cc(C#N)c(=O)[nH]c2=O)C[C@@H]1O] | 1-((2R,4S,5R)-5-ethyl-4-hydroxy-5- (hydroxymethyl)tetrahydrofuran-2-yl)-2,4- dioxo-1,2,3,4-tetrahydropyrimidine-5- carbonitrile |
| 99 | 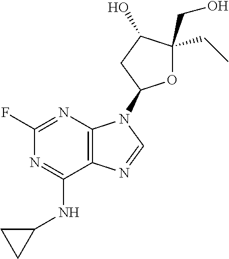 [SMILES: CC[C@]1(CO)O[C@@H](n2cnc3c(NC4CC4)nc(F)nc32)C[C@@H]1O] | (2R,3S,5R)-5-(6-(cyclopropylamino)-2- fluoro-9H-purin-9-yl)-2-ethyl-2- (hydroxymethyl)tetrahydrofuran-3-ol |
| 102 | 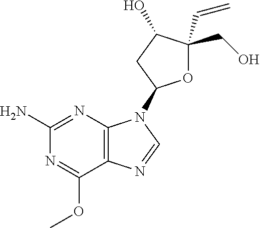 [SMILES: C=C[C@]1(CO)O[C@@H](n2cnc3c(OC)nc(N)nc32)C[C@@H]1O] | (2R,3S,5R)-5-(2-amino-6-methoxy-9H- purin-9-yl)-2-(hydroxymethyl)-2- vinyltetrahydrofuran-3-ol |
| 103 | 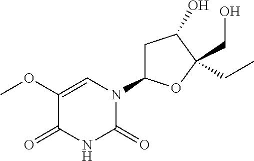 [SMILES: CC[C@]1(CO)O[C@@H](n2cc(OC)c(=O)[nH]c2=O)C[C@@H]1O] | 1-((2R,4S,5R)-5-ethyl-4-hydroxy-5- (hydroxymethyl)tetrahydrofuran-2-yl)-5- methoxypyrimidine-2,4(1H,3H)-dione |
| 104 | 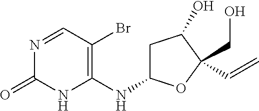 [SMILES: C=C[C@]1(CO)O[C@H](Nc2[nH]c(=O)ncc2Br)C[C@@H]1O] | 5-bromo-6-(((2S,4S,5R)-4-hydroxy-5- (hydroxymethyl)-5-vinyltetrahydrofuran-2- yl)amino)pyrimidin-2(1H)-one |
| 105 | 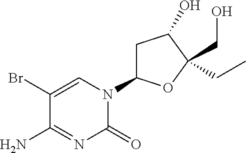 [SMILES: CC[C@]1(CO)O[C@@H](n2cc(Br)c(N)nc2=O)C[C@@H]1O] | 4-amino-5-bromo-1-((2R,4S,5R)-5-ethyl-4- hydroxy-5-(hydroxymethyl)tetrahydrofuran- 2-yl)pyrimidin-2(1H)-one |
| 106 | 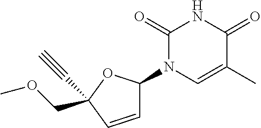 [SMILES: C#C[C@@]1(COC)C=C[C@H](n2cc(C)c(=O)[nH]c2=O)O1] | 1-((2R,5R)-5-ethynyl-5-(methoxymethyl)- 2,5-dihydrofuran-2-yl)-5-methylpyrimidine- 2,4(1H,3H)-dione |
| 107 | 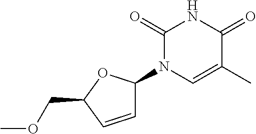 [SMILES: COC[C@@H]1C=C[C@H](n2cc(C)c(=O)[nH]c2=O)O1] | 1-((2R,5S)-5-(methoxymethyl)-2,5- dihydrofuran-2-yl)-5-methylpyrimidine- 2,4(1H,3H)-dione |
| 108 | 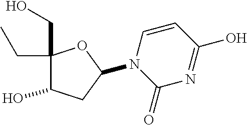 [SMILES: CC[C@]1(CO)O[C@@H](n2ccc(O)nc2=O)C[C@@H]1O] | 1-((2R,4S,5R)-5-ethyl-4-hydroxy-5- (hydroxymethyl)tetrahydrofuran-2-yl)-4- hydroxypyrimidin-2(1H)-one |
| 110 | 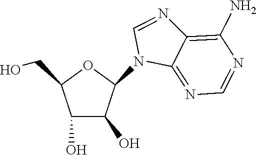 [SMILES: Nc1ncnc2c1ncn2[C@@H]1O[C@H](CO)[C@@H](O)[C@@H]1O] | (2R,3S,4S,5R)-2-(6-amino-9H-purin-9-yl)- 5-(hydroxymethyl)tetrahydrofuran-3,4-diol |
| 111 | 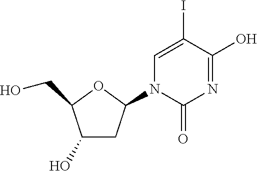 [SMILES: O=c1nc(O)c(I)cn1[C@H]1C[C@H](O)[C@@H](CO)O1] | 4-hydroxy-1-((2R,4S,5R)-4-hydroxy-5- (hydroxymethyl)tetrahydrofuran-2-yl)-5- iodopyrimidin-2(1H)-one |
| 114 | 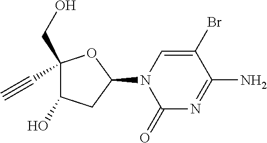 [SMILES: C#C[C@]1(CO)O[C@@H](n2cc(Br)c(N)nc2=O)C[C@@H]1O] | 4-amino-5-bromo-1-((2R,4S,5R)-5-ethynyl- 4-hydroxy-5- (hydroxymethyl)tetrahydrofuran-2- yl)pyrimidin-2(1H)-one |
| 115 | 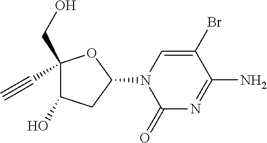 [SMILES: C#C[C@]1(CO)O[C@H](n2cc(Br)c(N)nc2=O)C[C@@H]1O] | 4-amino-5-bromo-1-((2S,4S,5R)-5-ethynyl- 4-hydroxy-5- (hydroxymethyl)tetrahydrofuran-2- yl)pyrimidin-2(1H)-one |
| 124 | 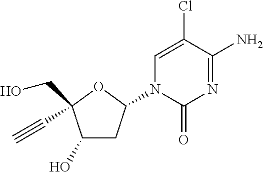 [SMILES: C#C[C@]1(CO)O[C@H](n2cc(Cl)c(N)nc2=O)C[C@@H]1O] | 4-amino-5-chloro-1-((2S,4S,5R)-5-ethynyl- 4-hydroxy-5- (hydroxymethyl)tetrahydrofuran-2- yl)pyrimidin-2(1H)-one |
| 125 | 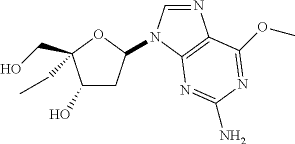 [SMILES: CC[C@]1(CO)O[C@@H](n2cnc3c(OC)nc(N)nc32)C[C@@H]1O] | (2R,3S,5R)-5-(2-amino-6-methoxy-9H- purin-9-yl)-2-ethyl-2- (hydroxymethyl)tetrahydrofuran-3-ol |
| 128 | 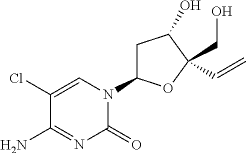 [SMILES: C=C[C@]1(CO)O[C@@H](n2cc(Cl)c(N)nc2=O)C[C@@H]1O] | 4-amino-5-chloro-1-((2R,4S,5R)-4-hydroxy- 5-(hydroxymethyl)-5-vinyltetrahydrofuran- 2-yl)pyrimidin-2(1H)-one |
| 132 | 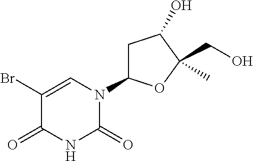 [SMILES: C[C@]1(CO)O[C@@H](n2cc(Br)c(=O)[nH]c2=O)C[C@@H]1O] | 5-bromo-1-((2R,4S,5R)-4-hydroxy-5- (hydroxymethyl)-5-methyltetrahydrofuran- 2-yl)pyrimidine-2,4(1H,3H)-dione |
| 133 | 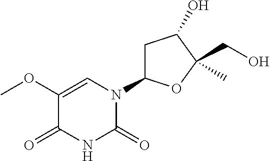 [SMILES: COc1cn([C@H]2C[C@H](O)[C@@](C)(CO)O2)c(=O)[nH]c1=O] | 1-((2R,4S,5R)-4-hydroxy-5- (hydroxymethyl)-5-methyltetrahydrofuran- 2-yl)-5-methoxypyrimidine-2,4(1H,3H)- dione |
| 134 | 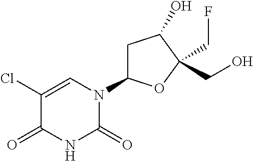 [SMILES: O=c1[nH]c(=O)n([C@H]2C[C@H](O)[C@](CO)(CF)O2)cc1Cl] | 5-chloro-1-((2R,4S,5R)-5-(fluoromethyl)-4- hydroxy-5-(hydroxymethyl)tetrahydrofuran- 2-yl)pyrimidine-2,4(1H,3H)-dione |
| 135 | 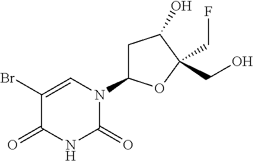 [SMILES: O=c1[nH]c(=O)n([C@H]2C[C@H](O)[C@](CO)(CF)O2)cc1Br] | 5-bromo-1-((2R,4S,5R)-5-(fluoromethyl)-4- hydroxy-5-(hydroxymethyl)tetrahydrofuran- 2-yl)pyrimidine-2,4(1H,3H)-dione |
| 136 | 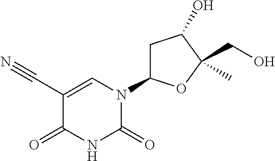 [SMILES: C[C@]1(CO)O[C@@H](n2cc(C#N)c(=O)[nH]c2=O)C[C@@H]1O] | 1-((2R,4S,5R)-4-hydroxy-5- (hydroxymethyl)-5-methyltetrahydrofuran- 2-yl)-2,4-dioxo-1,2,3,4- tetrahydropyrimidine-5-carbonitrile |
| 137 | 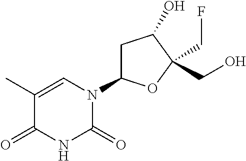 [SMILES: Cc1cn([C@H]2C[C@H](O)[C@](CO)(CF)O2)c(=O)[nH]c1=O] | 1-((2R,4S,5R)-5-(fluoromethyl)-4-hydroxy- 5-(hydroxymethyl)tetrahydrofuran-2-yl)-5- methylpyrimidine-2,4(1H,3H)-dione |
| 143 | 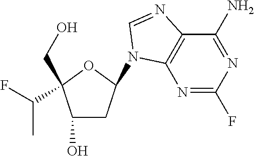 [SMILES: CC(F)[C@]1(CO)O[C@@H](n2cnc3c(N)nc(F)nc32)C[C@@H]1O] | (2R,3S,5R)-5-(6-amino-2-fluoro-9H-purin- 9-yl)-2-((R)-1-fluoroethyl)-2- (hydroxymethyl)tetrahydrofuran-3-ol |
| 146 | 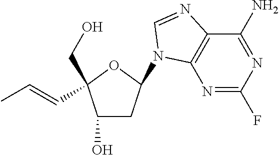 [SMILES: C/C=C/[C@]1(CO)O[C@@H](n2cnc3c(N)nc(F)nc32)C[C@@H]1O] | (2R,3S,5R)-5-(6-amino-2-fluoro-9H-purin- 9-yl)-2-(hydroxymethyl)-2-((E)-prop-1-en- 1-yl)tetrahydrofuran-3-ol |
| 147 | 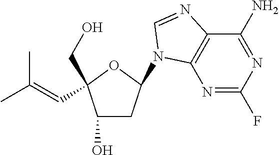 [SMILES: CC(C)=C[C@]1(CO)O[C@@H](n2cnc3c(N)nc(F)nc32)C[C@@H]1O] | (2R,3S,5R)-5-(6-amino-2-fluoro-9H-purin- 9-yl)-2-(hydroxymethyl)-2-(2-methylprop- 1-en-1-yl)tetrahydrofuran-3-ol |
| 149 | 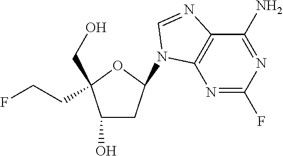 [SMILES: Nc1nc(F)nc2c1ncn2[C@H]1C[C@H](O)[C@](CO)(CCF)O1] | (2R,3S,5R)-5-(6-amino-2-fluoro-9H-purin- 9-yl)-2-(2-fluoroethyl)-2- (hydroxymethyl)tetrahydrofuran-3-ol |
| 150 | 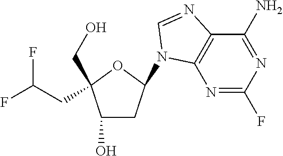 [SMILES: Nc1nc(F)nc2c1ncn2[C@H]1C[C@H](O)[C@](CO)(CC(F)F)O1] | (2R,3S,5R)-5-(6-amino-2-fluoro-9H-purin- 9-yl)-2-(2,2-difluoroethyl)-2- (hydroxymethyl)tetrahydrofuran-3-ol |
| 151 | 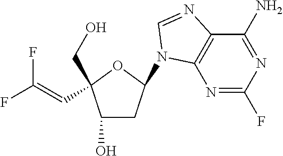 [SMILES: Nc1nc(F)nc2c1ncn2[C@H]1C[C@H](O)[C@@](C=C(F)F)(CO)O1] | (2R,3S,5R)-5-(6-amino-2-fluoro-9H-purin- 9-yl)-2-(2,2-difluorovinyl)-2- (hydroxymethyl)tetrahydrofuran-3-ol |
| 152 | 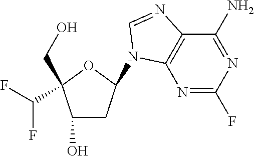 [SMILES: Nc1nc(F)nc2c1ncn2[C@H]1C[C@H](O)[C@](CO)(C(F)F)O1] | (2R,3S,5R)-5-(6-amino-2-fluoro-9H-purin- 9-yl)-2-(difluoromethyl)-2- (hydroxymethyl)tetrahydrofuran-3-ol |
| 153 | 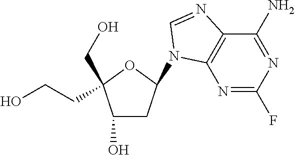 [SMILES: Nc1nc(F)nc2c1ncn2[C@H]1C[C@H](O)[C@](CO)(CCO)O1] | (2R,3S,5R)-5-(6-amino-2-fluoro-9H-purin- 9-yl)-2-(2-hydroxyethyl)-2- (hydroxymethyl)tetrahydrofuran-3-ol |
| 154 | 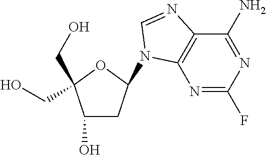 | ((3S,5R)-5-(6-amino-2-fluoro-9H-purin-9- yl)-3-hydroxytetrahydrofuran-2,2- diyl)dimethanol |
| 156 | 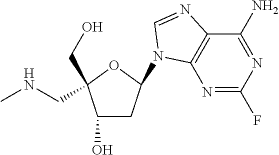 [SMILES: CNC[C@]1(CO)O[C@@H](n2cnc3c(N)nc(F)nc32)C[C@@H]1O] | (2R,3S,5R)-5-(6-amino-2-fluoro-9H-purin- 9-yl)-2-(hydroxymethyl)-2- ((methylamino)methyl)tetrahydrofuran-3-ol |
| 157 | 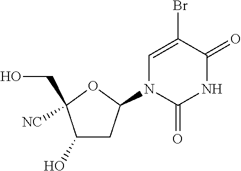 [SMILES: N#C[C@]1(CO)O[C@@H](n2cc(Br)c(=O)[nH]c2=O)C[C@@H]1O] | (2R,3S,5R)-5-(5-bromo-2,4-dioxo-3,4- dihydropyrimidin-1(2H)-yl)-3-hydroxy-2- (hydroxymethyl)tetrahydrofuran-2- carbonitrile |
| 158 | 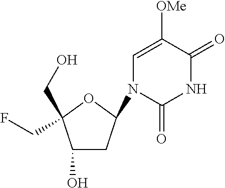 [SMILES: COc1cn([C@H]2C[C@H](O)[C@](CO)(CF)O2)c(=O)[nH]c1=O] | 1-((2R,4S,5R)-5-(fluoromethyl)-4-hydroxy- 5-(hydroxymethyl)tetrahydrofuran-2-yl)-5- methoxypyrimidine-2,4(1H,3H)-dione |
| 159 | 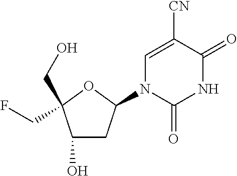 [SMILES: N#Cc1cn([C@H]2C[C@H](O)[C@](CO)(CF)O2)c(=O)[nH]c1=O] | 1-((2R,4S,5R)-5-(fluoromethyl)-4-hydroxy- 5-(hydroxymethyl)tetrahydrofuran-2-yl)- 2,4-dioxo-1,2,3,4-tetrahydropyrimidine-5- carbonitrile |
| 160 | 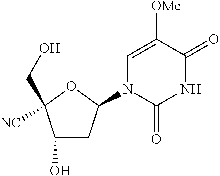 [SMILES: COc1cn([C@H]2C[C@H](O)[C@@](C#N)(CO)O2)c(=O)[nH]c1=O] | (2R,3S,5R)-3-hydroxy-2-(hydroxymethyl)- 5-(5-methoxy-2,4-dioxo-3,4- dihydropyrimidin-1(2H)-yl)tetrahydrofuran- 2-carbonitrile |
| 162 | 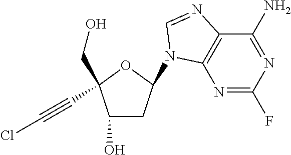 [SMILES: Nc1nc(F)nc2c1ncn2[C@H]1C[C@H](O)[C@@](C#CCl)(CO)O1] | (2R,3S,5R)-5-(6-amino-2-fluoro-9H-purin- 9-yl)-2-(chloroethynyl)-2- (hydroxymethyl)tetrahydrofuran-3-ol |
| 163 | 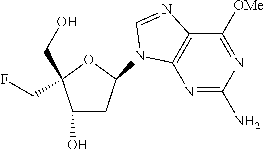 [SMILES: COc1nc(N)nc2c1ncn2[C@H]1C[C@H](O)[C@](CO)(CF)O1] | (2R,3S,5R)-5-(2-amino-6-methoxy-9H- purin-9-yl)-2-(fluoromethyl)-2- (hydroxymethyl)tetrahydrofuran-3-ol |
| 164 | 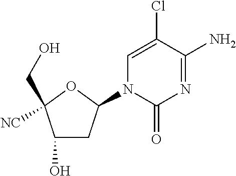 [SMILES: N#C[C@]1(CO)O[C@@H](n2cc(Cl)c(N)nc2=O)C[C@@H]1O] | (2R,3S,5R)-5-(4-amino-5-chloro-2- oxopyrimidin-1(2H)-yl)-3-hydroxy-2- (hydroxymethyl)tetrahydrofuran-2- carbonitrile |
| 166 | 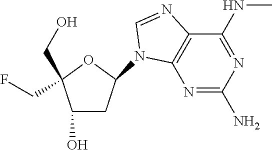 [SMILES: CNc1nc(N)nc2c1ncn2[C@H]1C[C@H](O)[C@](CO)(CF)O1] | (2R,3S,5R)-5-(2-amino-6-(methylamino)- 9H-purin-9-yl)-2-(fluoromethyl)-2- (hydroxymethyl)tetrahydrofuran-3-ol |
| 168 | 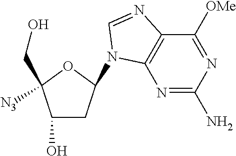 [SMILES: COc1nc(N)nc2c1ncn2[C@H]1C[C@H](O)[C@](CO)(N=[N+]=[N-])O1] | (2R,3S,5R)-5-(2-amino-6-methoxy-9H- purin-9-yl)-2-azido-2- (hydroxymethyl)tetrahydrofuran-3-ol |
| 169 | 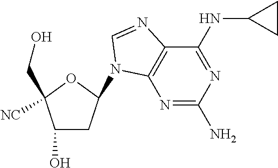 [SMILES: N#C[C@]1(CO)O[C@@H](n2cnc3c(NC4CC4)nc(N)nc32)C[C@@H]1O] | (2R,3S,5R)-5-(2-amino-6- (cyclopropylamino)-9H-purin-9-yl)-3- hydroxy-2-(hydroxymethyl)tetrahydrofuran- 2-carbonitrile |
| 172 | 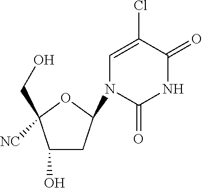 [SMILES: N#C[C@]1(CO)O[C@@H](n2cc(Cl)c(=O)[nH]c2=O)C[C@@H]1O] | (2R,3S,5R)-5-(5-chloro-2,4-dioxo-3,4- dihydropyrimidin-1(2H)-yl)-3-hydroxy-2- (hydroxymethyl)tetrahydrofuran-2- carbonitrile |
| 173 | 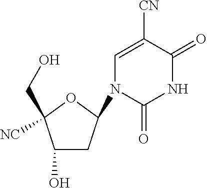 [SMILES: N#Cc1cn([C@H]2C[C@H](O)[C@@](C#N)(CO)O2)c(=O)[nH]c1=O] | 1-((2R,4S,5R)-5-cyano-4-hydroxy-5- (hydroxymethyl)tetrahydrofuran-2-yl)-2,4- dioxo-1,2,3,4-tetrabydropyrimidine-5- carbonitrile |
| 174 | 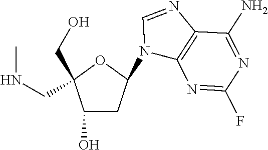 [SMILES: CNC[C@]1(CO)O[C@@H](n2cnc3c(N)nc(F)nc32)C[C@@H]1O] | (2R,3S,5R)-5-(6-amino-2-fluoro-9H-purin- 9-yl)-2-(hydroxymethyl)-2- ((methylamino)methyl)tetrahydrofuran-3-ol |
| 175 | 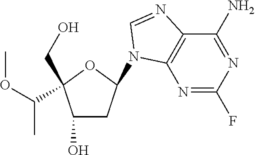 [SMILES: COC(C)[C@]1(CO)O[C@@H](n2cnc3c(N)nc(F)nc32)C[C@@H]1O] | (2R,3S,5R)-5-(6-amino-2-fluoro-9H-purin- 9-yl)-2-(hydroxymethyl)-2-((R)-1- methoxyethyl)tetrahydrofuran-3-ol |
| 176 | 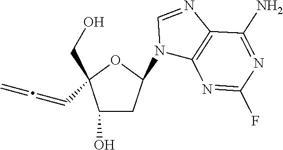 [SMILES: C=C=C[C@]1(CO)O[C@@H](n2cnc3c(N)nc(F)nc32)C[C@@H]1O] | (2R,3S,5R)-5-(6-amino-2-fluoro-9H-purin- 9-yl)-2-(hydroxymethyl)-2-(propa-1,2-dien- 1-yl)tetrahydrofuran-3-ol |
| 177 | 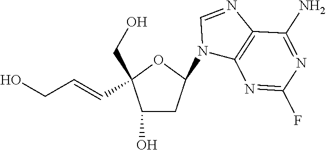 [SMILES: Nc1nc(F)nc2c1ncn2[C@H]1C[C@H](O)[C@@](/C=C/CO)(CO)O1] | (2R,3S,5R)-5-(6-amino-2-fluoro-9H-purin- 9-yl)-2-(hydroxymethyl)-2-((E)-3- hydroxyprop-1-en-1-yl)tetrahydrofuran-3-ol |
| 178 | 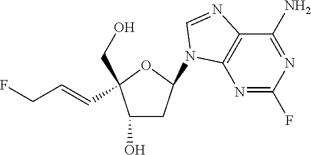 [SMILES: Nc1nc(F)nc2c1ncn2[C@H]1C[C@H](O)[C@@](/C=C/CF)(CO)O1] | (2R,3S,5R)-5-(6-amino-2-fluoro-9H-purin- 9-yl)-2-((E)-3-fluoroprop-1-en-1-yl)-2- (hydroxymethyl)tetrahydrofuran-3-ol |
| 179 | 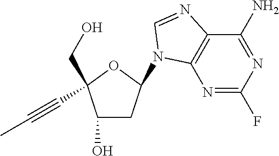 [SMILES: CC#C[C@]1(CO)O[C@@H](n2cnc3c(N)nc(F)nc32)C[C@@H]1O] | (2R,3S,5R)-5-(6-amino-2-fluoro-9H-purin- 9-yl)-2-(hydroxymethyl)-2-(prop-1-yn-1- yl)tetrahydrofuran-3-ol |
| 180 | 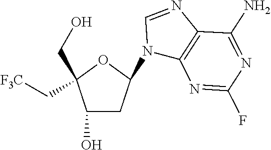 [SMILES: Nc1nc(F)nc2c1ncn2[C@H]1C[C@H](O)[C@](CO)(CC(F)(F)F)O1] | (2R,3S,5R)-5-(6-amino-2-fluoro-9H-purin- 9-yl)-2-(hydroxymethyl)-2-(2,2,2- trifluoroethyl)tetrahydrofuran-3-ol |
| 181 | 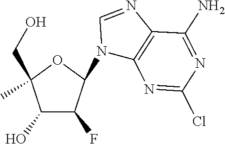 [SMILES: C[C@]1(CO)O[C@@H](n2cnc3c(N)nc(Cl)nc32)[C@@H](F)[C@@H]1O] | (2R,3R,4S,5R)-5-(6-amino-2-chloro-9H- purin-9-yl)-4-fluoro-2-(hydroxymethyl)-2- methyltetrahydrofuran-3-ol |
| 182 | 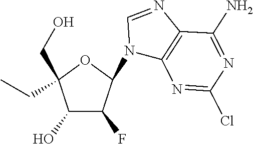 [SMILES: CC[C@]1(CO)O[C@@H](n2cnc3c(N)nc(Cl)nc32)[C@@H](F)[C@@H]1O] | (2R,3R,4S,5R)-5-(6-amino-2-chloro-9H- purin-9-yl)-2-ethyl-4-fluoro-2- (hydroxymethyl)tetrahydrofuran-3-ol |
| 190 | 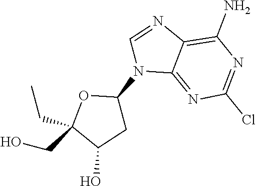 [SMILES: CC[C@]1(CO)O[C@@H](n2cnc3c(N)nc(Cl)nc32)C[C@@H]1O] | (2R,3S,5R)-5-(6-amino-2-chloro-9H-purin- 9-yl)-2-ethyl-2- (hydroxymethyl)tetrahydrofuran-3-ol |
| 191 | 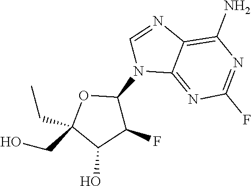 [SMILES: CC[C@]1(CO)O[C@@H](n2cnc3c(N)nc(F)nc32)[C@@H](F)[C@@H]1O] | (2R,3R,4S,5R)-5-(6-amino-2-fluoro-9H- purin-9-yl)-2-ethyl-4-fluoro-2- (hydroxymethyl)tetrahydrofuran-3-ol |
| 192 | 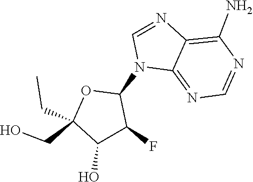 [SMILES: CC[C@]1(CO)O[C@@H](n2cnc3c(N)ncnc32)[C@@H](F)[C@@H]1O] | (2R,3R,4S,5R)-5-(6-amino-9H-purin-9-yl)- 2-ethyl-4-fluoro-2- (hydroxymethyl)tetrahydrofuran-3-ol |
| 193 | 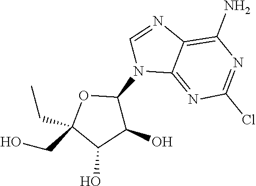 [SMILES: CC[C@]1(CO)O[C@@H](n2cnc3c(N)nc(Cl)nc32)[C@@H](O)[C@@H]1O] | (2R,3S,4S,5R)-5-(6-amino-2-chloro-9H- purin-9-yl)-2-ethyl-2- (hydroxymethyl)tetrahydrofuran-3,4-diol |
| 194 | 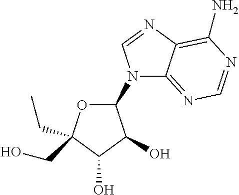 [SMILES: CC[C@]1(CO)O[C@@H](n2cnc3c(N)ncnc32)[C@@H](O)[C@@H]1O] | (2R,3S,4S,5R)-5-(6-amino-9H-purin-9-yl)- 2-ethyl-2-(hydroxymethyl)tetrahydrofuran- 3,4-diol |
| 195 | 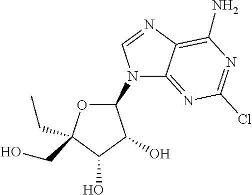 [SMILES: CC[C@]1(CO)O[C@@H](n2cnc3c(N)nc(Cl)nc32)[C@H](O)[C@@H]1O] | (2R,3S,4R,5R)-5-(6-amino-2-chloro-9H- purin-9-yl)-2-ethyl-2- (hydroxymethyl)tetrahydrofuran-3,4-diol |
| 196 | 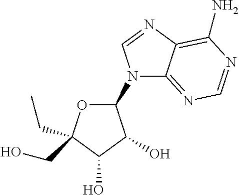 [SMILES: CC[C@]1(CO)O[C@@H](n2cnc3c(N)ncnc32)[C@H](O)[C@@H]1O] | (2R,3S,4R,5R)-5-(6-amino-9H-purin-9-yl)- 2-ethyl-2-(hydroxymethyl)tetrahydrofuran- 3,4-diol |
| 197 | 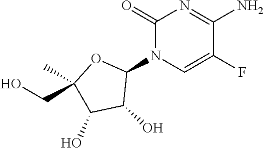 [SMILES: C[C@]1(CO)O[C@@H](n2cc(F)c(N)nc2=O)[C@H](O)[C@@H]1O] | 4-amino-1-((2R,3R,4S,5R)-3,4- dihydroxy-5-(hydroxymethyl)-5- methyltetrahydrofuran-2-yl)-5- fluoropyrimidin-2(1H)-one |
| 198 | 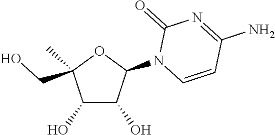 [SMILES: C[C@]1(CO)O[C@@H](n2ccc(N)nc2=O)[C@H](O)[C@@H]1O] | 4-amino-1-((2R,3R,4S,5R)-3,4- dihydroxy-5-(hydroxymethyl)-5- methyltetrahydrofuran-2-yl)pyrimidin- 2(1H)-one |
| 199 | 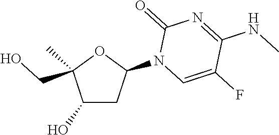 [SMILES: CNc1nc(=O)n([C@H]2C[C@H](O)[C@@](C)(CO)O2)cc1F] | 5-fluoro-1-((2R,4S,5R)-4-hydroxy-5- (hydroxymethyl)-5- methyltetrahydrofuran-2-yl)-4- (methylamino)pyrimidin-2(1H)-one |
| 200 | 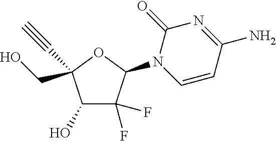 [SMILES: C#C[C@]1(CO)O[C@@H](n2ccc(N)nc2=O)C(F)(F)[C@@H]1O] | 4-amino-1-((2R,4R,5R)-5-ethynyl-3,3- difluoro-4-hydroxy-5- (hydroxymethyl)tetrahydrofuran-2- yl)pyrimidin-2(1H)-one |
| 201 | 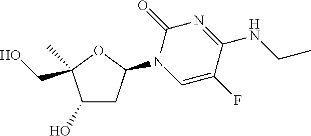 [SMILES: CCNc1nc(=O)n([C@H]2C[C@H](O)[C@@](C)(CO)O2)cc1F] | 4-(ethylamino)-5-fluoro-1-((2R,4S,5R)- 4-hydroxy-5-(hydroxymethyl)-5- methyltetrahydrofuran-2-yl)pyrimidin- 2(1H)-one |
| 202 | 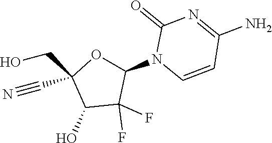 [SMILES: N#C[C@]1(CO)O[C@@H](n2ccc(N)nc2=O)C(F)(F)[C@@H]1O] | (2R,3R,SR)-5-(4-amino-2-oxopyrimidin- 1(2H)-yl)-4,4-difluoro-3-hydroxy-2- (hydroxymethyl)tetrahydrofuran-2- carbonitrile |
| 203 | 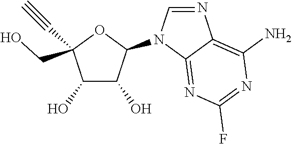 [SMILES: C#C[C@]1(CO)O[C@@H](n2cnc3c(N)nc(F)nc32)[C@H](O)[C@@H]1O] | (2R,3S,4R,SR)-5-(6-amino-2-fluoro-9H- purin-9-yl)-2-ethynyl-2- (hydroxymethyl)tetrahydrofuran-3,4-diol |
| 204 | 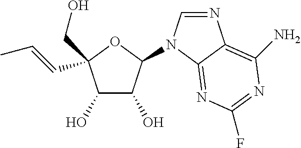 [SMILES: C/C=C/[C@]1(CO)O[C@@H](n2cnc3c(N)nc(F)nc32)[C@H](O)[C@@H]1O] | (2R,3S,4R,5R)-5-(6-amino-2-fluoro-9H- purin-9-yl)-2-(hydroxymethyl)-2-((E)- prop-1-en-1-yl)tetrahydrofuran-3,4-diol |
| 205 | 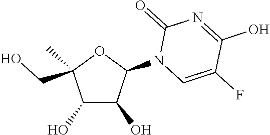 [SMILES: C[C@]1(CO)O[C@@H](n2cc(F)c(O)nc2=O)[C@@H](O)[C@@H]1O] | 1-((2R,3S,4S,5R)-3,4-dihydroxy-5- (hydroxymethyl)-5- methyltetrahydrofuran-2-yl)-5-fluoro-4- hydroxypyrimidin-2(1H)-one |
| 206 | 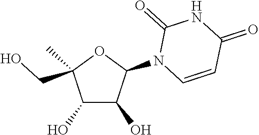 [SMILES: C[C@]1(CO)O[C@@H](n2ccc(=O)[nH]c2=O)[C@@H](O)[C@@H]1O] | 1-((2R,3S,4S,5R)-3,4-dihydroxy-5- (hydroxymethyl)-5- methyltetrahydrofuran-2-yl)pyrimidine- 2,4(1H,3H)-dione |
| 207 | 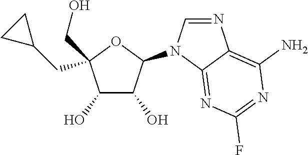 [SMILES: Nc1nc(F)nc2c1ncn2[C@@H]1O[C@@](CO)(CC2CC2)[C@@H](O)[C@H]1O] | (2R,3S,4R,5R)-5-(6-amino-2-fluoro-9H- purin-9-yl)-2-(cyclopropylmethyl)-2- (hydroxymethyl)tetrahydrofuran-3,4-diol |
| 208 | 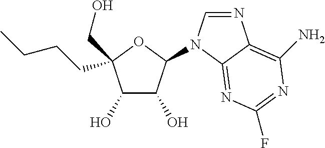 [SMILES: CCCC[C@]1(CO)O[C@@H](n2cnc3c(N)nc(F)nc32)[C@H](O)[C@@H]1O] | (2R,3S,4R,SR)-5-(6-amino-2-fluoro-9H- purin-9-yl)-2-butyl-2- (hydroxymethyl)tetrahydrofuran-3,4-diol |
| 209 | 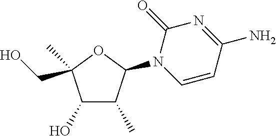 [SMILES: C[C@H]1[C@H](n2ccc(N)nc2=O)O[C@](C)(CO)[C@H]1O] | 4-amino-1-((2R,3R,4S,5R)-4-hydroxy-5- (hydroxymethyl)-3,5- dimethyltetrahydrofuran-2-yl)pyrimidin- 2(1H)-one |
| 210 | 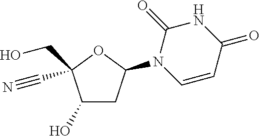 [SMILES: N#C[C@]1(CO)O[C@@H](n2ccc(=O)[nH]c2=O)C[C@@H]1O] | (2R,3S,5R)-5-(2,4-dioxo-3,4- dihydropyrimidin-1(2H)-yl)-3-hydroxy- 2-(hydroxymethyl)tetrahydrofuran-2- carbonitrile |
| 211 | 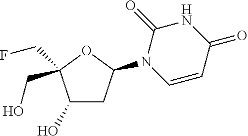 [SMILES: O=c1ccn([C@H]2C[C@H](O)[C@](CO)(CF)O2)c(=O)[nH]1] | 1-((2R,4S,5R)-5-(fluoromethyl)-4- hydroxy-5- (hydroxymethyl)tetrahydrofuran-2- yl)pyrimidine-2,4(1H,3H)-dione |
| 212 | 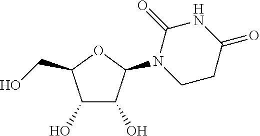 [SMILES: O=C1CCN([C@@H]2O[C@H](CO)[C@@H](O)[C@H]2O)C(=O)N1] | tetrahydrouridine |
| 213 | 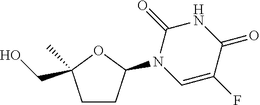 [SMILES: C[C@@]1(CO)CC[C@H](n2cc(F)c(=O)[nH]c2=O)O1] | 5-fluoro-1-((2R,5S)-5-(hydroxymethyl)- 5-methyltetrahydrofuran-2- yl)pyrimidine-2,4(1H,3H)-dione |
| 214 | 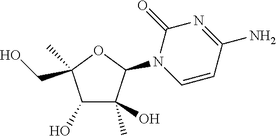 [SMILES: C[C@@]1(O)[C@H](n2ccc(N)nc2=O)O[C@](C)(CO)[C@H]1O] | 4-amino-1-((2R,3S,4S,5R)-3,4- dihydroxy-5-(hydroxymethyl)-3,5- dimethyltetrahydrofuran-2-yl)pyrimidin- 2(1H)-one |
| 215 | 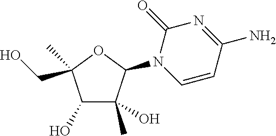 [SMILES: C[C@]1(O)[C@H](n2ccc(N)nc2=O)O[C@](C)(CO)[C@H]1O] | 4-amino-1-((2R,3R,4S,5R)-3,4- dihydroxy-5-(hydroxymethyl)-3,5- dimethyltetrahydrofuran-2-yl)pyrimidin- 2(1H)-one |
| 216 | 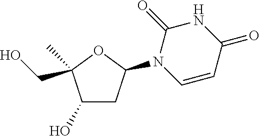 [SMILES: C[C@]1(CO)O[C@@H](n2ccc(=O)[nH]c2=O)C[C@@H]1O] | 1-((2R,4S,5R)-4-hydroxy-5- (hydroxymethyl)-5- methyltetrahydrofuran-2-yl)pyrimidine- 2,4(1H,3H)-dione |
| 217 | 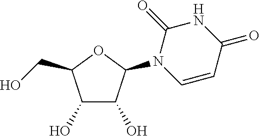 [SMILES: O=c1ccn([C@@H]2O[C@H](CO)[C@@H](O)[C@H]2O)c(=O)[nH]1] | uracil 1-beta-d-arabinofuranoside |
| 218 | 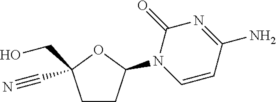 [SMILES: N#C[C@@]1(CO)CC[C@H](n2ccc(N)nc2=O)O1] | (2R,5R)-5-(4-amino-2-oxopyrimidin- 1(2H)-yl)-2- (hydroxymethyl)tetrahydrofuran-2- carbonitrile |
| 220 | 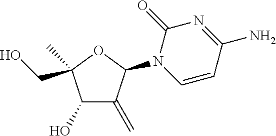 [SMILES: C=C1[C@H](n2ccc(N)nc2=O)O[C@](C)(CO)[C@H]1O] | 4-amino-1-((2R,4S,5R)-4-hydroxy-5- (hydroxymethyl)-5-methyl-3- methylenetetrahydrofuran-2- yl)pyrimidin-2(1H)-one |
| 222 | 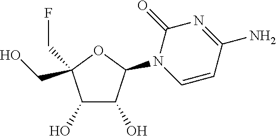 [SMILES: Nc1ccn([C@@H]2O[C@@](CO)(CF)[C@@H](O)[C@H]2O)c(=O)n1] | 4-amino-1-((2R,3R,4S,5R)-5- (fluoromethyl)-3,4-dihydroxy-5- (hydroxymethyl)tetrahydrofuran-2- yl)pyrimidin-2(1H)-one |
| 223 | 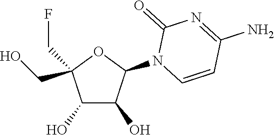 [SMILES: Nc1ccn([C@@H]2O[C@@](CO)(CF)[C@@H](O)[C@@H]2O)c(=O)n1] | 4-amino-1-((2R,3S,4S,5R)-5- (fluoromethyl)-3,4-dihydroxy-5- (hydroxymethyl)tetrahydrofuran-2- yl)pyrimidin-2(1H)-one |
| 224 | 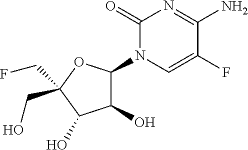 [SMILES: Nc1nc(=O)n([C@@H]2O[C@@](CO)(CF)[C@@H](O)[C@@H]2O)cc1F] | 4-amino-5-fluoro-1-((2R,3S,4S,5R)-5- (fluoromethyl)-3,4-dihydroxy-5- (hydroxymethyl)tetrahydrofuran-2- yl)pyrimidin-2(1H)-one |
| 225 | 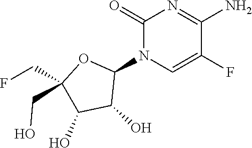 [SMILES: Nc1nc(=O)n([C@@H]2O[C@@](CO)(CF)[C@@H](O)[C@H]2O)cc1F] | 4-amino-5-fluoro-1-((2R,3R,4S,5R)-5- (fluoromethyl)-3,4-dihydroxy-5- (hydroxymethyl)tetrahydrofuran-2- yl)pyrimidin-2(1H)-one |
| 226 | 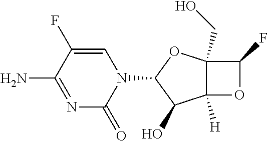 [SMILES: Nc1nc(=O)n([C@@H]2O[C@@]3(CO)[C@@H](F)O[C@H]3[C@H]2O)cc1F] | 4-amino-5-fluoro-1-((1R,3R,4R,5S,7R)- 7-fluoro-4-hydroxy-1-(hydroxymethyl)- 2,6-dioxabicyclo[3.2.0]heptan-3- yl)pyrimidin-2(1H)-one |
| 227 | 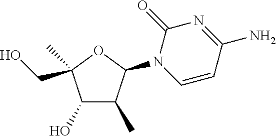 [SMILES: C[C@@H]1[C@H](n2ccc(N)nc2=O)O[C@](C)(CO)[C@H]1O] | 4-amino-1-((2R,3S,4S,5R)-4-hydroxy-5- (hydroxymethyl)-3,5- dimethyltetrahydrofuran-2-yl)pyrimidin- 2(1H)-one |
| 229 | 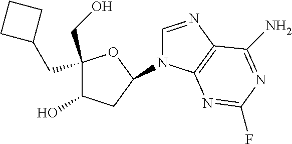 [SMILES: Nc1nc(F)nc2c1ncn2[C@H]1C[C@H](O)[C@](CO)(CC2CCC2)O1] | (2R,3S,5R)-5-(6-amino-2-fluoro-9H- purin-9-yl)-2-(cyclobutylmethyl)-2- (hydroxymethyl)tetrahydrofuran-3-ol |
| 230 | 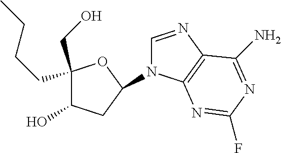 [SMILES: CCCC[C@]1(CO)O[C@@H](n2cnc3c(N)nc(F)nc32)C[C@@H]1O] | (2R,3S,5R)-5-(6-amino-2-fluoro-9H- purin-9-yl)-2-butyl-2- (hydroxymethyl)tetrahydrofuran-3-ol |
| 231 | 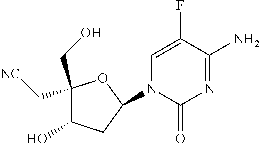 [SMILES: N#CC[C@]1(CO)O[C@@H](n2cc(F)c(N)nc2=O)C[C@@H]1O] | 2-((2R,3S,SR)-5-(4-amino-5-fluoro-2- oxopyrimidin-1(2H)-yl)-3-hydroxy-2- (hydroxymethyl)tetrahydrofuran-2- yl)acetonitrile |
| 232 | 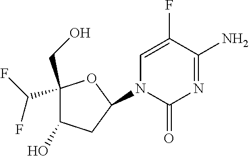 [SMILES: Nc1nc(=O)n([C@H]2C[C@H](O)[C@](CO)(C(F)F)O2)cc1F] | 4-amino-1-((2R,4S,5R)-5- (difluoromethyl)-4-hydroxy-5- (hydroxymethyl)tetrahydrofuran-2-yl)-5- fluoropyrimidin-2(1H)-one |
| 233 | 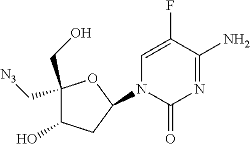 [SMILES: [N-]=[N+]=NC[C@]1(CO)O[C@@H](n2cc(F)c(N)nc2=O)C[C@@H]1O] | 4-amino-1-((2R,4S,5R)-5- (azidomethyl)-4-hydroxy-5- (hydroxymethyl)tetrahydrofuran-2-yl)-5- fluoropyrimidin-2(1H)-one |
| 234 | 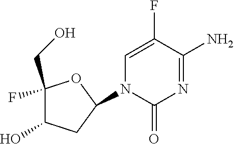 [SMILES: Nc1nc(=O)n([C@H]2C[C@H](O)[C@@](F)(CO)O2)cc1F] | 4-amino-5-fluoro-1-((2R,4S,5S)-5- fluoro-4-hydroxy-5- (hydroxymethyl)tetrahydrofuran-2- yl)pyrimidin-2(1H)-one |
| 234 | 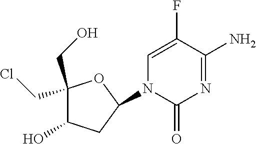 [SMILES: Nc1nc(=O)n([C@H]2C[C@H](O)[C@](CO)(CCl)O2)cc1F] | 4-amino-1-((2R,4S,5R)-5- (chloromethyl)-4-hydroxy-5- (hydroxymethyl)tetrahydrofuran-2-yl)-5- fluoropyrimidin-2(1H)-one |
| 236 | 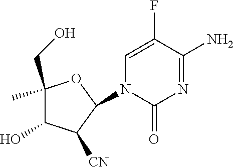 [SMILES: C[C@]1(CO)O[C@@H](n2cc(F)c(N)nc2=O)[C@@H](C#N)[C@@H]1O] | (2R,3S,4S,5R)-2-(4-amino-5-fluoro-2- oxopyrimidin-1(2H)-yl)-4-hydroxy-5- (hydroxymethyl)-5- methyltetrahydrofuran-3-carbonitrile |
| 237 | 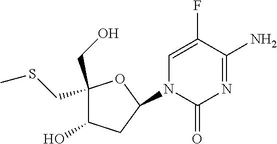 [SMILES: CSC[C@]1(CO)O[C@@H](n2cc(F)c(N)nc2=O)C[C@@H]1O] | 4-amino-5-fluoro-1-((2R,4S,5R)-4- hydroxy-5-(hydroxymethyl)-5- ((methylthio)methyl)tetrahydrofuran-2- yl)pyrimidin-2(1H)-one |
| 238 | 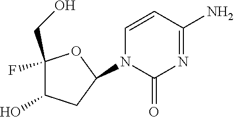 [SMILES: Nc1ccn([C@H]2C[C@H](O)[C@@](F)(CO)O2)c(=O)n1] | 4-amino-1-((2R,4S,5S)-5-fluoro-4- hydroxy-5- (hydroxymethyl)tetrahydrofuran-2- yl)pyrimidin-2(1H)-one |
| 239 | 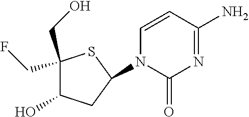 [SMILES: Nc1ccn([C@H]2C[C@H](O)[C@](CO)(CF)S2)c(=O)n1] | 4-amino-1-((2R,4S,5R)-5- (fluoromethyl)-4-hydroxy-5- (hydroxymethyl)tetrahydrothiophen-2- yl)pyrimidin-2(1H)-one |
| 240 | 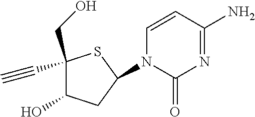 [SMILES: C#C[C@]1(CO)S[C@@H](n2ccc(N)nc2=O)C[C@@H]1O] | 4-amino-1-((2R,4S,5R)-5-ethynyl-4- hydroxy-5- (hydroxymethyl)tetrahydrothiophen-2- yl)pyrimidin-2(1H)-one |
| 241 | 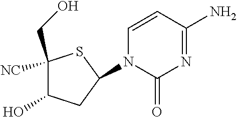 [SMILES: N#C[C@]1(CO)S[C@@H](n2ccc(N)nc2=O)C[C@@H]1O] | (2R,3S,5R)-5-(4-amino-2-oxopyrimidin- 1(2H)-yl)-3-hydroxy-2- (hydroxymethyl)tetrahydrothiophene-2- carbonitrile |
| 242 | 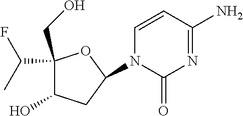 [SMILES: CC(F)[C@]1(CO)O[C@@H](n2ccc(N)nc2=O)C[C@@H]1O] | 4-amino-1-((2R,4S,5R)-5-(1- fluoroethyl)-4-hydroxy-5- (hydroxymethyl)tetrahydrofuran-2- yl)pyrimidin-2(1H)-one |
| 243 | 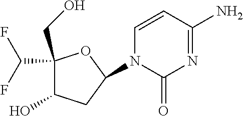 [SMILES: Nc1ccn([C@H]2C[C@H](O)[C@](CO)(C(F)F)O2)c(=O)n1] | 4-amino-1-((2R,4S,5R)-5- (difluoromethyl)-4-hydroxy-5- (hydroxymethyl)tetrahydrofuran-2- yl)pyrimidin-2(1H)-one |
| 244 | 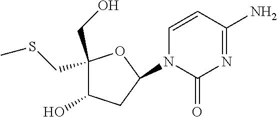 [SMILES: CSC[C@]1(CO)O[C@@H](n2ccc(N)nc2=O)C[C@@H]1O] | 4-amino-1-((2R,4S,5R)-4-hydroxy-5- (hydroxymethyl)-5- ((methylthio)methyl)tetrahydrofuran-2- yl)pyrimidin-2(1H)-one |
| 245 | 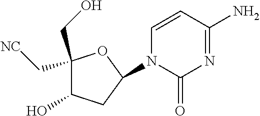 [SMILES: N#CC[C@]1(CO)O[C@@H](n2ccc(N)nc2=O)C[C@@H]1O] | 2-((2R,3S,5R)-5-(4-amino-2- oxopyrimidin-1(2H)-yl)-3-hydroxy-2- (hydroxymethyl)tetrahydrofuran-2- yl)acetonitrile |
| 246 | 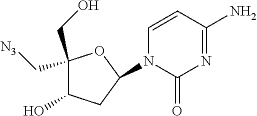 [SMILES: [N-]=[N+]=NC[C@]1(CO)O[C@@H](n2ccc(N)nc2=O)C[C@@H]1O] | 4-amino-1-((2R,4S,5R)-5- (azidomethyl)-4-hydroxy-5- (hydroxymethyl)tetrahydrofuran-2- yl)pyrimidin-2(1H)-one |
| 247 | 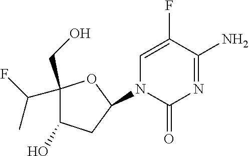 [SMILES: CC(F)[C@]1(CO)O[C@@H](n2cc(F)c(N)nc2=O)C[C@@H]1O] | 4-amino-5-fluoro-1-((2R,48,5R)-5-(1- fluoroethyl)-4-hydroxy-5- (hydroxymethyl)tetrahydrofuran-2- yl)pyrimidin-2(1H)-one |
| 248 | 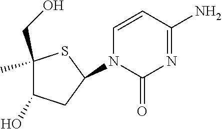 [SMILES: C[C@]1(CO)S[C@@H](n2ccc(N)nc2=O)C[C@@H]1O] | 4-amino-1-((2R,4S,5R)-4-hydroxy-5- (hydroxymethyl)-5- methyltetrahydrothiophen-2- yl)pyrimidin-2(1H)-one |
| 249 | 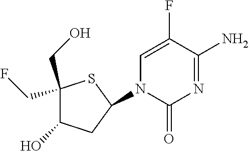 [SMILES: Nc1nc(=O)n([C@H]2C[C@H](O)[C@](CO)(CF)S2)cc1F] | 4-amino-5-fluoro-1-((2R,4S,5R)-5- (fluoromethyl)-4-hydroxy-5- (hydroxymethyl)tetrahydrothiophen-2- yl)pyrimidin-2(1H)-one |
| 250 | 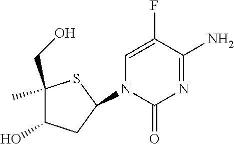 [SMILES: C[C@]1(CO)S[C@@H](n2cc(F)c(N)nc2=O)C[C@@H]1O] | 4-amino-5-fluoro-1-((2R,4S,5R)-4- hydroxy-5-(hydroxymethyl)-5- methyltetrahydrothiophen-2- yl)pyrimidin-2(1H)-one |
| 251 | 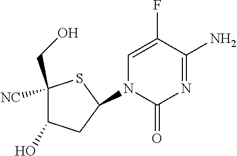 [SMILES: N#C[C@]1(CO)S[C@@H](n2cc(F)c(N)nc2=O)C[C@@H]1O] | (2R,3S,5R)-5-(4-amino-5-fluoro-2- oxopyrimidin-1(2H)-yl)-3-hydroxy-2- (hydroxymethyl)tetrahydrothiophene-2- carbonitrile |
| 252 | 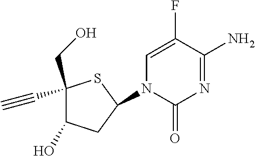 [SMILES: C#C[C@]1(CO)S[C@@H](n2cc(F)c(N)nc2=O)C[C@@H]1O] | 4-amino-1-((2R,4S,5R)-5-ethynyl-4- hydroxy-5- (hydroxymethyl)tetrahydrothiophen-2- yl)-5-fluoropyrimidin-2(1H)-one |
| 253 | 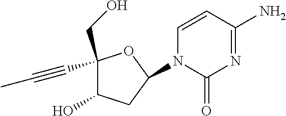 [SMILES: CC#C[C@]1(CO)O[C@@H](n2ccc(N)nc2=O)C[C@@H]1O] | 4-amino-1-((2R,4S,5R)-4-hydroxy-5- (hydroxymethyl)-5-(prop-1-yn-1- yl)tetrahydrofuran-2-yl)pyrimidin- 2(1H)-one |
| 254 | 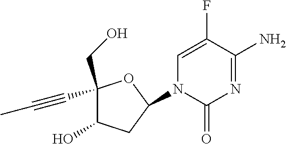 [SMILES: CC#C[C@]1(CO)O[C@@H](n2cc(F)c(N)nc2=O)C[C@@H]1O] | 4-amino-5-fluoro-1-((2R,4S,5R)-4- hydroxy-5-(hydroxymethyl)-5-(prop-1- yn-1-yl)tetrahydrofuran-2-yl)pyrimidin- 2(1H)-one |
| 255 | 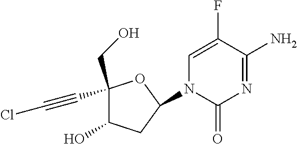 [SMILES: Nc1nc(=O)n([C@H]2C[C@H](O)[C@@](C#CCl)(CO)O2)cc1F] | 4-amino-1-((2R,45,5R)-5- (chloroethynyl)-4-hydroxy-5- (hydroxymethyl)tetrahydrofuran-2-yl)-5- fluoropyrimidin-2(1H)-one |
| 256 | 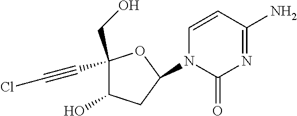 [SMILES: Nc1ccn([C@H]2C[C@H](O)[C@@](C#CCl)(CO)O2)c(=O)n1] | 4-amino-1-((2R,4S,5R)-5- (chloroethynyl)-4-hydroxy-5- (hydroxymethyl)tetrahydrofuran-2- yl)pyrimidin-2(1H)-one |

In one embodiment, Compounds of the Disclosure are compounds of Table 3, or a pharmaceutically acceptable salt or solvate thereof, or a tautomer thereof.

TABLE 3

| Cpd. | | |
|---|---|---|
| No. | Structure | Chemical Name |
| 142 | 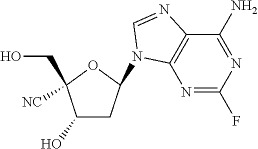 [SMILES: N#C[C@]1(CO)O[C@@H](n2cnc3c(N)nc(F)nc32)C[C@@H]1O] | (2R,3S,5R)-5-(6- amino-2-fluoro-9H- purin-9- yl)-3-hydroxy-2- (hydroxymethyl) tetrahydrofuran-2- carbonitrile |

Tables 1-3 provide chemical structures and the associated chemical names generated by Chemdraw® Professional version 20.1.1.125. In the event of any ambiguity between their chemical structure and chemical name, Compounds of the Disclosure are defined by their structure.

In another embodiment, the disclosure provides a pharmaceutical composition comprising a Compound of the Disclosure, or a pharmaceutically acceptable salt or solvate thereof, or a tautomer thereof, and one or more pharmaceutically acceptable excipients.

### II. Therapeutic Methods and Uses

Compounds of the Disclosure inhibit LINE-1 retrotransposition. As such, these compounds can be used to treat diseases, disorders, or conditions wherein, for example, LINE-1 retrotransposition plays a causative role.

A Compound of the Disclosure, or pharmaceutical composition thereof, can be administered to a subject in need thereof, e.g., a subject already suffering from a disease, condition, or disorder; a subject suspected of having a disease, condition, or disorder; or a subject at risk of acquiring a disease, condition, or disorder. When a Compound of the Disclosure is administered to a subject at risk of acquiring a disease, condition, or disorder, the intention is to try to avoid the disease, condition, or disorder in the subject, e.g. by preventing or reducing the LINE-1 retrotransposition activity that causes the disease, condition, or disorder.

In one embodiment, the disclosure provides a method of treating or preventing a disease, disorder, or condition in a subject in need thereof, and/or treating or preventing a symptom of the disease, disorder, or condition, the method comprising administering a therapeutically effective amount of a Compound of the Disclosure, or pharmaceutical composition thereof, to the subject.

In another embodiment, the disclosure provides a method of treating a disease, disorder, or condition in a subject in need thereof, the method comprising administering a therapeutically effective amount of a Compound of the Disclosure, or pharmaceutical composition thereof, to the subject.

In another embodiment, the disclosure provides a method of preventing a disease, disorder, or condition in a subject in need thereof, the method comprising administering a therapeutically effective amount of a Compound of the Disclosure, or pharmaceutical composition thereof, to the subject.

In another embodiment, the disclosure provides a method of treating a symptom of a disease, disorder, or condition in a subject in need thereof, the method comprising administering a therapeutically effective amount of a Compound of the Disclosure, or pharmaceutical composition thereof, to the subject.

In another embodiment, the disclosure provides a method of preventing a symptom of a disease, disorder, or condition in a subject in need thereof, the method comprising administering a therapeutically effective amount of a Compound of the Disclosure, or pharmaceutical composition thereof, to the subject.

In another embodiment, the disclosure provides a Compound of the Disclosure, or pharmaceutical composition thereof, for use in treating or preventing a disease, disorder, or condition in a subject and/or treating or preventing a symptom of the disease, disorder, or condition.

In another embodiment, the disclosure provides a Compound of the Disclosure, or pharmaceutical composition thereof, for use in treating a disease, disorder, or condition in a subject.

In another embodiment, the disclosure provides a Compound of the Disclosure, or pharmaceutical composition thereof, for use in preventing a disease, disorder, or condition in a subject.

In another embodiment, the disclosure provides a Compound of the Disclosure, or pharmaceutical composition thereof, for use in treating a symptom of a disease, disorder, or condition in a subject.

In another embodiment, the disclosure provides a Compound of the Disclosure, or pharmaceutical composition thereof, for use preventing a symptom of a disease, disorder, or condition in a subject.

In another embodiment, the disclosure provides the use of a Compound of the Disclosure, or pharmaceutical composition thereof, in the manufacture of a medicament for treating or preventing a disease, disorder, or condition in a subject and/or treating or preventing a symptom of the disease, disorder, or condition.

In another embodiment, the disclosure provides the use of a Compound of the Disclosure, or pharmaceutical composition thereof, in the manufacture of a medicament for treating a disease, disorder, or condition in a subject.

In another embodiment, the disclosure provides the use of a Compound of the Disclosure, or pharmaceutical composition thereof, in the manufacture of a medicament for preventing a disease, disorder, or condition in a subject.

In another embodiment, the disclosure provides the use of a Compound of the Disclosure, or pharmaceutical composition thereof, in the manufacture of a medicament for treating a symptom of a disease, disorder, or condition in a subject.

In another embodiment, the disclosure provides the use of a Compound of the Disclosure, or pharmaceutical composition thereof, in the manufacture of a medicament for preventing a symptom of the disease, disorder, or condition in a subject.

In another embodiment, the subject is (a) not infected with the HIV virus, (b) not suspected of being infected with the HIV virus, (c) not being treated for the HIV virus, and/or (d) not being treated to prevent the HIV virus.

In another embodiment, the disclosure provides a method of inhibiting a LINE-1 retrotransposition event that causes a disease, disorder, or condition in a subject in need thereof, the method comprising administering to the subject a therapeutically effective amount of a Compound of the Disclosure.

### III. Diseases, Disorders, and Conditions

Without wishing to be bound by any particular theory, Compounds of the Disclosure can be used to treat or prevent diseases, disorders, or conditions in a subject because they inhibit LINE-1 retrotransposition.

In one embodiment, Compounds of the Disclosure inhibit human LINE-1 retrotransposition activity with a half maximal inhibitory concentration (IC₅₀) of 1 μM or less in an in vitro HeLa cell-based dual-luciferase assay as described in EXAMPLE 35, see below. See also Jones et al., (2008) PLoS ONE 3(2): e1547. doi:10.1371/journal.pone.0001547; Xie et al., (2011) Nucleic Acids Res. 39(3): e16. doi: 10.1093/nar/gkq1076; Kopera et al., Method. Mol Biol 1400:139-156 (2016). In another embodiment, the IC₅₀ is 0.5 μM or less. In another embodiment, the IC₅₀ is 0.25 μM or less. In another embodiment, the IC₅₀ is 0.15 μM or less. In another embodiment, the IC₅₀ is 0.1 μM or less. In another embodiment, the IC₅₀ is 0.05 μM or less. In another embodiment, the IC₅₀ is 0.01 μM or less. In another embodiment, the IC₅₀ is 0.005 μM or less.

In one embodiment, the disease, disorder, or condition, and/or symptom(s) thereof is caused by a pathophysiological retrotransposon-associated process, wherein the disease, disorder, or condition is not cancer and/or not an infectious disease.

In another embodiment, the disease, disorder, or condition is a neurodegenerative disease. See, e.g., Dugger and Dickson, Cold Spring Harb Perspect Biol 2016; 9:a028035. Exemplary neurodegenerative diseases include, but are not limited to, Alzheimer's disease, amyotrophic lateral sclerosis (ALS), Parkinson's disease, dementia with Lewy Bodies (DLB), multi systems atrophy (MSA), Huntington's disease, frontotemporal lobar degeneration (FTLD), mild cognitive impairment (MCI), corticobasal degeneration (CDB), progressive supra nuclear palsy (PSP), Rett Syndrome, peripheral degenerative disease, or Aicardi-Goutieres syndrome (AGS). In another embodiment, the frontotemporal lobar degeneration is frontotemporal dementia. See, e.g., Mohandas and Rajmohan, Indian J Psychiatry 51(Suppl 1):S65-S69 (2009).

In another embodiment, symptoms of the neurodegenerative disease include, but are not limited to, memory loss, forgetfulness, apathy, anxiety, agitation, a loss of inhibition, or mood changes,

In another embodiment, the disease, disorder, or condition is an autoimmune disease. See, e.g., Wang et al., J Intern Med 278:369-395 (2016). Exemplary autoimmune diseases include, but are not limited to, lupus, rheumatoid arthritis (RA), Sjogrens syndrome, or multiple sclerosis (MS).

In another embodiment, symptoms of the autoimmune disease include, but are not limited to, fatigue, achy muscles, swelling and redness, low-grade fever, trouble concentrating, numbness and tingling in the hands and feet, hair loss, or skin rash.

In another embodiment, the disease, disorder, or condition is an age-associated disease. See, e.g., Franceschi et al., Front. Med. 5:61. doi: 10.3389/fmed.2018.00061; De Cecco et al., Nature 5666:73-78 (2019); US2021/0106586. Exemplary age-associated diseases include, but are not limited to, Alzheimer's disease, Parkinson's disease, atherosclerosis, osteoarthritis, osteoporosis, rheumatoid arthritis (RA), macular degeneration, peripheral degenerative disease, or skin aging. In one embodiment, the subject having the age-associated disease is at least 40 years old. In one embodiment, the subject having the age-associated disease is at least 45 years old. In one embodiment, the subject having the age-associated disease is at least 50 years old. In one embodiment, the subject having the age-associated disease is at least 55 years old. In one embodiment, the subject having the age-associated disease is at least 60 years old. In one embodiment, the subject having the age-associated disease is at least 65 years old. In one embodiment, the subject having the age-associated disease is at least 70 years old. In one embodiment, the subject having the age-associated disease is at least 75 years old.

In another embodiment, the disease, disorder, or condition is autism spectrum disorder (ASD), cardiovascular dysfunction, hearing loss, hematopoietic stem cell function, pulmonary fibrosis, schizophrenia, or vision loss.

In another embodiment, the disease, disorder, or condition is wound healing in a subject in need thereof.

In another embodiment, the disease, disorder, or condition is tissue regeneration in a subject in need thereof.

In another embodiment, the disease, disorder, or condition is Alzheimer's disease.

In another embodiment, the symptom of Alzheimer's disease any one or more of memory loss, misplacing items, forgetting the names of places or objects, repeating questions, being less flexible, confusion, disorientation, obsessive behavior, compulsive behavior, delusions, aphasia, disturbed sleep, mood swings, depression, anxiety, frustration, agitation, difficulty in performing spatial tasks, agnosia, difficulty with ambulation, weight loss, loss of speech, loss of short term memory, or loss of long term memory, and combinations thereof.

In another embodiment, the symptom(s) of Alzheimer's disease are determined using the cognitive subscale of the Alzheimer's disease Assessment Scale (ADAS-cog), the Clinician's Interview-Based Impression of Change (CIBIC-plus), or the Activities of Daily Living Scale (ADL).

In another embodiment, the disease, disorder, or condition is Alzheimer's disease, and one or more optional therapeutic agents are administered to the subject. In another embodiment, the optional therapeutic agents are donepezil, galantamine, rivastigmine, memantine, bapineuzumab, ABBV-8E12, CTS-21166, verubecestat (MK-8931), lanabecestat (AZD3293), LY2886721, nicotinamide, MPT0G211 or adacanumab-avwa.

In another embodiment, the disease, disorder, or condition is amyotrophic lateral sclerosis.

In another embodiment, the disease, disorder, or condition is amyotrophic lateral sclerosis and one or more optional therapeutic agents are administered to the subject. In another embodiment, the optional therapeutic agents are edaravone, riluzole, raltegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-25 870810, MK-0518, BMS-538158, or GSK364735C.

In another embodiment, the disease, disorder, or condition is ataxia-telangiectasia.

In another embodiment, the disease, disorder, or condition is age-related macular degeneration, systemic lupus erythematosus, IFN-associated autoimmune disease, e.g., rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, or superimposed autoimmune hepatitis, Fanconi Anemia, idiopathic pulmonary fibrosis, or cardiovascular disease. In another embodiment, the systemic disease is age-related macular degeneration. In another embodiment, the systemic disease is systemic lupus erythematosus. In another embodiment, the systemic disease is an IFN-associated autoimmune disease, e.g., psoriasis. In another embodiment, the systemic disease is Fanconi Anemia. In another embodiment, the systemic disease is idiopathic pulmonary fibrosis. In another embodiment, the systemic disease is cardiovascular disease.

In another embodiment, the disease, disorder, or condition is Rett Syndrome, AGS, ataxia-telangiectasia, ASD, schizophrenia, cocaine or methamphetamine abuse, FTLD, or ALS. See, e.g., Terry and Devine, Front. Genet., 8 Jan. 2020, https://doi.org/10.3389/fgene.2019.01244.

In one embodiment, a Compound of the Disclosure is administered to a subject having a disease, disorder, or condition as a single agent.

In another embodiment, a Compound of the Disclosure is administered to a subject having a disease, disorder, or condition in combination with one or more optional therapeutic agents. See, e.g., Durães et al., Pharmaceuticals 2018, 11, 44; doi:10.3390/ph11020044 for optionally therapeutic agents to treat neurodegenerative diseases.

In another embodiment, a Compound of the Disclosure is administered to a subject having a disease, disorder, or condition in combination with one optional therapeutic agent. In another embodiment, a Compound of the Disclosure is administered to a subject having a disease, disorder, or condition in combination with two optional therapeutic agents. In another embodiment, a Compound of the Disclosure is administered to a subject having a disease, disorder, or condition in combination with three optional therapeutic agents.

The Compound of the Disclosure and the one or more optional therapeutic agents can be administered in combination under one or more of the following conditions: at different periodicities, at different durations, at different concentrations, by different administration routes, etc.

In one embodiment, the Compound of the Disclosure and the one or more optional therapeutic agents are administered in combination to a subject as part of a single pharmaceutical composition.

In another embodiment, the Compound of the Disclosure and the one or more optional therapeutic agents are administered in combination to a subject separately, e.g., as two or more separate pharmaceutical compositions. In this case, two separate pharmaceutical compositions—one comprising the Compound of the Disclosure and one comprising the optional therapeutic agent—are administered to a subject. The separate pharmaceutical compositions can be administered to the subject, for example, at different periodicities, at different durations, or by the same or different administration routes, e.g., the Compound of the Disclosure can be administered orally and the optionally therapeutic agent can be administered intravenously.

In another embodiments, the Compound of the Disclosure is administered to the subject prior to the one or more optional therapeutic agents, e.g., 0.5, 1, 2, 3, 4, 5, 10, 12, or 18 hours, 1, 2, 3, 4, 5, or 6 days, or 1, 2, 3, or 4 weeks prior to the administration of the one or more optional therapeutic agents.

In another embodiments, the Compound of the Disclosure is administered to the subject after the one or more optional therapeutic agents, e.g., 0.5, 1, 2, 3, 4, 5, 10, 12, or 18 hours, 1, 2, 3, 4, 5, or 6 days, or 1, 2, 3, or 4 weeks after the administration of the one or more optional therapeutic agents.

In another embodiments, the Compound of the Disclosure and the one or more optional therapeutic agents are administered concurrently.

In one embodiment, the Compound of the Disclosure is administered to the subject according to a continuous dosing schedule.

In one embodiment, the Compound of the Disclosure is administered to the subject according to an intermittent dosing schedule.

In one embodiment, the Compound of the Disclosure is orally administered to the subject.

The therapeutic methods provided herein comprise administering a Compound of the Disclosure to a subject having a disease, disorder, or condition in an amount which is effective to achieve its intended purpose. While individual needs vary, determination of optimal ranges of effective amounts of each component is within the skill of the art. Typically, the Compound of the Disclosure is administered in an amount from about 0.01 mg/kg to about 500 mg/kg, about 0.05 mg/kg to about 100 mg/kg, about 0.05 mg/kg to about 50 mg/kg, or about 0.05 mg/kg to about 10 mg/kg. In one embodiment, the Compound of the Disclosure is administered once a day. In another embodiment, the Compound of the Disclosure is administered twice a day. In one embodiment, the Compound of the Disclosure is administered three times a day. In one embodiment, the Compound of the Disclosure is administered four times a day. These dosages are exemplary, but there can be individual instances in which higher or lower dosages are merited, and such are within the scope of this disclosure. In practice, the physician determines the actual dosing regimen that is most suitable for an individual subject, which can vary with the age, weight, and response of the particular subject.

The unit dose may comprise from about 0.01 mg to about 1000 mg, e.g., about 1 mg to about 500 mg, e.g., about 1 mg to about 250 mg, e.g., about 1 mg to about 100 mg of the Compound of the Disclosure. For example, the unit oral dose of the Compound of the Disclosure may comprise, for example, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, or 100 mg. The unit dose may be administered one or more times daily, e.g., as one or more tablets or capsules. The unit dose may also be administered by any suitable route, e.g., orally, by IV, inhalation or subcutaneously to the subject. In practice, the physician determines the actual dosing regimen that is most suitable for an individual subject, which can vary with the age, weight, and response of the particular subject.

In one embodiment, the Compound of the Disclosure is administered to a subject in an amount from about 0.1 mg to about 100 mg once a day, twice a day, three times a day, or four times a day. In another embodiment, the Compound of the Disclosure is administered to a subject in an amount from about 1 mg to about 50 mg per day.

In one embodiment, the Compound of the Disclosure is administered to the subject in a single dose. In another embodiment, the Compound of the Disclosure is administered to the subject in two divided doses. In another embodiment, the Compound of the Disclosure is administered to the subject in three divided doses. In another embodiment, the Compound of the Disclosure is administered to the subject in four divided doses.

The Compound of the Disclosure can be administered to a subject in the form of a raw chemical or as part of a pharmaceutical composition containing the Compound of the Disclosure combined with a suitable pharmaceutically acceptable carrier. Such a carrier can be selected from pharmaceutically acceptable excipients, vehicles, and auxiliaries. The term “pharmaceutically acceptable carrier,” “pharmaceutically acceptable vehicle,” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

A pharmaceutical composition comprising the Compound of the Disclosure can contain from about 0.01 to 99 percent by weight, e.g., from about 0.25 to 75 percent by weight, of the Compound of the Disclosure, e.g., about 1%, about 5%, about 10%, about 15%, about 20%, about 25%, about 30%, about 35%, about 40%, about 45%, about 50%, about 55%, about 60%, about 65%, about 70%, or about 75% by weight of the Compound of the Disclosure.

The Compound of the Disclosure, or pharmaceutical composition comprising the Compound of the Disclosure, can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration to a subject. Dosage forms depend on the route administration. Dosage forms include, but are not limited to, tablets, dragees, slow release lozenges, capsules, liquid solutions, liquid suspensions, oral/nasal spray, transdermal patch, thin dissolvable film, ointments, sustained or controlled release implants, mouth rinses and mouth washes, gels, hair rinses, hair gels, and shampoos, and suppositories, as well as suitable solutions for administration by intravenous infusion, and suitable suspensions for administration subcutaneous injection, and suitable powders for reconstitution. Parenteral administration can be accomplished using a needle and syringe or using other technique known in the art. In one embodiment, the Compound of the Disclosure is administered orally to the subject. In one embodiment, the Compound of the Disclosure is administered subcutaneously to the subject. In one embodiment, the Compound of the Disclosure is administered intravenously to the subject.

The Compound of the Disclosure and pharmaceutical compositions comprising the Compound of the Disclosure may be administered to any subject which may experience the beneficial effects of inhibiting LINE-1 retrotransposition activity. The term “subject” as used herein refers to any human or animal that is in need of or might benefit from administration of the Compound of the Disclosure. Foremost among such subjects are mammals, e.g., humans, although the methods and compositions provided herein are not intended to be so limited. Other subjects include veterinary animals, e.g., cows, sheep, pigs, horses, dogs, cats and the like. In one embodiment, the subject is a human. In one embodiment, the subject is an animal. In another embodiment, the subject is a human having a disease, condition, or disorder responsive to LINE-1 inhibition.

The pharmaceutical preparations provided herein are manufactured by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, crosslinked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of a Compound of the Disclosure may be administered to a subject. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers and other additives.

Therapeutically effective amounts of a Compound of the Disclosure formulated in accordance with standard pharmaceutical practices are administered to a subject in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of the Compound of the Disclosure that is sufficient to maintain therapeutic effects.

Toxicity and therapeutic efficacy of the Compound of the Disclosure can be determined by standard pharmaceutical procedures in cell cultures or experimental animals, e.g., for determining the maximum tolerated dose (MTD) of a compound, which defines as the highest dose that causes no toxicity in a subject. The dose ratio between the maximum tolerated dose and therapeutic effects is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of the Compound of the Disclosure required for use in therapy varies with the nature of the disease being treated, the length of time that activity is desired, and the age and the condition of the subject, and ultimately is determined by the attendant physician. For example, dosage amounts and intervals can be adjusted individually to provide plasma levels of a Compound of the Disclosure that are sufficient to maintain the desired therapeutic effects. The desired dose conveniently can be administered in a single dose, or as multiple doses administered at appropriate intervals, for example as one, two, three, four or more subdoses per day.

### IV. Kits

In another embodiment, the present disclosure provides kits comprising a Compound of the Disclosure, or a pharmaceutical composition thereof, and instructions for administering the compound or composition to a subject having a disease, disorder, or condition.

In another embodiment, the present disclosure provides kits comprising a Compound of the Disclosure, or a pharmaceutical composition thereof, packaged in a manner that facilitates their use to practice methods of the present disclosure.

In one embodiment, the kit includes a Compound of the Disclosure, or a pharmaceutical composition thereof, packaged in a container, such as a sealed bottle or vessel, with a label affixed to the container or included in the kit that describes use of the compound or composition to practice the method of the disclosure. In one embodiment, the compound or composition is packaged in a unit dosage form. The kit may include a single dose or multiple doses of a Compound of the Disclosure, or a pharmaceutical composition thereof.

In another embodiment, the kit includes a Compound of the Disclosure, or a composition thereof, and one or more optional therapeutic agents.

### V. Biomarkers

In another embodiment, present disclosure provides methods of treating a subject having a disease, condition, or disorder, the method comprising (a) determining whether a biomarker is present or absent in a biological sample taken from the subject; and (b) administering a therapeutically effective amount of a Compound of the Disclosure to the subject if the biomarker is present in the biological sample.

The term “biomarker” as used herein refers to any biological compound, such as a gene, a protein, a fragment of a protein, a peptide, a polypeptide, a nucleic acid, etc., or chromosome abnormality, such as a chromosome translocation, that can be detected and/or quantified in a subject in vivo or in a biological sample obtained from a subject. A biomarker can be the entire intact molecule, or it can be a portion or fragment thereof. In one embodiment, the expression level of the biomarker is measured. The expression level of the biomarker can be measured, for example, by detecting the protein or RNA, e.g., mRNA, level of the biomarker. In some embodiments, portions or fragments of biomarkers can be detected or measured, for example, by an antibody or other specific binding agent. In some embodiments, a measurable aspect of the biomarker is associated with a given state of the subject, such as the subject's age. For biomarkers that are detected at the protein or RNA level, such measurable aspects may include, for example, the presence, absence, or concentration, i.e., expression level, of the biomarker in the subject, or biological sample obtained from the subject. For biomarkers that are detected at the nucleic acid level, such measurable aspects may include, for example, allelic versions of the biomarker or type, rate, and/or degree of mutation of the biomarker, also referred to herein as mutation status.

For biomarkers that are detected based on expression level of protein or RNA, expression level measured between different phenotypic statuses can be considered different, for example, if the mean or median expression level of the biomarker in the different groups is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers, alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes. The term “overexpression” indicates that the expression level of the biomarker in the subject having a disease, condition, or disorder is above the mean or median expression level of the biomarker in, e.g., a normal undiseased subject.

Biomarkers include, but are not limited to, retrotransposon RNA, retrotransposon reverse transcriptase e.g., ORF1p, ORF2p, and/or retrotransposon DNA. In one embodiment, the measurable aspect of the biomarker is its expression status. In another embodiment, the measurable aspect of the biomarker is elevated levels of the biomarker. In one embodiment, the measurable aspect of the biomarker is its mutation status.

In one embodiment, the biomarker is the expression level of LINE-1. Methods of determining the expression level of LINE-1 are described in US 2020/0253888, and may comprise, for example, determining the level of ORF1p, determining the level of LINE-1 mRNA, determining the amount of LINE-1 in a cell sample of the subject, or determining the level of ORF2p, or a combination thereof.

In one embodiment, the biomarker is retrotransposon RNA expression which is differentially present in a subject of one phenotypic status, e.g., a subject having an age-associated disease or a neurodegenerative disease, as compared with another phenotypic status, e.g., a normal undiseased subject or a subject having a disease, disorder, or condition without overexpression retrotransposon RNA. In one embodiment, the biomarker is overexpression of retrotransposon RNA.

In one embodiment, the biomarker is LINE-1 RNA expression which is differentially present in a subject of one phenotypic status, e.g., a subject having an age-associated disease or a neurodegenerative disease, as compared with another phenotypic status, e.g., a normal undiseased subject or a subject having a disease, disorder, or condition without overexpression LINE-1 RNA. In one embodiment, the biomarker is overexpression of LINE-1 RNA.

In another embodiment, the biomarker is retrotransposon reverse transcriptase which is differentially present in a subject of one phenotypic status, e.g., a subject having an age-associated disease or a neurodegenerative disease, as compared with another phenotypic status, e.g., a normal undiseased subject or a subject having a disease, disorder, or condition without overexpression of the retrotransposon reverse transcriptase. In one embodiment, the biomarker is overexpression of retrotransposon reverse transcriptase.

In another embodiment, the biomarker is ORF1p expression which is differentially present in a subject of one phenotypic status, e.g., a subject having an age-associated disease or a neurodegenerative disease, as compared with another phenotypic status, e.g., a normal undiseased subject or a subject having a disease, disorder, or condition without overexpression ORF1p. In one embodiment, the biomarker is overexpression of ORF1p.

In another embodiment, the biomarker is ORF2p expression which is differentially present in a subject of one phenotypic status, e.g., a subject having an age-associated disease or a neurodegenerative disease, as compared with another phenotypic status, e.g., a normal undiseased subject or a subject having a disease, disorder, or condition without overexpression ORF2p. In one embodiment, the biomarker is overexpression of ORF2p.

Biomarker standards can be predetermined, determined concurrently, or determined after a biological sample is obtained from the subject. Biomarker standards for use with the methods described herein can, for example, include data from samples from subjects without a neurodegenerative disease; data from samples from subjects with a neurodegenerative disease. Comparisons can be made to establish predetermined threshold biomarker standards for different classes of subjects, e.g., diseased vs. non-diseased subjects. The standards can be run in the same assay or can be known standards from a previous assay.

In one embodiment, the biomarker is retrotransposon DNA expression which is differentially present in a subject of one phenotypic status, e.g., a subject having an age-associated disease or a neurodegenerative disease, as compared with another phenotypic status, e.g., a normal undiseased subject or a subject having a disease, disorder, or condition without overexpression retrotransposon DNA. In one embodiment, the biomarker is overexpression of retrotransposon DNA. In another embodiment, the biomarker is overexpression of retrotransposon nuclear DNA. In another embodiment, the biomarker is overexpression of retrotransposon cytoplasmic DNA.

In one embodiment, the biomarker is LINE-1 DNA expression which is differentially present in a subject of one phenotypic status, e.g., a subject having an age-associated disease or a neurodegenerative disease, as compared with another phenotypic status, e.g., a normal undiseased subject or a subject having a disease, disorder, or condition without overexpression LINE-1 DNA. In one embodiment, the biomarker is overexpression of LINE-1 DNA. In another embodiment, the biomarker is overexpression of LINE-1 nuclear DNA. In another embodiment, the biomarker is overexpression of LINE-1 cytoplasmic DNA.

A biomarker is differentially present between different phenotypic status groups if the mean or median expression or mutation levels of the biomarker is calculated to be different, i.e., higher or lower, between the groups. Thus, biomarkers provide an indication that a subject, e.g., a subject having ALS, belongs to one phenotypic status or another.

In addition to individual biological compounds, e.g., LINE-1 RNA, the term “biomarker” as used herein is meant to include groups, sets, or arrays of multiple biological compounds. For example, the combination of LINE-1 RNA overexpression and ORF1p overexpression may comprise a biomarker, or the overexpression of LINE-1 RNA and LINE-1 DNA may comprise a biomarker. The term “biomarker” may comprise one, two, three, four, five, six, seven, eight, nine, ten, fifteen, twenty, twenty five, thirty, or more, biological compounds. In embodiment, the biomarker comprises one, two, or three biological compounds.

The determination of the expression level or mutation status of a biomarker in a subject can be performed using any of the many methods known in the art. Any method known in the art for quantitating specific proteins and/or detecting biomarker expression, e.g., LINE-1 RNA expression, ORF1p expression, and/or ORF2p expression, or the expression or mutation levels of any other biomarker(s) in a patient or a biological sample may be used in the methods of the disclosure. Examples include, but are not limited to, PCR (polymerase chain reaction), or RT-PCR, flow cytometry, Northern blot, Western blot, ELISA (enzyme linked immunosorbent assay), RIA (radioimmunoassay), gene chip analysis of RNA expression, immunohistochemistry or immunofluorescence. See, e.g., Slagle et al. Cancer 83:1401 (1998). Certain embodiments of the disclosure include methods wherein biomarker RNA expression (transcription) is determined. Other embodiments of the disclosure include methods wherein protein expression in the biological sample is determined. See, e.g., Harlow et al., Antibodies: A Laboratory Manual, Cold Spring Harbor Laboratory, Cold Spring Harbor, NY, (1988); Ausubel et al., Current Protocols in Molecular Biology, John Wiley & Sons, New York 3rd Edition, (1995): Kamel and Al-Amodi, Genomics Proteomics Bioinformatics 15:220-235 (2017). For northern blot or RT-PCR analysis, RNA is isolated from tissue sample using RNAse free techniques. Such techniques are commonly known in the art.

In one embodiment of the disclosure, a biological sample is obtained from the subject and the biological sample is assayed for determination of a biomarker expression or mutation status.

In another embodiment of the disclosure, Northern blot analysis of biomarker transcription in a tumor cell sample is performed. Northern analysis is a standard method for detection and/or quantitation of mRNA levels in a sample. Initially, RNA is isolated from a sample to be assayed using Northern blot analysis. In the analysis, the RNA samples are first separated by size via electrophoresis in an agarose gel under denaturing conditions. The RNA is then transferred to a membrane, crosslinked and hybridized with a labeled probe. Typically, Northern hybridization involves polymerizing radiolabeled or nonisotopically labeled DNA, in vitro, or generation of oligonucleotides as hybridization probes. Typically, the membrane holding the RNA sample is prehybridized or blocked prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. Detection is accomplished using detectably labeled probes and a suitable detection method. Radiolabeled and non-radiolabeled probes and their use are well known in the art. The presence and or relative levels of expression of the biomarker being assayed can be quantified using, for example, densitometry.

In another embodiment, biomarker expression and/or mutation status is determined using RT-PCR. RT-PCR allows detection of the progress of a PCR amplification of a target gene in real time. Design of the primers and probes required to detect expression and/or mutation status of a biomarker of the disclosure is within the skill of a practitioner of ordinary skill in the art. RT-PCR can be used, for example, to determine the level of RNA encoding a biomarker of the disclosure in a tissue sample. In an embodiment of the disclosure, RNA from the biological sample is isolated, under RNAse free conditions, than converted to DNA by treatment with reverse transcriptase. Methods for reverse transcriptase conversion of RNA to DNA are well known in the art. A description of PCR is provided in the following references: Mullis et al., Cold Spring Harbor Symp. Quant. Biol. 51:263 (1986); EP 50,424; EP 84,796; EP 258,017; EP 237,362; EP 201,184; U.S. Pat. Nos. 4,683,202; 4,582,788; 4,683,194.

RT-PCR probes depend on the 5′-3′ nuclease activity of the DNA polymerase used for PCR to hydrolyze an oligonucleotide that is hybridized to the target amplicon (biomarker gene). RT-PCR probes are oligonucleotides that have a fluorescent reporter dye attached to the 5′ end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques.

In another embodiment of the disclosure, expression of proteins encoded by biomarkers are detected by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are detected using a primary antibody that specifically bind to the protein. The bound antibody can then detected by a secondary antibody that is conjugated with a detectable label (e.g., biotin, horseradish peroxidase or alkaline phosphatase). Detection of the secondary label signal indicates the presence of the protein.

In another embodiment of the disclosure, the expression of a protein encoded by a biomarker is detected by enzyme-linked immunosorbent assay (ELISA). In one embodiment of the disclosure, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the biomarker antigen protein.

In another embodiment of the disclosure, the expression of a protein, e.g., ORF1p, ORF2p, encoded by a biomarker is detected by s single molecule array assay (Simoa™).

In another embodiment of the disclosure, the expression of a protein, e.g., ORF1p, ORF2p, encoded by a biomarker is detected droplet digital ELISA (ddELISA). Using ddELISA, LINE-1/ORF1p protein can be measured in serum. See Cohen et al., ACS Nano 14:9491-9501 (2020).

### VI. Definitions

The term “pathophysiological retrotransposon-associated process” as used herein refers to a disordered physiological process relating to aberrant retrotransposition activity of at least one retrotransposon. Exemplary retrotransposons include, but are not limited to, LINE-1 and human endogenous retroviruses (HERVs), e.g., HERV-K and HERV-E. See, e.g., Saleh et al, (2019) Front. Neurol. 10:894. doi: 10.3389/fneur.2019.00894. Diseases, disorders, or conditions caused by a pathophysiological retrotransposon-associated process include, but are not limited to, neurodegenerative diseases, autoimmune diseases, age-associated diseases, autism spectrum disorder (ASD), cardiovascular dysfunction, hearing loss, hematopoietic stem cell function, pulmonary fibrosis, schizophrenia, or vision loss.

The term “pathophysiological LINE-1-associated process” as used herein refers to a disordered physiological process relating to aberrant LINE-1 (L1) retrotransposition activity. See, e.g., Saleh et al, (2019) Front. Neurol. 10:894. doi: 10.3389/fneur.2019.00894; Zhao et al., PLoS Genet 15(4): e1008043. https://doi.org/10.1371/journal.pgen.1008043; Bundo et al., Neuron 81:306-313 (2014).

The term “LINE-1 retrotransposition event that causes a disease, disorder, or condition” as used herein refers to any causal factor, e.g., aberrant transcription, alternative splicing, insertional mutagenesis, DNA damage, chromosomal translocation, increased expression of LINE-1 RNA, ORF1p (a 40 kDa RNA-binding protein), ORF2p (a ˜150 kDa protein with endonuclease (EN) and reverse transcriptase (RT) activities) associated with LINE-1 retrotransposition that results in or promotes a pathological condition, e.g., a disease or disorder, in a subject. See, e.g., Beck et al., Annu Rev Genomics Hum Genet 12:187-215 (2011); Pizarro and Cristofari (2016) Front. Cell Dev. Biol. 4:14, https://doi.org/10.3389/fcell.2016.00014. In one embodiment, the LINE-1 retrotransposition event is a somatic LINE-1 insertion. In another embodiment, LINE-1 retrotransposition event is increased expression of LINE-1 RNA in the subject.

The term “tautomer” as used herein refers to each of two or more isomers of a compound which exist together in equilibrium, and are interchanged by migration of an atom, e.g., a hydrogen, or group within the molecule. Certain Compounds of the Disclosure may exist as tautomers. In situations where tautomers are possible, the present disclosure includes all tautomeric forms. For example, as illustrated in Chart 1, both the lactim and lactam tautomers are encompassed, and both the amino and imino tautomers are encompassed.

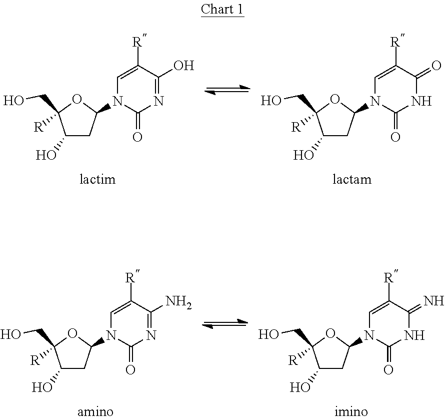

Likewise, as illustrated in Chart 2, both the lactim and lactam tautomers are encompassed, and both the amino and imino tautomers are encompassed.

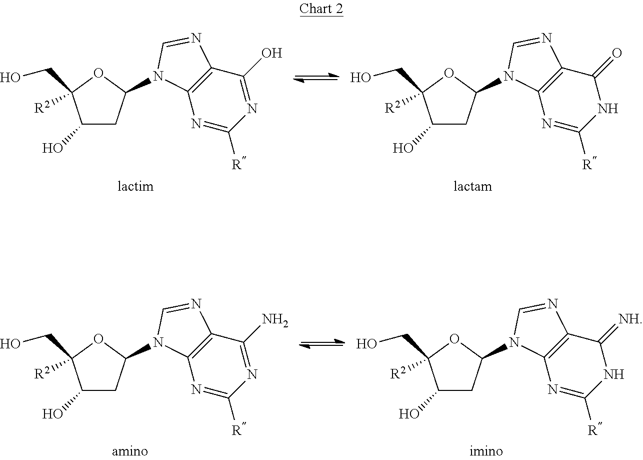

The equilibrium arrows in Charts 1 and 2 are not intended to show the position of the equilibrium, only that an equilibrium exists between the two tautomeric forms.

The term “biological sample” as used herein refers any tissue or fluid from a subject that is suitable for detecting a biomarker. Examples of useful biological samples include, but are not limited to, biopsied tissues and/or cells, e.g., lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for the expression level of a biological compound, e.g., LINE-1 RNA, ORF1p protein, ORF2p protein, using any technique known in the art. Such techniques include, but are not limited to, polymerase chain reaction (PCR) methodology, reverse transcription-polymerase chain reaction (RT-PCR) methodology, or cytoplasmic light chain immunofluorescence combined with fluorescence in situ hybridization (cIg-FISH). A biological sample can be obtained using techniques that are well within the scope of ordinary knowledge of a clinical practitioner. In one embodiment of the disclosure, the biological sample comprises a tissue or blood sample.

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

As used herein, the terms “treat,” “treating,” “treatment,” and the like refer to eliminating, reducing, or ameliorating a disease, disorder, or condition, and/or the symptoms associated therewith. Although not precluded, treating a disease, disorder, or condition does not require that the disease, disorder, or condition, and/or symptom(s) associated therewith be completely eliminated. However, in one embodiment, administration of a Compound of the Disclosure leads to complete elimination of the disease and associated symptoms.

As used herein, the terms “prevent,” “preventing,” “prevention” and the like refer to a method of preventing the onset of a disease, disorder, or condition and/or symptom(s) associated therewith, or barring a subject from acquiring the disease, disorder, or condition. The terms “prevent,” “preventing,” and “prevention” also include delaying the onset of disease, disorder, or condition and/or its attendant symptom(s), and reducing a subject's risk of acquiring the disease, disorder, or condition. The terms “prevent,” “preventing” and “prevention” also includes “prophylactic treatment,” which refers to reducing the probability of redeveloping the disease, disorder, or condition, or of a recurrence of a previously-controlled disease, disorder, or condition, in a subject who does not have, but is at risk of or is susceptible to, redeveloping the disease, disorder, or condition or a recurrence of the disease, disorder, or condition. The terms “prevent,” “preventing” and “prevention” also include delaying or reversing the progression of the underlying pathology of the disease, disorder, or condition, e.g., a mutation caused by a somatic LINE-1 insertion.

The term “therapeutically effective amount,” as used herein, refers to that amount of a Compound of the Disclosure and, optionally, one or more optional therapeutic agents sufficient to result in amelioration of one or more symptoms of a disease, disorder, or condition, or prevent advancement of a disease, disorder, or condition, or cause regression of a disease, disorder, or condition. For example, a therapeutically effective amount will refer to the amount of a Compound of the Disclosure that causes a therapeutic response, e.g., delay the progression of the disease, disorder, or condition in subject by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

The term “container” means any receptacle and closure therefore suitable for storing, shipping, dispensing, and/or handling the Compound of the Disclosure. Non-limiting exemplary containers include vials, ampules, bottles, and syringes.

The term “insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

In some embodiments, when administered in combination, two or more therapeutic agents can have a synergistic effect. The terms “synergy,” “synergistic,” “synergistically” and derivations thereof, such as in a “synergistic effect” or a “synergistic combination” or a “synergistic composition” as used herein refer to circumstances under which the biological activity of a combination of an agent and at least one additional therapeutic agent is greater than the sum of the biological activities of the respective agents when administered individually. For example, the term “synergistically effective” as used herein refers to the interaction between a Compound of the Disclosure and another therapeutic agent that causes the total effect of the drugs to be greater than the sum of the individual effects of each drug. Berenbaum, Pharmacological Reviews 41:93-141 (1989).

Synergy can be expressed in terms of a “Synergy Index (SI),” which generally can be determined by the method described by F. C. Kull et al. Applied Microbiology 9, 538 (1961), from the ratio determined by:

$${Q}_{a} {Q}_{A} + {Q}_{b} {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

The terms “intermittent dose administration,” “intermittent dosing schedule,” and similar terms as used herein refer to, i.e., not continuous, administration, of a Compound of the Disclosure to a subject.

Intermittent dose administration of a Compound of the Disclosure may maintain or efficacy achieved with continuous dosing, but with less side-effects, e.g., less body weight loss. Intermittent dose administration regimens useful in the present disclosure encompass any discontinuous administration regimen that provides a therapeutically effective amount of a Compound of the Disclosure to a subject in need thereof. Intermittent dosing regimens can use equivalent, lower, or higher doses of the Compound of the Disclosure than would be used in continuous dosing regimens. Advantages of intermittent dose administration of a Compound of the Disclosure include, but are not limited to, improved safety, decreased toxicity, e.g., decreased weight loss, increased exposure, increased efficacy, and/or increased subject compliance. These advantages may be realized when the Compound of the Disclosure is administered as a single agent or when administered in combination with one or more optional therapeutic agents. On the day a Compound of the Disclosure is scheduled to be administered to the subject, administration can occur in a single or in divided doses, e.g., once-a-day, twice-a-day, three times a day, four times a day or more. Dosing can also occur via any suitable route, e.g., orally, intravenously, or subcutaneously. In one embodiment, the Compound of the Disclosure is administered to the subject once (QD) or twice (BID) on the day the compound is scheduled to be administered.

In one embodiment, Compounds of the Disclosure are administered according to a continuous dosing schedule. In another embodiment, Compounds of the Disclosure are administered according to an intermittent dosing schedule.

The phrase “in combination” as used in connection with the administration of a Compound of the Disclosure and one or more optional therapeutic agents to a subject means that the Compound of the Disclosure and the one or more optional therapeutic agents can be administered to the subject together, e.g., as part of a single pharmaceutical composition or formulation, or separately, e.g., as part of two or more separate pharmaceutical compositions or formulations. The phrase “in combination” as used in connection with the administration of a Compound of the Disclosure and the one or more optional therapeutic agents to a subject is thus intended to embrace administration of the Compound of the Disclosure and the one or more optional therapeutic agents in a sequential manner, wherein the Compound of the Disclosure and the one or more optional therapeutic agents are administered to the subject at a different time, as well as administration concurrently, or in a substantially simultaneous manner, e.g., less than 30 minutes apart. Simultaneous administration can be accomplished, for example, by administering to the subject a single capsule having a fixed ratio of each of the Compound of the Disclosure and the one or more optional therapeutic agents or in multiple, single capsules for each of the Compound of the Disclosure and the one or more optional therapeutic agents. Sequential or substantially simultaneous administration of the Compound of the Disclosure and the one or more optional therapeutic agents can be accomplished by any appropriate route including, but not limited to, oral routes, intravenous routes, subcutaneous routes, intramuscular routes, etc. The Compound of the Disclosure and the one or more optional therapeutic agents can be administered by the same route or by different routes. For example, the one or more optional therapeutic agents and the Compound of the Disclosure of the combination may be administered orally. Alternatively, for example, the Compound of the Disclosure tor may be administered orally and the one or more optional therapeutic agents may be administered by intravenous injection. The Compound of the Disclosure and the one or more optional therapeutic agents may also be administered in alternation. In one embodiment, the Compound of the Disclosure and the one or more optional therapeutic agents are administered to a subject separately, e.g., as part of two or more separate pharmaceutical compositions or formulations.

### VII. Particular Embodiments

The disclosure provides the following particular embodiments.

Embodiment 1. A method of treating or preventing a disease, disorder, or condition caused by a pathophysiological retrotransposon-associated process in a subject in need thereof, and/or treating or preventing a symptom of the disease, disorder, or condition, the method comprising administering to the subject a therapeutically effective amount of a compound of:
(i) Table 1, or a pharmaceutically acceptable salt or solvate thereof, or a tautomer thereof;
(ii) Table 2, or a pharmaceutically acceptable salt or solvate thereof, or a tautomer thereof; or
(iii) Table 3, or a pharmaceutically acceptable salt or solvate thereof, or a tautomer thereof.

Embodiment 2. A method of inhibiting a LINE-1 retrotransposition event that causes a disease, disorder, or condition in a subject in need thereof, the method comprising administering to the subject a therapeutically effective amount of a compound of:
(i) Table 1, or a pharmaceutically acceptable salt or solvate thereof, or a tautomer thereof;
(ii) Table 2, or a pharmaceutically acceptable salt or solvate thereof, or a tautomer thereof; or
(iii) Table 3, or a pharmaceutically acceptable salt or solvate thereof, or a tautomer thereof.

Embodiment 3. A method of treating a disease, condition, or disorder in a subject, the method comprising:
(a) determining whether an overexpression of retrotransposon RNA, retrotransposon reverse transcriptase, or retrotransposon DNA is present or absent in a
biological sample taken from the subject; and
(b) administering a therapeutically effective amount of a compound of:
(i) Table 1, or a pharmaceutically acceptable salt or solvate thereof, or a tautomer thereof;
(ii) Table 2, or a pharmaceutically acceptable salt or solvate thereof, or a tautomer thereof; or
(iii) Table 3, or a pharmaceutically acceptable salt or solvate thereof, or a tautomer thereof,
to the subject if an overexpression of retrotransposon RNA, retrotransposon reverse transcriptase, or retrotransposon DNA is present in the biological sample.

Embodiment 4. A method of identifying whether a subject having a disease, condition, or disorder is a candidate for treatment with a compound of:
(i) Table 1, or a pharmaceutically acceptable salt or solvate thereof, or a tautomer thereof,
(ii) Table 2, or a pharmaceutically acceptable salt or solvate thereof, or a tautomer thereof; or
(iii) Table 3, or a pharmaceutically acceptable salt or solvate thereof, or a tautomer thereof,
the method comprising:
(a) determining whether an overexpression of retrotransposon RNA, retrotransposon reverse transcriptase, or retrotransposon DNA is present or absent in a biological sample taken from the subject; and
(b) identifying the subject as being a candidate for treatment if an overexpression of retrotransposon RNA, retrotransposon reverse transcriptase, or retrotransposon DNA is present; or
(c) identifying the subject as not being a candidate for treatment if an overexpression of retrotransposon RNA, retrotransposon reverse transcriptase, or retrotransposon DNA is absent.

Embodiment 5. A method of predicting treatment outcome in a subject having a disease, condition, or disorder, the method comprising determining whether an overexpression of retrotransposon RNA, retrotransposon reverse transcriptase, or retrotransposon DNA is present or absent in a biological sample taken from the subject, wherein:
(a) the presence of an overexpression of retrotransposon RNA, retrotransposon reverse transcriptase, or retrotransposon DNA in the biological sample indicates that administering a compound of:
(i) Table 1, or a pharmaceutically acceptable salt or solvate thereof, or a tautomer thereof;
(ii) Table 2, or a pharmaceutically acceptable salt or solvate thereof, or a tautomer thereof; or
(iii) Table 3, or a pharmaceutically acceptable salt or solvate thereof, or a tautomer thereof,
to the subject will likely cause a favorable therapeutic response; and
(b) the absence of an overexpression of retrotransposon RNA, retrotransposon reverse transcriptase, or retrotransposon DNA in the biological sample indicates that administering a compound of:
(i) Table 1, or a pharmaceutically acceptable salt or solvate thereof, or a tautomer thereof;
(ii) Table 2, or a pharmaceutically acceptable salt or solvate thereof, or a tautomer thereof; or
(iii) Table 3, or a pharmaceutically acceptable salt or solvate thereof, or a tautomer thereof,
to the subject will likely cause an unfavorable therapeutic response.

Embodiment 6. A method, comprising administering a therapeutically effective amount of a compound of:
(i) Table 1, or a pharmaceutically acceptable salt or solvate thereof, or a tautomer thereof:
(ii) Table 2, or a pharmaceutically acceptable salt or solvate thereof, or a tautomer thereof; or
(iii) Table 3, or a pharmaceutically acceptable salt or solvate thereof, or a tautomer thereof,
to a subject, wherein:
(a) the subject has a disease, condition, or disorder; and
(b) the disease, condition, or disorder is characterized as having an overexpression of retrotransposon RNA, retrotransposon reverse transcriptase, or retrotransposon DNA.

Embodiment 7. The method of any one of Embodiments 1-6, comprising a compound of Table 1, or a pharmaceutically acceptable salt or solvate thereof, or a tautomer thereof.

Embodiment 8. The method of any one of Embodiments 1-6, comprising a compound of Table 2, or a pharmaceutically acceptable salt or solvate thereof, or a tautomer thereof.

Embodiment 9. The method of Embodiment 7, wherein the compound of Table 1 is Cpd. No. 1, or a pharmaceutically acceptable salt or solvate thereof, or a tautomer thereof.

Embodiment 10. The method of Embodiment 7, wherein the compound of Table 1 is Cpd. No. 3, or a pharmaceutically acceptable salt or solvate thereof, or a tautomer thereof.

Embodiment 11. The method of Embodiment 7, wherein the compound of Table 1 is Cpd. 8, or a pharmaceutically acceptable salt or solvate thereof, or a tautomer thereof.

Embodiment 12. The method of Embodiment 7, wherein the compound of Table 1 is Cpd. No. 9, or a pharmaceutically acceptable salt or solvate thereof, or a tautomer thereof.

Embodiment 13. The method of Embodiment 12, wherein the compound of Table 1 is Cpd. No. 4, or a pharmaceutically acceptable salt or solvate thereof, or a tautomer thereof.

Embodiment 14. The method of any one of Embodiments 1-6, comprising a compound of Table 3, or a pharmaceutically acceptable salt or solvate thereof, or a tautomer thereof.

Embodiment 15. The method of any one of Embodiments 1-14 for treating the disease, disorder, or condition in a subject.

Embodiment 16. The method of any one of Embodiments 1-14 for preventing the disease, disorder, or condition in a subject.

Embodiment 17. The method of any one of Embodiments 1-14 for treating the symptom of a disease, disorder, or condition in a subject.

Embodiment 18. The method of any one of Embodiments 1-14 for preventing the symptom of a disease, disorder, or condition in a subject.

Embodiment 19. The method of any one of Embodiments 1-18, wherein the disease, disorder, or condition is a neurodegenerative disease.

Embodiment 20. The method of Embodiment 19, wherein the neurodegenerative disease is Alzheimer's disease, amyotrophic lateral sclerosis, Parkinson's disease, dementia with Lewy Bodies, multi systems atrophy, Huntington's disease, frontotemporal lobar degeneration, mild cognitive impairment, corticobasal degeneration, progressive supra nuclear palsy, Rett Syndrome, peripheral degenerative disease, or Aicardi-Goutières syndrome.

Embodiment 21. The method of any one of Embodiments 1-18, wherein the disease, disorder, or condition is an autoimmune disease.

Embodiment 22. The method of Embodiment 11, wherein the autoimmune disease is lupus, rheumatoid arthritis, Sjogrens syndrome, or multiple sclerosis.

Embodiment 23. The method of any one of Embodiments 1-18, wherein the disease, disorder, or condition is an age-associated disease.

Embodiment 24. The method of Embodiment 23, wherein the age-associated disease is Alzheimers disease, Parkinson's disease, atherosclerosis, osteoarthritis, osteoporosis, rheumatoid arthritis, macular degeneration, peripheral degenerative disease, or skin aging.

Embodiment 25. The method of any one of Embodiments 1-18, wherein the disease, disorder, or condition is autism spectrum disorder (ASD), cardiovascular dysfunction, hearing loss, hematopoietic stem cell function, pulmonary fibrosis, schizophrenia, or vision loss.

Embodiment 26. The method of any one of Embodiments 1-18, wherein the disease, disorder, or condition is progressive supra nuclear palsy.

Embodiment 27. The method of any one of Embodiments 1-18, wherein the disease, disorder, or condition is amyotrophic lateral sclerosis.

Embodiment 28. The method of any one of Embodiments 1-18, wherein the disease, disorder, or condition is Aicardi-Goutieres syndrome.

Embodiment 29. The method of any one of Embodiments 1-3 or 5-28 further comprising one or more optional therapeutic agents to the subject.

Embodiment 30. The method of any one of Embodiments 1-29, wherein the subject is (a) not infected with the HIV virus; (b) not suspected of being infected with the HIV virus; (c) not being treated for the HIV virus; and/or (d) not being treated to prevent the HIV virus.

Embodiment 31. The method of any one of Embodiments 1-30, wherein the compound inhibits human LINE-1 retrotransposition activity with a half maximal inhibitory concentration of 1 μM or less in an in vitro HeLa cell-based dual-luciferase assay.

Embodiment 32. A kit for carrying out the method of any one of claims 1-31, the kit comprising a compound of:
(i) Table 1, or a pharmaceutically acceptable salt or solvate thereof, or a tautomer thereof:
(ii) Table 2, or a pharmaceutically acceptable salt or solvate thereof, or a tautomer thereof; or
(iii) Table 3, or a pharmaceutically acceptable salt or solvate thereof, or a tautomer thereof, and instructions for administering the compound to a subject having a disease, condition, or disorder.

Embodiment 33. The kit of Embodiment 32 comprising a compound of Table 1, or a pharmaceutically acceptable salt or solvate thereof, or a tautomer thereof.

Embodiment 34. The kit of Embodiment 32 comprising a compound of Table 2, or a pharmaceutically acceptable salt or solvate thereof, or a tautomer thereof.

Embodiment 35. The kit of Embodiment 33, wherein the compound of Table 1 is Cpd. No. 1, or a pharmaceutically acceptable salt or solvate thereof, or a tautomer thereof.

Embodiment 36. The kit of Embodiment 33, wherein the compound of Table 1 is Cpd. No. 3, or a pharmaceutically acceptable salt or solvate thereof, or a tautomer thereof.

Embodiment 37. The kit of Embodiment 33, wherein the compound of Table 1 is Cpd. No. 8, or a pharmaceutically acceptable salt or solvate thereof, or a tautomer thereof.

Embodiment 38. The kit of Embodiment 33, wherein the compound of Table 1 is Cpd. No. 9, or a pharmaceutically acceptable salt or solvate thereof, or a tautomer thereof.

Embodiment 39. The kit of Embodiment 33, wherein the compound of Table 1 is Cpd. No. 4, or a pharmaceutically acceptable salt or solvate thereof, or a tautomer thereof.

Embodiment 40. The kit of Embodiment 32 comprising a compound of Table 3, or a pharmaceutically acceptable salt or solvate thereof, or a tautomer thereof.

Embodiment 41. A compound of:
(i) Table 1, or a pharmaceutically acceptable salt or solvate thereof, or a tautomer thereof, or a pharmaceutical composition thereof;
(ii) Table 2, or a pharmaceutically acceptable salt or solvate thereof, or a tautomer thereof, or a pharmaceutical composition thereof; or
(iii) Table 3, or a pharmaceutically acceptable salt or solvate thereof, or a tautomer thereof, or a pharmaceutical composition thereof,
for use in treating or preventing a disease, disorder, or condition caused by a pathophysiological retrotransposon-associated process in a subject in need thereof, and/or treating or preventing a symptom of the disease, disorder, or condition.

Embodiment 42. A compound of:
(i) Table 1, or a pharmaceutically acceptable salt or solvate thereof, or a tautomer thereof, or a pharmaceutical composition thereof;
(ii) Table 2, or a pharmaceutically acceptable salt or solvate thereof, or a tautomer thereof, or a pharmaceutical composition thereof; or
(iii) Table 3, or a pharmaceutically acceptable salt or solvate thereof, or a tautomer thereof; or a pharmaceutical composition thereof,
for use in inhibiting a LINE-1 retrotransposition event that causes a disease, disorder, or condition in a subject in need thereof.

Embodiment 43. A compound of:
(i) Table 1, or a pharmaceutically acceptable salt or solvate thereof, or a tautomer thereof, or a pharmaceutical composition thereof;
(ii) Table 2, or a pharmaceutically acceptable salt or solvate thereof, or a tautomer thereof, or a pharmaceutical composition thereof; or
(iii) Table 3, or a pharmaceutically acceptable salt or solvate thereof, or a tautomer thereof, or a pharmaceutical composition thereof,
for use in treating a disease, condition, or disorder is characterized as having an overexpression of retrotransposon RNA, retrotransposon reverse transcriptase, or retrotransposon DNA.

Embodiment 44. The compound of any one of Embodiments 41-43, comprising a compound of Table 1, or a pharmaceutically acceptable salt or solvate thereof, or a tautomer thereof.

Embodiment 45. The compound of any one of Embodiments 41-43, comprising a compound of Table 2, or a pharmaceutically acceptable salt or solvate thereof, or a tautomer thereof.

Embodiment 46. The compound of Embodiment 44, wherein the compound of Table 1 is Cpd. No. 1, or a pharmaceutically acceptable salt or solvate thereof, or a tautomer thereof.

Embodiment 47. The compound of Embodiment 44, wherein the compound of Table 1 is Cpd. No. 3, or a pharmaceutically acceptable salt or solvate thereof, or a tautomer thereof.

Embodiment 48. The compound of Embodiment 44, wherein the compound of Table 1 is Cpd. No. 8, or a pharmaceutically acceptable salt or solvate thereof, or a tautomer thereof.

Embodiment 49. The compound of Embodiment 44, wherein the compound of Table 1 is Cpd. No. 9, or a pharmaceutically acceptable salt or solvate thereof, or a tautomer thereof.

Embodiment 50. The compound of Embodiment 44, wherein the compound of Table 1 is Cpd. No. 4, or a pharmaceutically acceptable salt or solvate thereof, or a tautomer thereof.

Embodiment 51. The compound of any one of Embodiments 41-43, comprising a compound of Table 3, or a pharmaceutically acceptable salt or solvate thereof, or a tautomer thereof.

Embodiment 52. The compound or pharmaceutical composition for use of any one of Embodiments 41 or 44-51 for treating the disease, disorder, or condition in a subject.

Embodiment 53. The compound or pharmaceutical composition for use of any one of Embodiments 41 or 44-51 for preventing the disease, disorder, or condition in a subject.

Embodiment 54. The compound or pharmaceutical composition for use of any one of Embodiments 41 or 44-51, for treating the symptom of a disease, disorder, or condition in a subject.

Embodiment 55. The compound or pharmaceutical composition for use of any one of Embodiments 41 or 44-51, for preventing the symptom of a disease, disorder, or condition in a subject.

Embodiment 56. The compound or pharmaceutical composition for use of any one of Embodiments 41-55, wherein the disease, disorder, or condition is a neurodegenerative disease.

Embodiment 57. The compound or pharmaceutical composition for use of Embodiment 56, wherein the neurodegenerative disease is Alzheimer's disease, amyotrophic lateral sclerosis, Parkinson's disease, dementia with Lewy Bodies, multi systems atrophy, Huntington's disease, frontotemporal lobar degeneration, mild cognitive impairment, corticobasal degeneration, progressive supra nuclear palsy, Rett Syndrome, peripheral degenerative disease, or Aicardi-Goutières syndrome.

Embodiment 58. The compound or pharmaceutical composition for use of any one of Embodiments 41-55, wherein the disease, disorder, or condition is an autoimmune disease.

Embodiment 59. The compound or pharmaceutical composition for use of Embodiment 58, wherein the autoimmune disease is lupus, rheumatoid arthritis, Sjogrens syndrome, or multiple sclerosis.

Embodiment 60. The compound or pharmaceutical composition for use of any one of Embodiments 41-55, wherein the disease, disorder, or condition is an age-associated disease.

Embodiment 61. The compound or pharmaceutical composition for use of Embodiment 60, wherein the age-associated disease is Alzheimer's disease, Parkinson's disease, atherosclerosis, osteoarthritis, osteoporosis, rheumatoid arthritis, macular degeneration, peripheral degenerative disease, or skin aging.

Embodiment 62. The compound or pharmaceutical composition for use of any one of Embodiments 41-55, wherein the disease, disorder, or condition is autism spectrum disorder (ASD), cardiovascular dysfunction, hearing loss, hematopoietic stem cell function, pulmonary fibrosis, schizophrenia, or vision loss.

Embodiment 63. The compound or pharmaceutical composition for use of any one of Embodiments 41-55, wherein the disease, disorder, or condition is progressive supra nuclear palsy.

Embodiment 64. The compound or pharmaceutical composition for use of any one of Embodiments 41-55, wherein the disease, disorder, or condition is amyotrophic lateral sclerosis.

Embodiment 65. The compound or pharmaceutical composition for use of any one of Embodiments 41-55, wherein the disease, disorder, or condition is Aicardi-Goutieres syndrome.

Embodiment 66. The compound or pharmaceutical composition for use of any one of Embodiments 41-55 wherein one or more optional therapeutic agents is to be administered to the subject.

Embodiment 67. The compound or pharmaceutical composition for use of any one of Embodiments 41-66, wherein the subject is (a) not infected with the HIV virus; (b) not suspected of being infected with the HIV virus; (c) not being treated for the HIV virus; and/or (d) not being treated to prevent the HIV virus.

Embodiment 68. The compound or pharmaceutical composition for use of any one of Embodiments 41-67, wherein the compound inhibits human LINE-1 retrotransposition activity with a half maximal inhibitory concentration of 1 μM or less in an in vitro HeLa cell-based dual-luciferase assay.

Embodiment 69. The compound or pharmaceutical composition for use of Embodiment 68, wherein the compound inhibits human LINE-1 retrotransposition activity with a half maximal inhibitory concentration of 0.25 μM or less in an in vitro HeLa cell-based dual-luciferase assay.

Embodiment 70. The compound for use of any one of Embodiments 41-69.

Embodiment 71. The pharmaceutical composition for use of any one of Embodiments 41-69.

Embodiment 82. Use of a compound of:
(i) Table 1, or a pharmaceutically acceptable salt or solvate thereof, or a tautomer thereof;
(ii) Table 2, or a pharmaceutically acceptable salt or solvate thereof, or a tautomer thereof, or a pharmaceutical composition thereof; or
(iii) Table 3, or a pharmaceutically acceptable salt or solvate thereof, or a tautomer thereof, or a pharmaceutical composition thereof,
for the manufacture of a medicament for treating or preventing a disease, disorder, or condition caused by a pathophysiological retrotransposon-associated process in a subject in need thereof, and/or treating or preventing a symptom of the disease, disorder, or condition.

Embodiment 83. Use of a compound of:
(i) Table 1, or a pharmaceutically acceptable salt or solvate thereof, or a tautomer thereof;
(ii) Table 2, or a pharmaceutically acceptable salt or solvate thereof, or a tautomer thereof, or a pharmaceutical composition thereof; or
(iii) Table 3, or a pharmaceutically acceptable salt or solvate thereof, or a tautomer thereof, or a pharmaceutical composition thereof,
for the manufacture of a medicament for inhibiting a LINE-1 retrotransposition event that causes a disease, disorder, or condition in a subject in need thereof.

Embodiment 84. Use of a compound of:
(i) Table 1, or a pharmaceutically acceptable salt or solvate thereof, or a tautomer thereof;
(ii) Table 2, or a pharmaceutically acceptable salt or solvate thereof, or a tautomer thereof; or a pharmaceutical composition thereof; or
(iii) Table 3, or a pharmaceutically acceptable salt or solvate thereof, or a tautomer thereof, or a pharmaceutical composition thereof,
for the manufacture of a medicament for treating a disease, condition, or disorder is characterized as having an overexpression retrotransposon RNA, retrotransposon reverse transcriptase, or retrotransposon DNA.

Embodiment 85. The use of any one of Embodiments 82-84 comprising a compound of Table 1, or a pharmaceutically acceptable salt or solvate thereof, or a tautomer thereof.

Embodiment 86. The use of Embodiment 85 comprising a compound of Table 2, or a pharmaceutically acceptable salt or solvate thereof, or a tautomer thereof.

Embodiment 87. The use of Embodiment 86, wherein the compound of Table 1 is Cpd. No. 1, or a pharmaceutically acceptable salt or solvate thereof, or a tautomer thereof.

Embodiment 88. The use of Embodiment 87, wherein the compound of Table 1 is Cpd. No. 3, or a pharmaceutically acceptable salt or solvate thereof, or a tautomer thereof.

Embodiment 89. The use of Embodiment 88, wherein the compound of Table 1 is Cpd. No. 8, or a pharmaceutically acceptable salt or solvate thereof, or a tautomer thereof.

Embodiment 90. The use of Embodiment 89, wherein the compound of Table 1 is Cpd. No. 9, or a pharmaceutically acceptable salt or solvate thereof, or a tautomer thereof.

Embodiment 91. The use of Embodiment 90, wherein the compound of Table 1 is Cpd. No. 4, or a pharmaceutically acceptable salt or solvate thereof, or a tautomer thereof.

Embodiment 92. The use of any one of Embodiments 82-84 comprising a compound of Table 3, or a pharmaceutically acceptable salt or solvate thereof, or a tautomer thereof.

Embodiment 93. The use of any one of Embodiments 82 or 85-92 for treating the disease, disorder, or condition in a subject.

Embodiment 94. The use of any one of Embodiments 82 or 85-92 for preventing the disease, disorder, or condition in a subject.

Embodiment 95. The use of any one of Embodiments 82 or 85-92, for treating the symptom of a disease, disorder, or condition in a subject.

Embodiment 96. The use of any one of Embodiments 82 or 85-92, for preventing the symptom of a disease, disorder, or condition in a subject.

Embodiment 97. The use of any one of Embodiments 82-96, wherein the disease, disorder, or condition is a neurodegenerative disease.

Embodiment 98. The use of Embodiment 97, wherein the neurodegenerative disease is Alzheimer's disease, amyotrophic lateral sclerosis, Parkinson's disease, dementia with Lewy Bodies, multi systems atrophy, Huntington's disease, frontotemporal lobar degeneration, mild cognitive impairment, corticobasal degeneration, progressive supra nuclear palsy, Rett Syndrome, peripheral degenerative disease, or Aicardi-Goutieres syndrome.

Embodiment 99. The use of any one of Embodiments 82-96, wherein the disease, disorder, or condition is an autoimmune disease.

Embodiment 100. The use of Embodiment 99, wherein the autoimmune disease is lupus, rheumatoid arthritis, Sjogrens syndrome, or multiple sclerosis.

Embodiment 101. The use of any one of Embodiments 82-96, wherein the disease, disorder, or condition is an age-associated disease.

Embodiment 102. The use of Embodiment 101, wherein the age-associated disease is Alzheimer's disease, Parkinson's disease, atherosclerosis, osteoarthritis, osteoporosis, rheumatoid arthritis, macular degeneration, peripheral degenerative disease, or skin aging.

Embodiment 103. The use of any one of Embodiments 82-96, wherein the disease, disorder, or condition is autism spectrum disorder (ASD), cardiovascular dysfunction, hearing loss, hematopoietic stem cell function, pulmonary fibrosis, schizophrenia, or vision loss.

Embodiment 104. The use of any one of Embodiments 82-96, wherein the disease, disorder, or condition is progressive supra nuclear palsy.

Embodiment 105. The use of any one of Embodiments 82-96, wherein the disease, disorder, or condition is amyotrophic lateral sclerosis.

Embodiment 106. The use of any one of Embodiments 82-96, wherein the disease, disorder, or condition is Aicardi-Goutieres syndrome.

Embodiment 107. The use of any one of Embodiments 82-96 wherein one or more optional therapeutic agents is to be administered to the subject.

Embodiment 108. The use of any one of Embodiments 82-107, wherein the subject is (a) not infected with the HIV virus; (b) not suspected of being infected with the HIV virus; (c) not being treated for the HIV virus, and/or (d) not being treated to prevent the HIV virus.

Embodiment 109. The use of any one of Embodiments 82-108, wherein the compound inhibits human LINE-1 retrotransposition activity with a half maximal inhibitory concentration of 1 μM or less in an in vitro HeLa cell-based dual-luciferase assay.

Embodiment 110. The method of any one of Embodiments 3-6, wherein the retrotransposon RNA is LINE-1 RNA.

Embodiment 111. The method of any one of Embodiments 3-6, wherein the retrotransposon reverse transcriptase is ORF2p.

Embodiment 112. The method of any one of Embodiments 3-6, wherein the retrotransposon DNA is LINE-1 DNA.

Embodiment 113. The compound for use of Embodiment 43, wherein the retrotransposon RNA is LINE-1 RNA.

Embodiment 114. The compound for use of Embodiment 43, wherein the retrotransposon reverse transcriptase is ORF2p.

Embodiment 115. The compound for use of Embodiment 43, wherein the retrotransposon DNA is LINE-1 DNA.

Embodiment 116. The use of Embodiment 84, wherein the retrotransposon RNA is LINE-1 RNA.

Embodiment 117. The use of Embodiment 84, wherein the retrotransposon reverse transcriptase is ORF2p.

Embodiment 118. The use of Embodiment 84, wherein the retrotransposon DNA is LINE-1 DNA.

Embodiment 119. The method of any one of Embodiments 1-18, wherein the disease, disorder, or condition is ataxia-telangiectasia.

Embodiment 120. The method of any one of Embodiments 1-18, wherein the disease, disorder, or condition is age-related macular degeneration.

Embodiment 121. The method of any one of Embodiments 1-18, wherein the disease, disorder, or condition is systemic lupus erythematosus.

Embodiment 122. The method of any one of Embodiments 1-18, wherein the disease, disorder, or condition is IFN-associated autoimmune disease.

Embodiment 123. The method of Embodiment 122, wherein the IFN-associated autoimmune disease is psoriasis.

Embodiment 124. The method of any one of Embodiments 1-18, wherein the disease, disorder, or condition is Fanconi Anemia.

Embodiment 125. The method of any one of Embodiments 1-18, wherein the disease, disorder, or condition is idiopathic pulmonary fibrosis.

Embodiment 126. The method of any one of Embodiments 1-18, wherein the disease, disorder, or condition is cardiovascular disease.

Embodiment 127. The compound for use of any one of Embodiments 41-55, wherein the disease, disorder, or condition is ataxia-telangiectasia.

Embodiment 128. The compound for use of any one of Embodiments 41-55, wherein the disease, disorder, or condition is age-related macular degeneration.

Embodiment 129. The compound for use of any one of Embodiments 41-55, wherein the disease, disorder, or condition is systemic lupus erythematosus.

Embodiment 130. The compound for use of any one of Embodiments 41-55, wherein the disease, disorder, or condition is IFN-associated autoimmune disease.

Embodiment 131. The compound for use of Embodiment 130, wherein the IFN-associated autoimmune disease is psoriasis.

Embodiment 132. The compound for use of any one of Embodiments 41-55, wherein the disease, disorder, or condition is Fanconi Anemia.

Embodiment 133. The compound for use of any one of Embodiments 41-55, wherein the disease, disorder, or condition is idiopathic pulmonary fibrosis.

Embodiment 134. The compound for use of any one of Embodiments 41-55, wherein the disease, disorder, or condition is cardiovascular disease.

Embodiment 135. The use of any one of Embodiments 82-96, wherein the disease, disorder, or condition is ataxia-telangiectasia.

Embodiment 136. The use of any one of Embodiments 82-96, wherein the disease, disorder, or condition is age-related macular degeneration.

Embodiment 137. The compound for use of any one of Embodiments 82-96, wherein the disease, disorder, or condition is systemic lupus erythematosus.

Embodiment 138. The compound for use of any one of Embodiments 82-96, wherein the disease, disorder, or condition is IFN-associated autoimmune disease.

Embodiment 139. The compound for use of Embodiment 138, wherein the IFN-associated autoimmune disease is psoriasis.

Embodiment 140. The compound for use of any one of Embodiments 82-96, wherein the disease, disorder, or condition is Fanconi Anemia.

Embodiment 141. The compound for use of any one of Embodiments 82-96, wherein the disease, disorder, or condition is idiopathic pulmonary fibrosis.

Embodiment 142. The compound for use of any one of Embodiments 82-96, wherein the disease, disorder, or condition is cardiovascular disease.

Embodiment 143. The method, kit, compound for use, or use of any one of Embodiments 1, 32, 41, or 82, wherein the disease, disorder, or condition is not (i) cancer; or (ii) an infectious disease.

### EXAMPLES

The compounds of Tables 1 and 2 may be prepared as described in the EXAMPLES below, and, for example, in Nomura et al., J. Med. Chem. 42:2901-2908 (1999); Ohrui et al., J. Med. Chem. 43:4516-4525 (2000), JP Patent No. 6767011, and/or U.S. Pat. No. 10,933,067.

The abbreviations in Table 4 may be used in the EXAMPLES.

TABLE 4

| | |
|---|---|
| aq. | aqueous |
| ACN, MeCN, CH₃CN | acetonitrile |
| AIBN | azobisisobutyronitrile |
| AmF | ammonium formate |
| AmBic | ammonium bicarbonate |
| BSA, BTMSA | N,O-bis(trimethylsilyl)acetamide |
| CDC1₃ | chloroform-d |
| calc. | calculated |
| conc. | concentrate |
| DCM | methylene chloride or dichloromethane |
| DMF | dimethylformamide |
| DMAP | 4-dimethylaminopyridine |
| DMSO | dimethylsulfoxide |
| DMSO-d₆ | deutered dimethylsulfoxide |
| Et | ethyl |
| Et₂O | diethyl ether |
| ESI-MS | electrospray ionisation mass spectrometry |
| EtOAc | ethyl acetate |
| FA | formic acid |
| H₂ | hydrogen gas |
| H₂O | water |
| HCl | hydrochloric acid |
| hex. | hexanes |
| hep. | heptanes |
| HPLC | high pressure liquid chromatography |
| IPA | isopropanol |
| K₂CO₃ | potassium carbonate |
| LCMS | liquid chromatography mass spectrometry |
| M | molar |
| Me | methyl |
| MHz | megahertz |
| mg | milligram(s) |
| min. | minutes |
| mL | milliliter(s) |
| mM | millimolar |
| MeOH | methanol |
| MeOD | methanol-d₄ |
| MgSO₄ | magnesium sulfate |
| MS | mass spectrometer |
| μg | microgram(s) |
| μL | microliter(s) |
| μM | micromolar |
| μmol | micromole |
| N₂ | nitrogen gas |
| NaHCO₃ | sodium bicarbonate |
| NaI | Sodium iodide |
| Na₂CO₃ | sodium carbonate |
| Na₂SO₄ | sodium sulfate |
| NaOH | sodium hydroxide |
| NaOMe | sodium methoxide |
| NH₄Cl | ammonium chloride |
| ¹H NMR | proton nuclear magnetic resonance |
| ON | overnight |
| Pd/C | palladium on carbon |
| Ph | phenyl |
| psi | pound per square inch |
| quant. | quantitative |
| RT | retention time |
| rt | room temperature |
| r.b.f. (rbf) | round bottom flask |
| sat. | saturated |
| THF | tetrahydrofuran |
| TLC | thin layer chromatography |
| TTMSS | tris(trimethylsilyl)silane |
| UPLC | ultra performance liquid chromatography |
| UV | ultraviolet |
| Prep-HPLC | Preparative HPLC |

The following LC-MS Methods may be used in the EXAMPLES.

Method A: UPLC-MS method: Waters Acquity UPLC CSH C18, 1.8 μm, 2.1×30 mm at 40° C.; 5% to 100% B in 5.2 minutes; hold 100% B for 1.8 minutes, run time=7.0 min, flow 0.9 mL/min; Eluents: A=Milli-Q H₂O+10 mM Ammonium formate pH=3.8; B=MeCN. Waters Acquity UPLC system. UV Detector=Waters Acquity PDA, 198-360 nm. MS Detector=Waters SQD ESI.

Method B: Waters Acquity CSH C18, 3.5 μm, 4.6×30 mm at 40° C.; Iso 5% B for 0.5 min, 5% to 100% B in 5 minutes; hold 100% B for 1.5 minutes, run time=7.0 min, flow 0.9 mL/min; Eluents: A=Milli-Q H₂O+10 mM Ammonium formate pH=3.8; B=MeCN. Waters Alliance 2695 system. UV Detection: Waters 2996 PDA, 198-360 nm. MS Detector: Waters ZQ 2000, ESI

Method C: SHIMADZU LC20-MS2010: MERCK, RP-18e 25-2 mm at 50° C.; 1.5 ML/4 L TFA in water (solvent A) and 0.75 ML/4 LTFA in acetonitrile (solvent B), using the elution gradient 5%-95% (solvent B) over 0.7 minutes and holding at 95% for 0.4 minutes at a flow rate of 1.5 ml/min, run time=1.5 min; UV Detector=220 nm, 254 nm; MS Detector=ESI

Method D: LCMS-BT. SHIMADZU LC20-MS2020: X bridge Shield RP-18.5 μm, 2.1*50 mm at 50° C.; 0.8 mL/4 L NH₃·H₂O in water (solvent A) and acetonitrile (solvent B), using the elution gradient 0%-30% (solvent B) over 2 minutes and holding at 30% for 0.48 minutes at a flow rate of 1 ml/min, run time=3 min; UV Detector=220 nm, 254 nm; MS Detector=ESI

Method E: LCMS-AN: Agilent LC1200-MS6110: X timate C18 2.1*30 mm, 3 μm at 50° C.; 1.5 ML/4 L TFA in water (solvent A) and 0.75 ML/4 L TFA in acetonitrile (solvent B), using the elution gradient 0%-600% (solvent B) over 0.9 minutes and holding at 60% for 0.6 minutes at a flow rate of 1.2 ml/min, run time=2 min: UV Detector=220 nm: MS Detector=ESI.

Method F: LCMS-CI: Agilent LC1200-MS6110: Xbridge Shield RP-18.5 μm, 2.1*50 mm; at 30° C.; water (4 L)+NH₃·H₂O (0.8 mL) (solvent A) and acetonitrile (solvent B), using the elution gradient 0%-60% (solvent B) over 2.0 minutes and holding at 60% for 0.48 minutes at a flow rate of 1 ml/min, run time=3.0 min; UV Detector=220 nm, 254 nm; MS Detector=ESI.

Method G: UPLC-MS Method: Waters Acquity UPLC CSH C18, 1.8 μm, 2.1×30 mm at 40° C.; 5% to 100% B in 2.0 minutes; hold 100% B for 0.7 minutes, run time=2.7 min, flow 0.9 mL/min; Eluents: A=Milli-Q H₂O+10 mM Ammonium formate pH=3.8, B=MeCN. Waters Acquity UPLC system. UV Detector=Waters Acquity PDA, 198-360 nm. MS Detector=Waters SQD ESI.

Method H: UPLC-MS Method. Waters Acquity UPLC Agilent Poroshell 120 EC C18, 1.8 μm, 2.1×250 mm at 40° C.; 5% to 100% B in 5.0 minutes; hold 100% B for 2.0 minutes, run time=7.0 min, flow 0.5 mL/min; Eluents: A=Milli-Q H₂O+10 mM Ammonium formate pH=3.8; B=MeCN. Waters Acquity UPLC system. UV Detector=Waters Acquity PDA, 198-360 nm. MS Detector=Waters SQD ESI.

### Example 1

### Synthesis of 1-((2R,4S,5R)-4-hydroxy-5-(hydroxymethyl)-5-vinyltetrahydrofuran-2-yl)-5-methylpyrimidine-2,4(1H,3H)-dione (Cpd. No. 62)

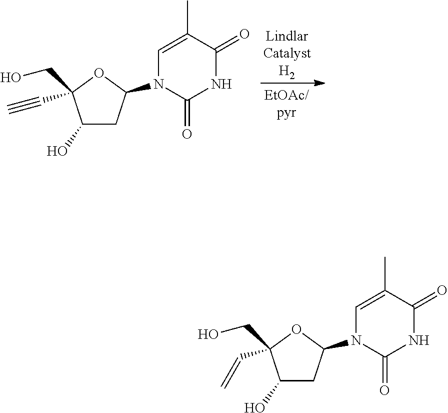 [SMILES: C#C[C@]1(CO)O[C@@H](n2cc(C)c(=O)[nH]c2=O)C[C@@H]1O ; C=C[C@]1(CO)O[C@@H](n2cc(C)c(=O)[nH]c2=O)C[C@@H]1O]

1-((2R,4S,5R)-5-Ethynyl-4-hydroxy-5-(hydroxymethyl)tetrahydrofuran-2-yl)-5-methylpyrimidine-2,4(1H,3H)-dione was prepared according to WO 2007/038507 A2.

1-((2R,4S,5R)-5-ethynyl-4-hydroxy-5-(hydroxymethyl)tetrahydrofuran-2-yl)-5-methylpyrimidine-2,4(1H,3H)-dione (18.0 mg, 67.6 μmol) was dissolved in 9:1 EtOAc/pyridine (653 μl/22.5 μl) in a 5-mL vial with a rubber septum. Then solid Lindlar Catalyst (18.0 mg, 67.6 μmol) was added and the reaction mixture was flushed with H₂ gas with a balloon for 30 min. This reaction was stirred till full conversion to the title compound was observed by LC-MS. Then, the reaction mixture was filtered over Celite® and rinsed with EtOAc. The desired product was purified by flash chromatography on silica gel (EtOAc), yielding 1-((2R,4S,5R)-4-hydroxy-5-(hydroxymethyl)-5-vinyltetrahydrofuran-2-yl)-5-methylpyrimidine-2,4(1H,3H)-dione (11 mg, 58%). LC-MS (ESI) m/z calcd for C₁₂H₁₆N₂O₅: 268.11. Found 267.3 [M−H]⁻. ¹H NMR: (400 MHz, CD₃OD) 9.01 (s, 1H), 7.23 (t, J=5.7 Hz, 1H), 6.99 (dd, J=17.3, 10.9 Hz, 1H). 6.51 (dd, J=17.3, 2.0 Hz, 1H), 6.30 (dd, J=10.9, 1.2 Hz, 1H), 5.63 (t, J=7.4 Hz, 1H), 4.65 (qAB, J=11.9, 5.0 Hz, 2H), 3.30 (dd, J=7.4, 5.8 Hz, 2H), 2.93 (s, 3H).

### Example 2

### Synthesis of 5-bromo-1-((2R,4S,5R)-4-hydroxy-5-(hydroxymethyl)-5-vinyltetrahydrofuran-2-yl)pyrimidine-2,4(1H,3H)-dione (Cpd. No. 64)

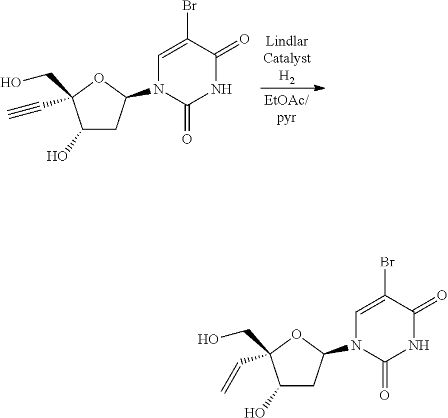 [SMILES: C#C[C@]1(CO)O[C@@H](n2cc(Br)c(=O)[nH]c2=O)C[C@@H]1O ; C=C[C@]1(CO)O[C@@H](n2cc(Br)c(=O)[nH]c2=O)C[C@@H]1O]

5-Bromo-1-((2R,4S,5R)-5-ethynyl-4-hydroxy-5-(hydroxymethyl)tetrahydrofuran-2-yl)pyrimidine-2,4(1H,3H)-dione was prepared according to WO 2007/038507 A2.

5-bromo-1-((2R,4S,5R)-5-ethynyl-4-hydroxy-5-(hydroxymethyl)tetrahydrofuran-2-yl)pyrimidine-2,4(1H,3H)-dione (3.3 mg, 9.97 μmol) was dissolved in 9:1 EtOAc/pyridine (96.3 μl/3.32 μl) in a 5-mL vial with a rubber septum. Then solid Lindlar Catalyst (3.30 mg, 9.97 μmol) was added and the reaction mixture was flushed by bubbling H₂ gas with a balloon for 30 min. This reaction was stirred till full conversion to the title compound was observed by LC-MS. Then, the reaction mixture was filtered over Celite® and rinsed with EtOAc. The desired product was purified by flash chromatography on silica gel (EtOAc), yielding 5-bromo-1-((2R,4S,5R)-4-hydroxy-5-(hydroxymethyl)-5-vinyltetrahydrofuran-2-yl)pyrimidine-2,4(1H,3H)-dione (2.70 mg, 81%). LC-MS (ESI) m/z calcd for C₁₁H₁₃BrN₂O₅: 332.00. Found 331.2 [M−H]⁻. ¹H NMR (400 MHz, CD₃OD) δ 8.67 (s, 1H), 6.12 (t, J=5.3 Hz, 1H), 5.92 (dd, J=17.3, 10.9 Hz, 1H), 5.46 (dd, J=17.3, 1.9 Hz, 1H), 5.28 (dd, J=10.9, 1.9 Hz, 1H), 4.55 (t, J=7.6 Hz, 1H), 3.69 (d, J=12.0 Hz, 1H), 3.53 (d, J=12.0 Hz, 1H), 2.27 (dd, J=7.7, 5.5 Hz, 2H).

### Example 3

### Synthesis of 1-((2R,4S,5R)-4-hydroxy-5-(hydroxymethyl)-5-vinyltetrahydrofuran-2-yl)-5-methoxypyrimidine-2,4(1H,3H)-dione (Cpd. No. 6)

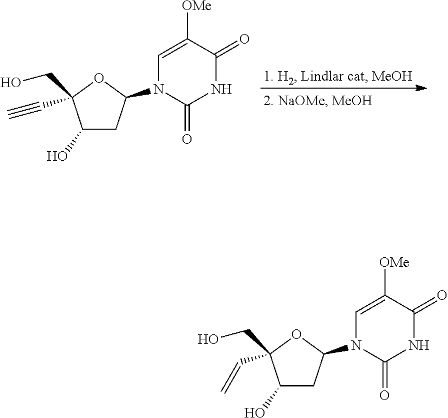 [SMILES: C#C[C@]1(CO)O[C@@H](n2cc(OC)c(=O)[nH]c2=O)C[C@@H]1O ; C=C[C@]1(CO)O[C@@H](n2cc(OC)c(=O)[nH]c2=O)C[C@@H]1O]

Step 1. 1-((2R,4S,5R)-5-Ethynyl-4-hydroxy-5-(hydroxymethyl)tetrahydrofuran-2-yl)-5-methoxypyrimidine-2,4(1H,3H)-dione was prepared according to WO 2007/038507 A2.

To a mixture of [(2R,3S,5R)-2-ethynyl-5-(5-methoxy-2,4-dioxo-pyrimidin-1-yl)-3-(4-methylbenzoyl)oxy-tetrahydrofuran-2-yl]methyl 4-methylbenzoate (15 mg, 28.93 μmol) in MeOH (2 mL) was added Lindlar catalyst (7 mg) in one portion at 25° C. under H₂ (15 psi). The resulting mixture was stirred at 25° C. for 1 hour. Then the mixture was filtered and the filtrate was concentrated to afford the compound (15 mg, 99%) as a white solid.

Step 2. To a mixture of [(2R,3S,5R)-5-(5-methoxy-2,4-dioxo-pyrimidin-1-yl)-3-(4-methylbenzoyl)oxy-2-vinyl-tetrahydrofuran-2-yl]methyl 4-methylbenzoate (15 mg, 28.82 μmol) in MeOH (5 mL) was added NaOMe (156 μg, 2.88 μmol) in one portion at 25° C. After stirring at 25° C. for 1 hour, the mixture was concentrated under reduced pressure. The residue was purified by prep-HPLC (basic) to afford the title compound (5 mg, 62%) as a white solid. LCMS (ESI): m/z 307.0 (M+Na)⁺. ¹H NMR (400 MHz, DMSO-d₆) δ 11.28 (s, 1H), 7.77 (s, 1H), 6.17-6.07 (m, 1H), 5.97-5.85 (m, 1H), 5.44 (t, J=4.8 Hz, 1H), 5.39-5.31 (m, 1H), 5.28 (d, J=5.2 Hz, 1H), 5.23-5.15 (m, 1H), 4.54-4.46 (m, 1H), 3.65-3.55 (m, 1H), 3.59 (s, 3H), 3.43-3.36 (m, 1H), 2.24-2.15 (m, 1H), 2.13-2.04 (m, 1H).

### Example 4

### Synthesis of 1-((2R,4S,5R)-4-hydroxy-5-(hydroxymethyl)-5-vinyltetrahydrofuran-2-yl)-2,4-dioxo-1,2,3,4-tetrahydropyrimidine-5-carbonitrile (Cpd. No. 69)

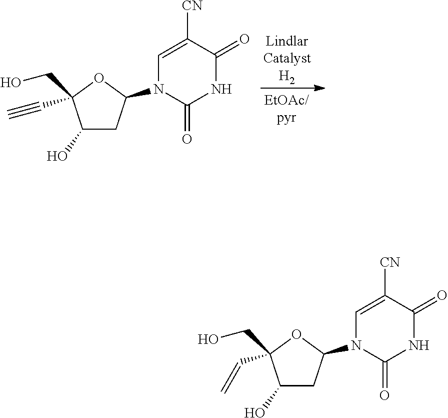 [SMILES: C#C[C@]1(CO)O[C@@H](n2cc(C#N)c(=O)[nH]c2=O)C[C@@H]1O ; C=C[C@]1(CO)O[C@@H](n2cc(C#N)c(=O)[nH]c2=O)C[C@@H]1O]

1-((2R,4S,5R)-5-ethynyl-4-hydroxy-5-(hydroxymethyl)tetrahydrofuran-2-yl)-2,4-dioxo-1,2,3,4-tetrahydropyrimidine-5-carbonitrile was prepared according to WO 2007/038507 A2 using 5-pyrimidinecarbonitrile. 1-((2R,4S,5R)-5-ethynyl-4-hydroxy-5-(hydroxymethyl)tetrahydrofuran-2-yl)-2,4-dioxo-1,2,3,4-tetrahydropyrimidine-5-carbonitrile (8.9 mg, 32.1 μmol) was dissolved in MeOH (321 μl) in a 5-mL vial with a rubber septum. Then solid Lindlar Catalyst (8.9 mg, 32.1 μmol) was added and the reaction mixture was flushed by bubbling H₂ gas with a balloon for 30 min. This reaction was stirred till full conversion to the title compound was observed by LC-MS. Then, the reaction mixture was filtered over Celite® and rinsed with EtOAc. The desired product was purified by flash chromatography on silica gel (EtOAc), yielding 1-((2R,4S,5R)-4-hydroxy-5-(hydroxymethyl)-5-vinyltetrahydrofuran-2-yl)-2,4-dioxo-1,2,3,4-tetrahydropyrimidine-5-carbonitrile (1.36 mg, 13%). LC-MS (ESI) m/z calcd for C₁₂H₁₃N₃O₅: 279.1. Found 278.1 [M−H]⁻. ¹H NMR (400 MHz, CD₃OD) δ 9.13 (d, J=4.5 Hz, 1H), 6.21-5.98 (m, 1H), 5.92 (dd, J=17.3, 11.0 Hz, 1H), 5.46 (dd, J=17.3, 1.9 Hz, 1H), 5.29 (dd, J=11.0, 1.9 Hz, 1H), 4.60-4.45 (m, 1H), 3.72 (dd, J=11.9, 6.9 Hz, 1H), 3.65-3.54 (m, 1H), 2.48-2.22 (m, 2H).

### Example 5

### Synthesis of 4-amino-5-bromo-1-((2R,4S,5R)-4-hydroxy-5-(hydroxymethyl)-5-vinyltetrahydrofuran-2-yl)pyrimidin-2(1H)-one (Cpd. No. 90)

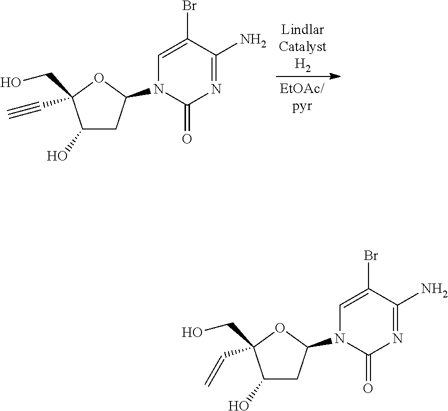 [SMILES: C#C[C@]1(CO)O[C@@H](n2cc(Br)c(N)nc2=O)C[C@@H]1O ; C=C[C@]1(CO)O[C@@H](n2cc(Br)c(N)nc2=O)C[C@@H]1O]

4-amino-5-bromo-1-((2R,4S,5R)-5-ethynyl-4-hydroxy-5-(hydroxymethyl)tetrahydrofuran-2-yl)pyrimidin-2(1H)-one was prepared according to WO 2007/038507 A2.

4-amino-5-bromo-1-((2R,4S,5R)-5-ethynyl-4-hydroxy-5-(hydroxymethyl)tetrahydrofuran-2-yl)pyrimidin-2(1H)-one (64.0 mg, 194 μmol) was dissolved in MeOH (1.94 mL) in a 5-mL vial with a rubber septum. Then solid Lindlar Catalyst (64 mg, 194 μmol) was added and the reaction mixture was flushed by bubbling H₂ gas with a balloon for 30 min. This reaction was stirred till full conversion to the title compound was observed by LC-MS. Then, the reaction mixture was filtered over Celite® and rinsed with MeOH. The desired product was purified by flash chromatography on silica gel using 0-100% 2-propanol in EtOAc, yielding 4-amino-5-bromo-1-((2R,4S,5R)-4-hydroxy-5-(hydroxymethyl)-5-vinyltetrahydrofuran-2-yl)pyrimidin-2(1H)-one (31.6 mg, 45%). LC-MS (ESI) m/z calcd for C₁₁H₁₄BrN₃O₄: 331.02. Found 334.0 [M+3H]⁺. ¹H NMR (400 MHz, CD₃OD) δ 8.70 (s, 1H), 6.06 (dd, J=6.9, 3.0 Hz, 1H), 6.02-5.82 (m, 1H), 5.46 (dd, J=17.3, 1.8 Hz, 1H), 5.36-5.19 (m, 1H), 4.59-4.43 (m, 1H), 4.44-4.36 (m, 1H), 3.70 (dd, J=12.0, 5.7 Hz, 1H), 3.55 (d, J=12.0 Hz, 1H), 2.38-2.24 (m, 1H), 2.18 (ddd, J=13.4, 7.1, 3.0 Hz, 1H).

### Example 6

### Synthesis of 2-amino-9-((2R,4S,5R)-4-hydroxy-5-(hydroxymethyl)-5-vinyltetrahydrofuran-2-yl)-1H-purin-6(9H)-one (Cpd. No. 9)

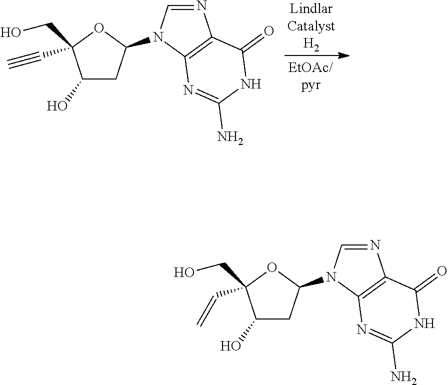 [SMILES: C#C[C@]1(CO)O[C@@H](n2cnc3c(=O)[nH]c(N)nc32)C[C@@H]1O ; C=C[C@]1(CO)O[C@@H](n2cnc3c(=O)[nH]c(N)nc32)C[C@@H]1O]

2-Amino-9-((2R,4S,5R)-5-ethynyl-4-hydroxy-5-(hydroxymethyl)tetrahydrofuran-2-yl)-1H-purin-6(9H)-one was prepared according to WO 2007/038507 A2.

2-amino-9-((2R,4S,5R)-5-ethynyl-4-hydroxy-5-(hydroxymethyl)tetrahydrofuran-2-yl)-1H-purin-6(9H)-one (125 mg, 429 μmol) was dissolved in EtOAc/pyridine (4.15 mL/143 μl) in a 5-mL vial with a rubber septum. Then solid Lindlar Catalyst (125 mg, 429 μmol) was added and the reaction mixture was flushed by bubbling H₂ gas with a balloon for 30 min. This reaction was stirred till full conversion to the title compound was observed by LC-MS. Then, the reaction mixture was filtered over Celite® and rinsed with MeOH. The desired product was purified by using SFC purification using a IC, 10×250 mm 5 um column and an isocratic gradient of 25% MeOH, 0.1% NH₄OH, 75% CO₂ with a 10 mL/min flowrate and a 20 min runtime, yielding 2-amino-9-((2R,4S,5R)-4-hydroxy-5-(hydroxymethyl)-5-vinyltetrahydrofuran-2-yl)-1H-purin-6(9H)-one (2.62 mg, 2%). LC-MS (ESI) m/z calcd for C₁₂H₁₅N₅O₄: 293.1. Found 292.2 [M−H]⁻. ¹H NMR (400 MHz, CD₃OD) δ 8.17 (d, J=99.1 Hz, 1H), 6.28 (dd, J=7.9, 3.6 Hz, 1H), 6.04 (ddd, J=17.3, 13.6, 11.1 Hz, 1H), 5.37-5.08 (m, 2H), 4.47 (dd, J=6.5, 2.9 Hz, 1H), 3.58-3.37 (m, 2H), 3.04-2.84 (m, 1H), 2.37-2.23 (m, 1H).

### Example 7

### Synthesis of 4-amino-5-bromo-1-((4S,5R)-5-ethyl-4-hydroxy-5-(hydroxymethyl)tetrahydrofuran-2-yl)pyrimidin-2(1H)-one (Cpd. No. 70)

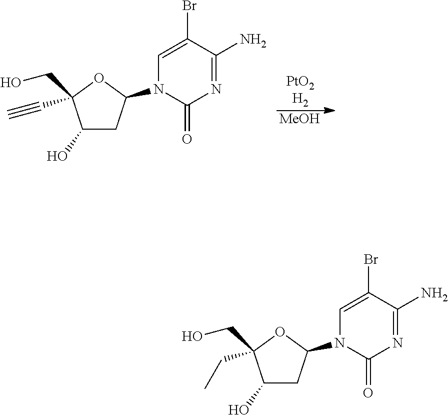 [SMILES: "MeOH" ; C#C[C@]1(CO)O[C@@H](n2cc(Br)c(N)nc2=O)C[C@@H]1O ; CC[C@]1(CO)O[C@@H](n2cc(Br)c(N)nc2=O)C[C@@H]1O]

4-Amino-5-bromo-1-((4S,5R)-5-ethynyl-4-hydroxy-5-(hydroxymethyl)tetrahydrofuran-2-yl)pyrimidin-2(1H)-one was synthesized according the procedure described in WO 2007/038507 A2.

4-amino-5-bromo-1-((4S,5R)-5-ethynyl-4-hydroxy-5-(hydroxymethyl)tetrahydrofuran-2-yl)pyrimidin-2(1H)-one (5.45 mg, 16.4 μmol) was dissolved in MeOH in a 5-mL vial with a rubber septum. Then solid PtO₂ (0.500 mg, 2.20 μmol) was added and the reaction mixture was flushed with H₂ balloon for 30 min. This reaction was stirred till full conversion to the title compound was observed by LC-MS. Then the reaction mixture was filtered through Celite® and washed with MeOH and the solvent was removed under reduced pressure. The crude mixture was purified by column chromatography on silica gel (EtOAc/hep 1:1 to 100% EtOAc) yielding 4-amino-5-bromo-1-((4S,5R)-5-ethyl-4-hydroxy-5-(hydroxymethyl)tetrahydrofuran-2-yl)pyrimidin-2(1H)-one (0.750 mg, 13%) as a mixture of anomers an off-white powder. LC-MS (ESI) m/z calcd for C₁₁H₁₅BrN₃O₄: 332.03. Found 332.2 [M−H]−. ¹H NMR (400 MHz, CD₃OD) δ 8.55 (s, 1H), 6.11-6.03 (m, 1H), 4.47-4.34 (m, 1H), 4.12-4.00 (m, 1H), 3.70 (d, J=11.7 Hz, 1H), 3.61-3.44 (m, 1H), 2.43 (ddd, J=26.1, 18.8, 13.1 Hz, 1H), 2.23 (ddd, J=13.8, 6.9, 5.5 Hz, 1H), 1.86 (dd, J=15.3, 7.5 Hz, 1H), 1.68 (t, J=7.6 Hz, 1H), 0.96 (tt, J=22.6, 11.3 Hz, 3H).

### Example 8

### Synthesis of 1-((4S,5R)-5-ethyl-4-hydroxy-5-(hydroxymethyl)tetrahydrofuran-2-yl)-5-methoxypyrimidine-2,4(1H,3H)-dione (Cpd. No. 68)

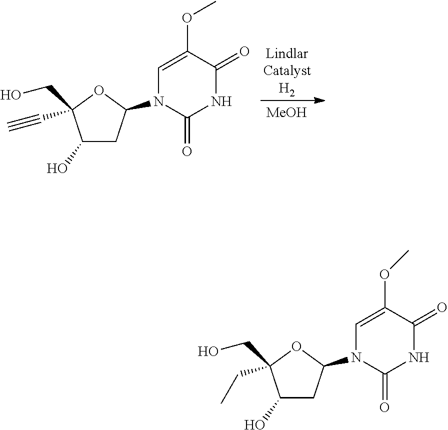 [SMILES: "MeOH" ; C#C[C@]1(CO)O[C@@H](n2cc(OC)c(=O)[nH]c2=O)C[C@@H]1O ; CC[C@]1(CO)O[C@@H](n2cc(OC)c(=O)[nH]c2=O)C[C@@H]1O]

1-((4S,5R)-5-Ethynyl-4-hydroxy-5-(hydroxymethyl)tetrahydrofuran-2-yl)-5-methoxypyrimidine-2,4(1H,3H)-dione was synthesized according the procedure described in WO 2007/038507 A2.

1-((4S,5R)-5-ethynyl-4-hydroxy-5-(hydroxymethyl)tetrahydrofuran-2-yl)-5-methoxypyrimidine-2,4(1H,3H)-dione (7.20 mg, 28.7 μmol) was dissolved in MeOH in a 5-mL vial with a rubber septum. Then solid Lindlar Catalyst (7.20 mg, 28.7 μmol) was added and the reaction mixture was flushed with H₂ balloon for 30 min. This reaction was stirred till full conversion to the title compound was observed by LC-MS. Then the reaction mixture was filtered through Celite® and washed with MeOH and the solvent was removed under reduced pressure, affording 4-amino-1-((4S,5R)-5-ethyl-4-hydroxy-5-(hydroxymethyl)tetrahydrofuran-2-yl)-5-methoxypyrimidin-2(1H)-one (5.75 mg, 66%) as a mixture of anomers an white powder. LC-MS (ESI) m/z calcd for C₁₂H1₈N₃O₅: 284.13. Found 284.4 [M−H]−. ¹H NMR (400 MHz, CD₃OD) δ 7.82 (d, J=22.7 Hz, 1H), 6.38-6.07 (m, 1H), 4.55-4.10 (m, 1H), 3.64-3.37 (m, 2H), 3.33 (s, 3H), 2.33 (td, J=7.0, 6.0 Hz, 1H), 1.95-1.75 (m, 1H), 1.62 (dd, J=41.8, 7.6 Hz, 1H), 1.54 (m, 1H), 0.96 (dt, J=11.9, 7.6 Hz, 3H).

### Example 9

### Synthesis of (2R,3S,5R)-5-(6-(cyclopropylamino)-9H-purin-9-yl)-2-(hydroxymethyl)-2-vinyltetrahydrofuran-3-ol (Cpd. No. 94)

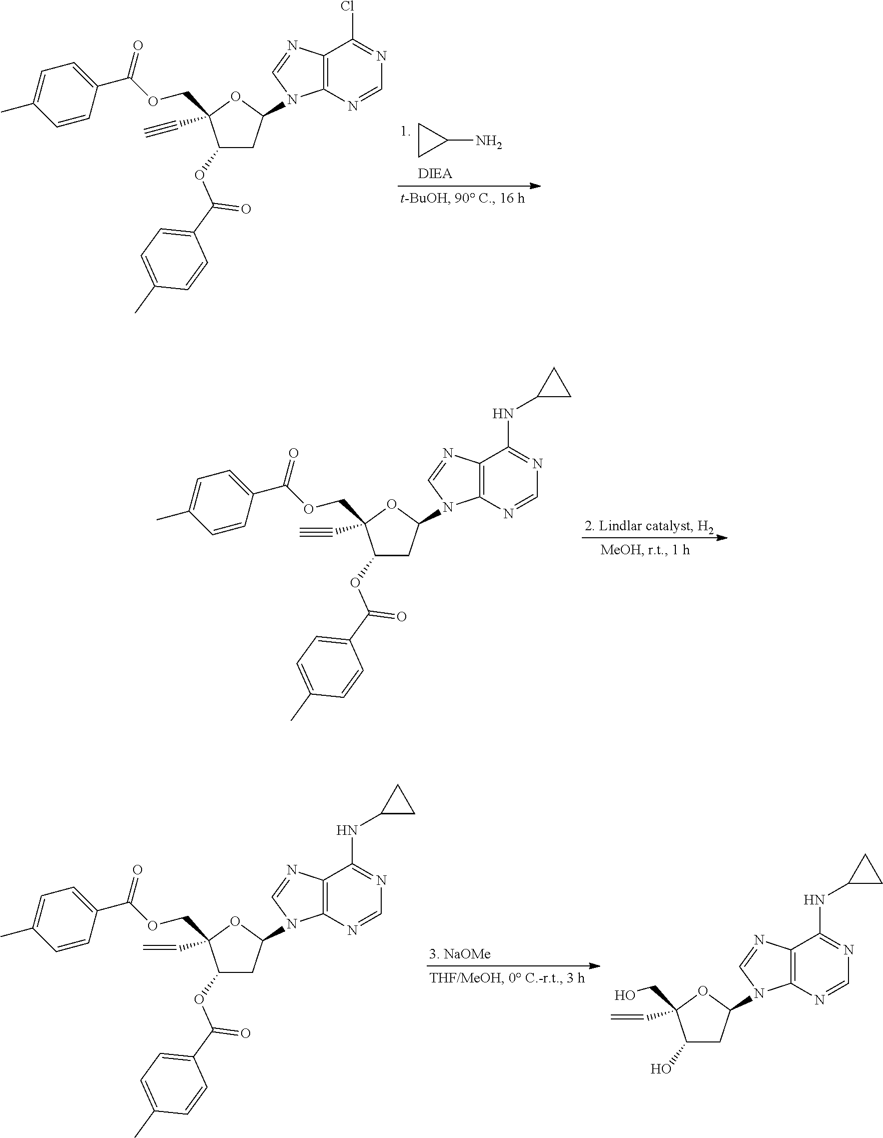

((2R,3S,5R)-5-(6-chloro-9H-purin-9-yl)-2-ethynyl-2-(((4-methylbenzoyl)oxy)methyl)tetrahydrofuran-3-yl 4-methylbenzoate was synthesized according the procedure described in WO 2007/038507 A2 using N-cyclopropyl-9H-purin-6-amine.

Step 1. To a mixture of [(2R,3S,5R)-5-(6-chloropurin-9-yl)-2-ethynyl-3-(4-methylbenzoyl)oxy-tetrahydrofuran-2-yl]methyl 4-methylbenzoate (0.05 g, 94.17 μmol) and cyclopropanamine (8 mg, 141.25 μmol) in t-BuOH (2 mL) was added DIEA (24 mg, 188.34 μmol) in one portion at 25° C. under N₂. The resulting mixture was stirred at 90° C. for 16 hours. After cooling to room temperature, the mixture was concentrated under reduced pressure. The residue was purified by pre-TLC (50% EtOAc in petroleum ether) to afford the compound (45 mg, 87%) as a colorless oil. ¹H NMR (400 MHz, CDCl₃) δ 8.66 (s, 1H), 8.24 (s, 1H), 8.06-8.00 (m, 2H), 7.87-7.80 (m, 2H), 7.30 (d, J=8.0 Hz, 2H), 7.19 (d, J=8.0 Hz, 2H), 6.65-6.55 (m, 1H), 6.21-6.13 (m, 1H), 4.91 (d, J=12.0 Hz, 1H), 4.63 (d, J=12.0 Hz, 1H), 3.47-3.30 (m, 1H), 3.00-2.90 (m, 1H), 2.74 (s, 1H), 2.44 (d, J=16.0 Hz, 6H).

Step 2. To a solution of [(2R,3S,5R)-5-[6-(cyclopropylamino)purin-9-yl]-2-ethynyl-3-(4-methylbenzoyl)oxy-tetrahydrofuran-2-yl]methyl 4-methylbenzoate (45 mg, 81.58 μmol) in MeOH (2 mL) was added Lindlar catalyst (17 mg) in one portion at 25° C. under H₂ (15 psi). The resulting mixture was stirred at 25° C. for 1 hour. The mixture was filtered and concentrated directly to afford the compound (45 mg, 99%) as a white solid.

Step 3. To a mixture of [(2R,3S,5R)-5-[6-(cyclopropylamino)purin-9-yl]-3-(4-methylbenzoyl)oxy-2-vinyl-tetrahydrofuran-2-yl]methyl 4-methylbenzoate (45 mg, 81.29 μmol) in MeOH (5 mL) was added NaOMe (439 μg, 8.13 μmol) in one portion at 25° C. After stirring at 25° C. for 1 hour. The mixture was concentrated under reduced pressure. The residue was purified by prep-HPLC (acetonitrile 0-30%/0.225% formic acid in water) to afford the title compound (13.6 mg, 53%) as a white solid. ¹H NMR (400 MHz, DMSO-d6) δ 8.37 (s, 1H), 8.24 (s, 1H), 7.96 (s, 1H), 6.40-6.30 (m, 1H), 6.03-5.91 (m, 1H), 5.44-5.32 (m, 2H), 5.28 (d, J=5.2 Hz, 1H), 5.23-5.16 (m, 1H), 4.68-4.61 (m, 1H), 3.56-3.41 (m, 2H), 2.66-2.61 (m, 1H), 2.31-2.21 (m, 1H), 0.76-0.68 (m, 2H), 0.65-0.55 (m, 2H).

### Example 10

### Synthesis of (2R,3S,5R)-5-(6-(cyclopropylamino)-2-fluoro-9H-purin-9-yl)-2-(hydroxymethyl)-2-vinyltetrahydrofuran-3-ol (Cpd. No. 5)

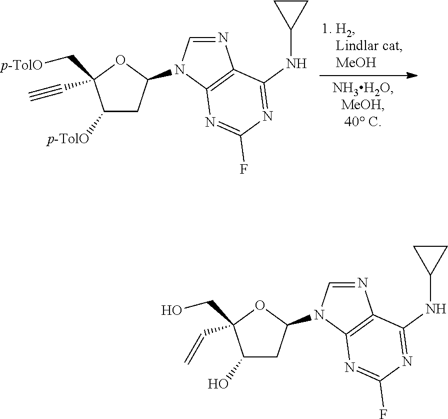

[(2R,3S,5R)-5-[6-(cyclopropylamino)-2-fluoro-purin-9-yl]-2-ethynyl-3-(4-methylbenzoyl)oxy-tetrahydrofuran-2-yl]methyl 4-methylbenzoate was synthesized according the procedure described in WO 2007/038507 A2 using N-cyclopropyl-2-fluoro-9H-purin-6-amine.

Step 1. To a solution of [(2R,3S,5R)-5-[6-(cyclopropylamino)-2-fluoro-purin-9-yl]-2-ethynyl-3-(4-methylbenzoyl)oxy-tetrahydrofuran-2-yl]methyl 4-methylbenzoate (50 mg, 87.78 μmol) in EtOAc (3 mL) was added Lindlar catalyst (18 mg, 8.78 μmol, 10% purity) in one portion at 25° C. The mixture was degassed with H₂ for three times and stirred at 25° C. under H₂ (15 psi) for 1 h. After that, the reaction mixture was filtered, the filtrate was concentrated to afford the compound (46 mg, 92%) as a white solid. LCMS (ESI): m/z 594.1 (M+Na)+.

Step 2. To a solution of [(2R,3S,5R)-5-[6-(cyclopropylamino)-2-fluoro-purin-9-yl]-3-(4-methylbenzoyl)oxy-2-vinyl-tetrahydrofuran-2-yl]methyl 4-methylbenzoate (45 mg, 78.73 μmol) in MeOH (3 mL) was added NH₃·H₂O (1 mL, 6.49 mmol, 25% purity) at 25° C. The mixture was stirred at 40° C. for 16 h. After that, the reaction mixture was concentrated. The residue was purified by reversed-phase HPLC (acetonitrile 1-27%/0.225% formic acid in water) to the title compound (14.1 mg, 53%) as a white solid. ¹H NMR (400 MHz, DMSO-d6) δ 8.54 (br s, 1H), 8.37 (s, 1H), 6.28-6.20 (m, 1H), 6.00-5.90 (m, 1H), 5.42-5.36 (m, 1H), 5.30 (br d, J=4.8 Hz, 1H), 5.26-5.18 (m, 1H), 5.10 (br t, J=5.6 Hz, 1H), 4.71-4.59 (m, 1H), 3.55-3.45 (m, 2H), 2.93 (br s, 1H), 2.61-2.70 (m, 1H), 2.31-2.22 (m, 1H), 0.79-0.60 (m, 4H).

### Example 11

### Synthesis of (2R,3S,5R)-5-(2-amino-6-(cyclopropylamino)-9H-purin-9-yl)-2-(hydroxymethyl)-2-vinyltetrahydrofuran-3-ol (Cpd. No. 4)

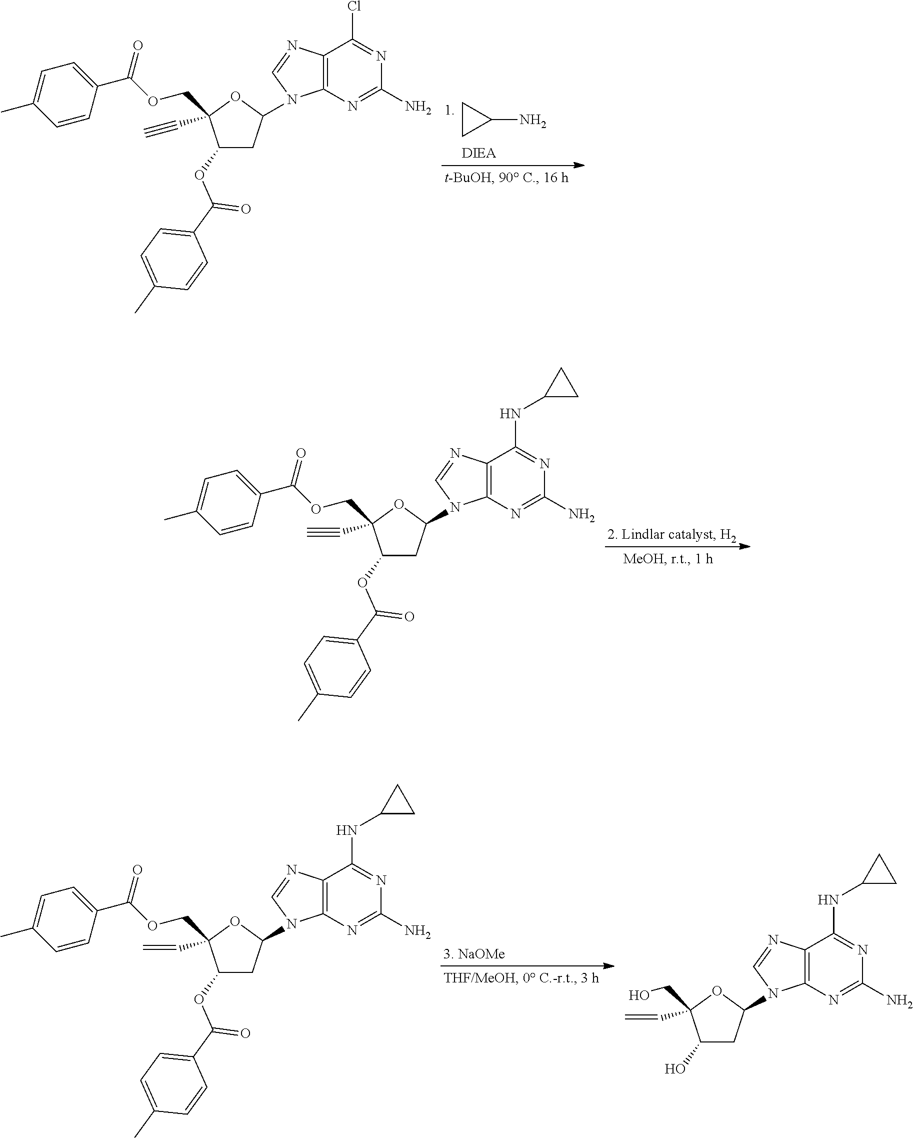

(2R,3S,5R)-5-(2-amino-6-(cyclopropylamino)-9H-purin-9-yl)-2-ethynyl-2-(hydroxymethyl)tetrahydrofuran-3-ol was synthesized according the procedure described in WO 2007/038507 A2 using N⁶-cyclopropyl-9H-purine-2,6-diamine.

Step 1. To a mixture of [(2R,3S)-5-(2-amino-6-chloro-purin-9-yl)-2-ethynyl-3-(4-methylbenzoyl)oxy-tetrahydrofuran-2-yl]methyl 4-methylbenzoate (0.20 g, 366.32 μmol) and cyclopropanamine (31 mg, 549.48 μmol) in t-BuOH (15 mL) was added DIEA (95 mg, 732.64 μmol) in one portion at 25° C. under N₂. The resulting mixture was stirred at 90° C. for 16 hours. After cooling to room temperature, the reaction mixture was concentrated under reduced pressure. The residue was purified by pre-TLC (50% EtOAc in petroleum ether) to afford the compound (0.04 g, 19%) as a colorless oil. ¹H NMR (400 MHz, CDCl₃): δ 8.05-7.97 (d, J=8.4 Hz, 2H), 7.90 (d, J=8.0 Hz, 2H), 7.59 (s, 1H), 7.30 (s, 2H), 7.21 (d, J=7.6 Hz, 2H), 6.43-6.37 (m, 1H), 6.20-6.13 (m, 1H), 5.04 (d, J=11.6 Hz, 1H), 4.59 (d, J=11.6 Hz, 1H), 3.42-3.33 (m, 1H), 3.13-2.97 (m, 1H), 2.81-2.72 (m, 1H), 2.68 (s, 1H), 2.43 (d, J=16.4 Hz, 6H), 0.96-0.85 (m, 2H), 0.67-0.60 (m, 2H).

Step 2. To a solution of [(2R, 3S, 5R)-5-[2-amino-6-(cyclopropylamino)purin-9-yl]-2-ethynyl-3-(4-methylbenzoyl)oxy-tetrahydrofuran-2-yl]methyl 4-methylbenzoate (0.02 g, 35.30 μmol) in MeOH (2 mL) was added Lindlar catalyst (7 mg) in one portion at 25° C. under H₂ (15 psi). The resulting mixture was stirred at 25° C. for 1 hour. Then the mixture was filtered and concentrated to afford the compound (0.02 g, 99%) as a white solid.

Step 3. To a solution of [(2R,3S,5R)-5-[2-amino-6-(cyclopropylamino)purin-9-yl]-3-(4-methylbenzoyl)oxy-2-vinyl-tetrahydrofuran-2-yl]methyl 4-methylbenzoate (0.02 g, 35.17 μmol) in MeOH (5 mL) was added NaOMe (190 μg, 3.52 μmol) in one portion at 25° C. and stirred for 18 hours. Then the mixture was concentrated. The residue was purified by prep-HPLC (acetonitrile 4-34%/0.05% NH₃·H₂O in water) to afford the title compound (14 mg, 53%) as a white solid. ¹H NMR (400 MHz, DMSO-d6) δ 7.95 (s, 1H), 7.37 (s, 1H), 6.17 (t, J=6.4 Hz, 1H), 6.03-5.90 (m, 1H), 5.81 (s, 2H), 5.43 (s, 1H), 5.37-5.31 (m, 1H), 5.22 (s, 1H), 5.20-5.15 (m, 1H), 4.58 (t, J=6.4 Hz, 1H), 3.55-3.47 (m, 1H), 3.45-3.38 (m, 1H), 2.57-2.52 (m, 1H), 2.22-2.14 (m, 1H), 0.69-0.62 (m, 2H), 0.62-0.55 (m, 2H).

### Example 12

### Synthesis of 4-amino-5-fluoro-1-((2R,4S,5R)-4-hydroxy-5-(hydroxymethyl)-5-vinyltetrahydrofuran-2-yl)pyrimidin-2(1H)-one (Cpd. No. 8)

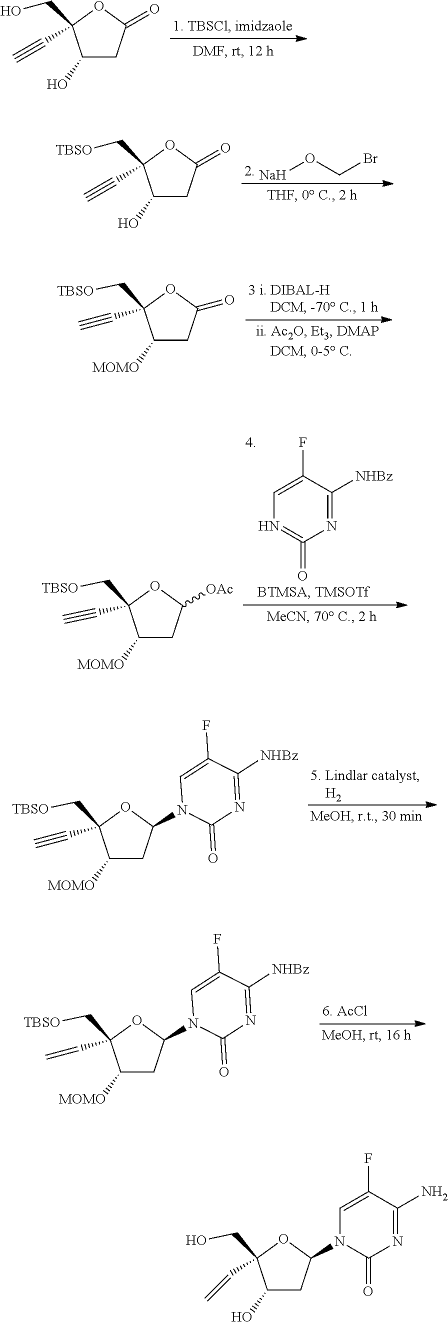

(4S,5R)-5-ethynyl-4-hydroxy-5-(hydroxymethyl)tetrahydrofuran-2-one was synthesized according the procedure described in WO 2007/038507 A2.

Step 1. To a solution of (4S,5R)-5-ethynyl-4-hydroxy-5-(hydroxymethyl)tetrahydrofuran-2-one (1 g, 6.4 mmol) and imidazole (1.31 g, 19.2 mmol) in DMF (30 mL) was added TBSCl (1.06 g, 7.1 mmol) at 20° C. The resulting mixture was stirred at 20° C. for 16 hours. After that, the reaction was diluted with EtOAc (80 mL) and washed with brine (50 mL×2). The layers were separated, the organic layer was concentrated. The residue was purified by column chromatography on silica gel (20% to 30% EtOAc in petroleum ether) to afford the compound (0.7 g, 40%) as a white solid. ¹H NMR (400 MHz, CDCl₃) δ 4.53-4.50 (m, 1H), 3.95-3.87 (m, 2H), 2.99-2.93 (m, 1H), 2.83 (s, 1H), 2.61-2.55 (m, 1H), 2.39-2.37 (m, 1H), 0.89 (s, 9H), 0.10 (s, 6H).

Step 2. To a solution of (4S,5R)-5-(((tert-butyldimethylsilyl)oxy)methyl)-5-ethynyl-4 hydroxydihydrofuran-2(3H)-one (3 g, 11 mmol) in THF (50 mL) was added NaH (665 mg, 16 mmol, 60% dispersion in mineral oil) at 0° C. After stirring 0° C. for 10 min, MOMBr (2.77 g, 22 mmol) was added. The resulting mixture was stirred at 25° C. for additional 3 hours. After that, the reaction mixture was poured into water (50 mL) and extracted with DCM (50 mL×2). The organic layer was washed with brine (50 mL×2), concentrated. The residue was purified by column chromatography on silica gel (10% to 20% EtOAc in petroleum ether) to afford the compound (2.1 g, 60%) as a colorless oil. ¹H NMR (400 MHz, CDCl₃) δ 4.81-4.69 (m, 2H), 4.56-4.53 (m, 1H), 3.95-3.85 (m, 2H), 3.42 (s, 3H), 2.97-2.91 (m, 1H), 2.72 (s, 1H), 2.68-2.62 (m, 1H), 0.89 (s, 9H), 0.10 (s, 6H).

Step 3. To a solution of (4S,5R)-5-[[tert-butyl(dimethyl)silyl]oxymethyl]-5-ethynyl-4-(methoxymethoxy)tetrahydrofuran-2-one (1.1 g, 3.5 mmol) in DCM (30 mL) was added DIBAL-H (4.2 mL, 4.2 mmol) dropwise at −70° C. The mixture was stirred for 30 min. After that, the reaction was quenched with methanol (5 mL), washed with aqueous citric acid solution (10 wt %, 30 mL) and brine (30 mL×2), concentrated to give 1.1 g crude (4S,5R)-5-[[tert-butyl(dimethyl)silyl]oxymethyl]-5-ethynyl-4-(methoxymethoxy)tetrahydrofuran-2-ol as a yellow oil. It was dissolved in DCM (20 mL). Et₃N (457 mg, 4.52 mmol), DMAP (42 mg) add Ac₂O (426 mg, 4.17 mmol) were added sequentially at 0° C. After stirring at 25° C. for 1 hour, the reaction mixture was diluted with MTBE (100 mL), washed with aqueous citric acid solution (10 wt %, 50 mL) and brine (50×2 mL), concentrated under reduce pressure. The residue was purified by column chromatography on silica gel (10% to 30% EtOAc in petroleum ether) to afford the compound (0.9 g, 72%) as a white solid.

Step 4. A mixture of N-(5-fluoro-2-hydroxy-pyrimidin-4-yl)benzamide (100 mg, 0.43 mmol) and BTMSA (219 mg, 1.29 mmol) in MeCN (10 mL) was stirred at 70° C. for 1 hour, then cooled to 20° C., TMSOTf (148 mg, 0.66 mmol) and a solution of [(4S,5R)-5-[[tert-butyl(dimethyl)silyl]oxymethyl]-5-ethynyl-4-(methoxymethoxy)tetrahydrofuran-2-yl]acetate (0.12 g, 0.33 mmol) in MeCN (5 mL) were added sequentially. After stirring at 20° C. for 3 hours, the reaction mixture was poured into water (50 mL), extracted with EtOAc (50 mL×2), concentrated. The residue was purified by pre-TLC (30% EtOAc in petroleum ether) to afford the compound (40 mg, 22%) as a white solid. ¹H NMR (400 MHz, CDCl₃) δ 13.10 (s, 1H), 8.30-8.19 (m, 31H), 7.58-7.54 (m, 1H), 7.48-7.44 (m, 2H), 6.28-6.26 (m, 1H), 4.75-4.71 (m, 2H), 4.43 (t, J=7.6 Hz, 1H), 4.10-4.07 (m, 1H), 3.94-3.91 (m, 1H), 3.42 (s, 3H), 2.76-2.71 (m, 1H), 2.67 (s, 1H), 2.40-2.35 (m, 1H), 0.97 (s, 9H), 0.18 (s, 6H). LCMS (ESI): m/z 532.5 (M+H)+.

Step 5. A mixture of N-[1-[(2R,4S,5R)-5-[[tert-butyl(dimethyl)silyl]oxymethyl]-5-ethynyl-4-(methoxymethoxy)tetrahydrofuran-2-yl]-5-fluoro-2-oxo-pyrimidin-4-yl]benzamide (10 mg, 18 μmol) and Lindlar catalyst (10 mg) in MeOH (5 mL) was stirred at 20° C. for 2 hours under H₂ (15 psi). The reaction mixture was filtrated and concentrated to afford the compound (10 mg, 99%) as a white solid. LCMS (ESI): m/z 534.1 (M+H)+

Step 6. To a solution of N-[1-[(2R,4S,5R)-5-[[tert-butyl(dimethyl)silyl]oxymethyl]-4-(methoxymethoxy)-5-vinyl-tetrahydrofuran-2-yl]-5-fluoro-2-oxo-pyrimidin-4-yl]benzamide (80 mg, 0.15 mmol) in MeOH (5 mL) was added acetyl chloride (118 mg, 1.5 mmol) at 20° C. The resulting mixture was stirred at 20° C. for 16 hours. After that the reaction mixture was filtered and concentrated. The residue was purified by prep-HPLC (FA) to afford the title compound (6.7 mg, 16%) as a white solid. ¹H NMR (400 MHz, DMSO-d₆) δ 8.32 (d, J=7.6 Hz, 1H), 7.70 (s, 1H), 7.46 (s, 1H), 6.00-5.97 (m, 1H), 5.93-5.85 (m, 1H), 5.38-5.31 (m, 2H), 5.23-5.18 (m, 2H), 4.40 (t, J=7.6 Hz, 1H), 3.60-3.56 (m, 1H), 3.40-3.37 (m, 1H), 2.13-2.04 (m, 1H). LCMS (ESI): m/z 294.0 (M+Na)⁺.

### Example 13

### Synthesis of (2R,3S,5R)-5-(6-(cyclopropylamino)-9H-purin-9-yl)-2-ethyl-2-(hydroxymethyl)tetrahydrofuran-3-ol (Cpd. No. 96)

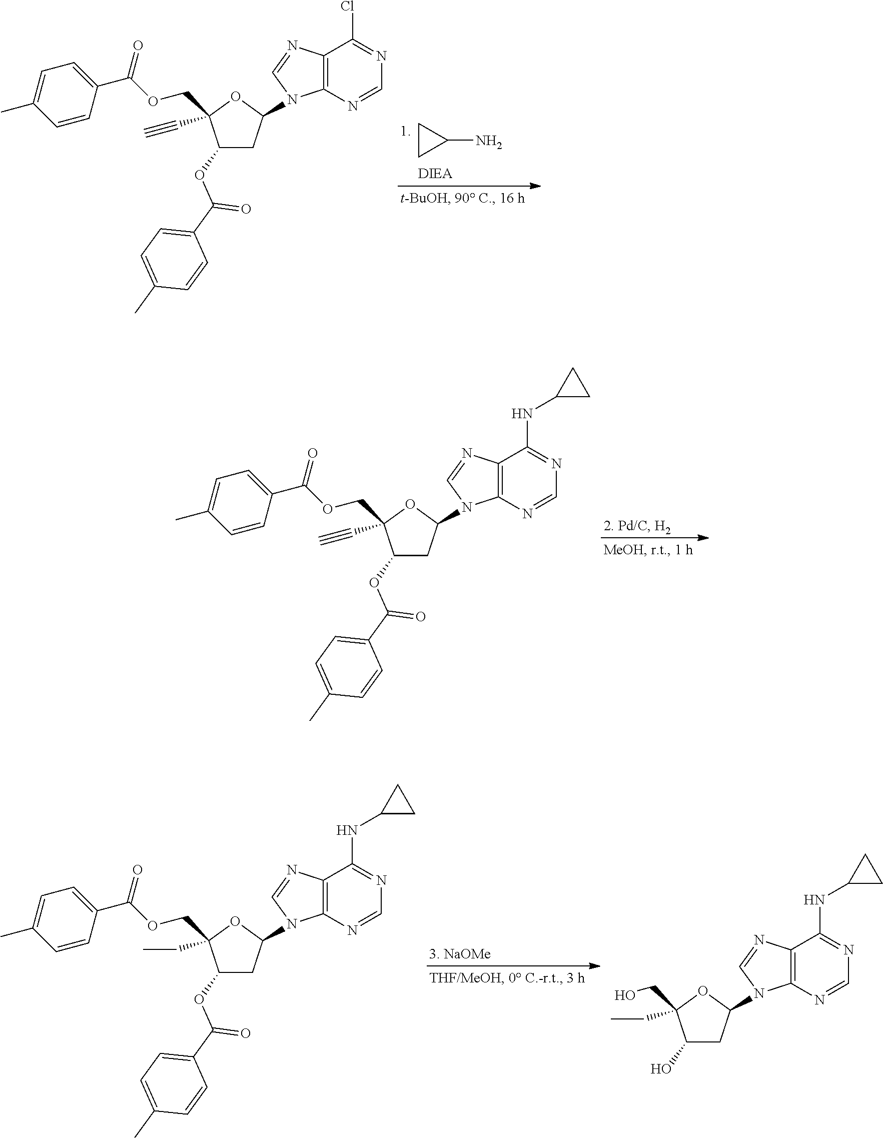

((2R,3S,5R)-5-(6-Chloro-9H-purin-9-yl)-2-ethynyl-2-(((4-methylbenzoyl)oxy)methyl)tetrahydrofuran-3-yl 4-methylbenzoate was synthesized according the procedure described in WO 2007/038507 A2 using N-cyclopropyl-9H-purin-6-amine.

Step 1. To a mixture of [(2R,3S,5R)-5-(6-chloropurin-9-yl)-2-ethynyl-3-(4-methylbenzoyl)oxy-tetrahydrofuran-2-yl]methyl 4-methylbenzoate (0.05 g, 94.17 μmol) and cyclopropanamine (8 mg, 141.25 μmol) in t-BuOH (2 mL) was added DIEA (24 mg, 188.34 μmol) in one portion at 25° C. under N₂. The resulting mixture was stirred at 90° C. for 16 hours. After cooling to room temperature, the mixture was concentrated under reduced pressure. The residue was purified by pre-TLC (50% EtOAc in petroleum ether) to afford the compound (45 mg, 87%) as a colorless oil. ¹H NMR (400 MHz, CDCl₃) δ 8.66 (s, 1H), 8.24 (s, 1H), 8.06-8.00 (m, 2H), 7.87-7.80 (m, 2H), 7.30 (d, J=8.0 Hz, 2H), 7.19 (d, J=8.0 Hz, 2H), 6.65-6.55 (m, 1H), 6.21-6.13 (m, 1H), 4.91 (d, J=12.0 Hz, 1H), 4.63 (d, J=12.0 Hz, 1H), 3.47-3.30 (m, 1H), 3.00-2.90 (m, 1H), 2.74 (s, 1H), 2.44 (d, J=16.0 Hz, 6H).

Step 2. To a mixture of (2R,3S,5R)-5-(6-(cyclopropylamino)-9H-purin-9-yl)-2-ethynyl-2-(((4-methylbenzoyl)oxy)methyl)tetrahydrofuran-3-yl 4-methylbenzoate (0.07 g, 126.91 μmol) in MeOH (5 mL) was added 10% Palladium on carbon (2 mg) in one portion at 25° C. under H₂ (15 psi). The resulting mixture was stirred at 25° C. for 1 hour. Then the mixture was filtered and concentrated directly to afford the compound (0.07 g, 99%) as a colorless oil.

Step 3. To a mixture of [(2R,3S,5R)-5-[6-(cyclopropylamino)purin-9-yl]-2-ethyl-3-(4-methylbenzoyl)oxy-tetrahydrofuran-2-yl]methyl 4-methylbenzoate (0.07 g, 125.98 μmol) in MeOH (5 mL) was added NaOMe (681 μg, 12.60 μmol) in one portion at 25° C. After stirring at 25° C. for 18 hours, the mixture was concentrated under reduced pressure. The residue was purified by prep-HPLC (acetonitrile 5-35%/0.05% NH₃·H₂O+10 mM NH₄HCO₃ in water) to afford the title compound (10 mg, 49%) as a white solid. ¹H NMR (400 MHz, DMSO-d6) δ 8.34 (s, 1H), 8.23 (s, 1H), 7.94 (s, 1H), 6.35-6.27 (m, 1H), 5.25-5.12 (m, 2H), 4.46-4.36 (m, 1H), 3.60-3.49 (m, 1H), 3.46-3.39 (m, 1H), 2.94-2.84 (m, 1H), 2.31-2.22 (m, 1H), 1.72-1.51 (m, 2H), 0.88 (t, J=7.5 Hz, 3H), 0.75-0.68 (m, 2H), 0.64-0.56 (m, 2H).

### Example 14

### Synthesis of (2R,3S,5R)-5-(2-amino-6-(cyclopropylamino)-9H-purin-9-yl)-2-ethyl-2-(hydroxymethyl)tetrahydrofuran-3-ol (Cpd. No. 97)

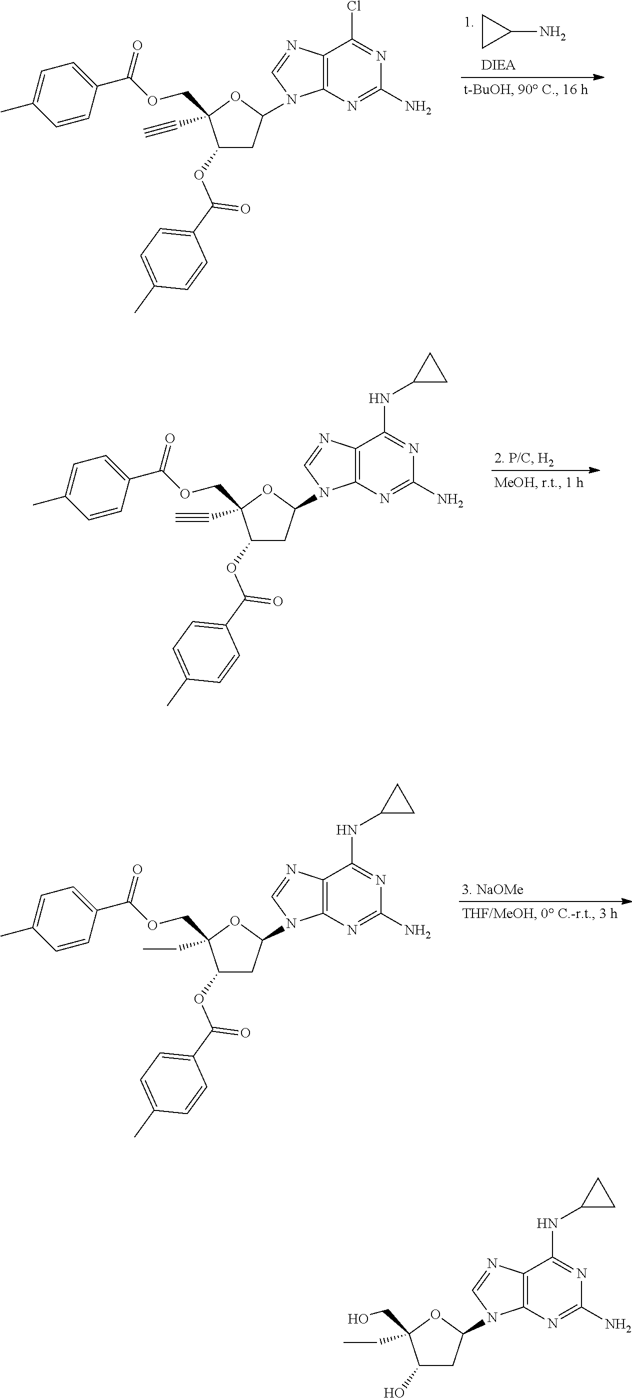

(2R,3S,5R)-5-(2-amino-6-(cyclopropylamino)-9H-purin-9-yl)-2-ethynyl-2-(hydroxymethyl)tetrahydrofuran-3-ol was synthesized according the procedure described in WO 2007/038507 A2 using N6-cyclopropyl-9H-purine-2,6-diamine.

Step 1. To a mixture of [(2R,3S)-5-(2-amino-6-chloro-purin-9-yl)-2-ethynyl-3-(4-methylbenzoyl)oxy-tetrahydrofuran-2-yl]methyl 4-methylbenzoate (0.20 g, 366.32 μmol) and cyclopropanamine (31 mg, 549.48 μmol) in t-BuOH (15 mL) was added DIEA (95 mg, 732.64 μmol) in one portion at 25° C. under N₂. The resulting mixture was stirred at 90° C. for 16 hours. After cooling to room temperature, the reaction mixture was concentrated under reduced pressure. The residue was purified by pre-TLC (50% EtOAc in petroleum ether) to afford the compound (0.04 g, 19%) as a colorless oil. ¹H NMR (400 MHz, CDCl₃): δ 8.05-7.97 (d, J=8.4 Hz, 2H), 7.90 (d, J=8.0 Hz, 2H), 7.59 (s, 1H), 7.30 (s, 2H), 7.21 (d, J=7.6 Hz, 2H), 6.43-6.37 (m, 1H), 6.20-6.13 (m, 1H), 5.04 (d, J=11.6 Hz, 1H), 4.59 (d, J=11.6 Hz, 1H), 3.42-3.33 (m, 1H), 3.13-2.97 (m, 1H), 2.81-2.72 (m, 1H), 2.68 (s, 1H), 2.43 (d, J=16.4 Hz, 6H), 0.96-0.85 (m, 2H), 0.67-0.60 (m, 2H).

Step 2. To a solution of [(2R,3S,5R)-5-[2-amino-6-(cyclopropylamino)purin-9-yl]-2-ethynyl-3-(4-methylbenzoyl)oxy-tetrahydrofuran-2-yl]methyl 4-methylbenzoate (0.02 g, 35.30 μmol) in MeOH (5 mL) was added 10% Palladium on carbon (24 mg) in one portion at 25° C. under H₂ (15 psi). The resulting mixture was stirred at 25° C. for 1 hour. Then the mixture was filtered and concentrated to afford the compound (0.02 g, 99%) as a white solid.

Step 3. To a solution of [(2R,3S,5R)-5-[2-amino-6-(cyclopropylamino)purin-9-yl]-2-ethyl-3-(4-methylbenzoyl)oxy-tetrahydrofuran-2-yl]methyl 4-methylbenzoate (0.02 g, 35.05 μmol) in MeOH (5 mL) was added NaOMe (189 μg, 3.50 μmol) in one portion at 25° C. The resulting mixture was stirred at 25° C. for 18 hours to afford the title compound. ¹H NMR (400 MHz, DMSO-d6) δ 7.91 (s, 1H), 7.34 (s, 1H), 6.21-6.10 (m, 1H), 5.80 (s, 2H), 5.17-5.26 (m, 1H), 5.10 (d, J=4.4 Hz, 1H), 4.43-4.30 (m, 1H), 3.52-3.45 (m, 1H), 3.44-3.39 (m, 1H), 2.81-2.69 (m, 1H), 2.23-2.13 (m, 1H), 1.69-1.49 (m, 2H), 0.86 (t, J=7.6 Hz, 3H), 0.70-0.63 (m, 2H), 0.61-0.54 (m, 2H).

### Example 15

### Synthesis of 1-((2R,4S,5R)-5-ethyl-4-hydroxy-5-(hydroxymethyl)tetrahydrofuran-2-yl)-4-hydroxy-2-oxo-1,2-dihydropyrimidine-5-carbonitrile (Cpd. No. 98)

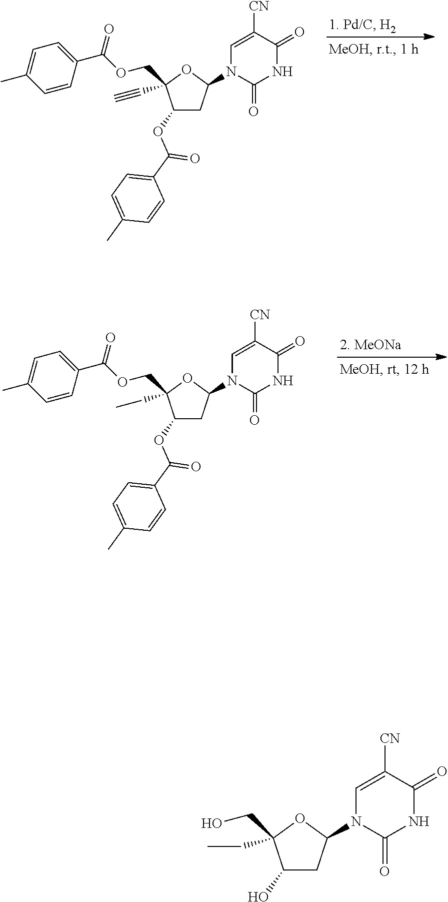

[(2R,3S,5R)-5-(5-Cyano-2,4-dioxo-pyrimidin-1-yl)-2-ethynyl-3-(4-methylbenzoyl)oxy-tetrahydrofuran-2-yl]methyl 4-methylbenzoate was synthesized according the procedure described in WO 2007/038507 A2.

Step 1. To a solution of [(2R,3S,5R)-5-(5-cyano-2,4-dioxo-pyrimidin-1-yl)-2-ethynyl-3-(4-methylbenzoyl)oxy-tetrahydrofuran-2-yl]methyl 4-methylbenzoate (5 mg, 97.37 μmol) in MeOH (3 mL) was added 10% palladium on carbon (10 mg, 83.33 μmol) in one portion at 25° C. under H₂ (15 psi). After stirring at 25° C. for 1 hour, the mixture was filtered and concentrated to afford the compound (25 mg, 50%) as a white oil.

Step 2. To a solution of [(2R,3S,5R)-5-(5-cyano-2,4-dioxo-pyrimidin-1-yl)-2-ethyl-3-(4-methylbenzoyl)oxy-tetrahydrofuran-2-yl]methyl 4-methylbenzoate (50 mg, 96.61 μmol) in MeOH (5 mL) was added NaOMe (521.94 μg, 9.66 μmol) in one portion at 25° C. After stirring at 25° C. for 1 hour, the mixture was concentrated. The residue was purified by prep-HPLC (acetonitrile 3-33%/0.225% formic acid in water) to afford the title compound (19 mg, 71%) as a white solid. ¹H NMR (400 MHz, DMSO-d₆) δ 8.91 (s, 1H), 5.94 (t. J=6.0 Hz, 1H), 5.32-5.25 (m, 1H), 5.17-5.12 (m, 1H), 4.31-4.23 (m, 1H), 3.60-3.52 (m, 1H), 3.45-3.39 (m, 1H), 2.37-2.22 (m, 2H), 1.65-1.55 (m, 1H), 1.54-1.39 (m, 1H), 0.86 (t, J=7.6 Hz, 3H).

### Example 16

### Synthesis of (9-((2R,4S,5R)-5-ethyl-4-hydroxy-5-(hydroxymethyl)tetrahydrofuran-2-yl)-9H-purin-6-ol (Cpd. No. 1)

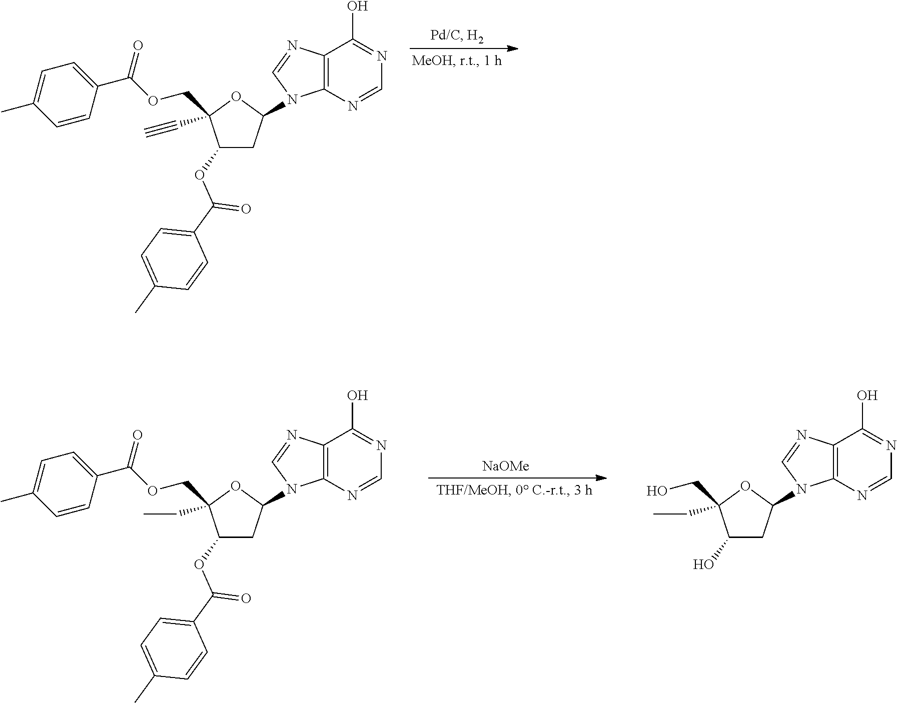 [SMILES: C#C[C@]1(COC(=O)c2ccc(C)cc2)O[C@@H](n2cnc3c(O)ncnc32)C[C@@H]1OC(=O)c1ccc(C)cc1 ; "Pd/C, H2" ; CC[C@]1(COC(=O)c2ccc(C)cc2)O[C@@H](n2cnc3c(O)ncnc32)C[C@@H]1OC(=O)c1ccc(C)cc1 ; CC[C@]1(CO)O[C@@H](n2cnc3c(O)ncnc32)C[C@@H]1O ; "NaOMe"]

[(2R,3S,5R)-2-Ethynyl-5-(6-hydroxypurin-9-yl)-3-(4-methylbenzoyl)oxy-tetrahydrofuran-2-yl]methyl 4-methylbenzoate was synthesized according the procedure described in WO 2007/038507 A2 using 9H-purin-6-ol.

Step 1. A mixture of [(2R,3S,5R)-2-ethynyl-5-(6-hydroxypurin-9-yl)-3-(4-methylbenzoyl)oxy-tetrahydrofuran-2-yl]methyl 4-methylbenzoate (50 mg, 97 μmol) and Lindlar catalyst (20 mg) in MeOH (5 mL) was stirred at 20° C. for 30 min under H₂ (15 psi). After that, the reaction mixture was filtrated and concentrated to afford the compound (40 mg, 79%) as a white solid.

Step 2. To a solution of [(2R,3S,5R)-2-ethyl-5-(6-hydroxypurin-9-yl)-3-(4-methylbenzoyl)oxy-tetrahydrofuran-2-yl]methyl 4-methylbenzoate (40 mg, 77 μmol) in THF (3 mL) was added NaOMe (2 mg, 38 μmol) in MeOH (2 mL) at 0° C. The resulting mixture was stirred at 25° C. for 16 hours. Then the reaction mixture was concentrated. The residue was purified by prep-HPLC (basic) to afford the title compound (4.3 mg, 20%) as a white solid. ¹H NMR (400 MHz, DMSO-d6) δ 8.22 (s, 1H), 7.99 (s, 1H), 6.26-6.23 (m, 1H), 4.41-4.38 (m, 1H), 3.52-3.33 (m, 2H), 2.83-2.76 (m, 1H), 2.30-2.24 (m, 1H), 1.68-1.53 (m, 2H), 0.87 (t, J=7.2 Hz, 3H). LCMS (ESI): m/z 302.9 (M+Na)+.

### Example 17

### Synthesis of (2R,3S,5R)-5-(6-(cyclopropylamino)-2-fluoro-9H-purin-9-yl)-2-ethyl-2-(hydroxymethyl)tetrahydrofuran-3-ol (Cpd. No. 99)

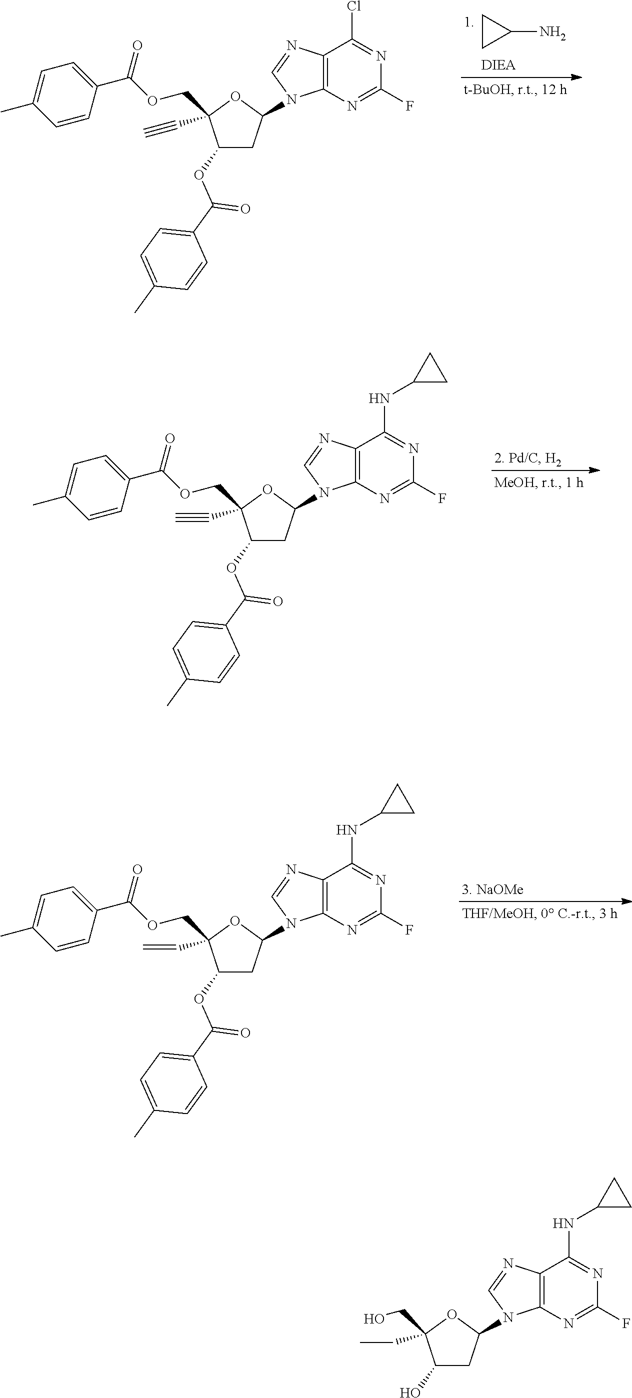

[(2R,3S,5R)-5-(6-Chloro-2-fluoro-purin-9-yl)-2-ethynyl-3-(4-methylbenzoyl)oxy-tetrahydrofuran-2-yl]methyl 4-methylbenzoate was synthesized according the procedure described in WO 2007/038507 A2 using N-cyclopropyl-2-fluoro-9H-purin-6-amine.

Step 1. To a solution of [(2R,3S,5R)-5-(6-chloro-2-fluoro-purin-9-yl)-2-ethynyl-3-(4-methylbenzoyl)oxy-tetrahydrofuran-2-yl]methyl 4-methylbenzoate (200 mg, 0.364 mmol) in t-BuOH (2 mL) was added DIPEA (63 μl, 0.364 mmol) and cyclopropanamine (22 mg, 0.383 mmol). The reaction mixture was stirred at 90° C. for 16 h. After cooling to room temperature, the mixture was diluted with water (10 mL), extracted with EtOAc (20 mL), washed with brine (20 mL×2), dried over Na₂SO₄ and concentrated. The residue was purified by was purified by column chromatography on silica gel (0-33% EtOAc in petroleum ether) to afford the compound (140 mg, 67%) as a white solid. ¹H NMR (400 MHz, CDCl₃) δ 8.02 (d, J=8.0 Hz, 2H), 7.93 (d, J=8.0 Hz, 2H), 7.85 (s, 1H), 7.29 (d, J=8.0 Hz, 2H), 7.23 (d, J=8.0 Hz, 2H), 6.51 (t, J=6.4 Hz, 1H), 6.08-6.02 (m, 1H), 4.83 (d, J=12.0 Hz, 1H), 4.66 (d, J=12.0 Hz, 1H), 3.23-3.16 (m, 1H), 3.04 (br s, 1H), 2.96-2.78 (m, 1H), 2.69 (s, 1H), 2.43 (d, J=15.2 Hz, 6H), 0.98-0.92 (m, 2H), 0.70-0.64 (m, 2H).

Step 2. To a mixture of [(2R,3S,5R)-5-[6-(cyclopropylamino)-2-fluoro-purin-9-yl]-2-ethynyl-3-(4-methylbenzoyl)oxy-tetrahydrofuran-2-yl]methyl 4-methylbenzoate (90 mg, 158.01 μmol) in EtOAc (5 mL) was added palladium on carbon (25 mg, 47.40 μmol, 20% purity) in one portion at 25° C. under H₂. The mixture was stirred at 25° C. for 16 h under H₂ (15 psi). After that, the reaction mixture was filtered and the filtrate was concentrated to afford the compound (75 mg, 83%) as a white solid.

Step 3. To a solution of [(2R,3S,5R)-5-[6-(cyclopropylamino)-2-fluoro-purin-9-yl]-2-ethyl-3-(4-methylbenzoyl)oxy-tetrahydrofuran-2-yl]methyl 4-methylbenzoate (70 mg, 122.03 μmol) in MeOH (3 mL) was added NH₃·H₂O (1 mL, 6.49 mmol, 25% purity) at 25° C. The resulting mixture was stirred at 40° C. for 16 h. After that, the reaction mixture was concentrated under reduced pressure. The residue was purified by reversed-phase HPLC (acetonitrile 1-27%/0.225% formic acid in water) to the title compound (6.5 mg, 16%) as a white solid. ¹H NMR (400 MHz, DMSO-d6) δ 8.53 (br s, 1H), 8.33 (s, 1H), 6.20 (t, J=6.8 Hz, 1H), 5.17 (d, J=4.8 Hz, 1H), 4.92 (t, J=5.2 Hz, 1H), 4.46-4.36 (m, 1H), 3.53-3.40 (m, 2H), 2.93 (br s, 1H), 2.84-2.76 (m, 1H), 2.36-2.23 (m, 1H), 1.70-1.49 (m, 2H), 0.92-0.83 (m, 3H), 0.75-0.61 (m, 4H).

### Example 18

### Synthesis of 9-((2R,4S,5R)-4-hydroxy-5-(hydroxymethyl)-5-vinyltetrahydrofuran-2-yl)-9H-purin-6-ol (Cpd. No. 100)

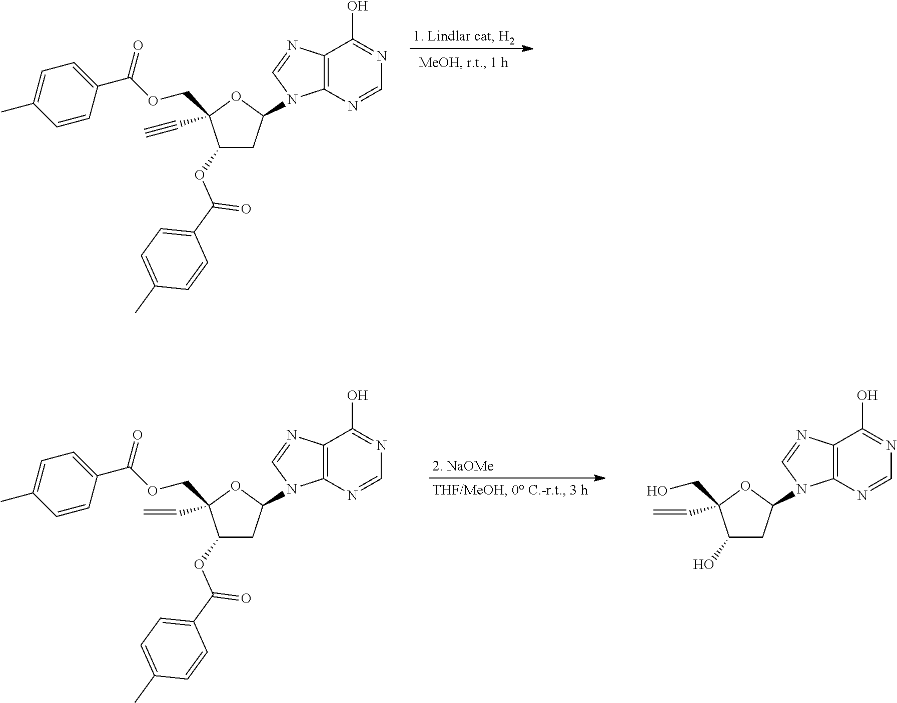

[(2R,3S,5R)-2-Ethynyl-5-(6-hydroxypurin-9-yl)-3-(4-methylbenzoyl)oxy-tetrahydrofuran-2-yl]methyl 4-methylbenzoate was synthesized according the procedure described in WO 2007/038507 A2 using 9H-purin-6-ol.

Step 1. A mixture of [(2R,3S,5R)-2-ethynyl-5-(6-hydroxypurin-9-yl)-3-(4-methylbenzoyl)oxy-tetrahydrofuran-2-yl]methyl 4-methylbenzoate (50 mg, 98 μmol) and Lindlar catalyst (20 mg) in MeOH (20 mL) was stirred at 20° C. for 30 min under H₂ (15 psi). Then the reaction mixture was filtrated. The filtrate was concentrated to afford the compound (35 mg, 69%) as a white solid.

Step 2. To a solution of [(2R,3S,5R)-5-(6-hydroxypurin-9-yl)-3-(4-methylbenzoyl)oxy-2-vinyl-tetrahydrofuran-2-yl]methyl 4-methylbenzoate (20 mg, 39 μmol) in THF (3 mL) was added NaOMe (1.1 mg, 19 μmol) in MeOH (2 mL) at 0° C. The resulting mixture was stirred at 25° C. for 16 hours. After that, the reaction mixture was concentrated. The residue was purified by prep-HPLC (basic) to afford the title compound (4 mg, 36%) as a white solid. ¹H NMR (400 MHz, DMSO-d6) δ 8.26 (s, 1H), 8.00 (s, 1H), 6.29-6.26 (m, 1H), 5.98-5.92 (m, 1H), 5.40-5.35 (m, 1H), 5.24-5.18 (m, 1H), 4.62 (t, J=6.4 Hz, 1H) 3.52-3.33 (m, 2H), 2.62-2.57 (m, 1H), 2.26-2.21 (m, 1H). LCMS (ESI): m/z 279.1 (M+H)+.

### Example 19

### Synthesis of 5-bromo-1-((2R,4S,5R)-5-ethyl-4-hydroxy-5-(hydroxymethyl)tetrahydrofuran-2-yl)-4-hydroxypyrimidin-2(1H)-one (Cpd. No. 2)

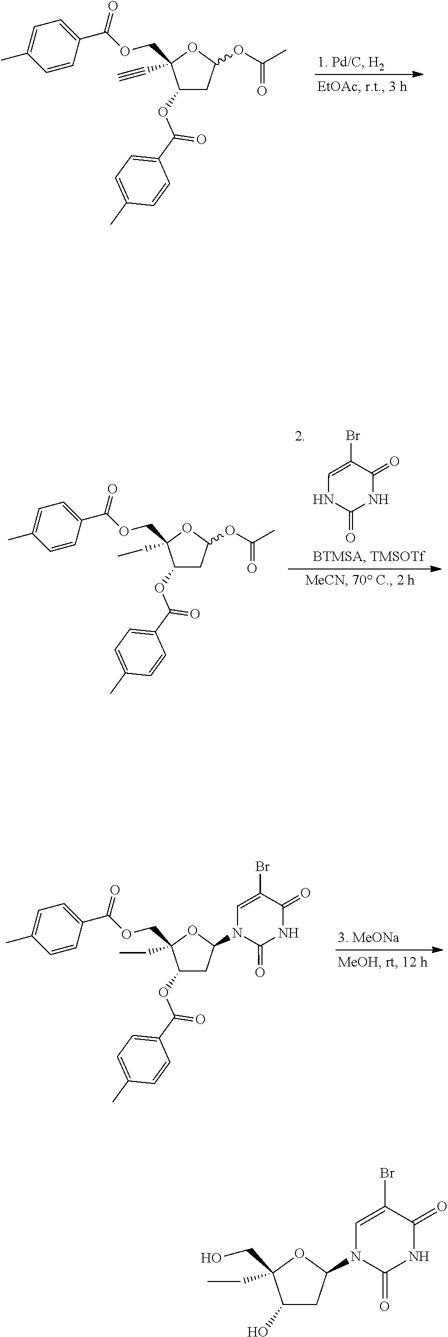

[(2R,3S)-5-Acetoxy-2-ethynyl-3-(4-methylbenzoyl)oxy-tetrahydrofuran-2-yl]methyl 4-methylbenzoate was synthesized according the procedure described in WO 2007/038507 A2.

Step 1. To a solution of [(2R,3S)-5-acetoxy-2-ethynyl-3-(4-methylbenzoyl)oxy-tetrahydrofuran-2-yl]methyl 4-methylbenzoate (200 mg, 458.24 μmol) in MeOH (10 mL) was added 10% palladium on carbon (9.00 mg) in one portion at 25° C. under H₂ (15 psi). The resulting mixture was stirred at 25° C. for 3 hours. After that, the reaction mixture was filtered and concentrated to afford the compound (0.2 g, 99%) as a white solid.

Step 2. To a solution of 5-bromo-1H-pyrimidine-2,4-dione (173.4 mg, 908.09 μmol) in MeCN (10 mL) was added BTMSA (309.5 mg, 1.82 mmol) in one portion at 25° C. under N₂. Then the mixture was stirred at 70° C. for 1 hour. After cooling to 25° C., TMSOTf (131.2 mg, 590.26 μmol) and a solution of [(2R,3S)-5-acetoxy-2-ethyl-3-(4-methylbenzoyl)oxy-tetrahydrofuran-2-yl]methyl 4-methylbenzoate (0.2 g, 454.05 μmol) in MeCN (5 mL) was added dropwise at 25° C. The resulting mixture was stirred at 25° C. for 1 hour. After that, the reaction was quenched by addition of 10 mL of aqueous citric acid solution (10 wt %), then extracted with EtOAc (20 mL). The layers was separated, the organic layer was washed with brine (10 mL), dried over Na₂SO₄, filtered and concentrated. The residue was purified by pre-TLC (50% EtOAc in petroleum ether) to afford the compound (52 mg, 20%) as a colorless oil. ¹H NMR (400 MHz, CDCl₃) δ 8.25 (s, 1H), 7.99-7.86 (m, 5H), 7.34-7.27 (m, 4H), 6.35-6.26 (m, 1H), 5.85-5.75 (m, 1H), 4.65-4.58 (m, 1H), 4.56-4.48 (m, 1H), 2.76-2.66 (m, 1H), 2.55-2.48 (m, 1H), 2.44 (d, J=8.8 Hz, 6H), 2.05-1.91 (m, 1H), 1.90-1.76 (m, 1H), 1.08 (t, J=7.6 Hz, 3H).

Step 3. To a solution of [(2R,3S,5R)-5-(5-bromo-2,4-dioxo-pyrimidin-1-yl)-2-ethyl-3-(4-methylbenzoyl)oxy-tetrahydrofuran-2-yl]methyl 4-methylbenzoate (0.02 g, 35.00 μmol) in MeOH (5 mL) was added NaOMe (189 μg, 3.50 μmol) in one portion at 25° C. The resulting mixture was stirred 25° C. for 1 hour. After that, the mixture was concentrated under reduced pressure. The residue was purified by prep-HPLC (acetonitrile 0-30%/0.225% formic acid in water) to afford the title compound (5.4 mg, 46%) as a white solid. ¹H NMR (400 MHz, DMSO-d6) δ 8.47 (s, 1H), 6.03 (t, J=6.4 Hz, 1H), 5.27-5.18 (m, 1H), 5.14 (d, J=4.8 Hz, 1H), 4.33-4.21 (m, 1H), 3.56-3.49 (m, 1H), 3.45-3.40 (m, 1H), 2.31-2.24 (m, 1H), 2.22-2.13 (m, 1H), 1.64-1.53 (m, 1H), 1.52-1.41 (m, 1H), 0.85 (t, J=7.6 Hz, 3H).

### Example 20

### Synthesis of (2R,3S,5R)-5-(2-amino-6-methoxy-9H-purin-9-yl)-2-(hydroxymethyl)-2-vinyltetrahydrofuran-3-ol (Cpd. No. 102)

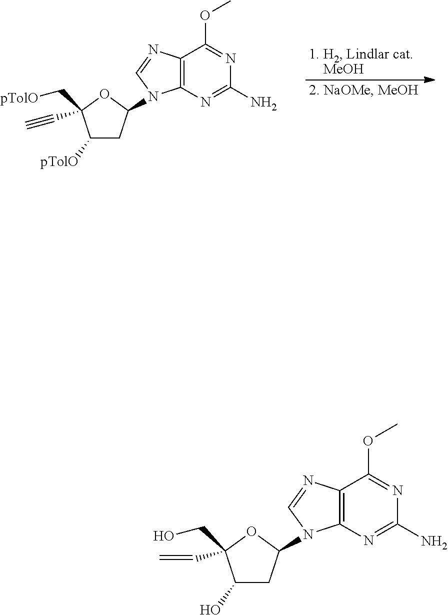

[(2R,3S,5R)-5-(2-Amino-6-methoxy-purin-9-yl)-2-ethynyl-3-(4-methylbenzoyl)oxy-tetrahydrofuran-2-yl]methyl 4-methylbenzoate was prepared according to WO 2007/038507 A2 using 6-methoxy-9H-purin-2-amine.

Step 1. To a solution of [(2R,3S,5R)-5-(2-amino-6-methoxy-purin-9-yl)-2-ethynyl-3-(4-methylbenzoyl)oxy-tetrahydrofuran-2-yl]methyl 4-methylbenzoate (20 mg, 36.93 μmol) in MeOH (2 mL) was added Lindlar catalyst (8 mg) in one portion at 25° C. under H₂ (15 psi). The resulting mixture was stirred at 25° C. for 1 hour. Then the mixture was filtered and the filtrate was concentrated to afford the compound (20 mg, 99%) as a white solid.

Step 2. To a mixture of [(2R,3S,5R)-5-(2-amino-6-methoxy-purin-9-yl)-3-(4-methylbenzoyl)oxy-2-vinyl-tetrahydrofuran-2-yl]methyl 4-methylbenzoate (17 mg, 31.27 μmol) in MeOH (5 mL) was added NaOMe (169 μg, 3.13 μmol) in one portion at 25° C. After stirring at 25° C. for 1 hour, the mixture was concentrated under reduced pressure. The residue was purified by prep-HPLC (basic) to afford the title compound (2.5 mg, 26%) as a white solid. LCMS (ESI): m/z 308.2 (M+H)+. ¹H NMR (400 MHz, CD₃OD) δ 8.61 (s, 1H), 6.46-6.39 (m, 1H), 6.06-6.95 (m, 1H), 5.62-5.51 (m, 1H), 5.38-5.31 (m, 1H), 4.65 (t, J=7.6 Hz, 1H), 4.08 (s, 3H), 3.76-3.67 (m, 1H), 3.61-3.56 (m, 1H), 2.53-2.39 (m, 2H).

### Example 21

### Synthesis of (2R,3S,5R)-5-(2-amino-6-methoxy-9H-purin-9-yl)-2-ethyl-2-(hydroxymethyl)tetrahydrofuran-3-ol (Cpd. No. 125)

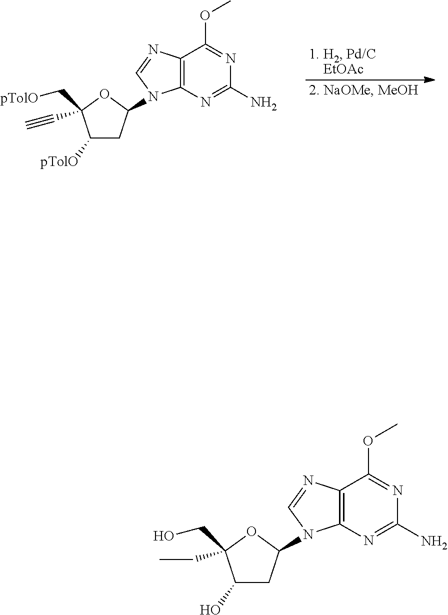

[(2R,3S,5R)-5-(2-amino-6-methoxy-purin-9-yl)-2-ethynyl-3-(4-methylbenzoyl)oxy-tetrahydrofuran-2-yl]methyl 4-methylbenzoate was prepared according to WO 2007/038507 A2 using 6-methoxy-9H-purin-2-amine.

Step 1. To a mixture of [(2R,3S,5R)-5-(2-amino-6-methoxy-purin-9-yl)-2-ethynyl-3-(4-methylbenzoyl)oxy-tetrahydrofuran-2-yl]methyl 4-methylbenzoate (0.1 g, 184.65 μmol) in EtOAc (5 mL) was added Pd/C (60 mg, 10% purity) under H₂ (15 psi). The mixture was stirred at 25° C. for 3 hours. After that, the mixture was filtered and concentrated to afford the title compound (80 mg, 79% yield) as a white solid.

Step 2. To a mixture of [(2R,3S,5R)-5-(2-amino-6-methoxy-purin-9-yl)-2-ethyl-3-(4-methylbenzoyl)oxy-tetrahydrofuran-2-yl]methyl 4-methylbenzoate (80 mg, 146.63 μmol) in MeOH (5 mL) was added NaOMe (1 mg, 14.66 μmol) in MeOH (2 mL) at 0° C. The resulting mixture was stirred at 25° C. for 16 hours. Then the reaction mixture was concentrated. The residue was purified by prep-HPLC (basic) to afford the title compound (7 mg, 15%) as a white solid. ¹H NMR (400 MHz, CD₃OD): δ 8.50 (s, 1H), 6.41 (t, J=6.4 Hz, 1H), 4.53 (t, J=5.6 Hz, 1H), 4.08 (s, 3H), 3.70-3.64 (m, 1H), 3.61-3.56 (m, 1H), 2.63-2.50 (m, 2H), 1.85-1.73 (m, 1H), 1.71-1.60 (m, 1H), 1.01 (t, J=7.6 Hz, 3H).

### Example 22

### Synthesis of 1-((2R,4S,5R)-5-ethyl-4-hydroxy-5-(hydroxymethyl)tetrahydrofuran-2-yl)-5-methoxypyrimidine-2,4(1H,3H)-dione (Cpd. No. 103)

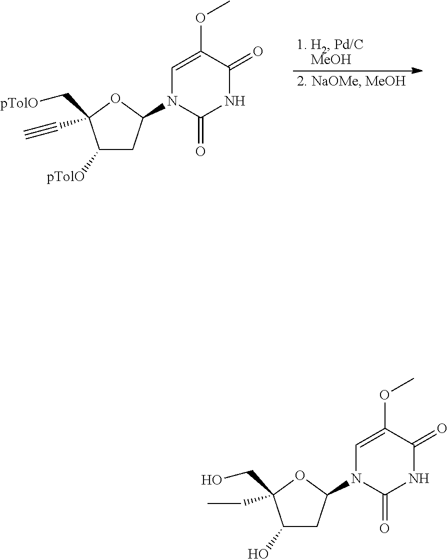

[(2R,3S,5R)-2-Ethynyl-5-(5-methoxy-2,4-dioxo-pyrimidin-1-yl)-3-(4-methylbenzoyl)oxy-tetrahydrofuran-2-yl]methyl 4-methylbenzoate was prepared according to WO 2007/038507 A2.

Step 1. To a mixture of [(2R,3S,5R)-2-ethynyl-5-(5-methoxy-2,4-dioxo-pyrimidin-1-yl)-3-(4-methylbenzoyl)oxy-tetrahydrofuran-2-yl]methyl 4-methylbenzoate (15 mg, 28.93 μmol) in MeOH (5 mL) 10% Palladium on carbon (9 mg) in one portion at 25° C. under H₂ (15 psi). The resulting mixture was stirred at 25° C. for 1 hour. Then the mixture was filtered and the filtrate was concentrated to afford the compound (15 mg, 99%) as a white solid.

Step 2. To a mixture of [(2R,3S,5R)-2-ethyl-5-(5-methoxy-2,4-dioxo-pyrimidin-1-yl)-3-(4-methylbenzoyl)oxy-tetrahydrofuran-2-yl]methyl 4-methylbenzoate (15 mg, 28.71 μmol) in MeOH (5 mL) was added NaOMe (155 μg, 2.87 μmol) in one portion at 25° C. After stirring at 25° C. for 18 hours, the mixture was concentrated under reduced pressure. The residue was purified by prep-HPLC (basic) to afford the title compound (2.3 mg, 28%) as a white solid. ¹H NMR (400 MHz, CD₃OD) δ 7.87 (s, 1H), 6.25 (t, J=6.4 Hz, 1H), 4.55-4.47 (m, 1H), 3.76-3.72 (m, 1H), 3.71 (s, 3H), 3.63-3.58 (m, 1H), 2.45-2.27 (m, 2H), 1.79-1.66 (m, 1H), 1.64-1.52 (m, 1H), 0.97 (t, J=7.6 Hz, 3H).

### Example 23

### Synthesis of 5-fluoro-1-((2R,4S,5R)-4-hydroxy-5-(hydroxymethyl)-5-vinyltetrahydrofuran-2-yl)pyrimidine-2,4(1H,3H)-dione (Cpd. No. 17)

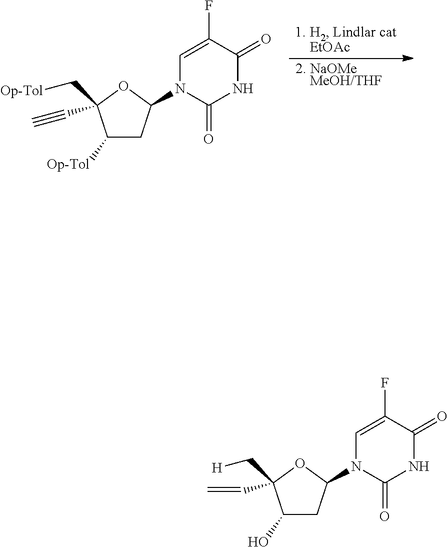

[(2R,3S,5R)-2-Ethynyl-5-(5-fluoro-4-hydroxy-2-oxo-pyrimidin-1-yl)-3-(4-methylbenzoyl)oxy-tetrahydrofuran-2-yl]methyl 4-methylbenzoate was prepared according to WO 2007/038507 A2 using 5-fluoropyrimidine-2,4(1H,3H)-dione.

Step 1. A mixture of [(2R,3S,5R)-2-ethynyl-5-(5-fluoro-4-hydroxy-2-oxo-pyrimidin-1-yl)-3-(4-methylbenzoyl)oxy-tetrahydrofuran-2-yl]methyl 4-methylbenzoate (30 mg, 59.23 μmol) and Lindlar catalyst (12 mg) in EtOAc (2 mL) was stirred at 20° C. for 10 min under H₂ (15 psi). After that, the reaction mixture was filtrated and the filtrate was concentrated to afford the compound (30 mg, 99%) as a white solid. LCMS (ESI): m/z 531.1 (M+Na)+

Step 2. A mixture of [(2R,3S,5R)-5-(5-fluoro-4-hydroxy-2-oxo-pyrimidin-1-yl)-3-(4-methylbenzoyl)oxy-2-vinyl-tetrahydrofuran-2-yl]methyl 4-methylbenzoate (30 mg, 59 μmol) and NaOMe (3.19 mg, 59 μmol) in MeOH (2 mL) and THF (3 mL) was stirred at 20° C. for 3 hours. After that, the reaction mixture was concentrated to give the crude product. It was purified by prep-HPLC (basic) to afford the title compound (4.3 mg, 27%) as a white solid. ¹H NMR (400 MHz, DMSO-d6) δ 8.26 (d, J=7.2 Hz, 1H), 6.06-6.04 (m, 1H), 5.90-5.80 (m, 1H), 5.48 (s, 1H), 5.35-5.30 (m, 2H), 5.20-5.17 (m, 1H), 4.42-4.31 (m, 1H), 3.68-3.33 (m, 2H), 2.21-2.04 (m, 2H). LCMS (ESI): m/z 295.0 (M+Na)+.

### Example 24

### Synthesis of 1-((2R,4S,5R)-5-ethyl-4-hydroxy-5-(hydroxymethyl)tetrahydrofuran-2-yl)-5-fluoropyrimidine-2,4(1H,3H)-dione (Cpd. No. 18)

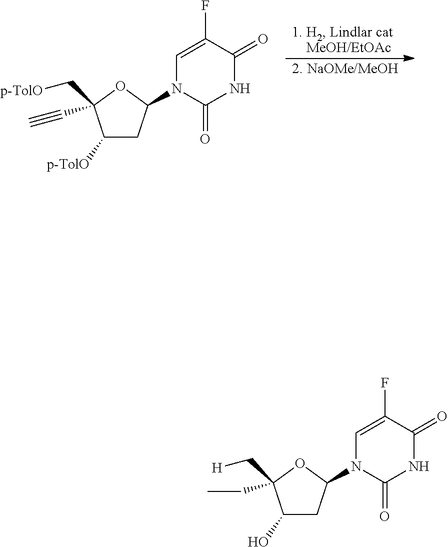

[(2R,3S,5R)-2-Ethynyl-5-(5-fluoro-4-hydroxy-2-oxo-pyrimidin-1-yl)-3-(4-methylbenzoyl)oxy-tetrahydrofuran-2-yl]methyl 4-methylbenzoate was prepared according to WO 2007/038507 A2 using 5-fluoropyrimidine-2,4(1H,3H)-dione.

Step 1. A mixture of [(2R,3S,5R)-2-ethynyl-5-(5-fluoro-4-hydroxy-2-oxo-pyrimidin-1-yl)-3-(4-methylbenzoyl)oxy-tetrahydrofuran-2-yl]methyl 4-methylbenzoate (50 mg, 98.72 μmol) and Lindlar catalyst (5 mg) in MeOH (5 mL) and EtOAc (5 mL) was stirred at 20° C. for 5 hours under H₂ (15 psi). After that, the reaction mixture was filtrated and the filtrate was concentrated to give the compound (40 mg, 79%) as a white solid. LCMS (ESI): m/z 533.1 (M+Na)+.

Step 2. A mixture of [(2R,3S,5R)-2-ethyl-5-(5-fluoro-4-hydroxy-2-oxo-pyrimidin-1-yl)-3-(4-methylbenzoyl)oxy-tetrahydrofuran-2-yl]methyl 4-methylbenzoate (40 mg, 78.35 μmol) and NaOMe (4.23 mg, 78.35 μmol) in MeOH (2 mL) and THF (3 mL) was stirred at 20° C. for 3 hours. Then the reaction mixture was concentrated to give the crude product. It was purified by prep-HPLC (basic) to afford the title compound (5.8 mg, 27%) as a white solid. ¹H NMR (400 MHz, DMSO-d6) δ 8.00 (d, J=7.2 Hz, 1H), 6.07 (t, J=6.8 Hz, 1H), 5.10-5.08 (m, 2H), 4.26 (t, J=6.0 Hz, 1H), 3.50-3.30 (m, 2H), 2.18-2.08 (m, 2H), 1.59-1.45 (m, 2H), 0.85 (t, J=7.6 Hz, 3H). LCMS (ESI): m/z 297.0 (M+Na)+.

### Example 25

### Synthesis of 4-amino-1-((2R,4R,5S)-5-ethynyl-4-hydroxy-5-(hydroxymethyl)tetrahydrofuran-2-yl)pyrimidin-2(1H)-one (Cpd. No. 77)

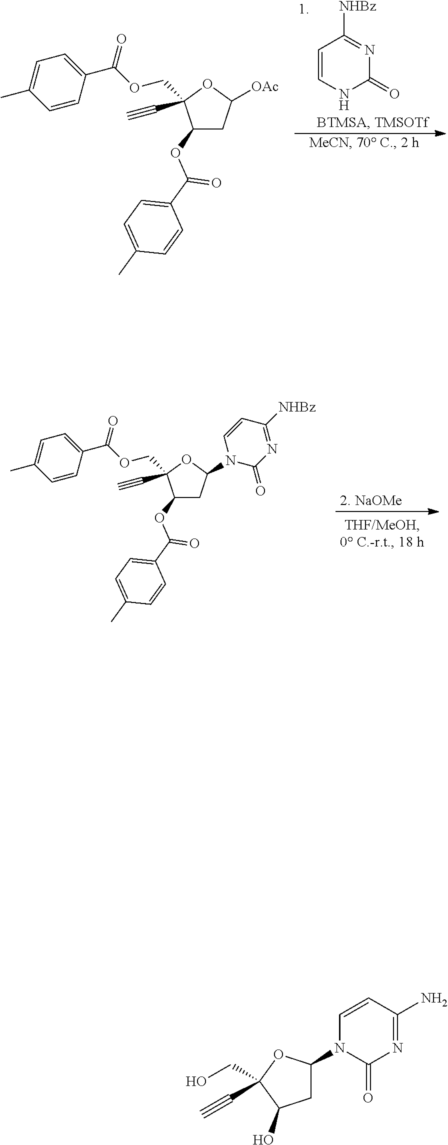

Step 1. To a mixture of N-(2-oxo-1H-pyrimidin-4-yl)benzamide (148 mg, 687.36 μmol) in MeCN (15 mL) was added BTMSA (234 mg, 1.37 mmol). The resulting mixture was heated at 70° C. for 1 hour. After cooling to ambient temperature, the TMSOTf (99 mg, 446.78 μmol) and a solution of [(2S,3R)-5-acetoxy-2-ethynyl-3-(4-methylbenzoyl)oxy-tetrahydrofuran-2-yl]methyl 4-methylbenzoate (150 mg, 343.68 μmol) were added sequentially. The resulting mixture was reheated at 70° C. for 1 hour. After cooling to ambient temperature, the reaction mixture was poured into water (10 mL), extracted with EtOAc (20 mL×2), and concentrated. The residue was purified by SFC (DAICEL CHIRALPAK AD, (250 mm*30 mm, 10 um); Supercritical CO₂/EtOH+NH4OH=45/55; 80 mL/min) to afford the compound (50 mg, 22%) as a white solid. 1 H NMR (400 MHz, CDCl₃): δ 8.71 (s, 1H), 8.49 (d, J=7.2 Hz, 1H), 7.97 (d, J=8.4 Hz, 2H), 7.92 (d, J=7.6 Hz, 2H), 7.79 (d, J=8.4 Hz, 2H), 7.67-7.62 (m, 1H), 7.58-7.52 (m, 2H), 7.30 (d, J=8.0 Hz, 2H), 7.20 (d, J=8.0 Hz, 2H), 6.39-6.30 (m, 1H), 5.92-5.85 (m, 1H), 4.70-4.61 (m, 2H), 3.28-3.14 (m, 1H), 2.79 (s, 1H), 2.73-2.64 (m, 1H), 2.45 (s, 3H), 2.37 (s, 3H).

Step 2. To a mixture of [(2S,3R,5R)-5-(4-benzamido-2-oxo-pyrimidin-1-yl)-2-ethynyl-3-(4-methylbenzoyl)oxy-tetrahydrofuran-2-yl]methyl 4-methylbenzoate (50 mg, 84.52 μmol) in MeOH (2 mL) was added NaOMe (9 mg, 169.03 μmol) at 0° C. The resulting mixture was stirred at 25° C. for 16 hours. Then the reaction mixture was concentrated. The residue was purified by prep-HPLC (acetonitrile 1-15%/0.225% formic acid in water) to afford the title compound (3.5 mg, 16%) as a white solid. ¹H NMR (400 MHz, DMSO-d6): δ 7.95 (s, 1H), 7.37 (s, 1H), 6.17 (t, J=6.4 Hz, 1H), 6.03-5.90 (m, 1H), 5.81 (s, 2H), 5.43 (s, 1H), 5.37-5.31 (m, 1H), 5.22 (s, 1H), 5.20-5.15 (m, 1H), 4.58 (t, J=6.4 Hz, 1H), 3.55-3.47 (m, 1H), 3.45-3.38 (m, 1H), 2.57-2.52 (m, 1H), 2.22-2.14 (m, 1H), 0.69-0.62 (m, 2H), 0.62-0.55 (m, 2H) [M+H]+ 252.1.

### Example 26

### Synthesis of 2-amino-9-[(2R,4R,5S)-5-ethynyl-4-hydroxy-5-(hydroxymethyl)tetrahydrofuran-2-yl]-1H-purin-6-one (Cpd. No. 79)

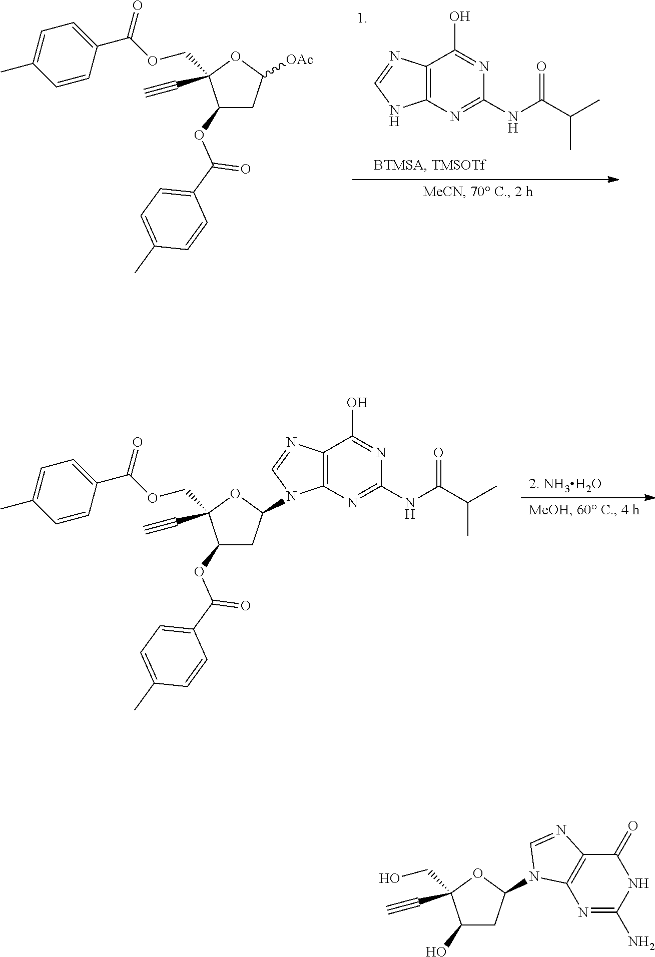

Step 1. A solution of N-(6-hydroxy-9H-purin-2-yl)-2-methyl-propanamide (84 mg, 378.05 μmol) in MeCN (10 mL) was degassed with N₂ for three times. Then trimethylsilyl (1E)-N-trimethylsilylethanimidate (349 mg, 1.72 mmol) was added. The resulting mixture was stirred at 70° C. for 1 h and then cooled to room temperature. Trimethylsilyl trifluoromethanesulfonate (88 mg, 395.23 μmol) was added and then [(2S,3R)-5-acetoxy-2-ethynyl-3-(4-methylbenzoyl)oxy-tetrahydrofuran-2-yl]methyl 4-methylbenzoate (150 mg, 343.68 μmol) in acetonitrile (2 mL) was added in one portion at 25° C. under N₂. The mixture was stirred at 70° C. for 16 h. After cooling to room temperature, the mixture was diluted with water (10 mL), extracted with EtOAc (20 mL), washed with brine (20 mL), dried over Na₂SO₄, filtered and concentrated. The residue was purified by prep-HPLC (acetonitrile 54-85%/0.225% formic acid in water) to afford the compound (25 mg, 15%) as a white solid. ¹H NMR (400 MHz, CDCl₃): δ 11.97 (s, 1H), 8.31 (s, 1H), 8.27 (br s, 1H), 7.97 (d, J=8.0 Hz, 2H), 7.84-7.73 (m, 2H), 7.30-7.21 (m, 4H), 6.34-6.27 (m, 1H), 5.89-5.81 (m, 1H), 4.73-4.66 (m, 1H), 4.63-4.57 (m, 1H), 3.15-3.06 (m, 1H), 2.96-2.86 (m, 1H), 2.75-2.71 (m, 1H), 2.67-2.56 (m, 1H), 2.42 (d, J=11.6 Hz, 6H), 1.28 (dd, J=2.0, 6.8 Hz, 6H).

Step 2. A solution of [(2S,3R,5R)-2-ethynyl-5-[6-hydroxy-2-(2-methylpropanoylamino)purin-9-yl]-3-(4-methylbenzoyl)oxy-tetrahydrofuran-2-yl]methyl 4-methylbenzoate (21 mg, 35.14 μmol) in MeOH (2 mL) was added ammonium hydroxide (30 mg, 105.42 μmol, 28 wt %). The solution was stirred at 60° C. for 16 h. After cooling to room temperature, the reaction mixture was concentrated. The residue was purified by prep-HPLC (acetonitrile 1-20%/0.225% formic acid in water) to afford the title compound (3.0 mg, 29%) as a white solid. ¹H NMR (400 MHz, DMSO-d6): δ 10.84 (br s, 1H), 8.24 (s, 1H), 6.50-6.41 (m, 1H), 6.15 (s, 2H), 5.52 (d, J=5.2 Hz, 1H), 5.36 (t, J=5.6 Hz, 1H), 4.53-4.41 (m, 1H), 3.72-3.53 (m, 2H), 3.51 (s, 1H), 2.46-2.34 (m, 2H). [M+Na]+ 313.9.

### Example 27

### Synthesis of (2R,3S,5R)-5-(4-amino-6-fluoro-3-methyl-1H-pyrazolo[3,4-d]pyrimidin-1-yl)-2-ethynyl-2-(hydroxymethyl)tetrahydrofuran-3-ol (Cpd. No. 75)

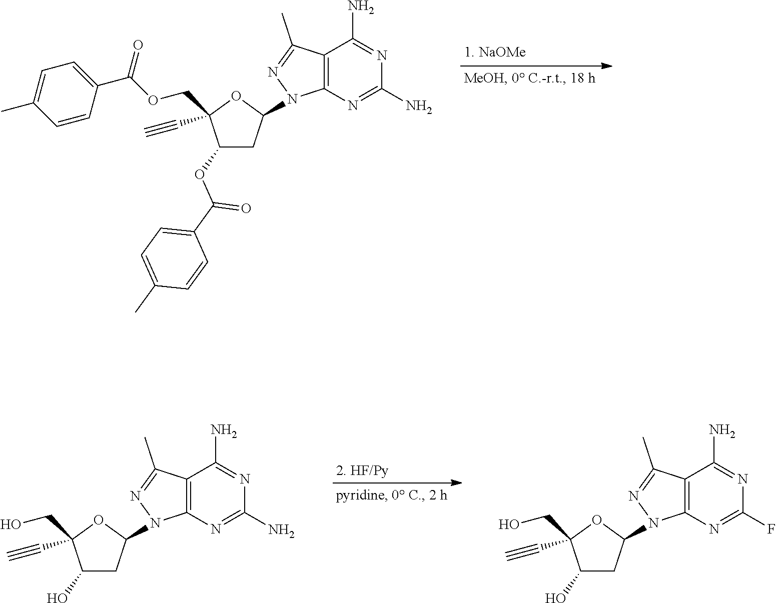 [SMILES: C#C[C@]1(COC(=O)c2ccc(C)cc2)O[C@@H](n2nc(C)c3c(N)nc(N)nc32)C[C@@H]1OC(=O)c1ccc(C)cc1 ; "1. NaOMe" ; C#C[C@]1(CO)O[C@@H](n2nc(C)c3c(N)nc(N)nc32)C[C@@H]1O ; "2. HF/Py" ; C#C[C@]1(CO)O[C@@H](n2nc(C)c3c(N)nc(F)nc32)C[C@@H]1O]

[(2R,3S,5R)-5-(4,6-Diamino-3-methyl-pyrazolo[3,4-d]pyrimidin-1-yl)-2-ethynyl-3-(4-methylbenzoyl)oxy-tetrahydrofuran-2-yl]methyl 4-methylbenzoate was prepared according to WO 2007/038507 A2 using 3-methyl-1H-pyrazolo[3,4-d]pyrimidine-4,6-diamine.

Step 1. To a mixture of [(2R,3S,5R)-5-(4,6-diamino-3-methyl-pyrazolo[3,4-d]pyrimidin-1-yl)-2-ethynyl-3-(4-methylbenzoyl)oxy-tetrahydrofuran-2-yl]methyl 4-methylbenzoate (120 mg, 221.99 μmol) in MeOH (15 mL) was added NaOMe (24 mg, 443.98 μmol) in one portion at 0° C. under N₂. The resulting mixture was stirred at 25° C. for 16 hours. Then the reaction mixture was concentrated. The residue was purified by pre-TLC (9% MeOH in dichloromethane (1% NH₄OH)) to afford the compound (60 mg, 89%) as a white solid. LCMS (ESI): m/z 327.1 (M+Na)+.

Step 2. To a mixture of (2R,3S,5R)-5-(4,6-diamino-3-methyl-pyrazolo[3,4-d]pyrimidin-1-yl)-2-ethynyl-2-(hydroxymethyl)tetrahydrofuran-3-ol (60 mg, 197.17 μmol) in pyridine (2 mL) was added pyridine-hydrofluoride (237 mg, 1.68 mmol, 70% purity) and tert-butyl nitrite (61 mg, 591.51 μmol) dropwised at 0° C. under N₂. The resulting mixture was stirred at 0° C. for 1 hour. After that, the mixture was diluted with DCM (15 mL) and H₂O (5 mL). The layers were separated and the aqueous layer was extracted with DCM (10 mL×3). The combined organic extracts were dried over Na₂SO₄, filtered, and concentrated in vacuo. The residue was further purified by prep-HPLC (acetonitrile 10-40%/0.225% formic acid in water) to afford the title compound (1.58 mg, 3%). ¹H NMR (400 MHz, DMSO-d6): δ 8.24 (s, 1H), 7.51 (s, 1H), 6.37-6.23 (m, 1H), 5.46 (d, J=6.0 Hz, 1H), 4.98 (t, J=6.0 Hz, 1H), 4.60-4.47 (m, 1H), 3.61-3.53 (m, 1H), 3.48-3.39 (m, 2H), 2.82-2.69 (m, 1H), 2.53 (s, 3H), 2.40-2.34 (m, 1H).

### Example 28

### Synthesis of (2R,3S,5S)-5-(4-amino-6-fluoro-3-methyl-1H-pyrazolo[3,4-d]pyrimidin-1-yl)-2-ethynyl-2-(hydroxymethyl)tetrahydrofuran-3-ol (Cpd. No. 3)

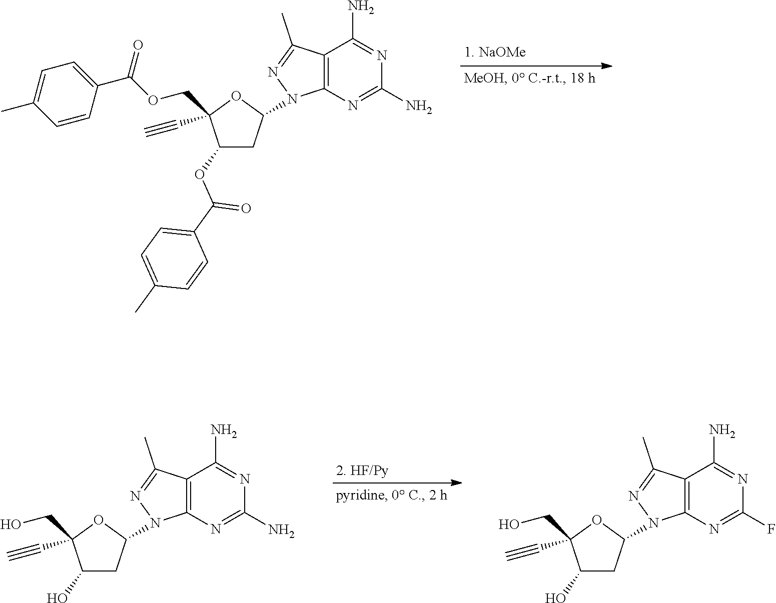 [SMILES: C#C[C@]1(COC(=O)c2ccc(C)cc2)O[C@H](n2nc(C)c3c(N)nc(N)nc32)C[C@@H]1OC(=O)c1ccc(C)cc1 ; "1. NaOMe" ; C#C[C@]1(CO)O[C@H](n2nc(C)c3c(N)nc(N)nc32)C[C@@H]1O ; "2. HF/Py" ; C#C[C@]1(CO)O[C@H](n2nc(C)c3c(N)nc(F)nc32)C[C@@H]1O]

[(2R,3S,5S)-5-(4,6-Diamino-3-methyl-pyrazolo[3,4-d]pyrimidin-1-yl)-2-ethynyl-3-(4-methylbenzoyl)oxy-tetrahydrofuran-2-yl]methyl 4-methylbenzoate was prepared according to WO 2007/038507 A2 using 3-methyl-1H-pyrazolo[3,4-d]pyrimidine-4,6-diamine.

Step 1. To a mixture of [(2R,3S,5S)-5-(4,6-diamino-3-methyl-pyrazolo[3,4-d]pyrimidin-1-yl)-2-ethynyl-3-(4-methylbenzoyl)oxy-tetrahydrofuran-2-yl]methyl 4-methylbenzoate (0.1 g, 184.99 μmol) in MeOH (15 mL) was added NaOMe (20 mg, 369.98 μmol) in one portion at 0° C. under N₂. The mixture was stirred for 18 hours at 25° C. Then the reaction mixture was concentrated, the residue was purified by pre-TLC eluting with 10% MeOH in DCM (1% NH₄OH) (Rf=0.3) to afford the compound (50 mg, 89%) as a white solid.

Step 2. To a mixture of (2R,3S,5S)-5-(4,6-diamino-3-methyl-pyrazolo[3,4-d]pyrimidin-1-yl)-2-ethynyl-2-(hydroxymethyl)tetrahydrofuran-3-ol (60 mg, 197.17 μmol) in pyridine (2 mL) was added HF-pyridine (237 mg, 1.68 mmol, 70% purity) and tert-butyl nitrite (61 mg, 591.51 μmol) dropwise at 0° C. under N₂. The resulting mixture was stirred at 0° C. for 1 hour. After that, the mixture was diluted with DCM (15 mL) and H₂O (5 mL). The layers were separated and the aqueous layer was extracted with DCM (10 mL). The combined organic extracts were dried over anhydrous Na₂SO₄, filtered, and concentrated in vacuo. The residue was purified by prep-HPLC (acetonitrile 10-40%/0.225% formic acid in water) to afford the title compound (5 mg, 8%) as a white solid. ¹H NMR (400 MHz, DMSO-d6) δ 8.27 (s, 1H), 7.53 (s, 1H), 6.27 (t, J=6.4 Hz, 1H), 5.73 (d, J=8.8 Hz, 1H), 5.21 (t, J=6.4 Hz, 1H), 4.38-4.30 (m, 1H), 3.48-3.40 (m, 3H), 2.79-2.71 (m, 1H), 2.68-2.62 (m, 1H), 2.53 (s, 3H). [M+H]+ 308.2.

### Example 29

### Synthesis of 4-amino-1-[(2S,5S)-5-ethynyl-5-(hydroxymethyl)-2H-furan-2-yl]-5-fluoro-pyrimidin-2-one (Cpd. No. 74)

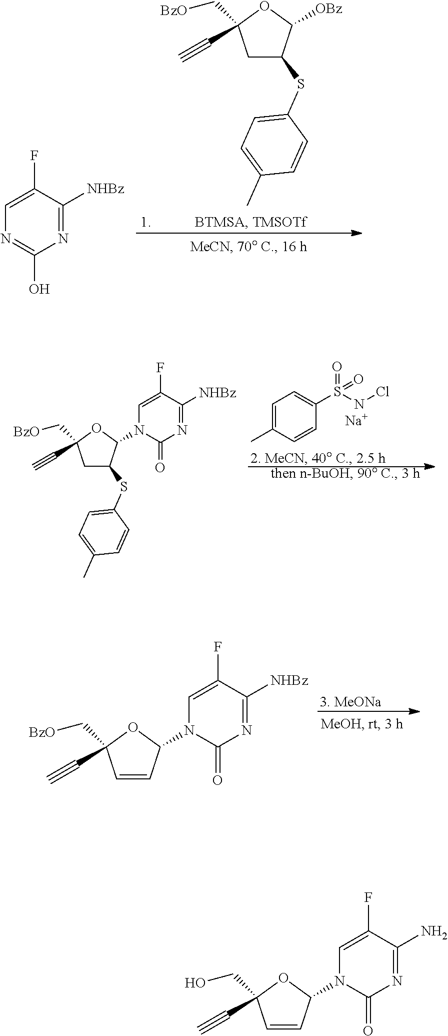

Step 1. A solution of N-(5-fluoro-2-hydroxy-pyrimidin-4-yl)benzamide (59 mg, 253.94 μmol) in acetonitrile (3 mL) was degassed with N₂ for three times. And then trimethylsilyl (1E)-N-trimethylsilylethanimidate (129 mg, 634.85 μmol) was added. The reaction mixture was stirred at 70° C. for 1 h and then cooled to room temperature. Trimethylsilyl trifluoromethanesulfonate (56 mg, 253.94 μmol) was added and then [(2S,4S,5R)-5-benzoyloxy-2-ethynyl-4-(p-tolylsulfanyl)tetrahydrofuran-2-yl]methyl benzoate (100 mg, 211.62 μmol) in acetonitrile (1 mL) was added at 25° C. under N₂. After stirring at 70° C. for 16 h, the reaction mixture was cooled to room temperature and diluted with water (10 mL), extracted with EtOAc (20 mL), washed with brine (20 mL), dried over Na₂SO₄, filtered and concentrated. The residue was purified by pre-TLC (25% EtOAc in petroleum ether) to afford the compound (21 mg, 17%) as a white solid. ¹H NMR (400 MHz, CDCl₃) δ 8.33-8.25 (m, 2H), 8.09 (d, J=7.2 Hz, 2H), 7.73-7.65 (m, 1H), 7.60-7.42 (m, 6H), 7.27 (s, 2H), 7.22 (d, J=5.6 Hz, 1H), 7.04 (d, J=7.6 Hz, 2H), 6.32-6.13 (m, 1H), 4.69 (d, J=11.6 Hz, 1H), 4.55 (d, J=12.0 Hz, 1H), 3.63-3.46 (m, 1H), 2.95-2.82 (m, 1H), 2.79 (s, 1H), 2.53-2.39 (m, 1H), 2.16 (s, 3H).

Step 2. A solution of [(2S,4S,5S)-5-(4-benzamido-5-fluoro-2-oxo-pyrimidin-1-yl)-2-ethynyl-4-(p-tolylsulfanyl)tetrahydrofuran-2-yl]methyl benzoate (40 mg, 68.54 μmol) in MeCN (2 mL) was added [chloro(p-tolylsulfonyl)amino]sodium (22 mg, 95.95 μmol). The reaction mixture was stirred at 50° C. for 2.5 h. The reaction mixture was added water (10 mL), extracted with EtOAc (20 mL), washed with brine (20 mL), dried over Na₂SO₄, filtered and concentrated. The residue was dissolved in n-BuOH (2 mL). Then the solution was stirred at 90° C. for another 3 h. After cooling to room temperature, the reaction mixture was concentrated. The residue was purified by pre-TLC (25% EtOAc in petroleum ether) to afford the compound (39 mg, 51%) as a white solid. LCMS (ESI): m/z 460.0 (M+H)+.

Step 3. A solution of [(2S,5S)-2-(4-benzamido-5-fluoro-2-oxo-pyrimidin-1-yl)-5-ethynyl-2H-furan-5-yl]methyl benzoate (20 mg, 43.53 μmol) in MeOH (1 mL) was added MeONa (3 mg, 53.53 μmol) at 0° C. The resulting mixture was stirred at 25° C. for 3 h. Then the reaction solution was concentrated. The residue was purified by prep-HPLC (acetonitrile 1-27%/0.225% formic acid in water) to afford the title compound (5.93 mg, 19%) as a white solid. ¹H NMR (400 MHz, DMSO-d6) δ 8.38 (br s, 1H), 7.92 (d, J=7.2 Hz, 1H), 7.85 (br s, 1H), 7.61 (br s, 1H), 6.92 (s, 1H), 6.33-6.26 (m, 1H), 6.03 (d, J=5.6 Hz, 1H), 3.76-3.56 (m, 3H). [M+Na]+ 273.8.

### Example 30

### Synthesis of 4-amino-1-((2S,4R,5S)-5-ethynyl-4-hydroxy-5-(hydroxymethyl)tetrahydrofuran-2-yl)pyrimidin-2(1H)-one (Cpd. No. 76)

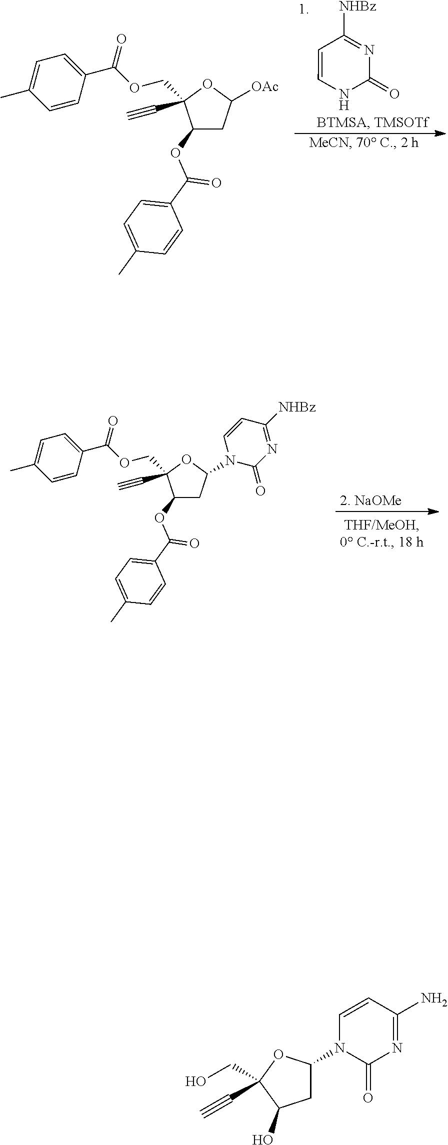

Step 1. To a mixture of N-(2-oxo-1H-pyrimidin-4-yl)benzamide (148 mg, 687.36 μmol) in MeCN (15 mL) was added BTMSA (234 mg, 1.37 mmol). The resulting mixture was heated at 70° C. for 1 hour. After cooling to ambient temperature, TMSOTf (99 mg, 446.78 μmol) and [(2S,3R)-5-acetoxy-2-ethynyl-3-(4-methylbenzoyl)oxy-tetrahydrofuran-2-yl]methyl 4-methylbenzoate (150 mg, 343.68 μmol) were added sequentially. The resulting mixture was reheated at 70° C. for 1 hour. After cooling to ambient temperature, the reaction mixture was poured into water (10 mL), extracted with EtOAc (20 mL×2), concentrated. The residue was purified by SFC (DAICEL CHIRALPAK AD, (250 mm*30 mm, 10 um); Supercritical CO₂/EtOH+NH₄OH=45/55; 80 mL/min) to afford the compound (20 mg, 22%) as a white solid.

Step 2. To a mixture of [(2S,3R,5S)-5-(4-benzamido-2-oxo-pyrimidin-1-yl)-2-ethynyl-3-(4-methylbenzoyl)oxy-tetrahydrofuran-2-yl]methyl 4-methylbenzoate (0.02 g, 33.81 μmol) in MeOH (2 mL) was added NaOMe (4 mg, 67.61 μmol) at 0° C. The resulting mixture was stirred at 25° C. for 16 hours. Then the reaction mixture was concentrated. The residue was purified by prep-HPLC (acetonitrile 1-17%/0.225% formic acid in water) to afford the title compound (3.8 mg, 16%) as a white solid. ¹H NMR (400 MHz, DMSO-d6) δ 8.37 (s, 2H), 7.80 (d, J=7.2 Hz, 1H), 7.23-7.07 (m, 2H), 6.20-6.10 (m, 1H), 5.73 (d, J 7.6 Hz, 1H), 4.24 (d, J=4.8 Hz, 1H), 3.74 (d, J=11.2 Hz, 1H), 3.63 (d, J=11.2 Hz, 1H), 3.51 (s, 1H), 2.78-2.69 (m, 1H), 1.85-1.77 (m, 1H). [2M+H]+ 503.1.

### Example 31

### Synthesis of 2-amino-9-((2S,4R,5S)-5-ethynyl-4-hydroxy-5-(hydroxymethyl)tetrahydrofuran-2-yl)-1H-purin-6(9H)-one (Cpd. No. 78)

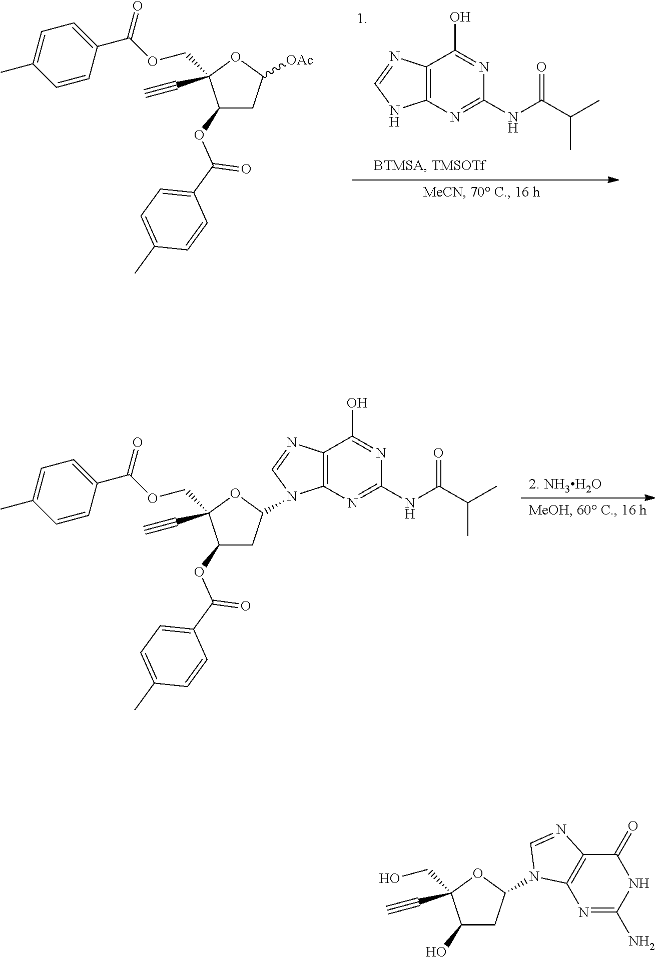

Step 1. A solution of N-(6-hydroxy-9H-purin-2-yl)-2-methyl-propanamide (84 mg, 378.05 μmol) in acetonitrile (10 mL) was degassed with N₂ for three times. And then trimethylsilyl (1E)-N-trimethylsilylethanimidate (349 mg, 1.72 mmol) was added. After stirring at 70° C. for 1 h, the reaction mixture was cooled to room temperature. Trimethylsilyl trifluoromethanesulfonate (88 mg, 395.23 μmol) was added and then [(2S,3R)-5-acetoxy-2-ethynyl-3-(4-methylbenzoyl)oxy-tetrahydrofuran-2-yl]methyl 4-methylbenzoate (150 mg, 343.68 μmol) in acetonitrile (2 mL) was added in one portion at 25° C. under N₂. After stirring at 70° C. for 16 h, the reaction mixture was cooled to room temperature and diluted with water (20 mL), extracted with EtOAc (30 mL), washed with brine (20 mL), dried over Na₂SO₄, filtered and concentrated. The residue was purified by prep-HPLC (acetonitrile 54-85%/0.225% formic acid in water) to afford the compound (21 mg, 10%) as a white solid. ¹H NMR (400 MHz, CD₃OD) δ 8.49-8.36 (m, 2H), 8.00 (d, J=8.0 Hz, 2H), 7.87 (d, J=8.0 Hz, 2H), 7.32 (d, J=8.0 Hz, 2H), 7.34-7.30 (m, 1H), 7.22 (d, J=8.0 Hz, 2H), 6.79-6.69 (m, 1H), 6.12-6.01 (m, 1H), 4.81 (d, J=11.6 Hz, 1H), 4.66 (d, J=11.6 Hz, 1H), 3.27 (s, 1H), 3.20-3.09 (m, 1H), 2.98-2.88 (m, 1H), 2.76-2.69 (m, 1H), 2.43 (s, 3H), 2.38 (s, 3H), 1.24-1.21 (m, 6H).

Step 2. A solution of [(2S,3R,5S)-2-ethynyl-5-[6-hydroxy-2-(2-methylpropanoylamino)purin-9-yl]-3-(4-methylbenzoyl)oxy-tetrahydrofuran-2-yl]methyl 4-methylbenzoate (21 mg, 35.14 μmol) in methyl alcohol (2 mL) was added ammonium hydroxide (300 mg, 1054.2 umol, 28% purity). The solution was stirred at 60° C. for 16 h. Then the reaction solution was cooled to room temperature and concentrated. The residue was purified by prep-HPLC (acetonitrile 1-20%/0.225% formic acid in water) to afford the title compound (3.0 mg, 29%) as a white solid. ¹H NMR (400 MHz, DMSO-d6) δ 10.84 (br s, 1H), 8.24 (s, 1H), 6.50-6.41 (m, 1H), 6.15 (s, 2H), 5.52 (d, J=5.2 Hz, 1H), 5.36 (t, J=5.6 Hz, 1H), 4.53-4.41 (m, 1H), 3.72-3.53 (m, 2H), 3.51 (s, 1H), 2.46-2.34 (m, 2H). [M+Na]+ 313.9.

### Example 32

### Synthesis of 1-((4S,5R)-5-ethynyl-4-hydroxy-5-(hydroxymethyl)tetrahydrofuran-2-yl)-1H-1,2,4-triazole-3-carboxamide (Cpd. No. 11)

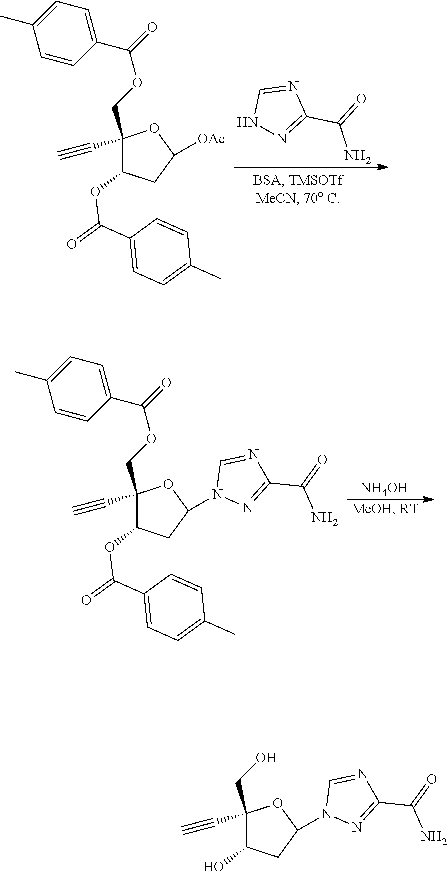

Step 1: (2R,3S)-5-(3-carbamoyl-1H-1,2,4-triazol-1-yl)-2-ethynyl-2-(((4-methylbenzoyl)oxy)methyl)tetrahydrofuran-3-yl 4-methylbenzoate. To a suspension of 1H-1,2,4-triazole-3-carboxamide (57.7 mg, 504 μmol) in MeCN (3.88 mL) was added N,O-bis(trimethylsilyl)acetamide (590 μL, 2.29 mmol), and the resulting mixture heated to 70° C. with stirring for ca. 1 h. The mixture was then cooled to 0° C., and a solution of (2R,3S)-5-acetoxy-2-ethynyl-2-(((4-methylbenzoyl)oxy)methyl)tetrahydrofuran-3-yl 4-methylbenzoate (200 mg, 458 μmol) in MeCN (3.88 mL) was added dropwise with stirring, followed by trimethylsilyl trifluoromethanesulfonate (127 μL, 687 μmol). The reaction mixture was then warmed to 70° C. with stirring for ca. 18 h before cooling to ambient temperature. The reaction was quenched with saturated aqueous NaHCO₃ solution (10 mL), diluted with saturated aqueous NaCl solution (12 mL), and the organics extracted with EtOAc (3×30 mL). The combined organics were dried (anhyd. Na₂SO₄), filtered, and concentrated in vacuo. The residue was purified by flash chromatography on SiO₂ (eluent: 50-100% EtOAc in heptanes) to afford the title compound (132 mg, 270 μmol, 59%) as a yellow oil, as a mixture of anomers. For the more polar anomer, LC-MS (ESI) m/z 489.2 [M+H]+. LC-MS RT=1.52 min; Method G. For the less polar anomer, LC-MS (ESI) m/z 489.2 [M+H]+. LC-MS RT=1.54 min; Method G.

Step 2: 1-((4S,5R)-5-ethynyl-4-hydroxy-5-(hydroxymethyl)tetrahydrofuran-2-yl)-1H-1,2,4-triazole-3-carboxamide. To a solution of (2R,3S)-5-(3-carbamoyl-1H-1,2,4-triazol-1-yl)-2-ethynyl-2-(((4-methylbenzoyl)oxy)methyl)tetrahydrofuran-3-yl 4-methylbenzoate (132 mg, 270 μmol) in MeOH (901 μmol) was added 28% aqueous NH₄OH (60 μL) dropwise, and the resulting mixture stirred at ambient temperature for ca. 18 h. After this time, an additional portion of 28% aqueous NH₄OH (60 μL) was added dropwise, and the mixture heated to 45° C. with stirring for ca. 4 h. The mixture was then cooled to ambient temperature and purified directly by reverse-phase chromatography on C18 (eluent: 0-100% MeCN in 10 mM aq. ammonium formate solution) to afford Cpd. No. 11 (61.3 mg, 243 μmol, 90%) as a yellow oil, as a ca. 2:1 mixture of anomers. For the major anomer, ¹H NMR (400 MHz, CD₃OD) δ 8.72 (s, 1H), 6.30 (dd, J=7.2, 3.0 Hz, 1H), 4.72 (dd, J=8.6, 6.8 Hz, 1H), 3.80 (d, J=12.2 Hz, 1H), 3.72 (d, J=12.2 Hz, 1H), 3.09 (s, 1H), 2.75 (ddd, J=13.3, 6.8, 3.0 Hz, 1H), 2.70-2.64 (m, 1H). For the minor anomer, ¹H NMR (400 MHz, CD₃OD) δ 8.79 (s, 1H), 6.24 (dd, J=7.1, 3.6 Hz, 1H), 4.47 (dd, J=6.7, 4.6 Hz, 1H), 3.73 (d, J=12.0 Hz, 1H), 3.67 (d, J=12.1 Hz, 1H), 3.15 (s, 1H), 2.93 (dt, J=13.9, 6.9 Hz, 1H), 2.61 (ddd, J=14.0, 4.6, 3.7 Hz, 1H). LC-MS (ESI) m/z 253.1 [M+H]+. LC-MS RT=0.13 min: Method A.

### Example 33

### Synthesis of 4-amino-5-bromo-1-((2R,4S,5R)-4-hydroxy-5-(hydroxymethyl)-5-methyltetrahydrofuran-2-yl)pyrimidin-2(1H)-one (Cpd. No. 15)

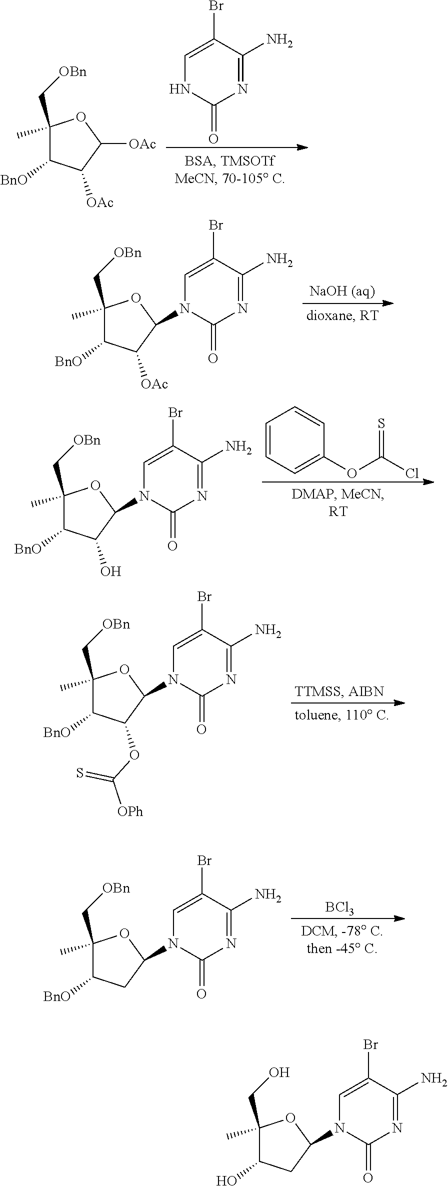

Step 1: (2R,3R,4S,5R)-2-(4-amino-5-bromo-2-oxopyrimidin-1(2H)-yl)-4-(benzyloxy)-5-((benzyloxy)methyl)-5-methyltetrahydrofuran-3-yl acetate. To a mixture of 4-amino-5-bromo-1H-pyrimidin-2-one (0.38 g, 2.00 mmol) in MeCN (15 mL) was added N,O-bis(trimethylsilyl)acetamide (1.22 g, 6.00 mmol) in one portion at 25° C. under N₂. Then the mixture was stirred at 70° C. for 1 hour. After cooling to 25° C., TMSOTf (390 mg, 1.76 mmol) and (3R,4S,5R)-4-(benzyloxy)-5-((benzyloxy)methyl)-5-methyltetrahydrofuran-2,3-diyl diacetate (0.5 g, 1.17 mmol) was added dropwise at 25° C. The resulting mixture was stirred at 25° C. for 16 hours. Then the reaction was quenched by addition of 10 wt % of aqueous citric acid solution (10 mL and extracted with EtOAc (20 mL). The organic layer was washed with brine (15 mL), dried over anhydrous Na₂SO₄, filtered and concentrated. The residue was purified by column chromatography on silica gel (column height: 250 mm, diameter: 100 mm, 100-200 mesh silica gel, DCM/MeOH=50/1) to give the title compound (0.64 g, 98% yield) as yellow oil. ¹H NMR (400 MHz, CDCl₃) δ 8.48 (s, 1H), 7.40-7.20 (m, 10H), 6.08 (d, J=2.4 Hz, 1H), 5.49-5.45 (m, 1H), 4.63 (d, J=12 Hz, 1H), 4.52-4.47 (m, 1H), 4.45-4.39 (m, 1H), 4.36 (d, J=11.6 Hz, 1H), 4.27 (d, J=6.0 Hz, 1H), 3.60 (d, J=10.4 Hz, 1H), 3.31 (d, J=10.4 Hz, 1H), 2.14 (s, 3H), 1.28 (s, 3H).

Step 2: 4-amino-1-((2R,3R,4S,5R)-4-(benzyloxy)-5-((benzyloxy)methyl)-3-hydroxy-5-methyltetrahydrofuran-2-yl)-5-bromopyrimidin-2(1H)-one. To a mixture of (2R,3R,4S,5R)-2-(4-amino-5-bromo-2-oxopyrimidin-1(2H)-yl)-4-(benzyloxy)-5-((benzyloxy)methyl)-5-methyltetrahydrofuran-3-yl acetate (0.638 g, 1.14 mmol) in MeOH (15 mL) was added NaOH (2 M, 2 mL) aqueous solution in one portion at 25° C. The mixture was stirred at 25° C. for 30 min. After that, the reaction mixture was concentrated. The residue was purified by column chromatography on silica gel (column height: 250 mm, diameter: 100 mm, 100-200 mesh silica gel, Dichloromethane/MeOH=100/0 to 50/1) to give the title compound (0.5 g, 85% yield) as a white solid. ¹H NMR (400 MHz, CDCl₃) δ 8.25 (s, 1H), 7.40-7.23 (m, 10H), 6.58 (s, 1H), 5.93 (d, J=3.6 Hz, 1H), 5.66 (s, 1H), 4.77 (d, J=12.0 Hz, 1H), 4.60-4.46 (m, 3H), 4.40-4.30 (m, 1H), 4.11 (d, J=5.6 Hz, 1H), 3.61-3.51 (m, 2H), 3.37 (d, J=10.4 Hz, 1H), 1.35 (s, 3H).

Step 3: O-((2R,3R,4S,5R)-2-(4-amino-5-bromo-2-oxopyrimidin-1(2H)-yl)-4-(benzyloxy)-5-((benzyloxy)methyl)-5-methyltetrahydrofuran-3-yl) O-phenyl carbonothioate. To a mixture of 4-amino-1-((2R,3R,4S,5R)-4-(benzyloxy)-5-((benzyloxy)methyl)-3-hydroxy-5-methyltetrahydrofuran-2-yl)-5-bromopyrimidin-2(1H)-one (0.5 g, 968.27 μmol) in MeCN (15 mL) was added DMAP (355 mg, 2.90 mmol), then O-phenyl chloromethanethioate (251 mg, 1.45 mmol) was added dropwise at 25° C. under N₂. After stirring at 25° C. for 1 hour, the mixture was concentrated under reduced pressure. The residue was purified by column chromatography on silica gel (column height: 250 mm, diameter: 100 mm, 100-200 mesh silica gel, DCM/MeOH=100/0 to 99/1) to give the title compound (0.55 g, 87% yield) as a light yellow solid. ¹H NMR (400 MHz, CDCl₃) δ 8.32 (s, 1H), 7.49-7.28 (m, 14H), 7.06-7.01 (m, 2H), 6.32 (d, J=3.2 Hz, 1H), 6.05-5.95 (m, 1H), 5.68 (s, 1H), 4.75 (d, J=11.6 Hz, 1H), 4.63-4.53 (m, 1H), 4.50-4.42 (m, 3H), 3.63 (d, J=10.8 Hz, 1H), 3.38 (d, J=10.4 Hz, 1H), 1.30 (s, 3H).

Step 4: 4-amino-1-((2R,4S,5R)-4-(benzyloxy)-5-((benzyloxy)methyl)-5-methyltetrahydrofuran-2-yl)-5-bromopyrimidin-2(1H)-one. To a mixture of O-((2R,3R,4S,5R)-2-(4-amino-5-bromo-2-oxopyrimidin-1(2H)-yl)-4-(benzyloxy)-5-((benzyloxy)methyl)-5-methyltetrahydrofuran-3-yl) O-phenyl carbonothioate (0.55 g, 842.84 umol) in toluene (12 mL) was added AIBN (69 mg, 421.42 umol) and TTMSS (1.05 g, 4.21 mmol) in one portion at 25° C. under N₂. The mixture was heated to 110° C. and stirred for 2 hours. Then the reaction mixture was cooled to 25° C. concentrated. The residue was purified by pre-TLC (DCM/MeOH=15/1) to give the title compound (170 mg, 40% yield) as colorless oil.

Step 5: 4-amino-5-bromo-1-((2R,4S,5R)-4-hydroxy-5-(hydroxymethyl)-5-methyltetrahydrofuran-2-yl)pyrimidin-2(1H)-one (Cpd. No. 15). To a mixture of 4-amino-1-((2R,4S,5R)-4-(benzyloxy)-5-((benzyloxy)methyl)-5-methyltetrahydrofuran-2-yl)-5-bromopyrimidin-2(1H)-one (0.15 g, 299.77 μmol) in DCM (9 mL) was added BCl₃ (1 M, 2.1 mL) dropwise at −78° C. under N₂. The reaction mixture was stirred at −78° C. for 15 min, then allowed to warm up to −40° C. and stirred for another 0.5 h. Then the reaction was quenched by addition of MeOH (1 mL) and NH₄OH (1 mL) at −40° C. After stirring at −40° C. for another 10 min, the mixture was allowed to warm to room temperature and stirred for 10 min. After that, the reaction mixture was concentrated directly. The residue was purified by pre-HPLC (basic) to give Cpd. No. 15 (2.7 mg, 3% yield) as a white solid. ¹H NMR (400 MHz, CD₃OD) δ 8.59 (s, 1H), 6.12-6.03 (m, 1H), 4.36 (t, J=6.8 Hz, 1H), 3.67-3.54 (m, 2H), 2.53-2.42 (m, 1H), 2.31-2.21 (m, 1H), 1.17 (s, 3H). LC-MS (ESI) m/z 661.0 [2M+Na]+. LC-MS RT=1.420 min; Method D.

### Example 34

### Synthesis of (2R,3S,5R)-5-(4-amino-5-bromo-2-oxopyrimidin-1(2H)-yl)-3-hydroxy-2-(hydroxymethyl)tetrahydrofuran-2-carbonitrile (Cpd. No. 16)

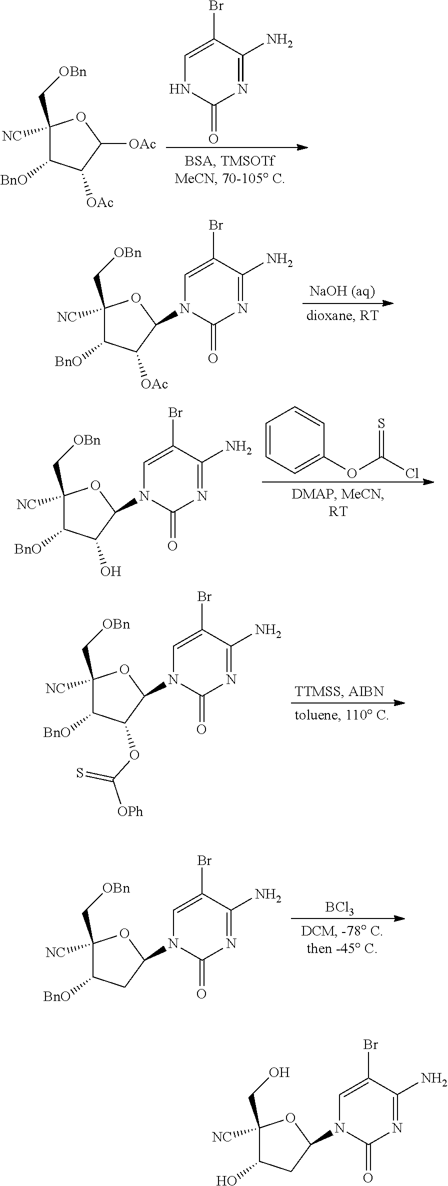

Step 1: (2R,3R,4S,5R)-2-(4-amino-5-bromo-2-oxopyrimidin-1(2H)-yl)-4-(benzyloxy)-5-((benzyloxy)methyl)-5-cyanotetrahydrofuran-3-yl acetate. To a solution of 4-amino-5-bromo-1H-pyrimidin-2-one (400 mg, 2.1 mmol) in acetonitrile (15 mL) was added N,O-bis(trimethylsilyl)acetamide (1.6 mL, 6.3 mmol), the reaction mixture was stirred at 70° C. for 1 h and cooled to room temperature. Trimethylsilyl trifluoromethanesulfonate (490 uL, 2.7 mmol) was added and then (3R,4S,5R)-4-(benzyloxy)-5-((benzyloxy)methyl)-5-cyanotetrahydrofuran-2,3-diyl diacetate (600 mg, 1.4 mmol) in acetonitrile (8 mL) was added. After stirring at 105° C. for 16 h, the reaction was quenched with water (20 mL), extracted with EtOAc (30 mL), washed with brine (20 mL) dried over Na₂SO₄, filtered, and concentrated. The residue was purified by silica gel chromatography eluting with 0-3% MeOH in DCM to afford the title product (302 mg, 0.53 mmol, 39% yield) as a brown oil. ¹H NMR (400 MHz, CDCl₃): δ 7.99 (s, 1H), 7.38-7.30 (m, 8H), 7.25-7.18 (m, 2H), 5.97 (s, 1H), 5.62 (d, J=5.2 Hz, 1H), 4.69 (d, J=11.6 Hz, 1H), 4.51-4.42 (m, 4H), 3.90 (d, J=10.8 Hz, 1H), 3.61 (d, J=10.8 Hz, 1H), 2.18 (s, 3H).

Step 2: (2R,3S,4R,5R)-5-(4-amino-5-bromo-2-oxopyrimidin-1(2H)-yl)-3-(benzyloxy)-2-((benzyloxy)methyl)-4-hydroxytetrahydrofuran-2-carbonitrile. To a solution of (2R,3R,4S,5R)-2-(4-amino-5-bromo-2-oxopyrimidin-1(2H)-yl)-4-(benzyloxy)-5-((benzyloxy)methyl)-5-cyanotetrahydrofuran-3-yl acetate (300 mg, 526.9 μmol) in dioxane (8 mL) was added aqueous NaOH (1 M, 1.0 mL) at 25° C. The resulting reaction mixture was stirred at 25° C. for 1 h. Then the reaction was quenched with water (5 mL), extracted with EtOAc (10 mL), washed with brine (10 mL), dried over Na₂SO₄, filtered and concentrated. The residue was purified by silica gel chromatography eluting with 0-3% MeOH in DCM to afford the title product (260 mg, 94% yield) as a white solid.

Step 3: A solution of (2R,3S,4R,5R)-5-(4-amino-5-bromo-2-oxo-pyrimidin-1-yl)-3-benzyloxy-2-(benzyloxymethyl)-4-hydroxy-tetrahydrofuran-2-carbonitrile (260.0 mg, 493.0 μmol) in acetonitrile (5 mL) was degassed with N₂ for three times. Then DMAP (78.3 mg, 640.9 μmol) and O-phenyl chloromethanethioate (170.2 mg, 986.0 μmol) were added. The resulting reaction mixture was stirred at 25° C. for 1 h. After that, the reaction mixture was diluted with water (5 mL), extracted with EtOAc (10 mL), washed with brine (10 mL), dried over Na₂SO₄, filtered and concentrated. The residue was purified by silica gel chromatography eluting with 0-3% MeOH in DCM to afford the title product (251.0 mg, 378 μmol, 76% yield) as a brown oil. LC-MS (ESI) m/z 663.0 [M+H]+. LC-MS RT=1.025 min; Method C.

Step 4: (2R,3S,5R)-5-(4-amino-5-bromo-2-oxopyrimidin-1(2H)-yl)-3-(benzyloxy)-2-((benzyloxy)methyl)tetrahydrofuran-2-carbonitrile. A solution of (2R,3S,4R,5R)-5-(4-amino-5-bromo-2-oxo-pyrimidin-1-yl)-3-benzyloxy-2-(benzyloxymethyl)-4-hydroxy-tetrahydrofuran-2-carbonitrile (250 mg, 376.8 μmol) in toluene (5 mL) was purged with N₂ for 5 min. Bis(trimethylsilyl)silyl-trimethyl-silane (281.1 mg, 1.13 mmol) and AIBN (29.0 mg, 176.8 μmol) were added. The reaction mixture was stirred at 110° C. for 2 h. The reaction mixture was concentrated. The residue was purified by silica gel chromatography eluting with 0-3% MeOH in DCM to afford the title product (95.0 mg, 186 μmol, 49% yield) as a white solid.

Step 5: (2R,3S,5R)-5-(4-amino-5-bromo-2-oxopyrimidin-1(2H)-yl)-3-hydroxy-2-(hydroxymethyl)tetrahydrofuran-2-carbonitrile (Cpd. No. 16). To a solution of (2R,3S,5R)-5-(4-amino-5-bromo-2-oxopyrimidin-1(2H)-yl)-3-(benzyloxy)-2-((benzyloxy)methyl)tetrahydrofuran-2-carbonitrile (95.0 mg, 186 μmol) in DCM (3 mL) was degassed with N₂ for three times and cooled to −78° C., then BCl₃ (1 M in DCM, 0.5 mL) was added dropwise. The resulting reaction mixture was stirred at −45° C. for 0.5 h. Then the reaction was quenched with MeOH (1 mL) at −45° C. and warmed to 0° C. The reaction was adjust pH>7 with 1 mL NH₃·H₂O (28% purity) and concentrated to residue. The residue was purified by pre-HPLC (acetonitrile 0-30%/NH₃·H₂O+NH₄HCO₃ in water) to afford the title compound (3.5 mg, 10.6 μmol, 6% yield) as a white solid. ¹H NMR (400 MHz, MeOD-d₄): δ 8.23 (s, 1H), 6.35-6.26 (m, 1H), 4.59 (t, J=6.8 Hz, 1H), 4.01-3.86 (m, 2H), 2.55-2.37 (m, 2H). LC-MS (ESI) m/z 331.0 [M+H]+. LC-MS RT=1.312 min; Method F.

### Example 35

### Synthesis of 1-((2R,5R)-5-ethynyl-5-(hydroxymethyl)-2,5-dihydrofuran-2-yl)-5-fluoropyrimidine-2,4(1H,3H)-dione (Cpd. No. 19)

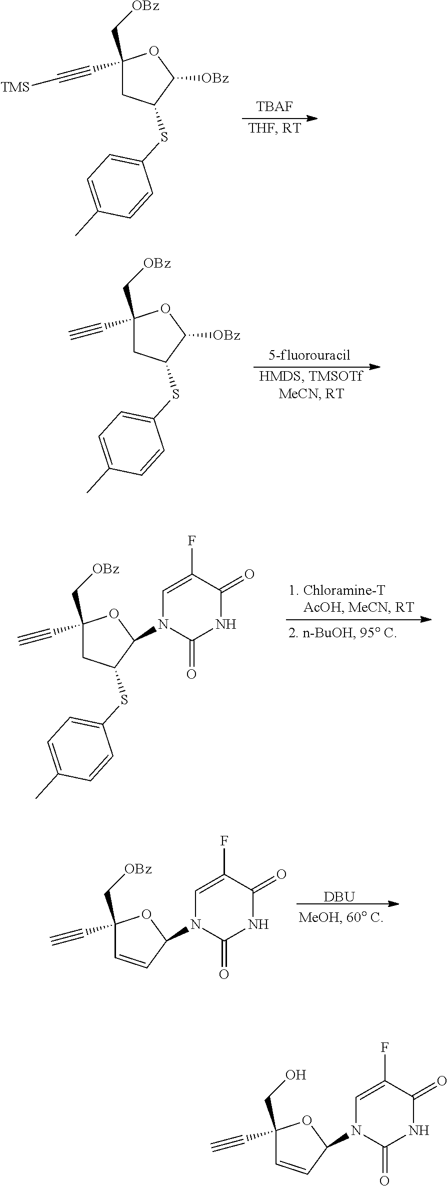

Step 1: ((2R,4R,5R)-5-(benzoyloxy)-2-ethynyl-4-(p-tolylthio)tetrahydrofuran-2-yl)methyl benzoate. A solution of TBAF (1M in THF, 15.4 mL, 15.4 mmol) was added to a stirring solution of ((2R,4R,5R)-5-(benzoyloxy)-4-(p-tolylthio)-2-((trimethylsilyl)ethynyl)tetrahydrofuran-2-yl)methyl benzoate (7.01 g, 12.9 mmol) in THE (50.0 mL) at ambient temperature, and the resulting mixture stirred at this temperature for ca. 1 h. The reaction was then quenched with a solution of 20% aqueous NH₄OAc, and the mixture concentrated in vacuo. The residue was purified by flash chromatography on SiO₂ (eluent: 0-30% EtOAc/hexanes) to furnish the title compound (2.5 g, 5.30 mmol, 41%) as a white solid. LC-MS (ESI) m/z 490.2 [M+H₂O]+. LC-MS RT=2.02 min; Method A.

Step 2: ((2R,4R,5R)-2-ethynyl-5-(5-fluoro-2,4-dioxo-3,4-dihydropyrimidin-1(2H)-yl)-4-(p-tolylthio)tetrahydrofuran-2-yl)methyl benzoate. A flame-dried round-bottomed flask equipped with a magnetic stirrer bar was charged with 5-fluorouracil (231 mg, 1.78 mmol) and MeCN (3.00 mL) under nitrogen. HMDS (201 μL, 1.78 mmol) and trimethylsilyl trifluoromethanesulfonate (511 μL, 2.79 mmol) were both added, and the resulting mixture stirred at ambient temperature for ca. 1 h. Solid ((2R,4R,5R)-5-(benzoyloxy)-2-ethynyl-4-(p-tolylthio)tetrahydrofuran-2-yl)methyl benzoate (600 mg, 1.27 mmol) was added in one portion, and the resulting mixture stirred at ambient temperature overnight. The reaction was quenched with a solution of K₃PO₄ (138 mg, 635 μmol) in water (1.00 mL). The organics were then extracted with EtOAc and dried (anhyd. Na₂SO₄), filtered and concentrated in vacuo. The residue was purified by flash chromatography on SiO₂ (eluent: 0-90% EtOAc in hexanes) to afford the title compound (510 mg, 1.06 mmol, 84%) as a white solid. LC-MS (ESI) m/z 479.3 [M−H]−. LC-MS RT=1.65 min; Method A.

Step 3: ((2R,5R)-2-ethynyl-5-(5-fluoro-2,4-dioxo-3,4-dihydropyrimidin-1(2H)-yl)-2,5-dihydrofuran-2-yl)methyl benzoate. ((2R,4R,5R)-2-ethynyl-5-(5-fluoro-2,4-dioxo-3,4-dihydropyrimidin-1(2H)-yl)-4-(p-tolylthio)tetrahydrofuran-2-yl)methyl benzoate (510 mg, 1.06 mmol) was dissolved in MeCN (8.00 mL) with stirring, and Chloramine-T trihydrate (366 mg, 1.27 mmol) and AcOH (6.14 μL, 106 μmol) were both added. The reaction mixture was stirred at ambient temperature for ca. 2.5 h, then the reaction was quenched by addition of 10 wt. % aqueous NH₄OAc solution (5 mL). The phases were separated, and the organics dried (anhyd. Na₂SO₄), filtered, and concentrated in vacuo. The residue was dissolved in in-BuOH (8.00 mL) and the resulting solution heated to 95° C. for ca. 3 h. The reaction mixture was then cooled to ambient temperature, the volatiles removed in vacuo, and the residue purified by flash chromatography on SiO₂ (eluent: 0-95% EtOAc in hexanes) to furnish the title compound (200 mg, 0.56 mmol, 53%) as a clear oil. LC-MS (ESI) m/z 374.4 [M+H₂O]+. LC-MS RT=0.98 min; Method A.

Step 4: 1-((2R,5R)-5-ethynyl-5-(hydroxymethyl)-2,5-dihydrofuran-2-yl)-5-fluoropyrimidine-2,4(1H,3H)-dione (Cpd. No. 19). ((2R,5R)-2-ethynyl-5-(5-fluoro-2,4-dioxo-3,4-dihydropyrimidin-1(2H)-yl)-2,5-dihydrofuran-2-yl)methyl benzoate (200 mg, 561 μmol) was dissolved in MeOH (25 mL) with stirring at ambient temperature, and DBU (4.3 μL, 28.1 μmol) was added. The resulting mixture was heated to 60° C. until the reaction was determined to be complete by LCMS. The mixture was cooled to ambient temperature, adsorbed onto silica gel, and purified by flash chromatography on SiO₂ (eluent: 0-10% MeOH in DCM) to furnish Cpd. No. 19 (76 mg, 300 μmol, 54%) as a white solid. ¹H NMR (400 MHz, DMSO-d6) δ 11.92 (s, 1H), 8.01 (d, J=7.2 Hz, 1H), 6.87 (dt, J=2.0, 1.5 Hz, 1H), 6.37 (dd, J=5.8, 2.0 Hz, 1H), 6.05 (dd, J=5.8, 1.3 Hz, 1H), 5.60 (t, J=5.8 Hz, 1H), 3.71 (dd, J=12.1, 5.5 Hz, 1H), 3.70 (s, 1H), 3.61 (dd, J=12.1, 6.1 Hz, 1H). LC-MS (ESI) m/z 251.3 [M−H]−. LC-MS RT=0.96 min; Method B.

### Example 36

### Synthesis of 1-((2S,5R)-5-ethyl-4-hydroxy-5-(hydroxymethyl)tetrahydrofuran-2-yl)pyrimidine-2,4(1H,3H)-dione (Cpd. No. 20)

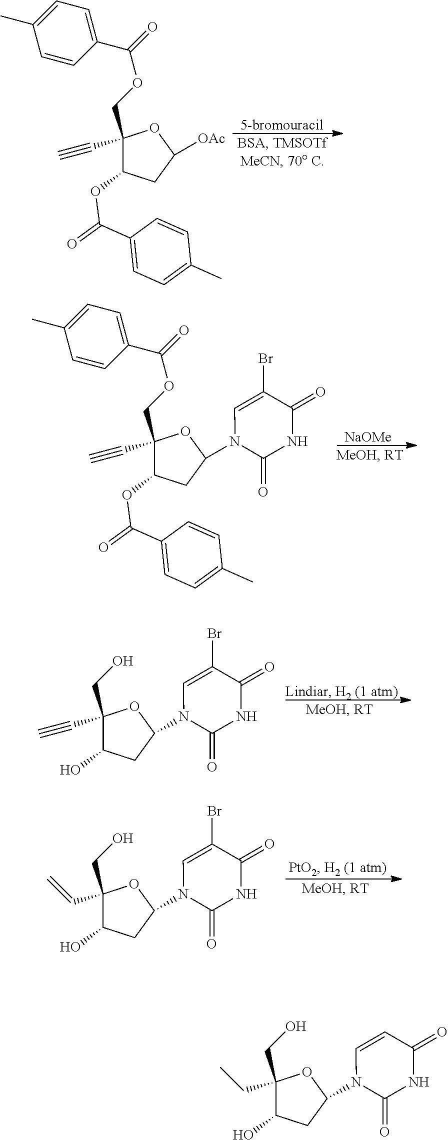

Step 1: (2R,3S)-5-(5-bromo-2,4-dioxo-3,4-dihydropyrimidin-1(2H)-yl)-2-ethynyl-2-(((4-methylbenzoyl)oxy)methyl)tetrahydrofuran-3-yl 4-methylbenzoate. To a solution of N,O-bis(trimethylsilyl)acetamide (104 μL, 687 μmol) in MeCN (2.00 mL) was added 5-bromouracil (44.7 mg, 229 μmol), and the resulting mixture heated to 70° C. with stirring for ca. 1 h. Trimethylsilyl trifluoromethanesulfonate (50.3 μL, 275 μmol) and (2R,3S)-5-acetoxy-2-ethynyl-2-(((4-methylbenzoyl)oxy)methyl)tetrahydrofuran-3-yl 4-methylbenzoate (100 mg, 229 μmol) were then added, and the reaction mixture was stirred at 70° C. overnight before cooling to ambient temperature. The reaction was partitioned between water and EtOAc. The combined organics were dried (anhyd. Na₂SO₄), filtered, and concentrated in vacuo. The residue was purified by flash chromatography on SiO₂ (eluent: 0-90% EtOAc in heptanes) to afford the title compound (121 mg, 213 μmol, 93%) as a clear oil, as a mixture of anomers. For the more polar anomer, LC-MS (ESI) m/z 568.3 [M+H]+. LC-MS RT=1.70 min; Method A. For the less polar anomer, LC-MS (ESI) m/z 567.3 [M−H]−. LC-MS RT=1.73 min; Method A.

Step 2: 5-bromo-1-((2S,4S,5R)-5-ethynyl-4-hydroxy-5-(hydroxymethyl)tetrahydrofuran-2-yl)pyrimidine-2,4(1H,3H)-dione. (2R,3S)-5-(5-bromo-2,4-dioxo-3,4-dihydropyrimidin-1(2H)-yl)-2-ethynyl-2-(((4-methylbenzoyl)oxy)methyl)tetrahydrofuran-3-yl 4-methylbenzoate (454 mg, 213 μmol) was dissolved in MeOH (2.00 mL), and a solution of NaOMe (25 wt. % in MeOH, 2.00 mL, 427 μmol) was added with stirring. The reaction mixture was stirred at ambient temperature until the reaction was determined to be complete by LCMS. The reaction mixture was adsorbed onto silica gel and purified by flash chromatography on SiO₂ (eluent: 0-20% MeOH in DCM) to afford the title compound (44.9 mg, 13.6 μmol, 64%) as a white solid, as a mixture of anomers. Further purification by prep-HPLC afforded the title compound (6.00 mg) as a single stereoisomer. ¹H NMR (400 MHz, DMSO-d6) δ 11.81 (br. s, 1H), 8.32 (s, 1H), 6.09 (dd, J=7.2, 3.4 Hz, 1H), 5.73 (d, J=4.4 Hz, 1H), 5.32 (t, J=6.3 Hz, 1H), 4.27 (dt, J=6.1, 3.9 Hz, 1H), 3.74 (s), 3.50 (dd, J=11.8, 5.9 Hz, 1H), 3.46 (dd, J=11.8, 6.8 Hz, 1H), 2.74-2.66 (m, 1H), 2.01 (dt, J=14.1, 3.5 Hz, 1H).

Step 3: 5-bromo-1-((2S,4S,5R)-4-hydroxy-5-(hydroxymethyl)-5-vinyltetrahydrofuran-2-yl)pyrimidine-2,4(1H,3H)-dione. In a glass vial equipped with a magnetic stirrer bar and capped with a rubber septum, 5-bromo-1-((2S,4S,5R)-5-ethynyl-4-hydroxy-5-(hydroxymethyl)tetrahydrofuran-2-yl)pyrimidine-2,4(1H,3H)-dione (6.00 mg, 18.1 μmol) was dissolved in MeOH (181 μL) with stirring. Lindlar catalyst (6.00 mg) was then added, and the vessel flushed with hydrogen gas from a balloon. The reaction mixture was then stirred under hydrogen (1 atm) at ambient temperature for ca. 3 h. The mixture was filtered through a short plug of Celite®, the filter cake washed with MeOH, and the filtrate concentrated in vacuo to furnish the crude title compound (4.85 mg, 14.5 μmol, 80%). ¹H NMR (400 MHz, CD₃OD) δ 8.45 (s, 1H), 6.34 (dd, J=7.8, 2.0 Hz, 1H), 6.12 (dd, J=17.4, 11.2 Hz, 1H), 5.44 (dd, J=17.4, 1.7 Hz, 1H), 5.35 (dd, J=11.2, 1.7 Hz, 1H), 4.40 (dd, J=5.9, 0.9 Hz, 1H), 3.51 (d, J=11.8 Hz, 1H), 3.43 (d, J=11.8 Hz, 1H), 2.97-2.89 (m, 1H), 2.06-2.00 (m, 1H).

Step 4: 1-((2S,4S,5R)-5-ethyl-4-hydroxy-5-(hydroxymethyl)tetrahydrofuran-2-yl)pyrimidine-2,4(1H,3H)-dione (Cpd. No. 20). In a glass vial equipped with a magnetic stirrer bar and capped with a rubber septum, 5-bromo-1-((2S,4S,5R)-4-hydroxy-5-(hydroxymethyl)-5-vinyltetrahydrofuran-2-yl)pyrimidine-2,4(1H,3H)-dione (6.04 mg, 18.1 μmol) was dissolved in MeOH (181 μL) with stirring. PtO₂ (2.06 mg, 9.06 μmol) was then added, and the vessel flushed with hydrogen gas from a balloon. The reaction mixture was then stirred under hydrogen (1 atm) at ambient temperature for ca. 4 h. The mixture was filtered through a short plug of Celite®, the filter cake washed with MeOH, and the filtrate concentrated in vacuo. Purification of the residue by flash chromatography on SiO₂ (eluent: 50-100% EtOAc in heptanes, then 100% IPA) furnished Cpd. No. 20 (2.45 mg, 9.6 μmol, 53%) as an amorphous white solid. ¹H NMR (400 MHz, CD₃OD) δ 8.00 (d, J=8.1 Hz, 1H), 6.15 (dd, J=7.9, 2.6 Hz, 1H), 5.67 (d, J=8.1 Hz, 1H), 4.22 (dd, J=6.0, 1.4 Hz, 1H), 3.46 (q, J=11.8 Hz, 2H), 2.87 (ddd, J=14.7, 7.9, 6.0 Hz, 1H), 2.01-1.92 (m, 1H), 1.59-1.46 (m, 1H). LC-MS (ESI) m/z 255.2 [M−H]−. LC-MS RT=1.05 min; Method H.

### Example 37

### Synthesis of 4-amino-5-bromo-1-((2R,4S,5R)-5-(fluoromethyl)-4-hydroxy-5-(hydroxymethyl)tetrahydrofuran-2-yl)pyrimidin-2(1H)-one (Cpd. No. 117)

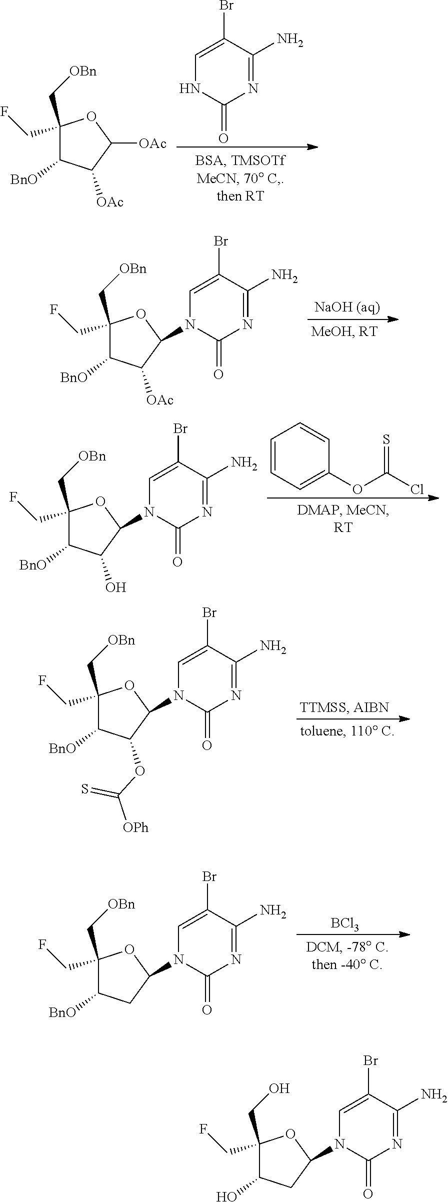

Step 1: (2R,3R,4S,5R)-2-(4-amino-5-bromo-2-oxopyrimidin-1(2H)-yl)-4-(benzyloxy)-5-((benzyloxy)methyl)-5-(fluoromethyl)tetrahydrofuran-3-yl acetate. To a mixture of 4-amino-5-bromo-1H-pyrimidin-2-one (0.26 g, 1.37 mmol) in MeCN (15 mL) was added trimethylsilyl (1E)-N-trimethylsilylethanimidate (835.14 mg, 4.11 mmol) in one portion at 25° C. under N₂. Then the mixture was stirred at 70° C. for 1 hour. After cooling to 25° C., TMSOTf (373.37 mg, 1.68 mmol) and (2S,3R,4S,5R)-4-(benzyloxy)-5-((benzyloxy)methyl)-5-(fluoromethyl)tetrahydrofuran-2,3-diyl diacetate (0.5 g, 1.12 mmol) were added dropwise at 25° C. The resulting mixture was stirred at 25° C. for 16 hours. Then the reaction was quenched by addition of 10 wt % of aqueous citric acid solution (10 mL) and then extracted by EtOAc (20 mL). The organic layer was washed with brine (15 mL), dried over anhydrous Na₂SO₄, filtered and concentrated. The residue was purified by column chromatography on silica gel (column height: 250 mm, diameter. 100 mm, 100-200 mesh silica gel) eluting with 2% MeOH in dichloromethane to give the title compound (0.326 g, 51% yield) as yellow oil. ¹H NMR (400 MHz, CDCl₃) δ 8.26 (s, 1H), 7.40-7.26 (m, 12H), 6.21 (d, J=4.0 Hz, 1H), 5.63 (s, 1H), 5.53-5.45 (m, 1H), 4.72-4.35 (m, 7H), 3.90-3.82 (m, 1H), 3.60-3.53 (m, 1H), 2.11 (s, 3H).

Step 2: 4-amino-1-((2R,3R,4S,5R)-4-(benzyloxy)-5-((benzyloxy)methyl)-5-(fluoromethyl)-3-hydroxytetrahydrofuran-2-yl)-5-bromopyrimidin-2(1H)-one. To a mixture of (2R,3R,4S,5R)-2-(4-amino-5-bromo-2-oxopyrimidin-1(2H)-yl)-4-(benzyloxy)-5-((benzyloxy)methyl)-5-(fluoromethyl)tetrahydrofuran-3-yl acetate (0.325 g, 563.83 μmol) in MeOH (15 mL) was added NaOH (2 M, 2 mL) aqueous solution in one portion at 25° C. The mixture was stirred at 25° C. for 30 min. The reaction mixture was concentrated directly. The residue was purified by column chromatography on silica gel (column height: 250 mm, diameter: 100 mm, 100-200 mesh silica gel) eluting with 2% MeOH in DCM to give the title compound (0.29 g, 96% yield) as a white solid. ¹H NMR (400 MHz, CDCl₃) δ 8.09 (s, 1H), 7.40-7.27 (m, 10H), 6.73 (s, 1H), 5.93 (d, J=4.8 Hz, 1H), 5.59 (s, 1H), 4.81-4.52 (m, 6H), 4.46-4.40 (m, 1H), 4.28 (d, J=5.6 Hz, 1H), 3.89-3.76 (m, 2H), 3.68-3.61 (m, 1H).

Step 3: O-((2R,3R,4S,5R)-2-(4-amino-5-bromo-2-oxopyrimidin-1(2H)-yl)-4-(benzyloxy)-5-((benzyloxy)methyl)-5-(fluoromethyl)tetrahydrofuran-3-yl) O-phenyl carbonothioate. To a solution of 4-amino-1-((2R,3R,4S,5R)-4-(benzyloxy)-5-((benzyloxy)methyl)-5-(fluoromethyl)-3-hydroxytetrahydrofuran-2-yl)-5-bromopyrimidin-2(1H)-one (0.29 g, 542.69 μmol) in MeCN (15 mL) was added DMAP (198.90 mg, 1.63 mmol), then O-phenyl chlorothionoformate (141 mg, 814.04 μmol) was added dropwise at 25° C. under N₂. After stirring at 25° C. for 1 hour, the mixture was concentrated directly. The residue was purified by column chromatography on silica gel (column height: 250 mm, diameter: 100 mm, 100-200 mesh silica gel) eluting with 1% MeOH in DCM) to give the title compound (0.266 g, 73% yield) as a light yellow solid. ¹H NMR (400 MHz, CDCl₃) δ 8.17 (s, 1H), 7.42-7.28 (m, 13H), 7.05-6.99 (m, 2H), 6.41 (d, J=5.2 Hz, 1H), 6.03 (t, J=5.2 Hz, 1H), 4.73 (d, J=11.2 Hz, 1H), 4.70-4.50 (m, 6H), 3.91-3.86 (m, 1H), 3.71-3.65 (m, 1H).

Step 4: 4-amino-1-((2R,4S,5R)-4-(benzyloxy)-5-((benzyloxy)methyl)-5-(fluoromethyl)tetrahydrofuran-2-yl)-5-bromopyrimidin-2(1H)-one. To a mixture of O-((2R,3R,4S,5R)-2-(4-amino-5-bromo-2-oxopyrimidin-1(2H)-yl)-4-(benzyloxy)-5-((benzyloxy)methyl)-5-(fluoromethyl)tetrahydrofuran-3-yl) O-phenyl carbonothioate (0.266 g, 396.69 μmol) in toluene (12 mL) was added AIBN (33 mg, 198.35 μmol) and TTMSS (493.21 mg, 1.98 mmol) in one portion at 25° C. under N₂. The mixture was heated to 110° C. and stirred for 2 hours. Then the reaction mixture was cooled to room temperature and concentrated directly. The residue was purified by prep-TLC (DCM/MeOH=15/1) to give the title compound (40 mg, 19% yield) as colorless oil.

Step 5: 4-amino-5-bromo-1-((2R,4S,5R)-5-(fluoromethyl)-4-hydroxy-5-(hydroxymethyl)tetrahydrofuran-2-yl)pyrimidin-2(1H)-one (Cpd. No. 117). To a mixture of 4-amino-1-((2R,4S,5R)-4-(benzyloxy)-5-((benzyloxy)methyl)-5-(fluoromethyl)tetrahydrofuran-2-yl)-5-bromopyrimidin-2(1H)-one (0.04 g, 77.16 μmol) in DCM (9 mL) was added BCl₃ (1 M in DCM, 0.5 mL) dropwise at −78° C. under N₂. The reaction mixture was stirred at −78° C. for 15 min, then allowed to warm up to −40° C. and stirred for another 0.5 h. Then the reaction was quenched by addition of MeOH (1 mL) and NH₄OH (1 mL) at −40° C. After stirring −40° C. for another 10 min, the mixture was warmed to room temperature and stirred for another 10 min. After that, the reaction mixture was concentrated directly. The residue was purified by prep-HPLC (basic) to give the title compound (1.6 mg, 6% yield) as a white solid. ¹H NMR (400 MHz, CDCl₃) δ 8.42 (s, 1H), 6.24 (t, J=6.4 Hz, 1H), 4.68-4.62 (m, 1H), 4.55-4.46 (m, 2H), 3.75-3.71 (m, 2H), 2.55-2.42 (m, 1H), 2.35-2.24 (m, 1H).

### Example 38

### Synthesis of 5-chloro-1-((2R,4S,5R)-4-hydroxy-5-(hydroxymethyl)-5-methyltetrahydrofuran-2-yl)pyrimidine-2,4(1H,3H)-dione (Cpd. No. 120)

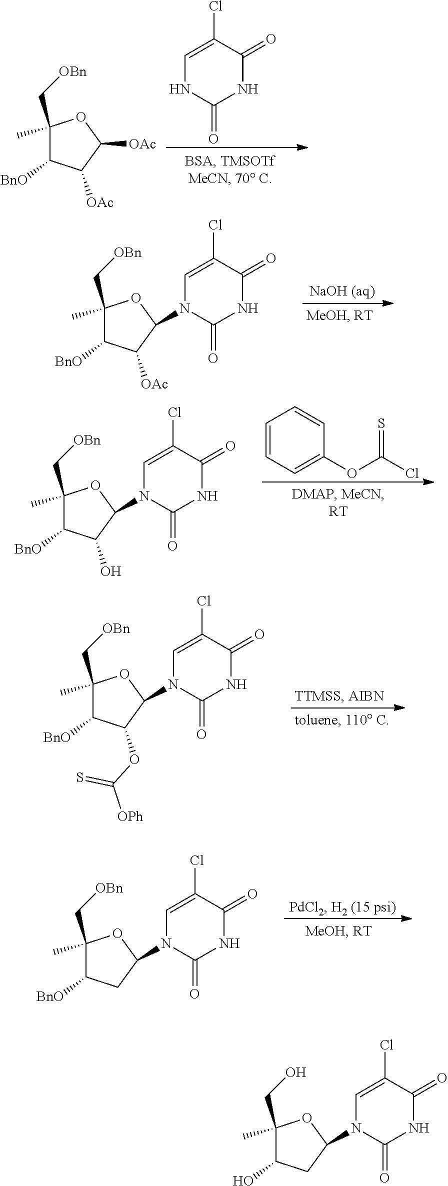

Step 1: (2R,3R,4S,5R)-4-(benzyloxy)-5-((benzyloxy)methyl)-2-(5-chloro-2,4-dioxo-3,4-dihydropyrimidin-1(2H)-yl)-5-methyltetrahydrofuran-3-yl acetate. A mixture of 5-chloro-1H-pyrimidine-2,4-dione (0.22 g, 1.50 mmol) and BTMSA (767.51 mg, 4.50 mmol) in MeCN (10 mL) was stirred at 70° C. for 1 hour, then the reaction mixture was cooled to room temperature. A solution of (0.5 g, 1.17 mmol) in MeCN (10 mL) and TMSOTf (520 mg, 2.34 mmol) were added, then the mixture was stirred at 30° C. for 16 hours. After that, the reaction solution was diluted with EtOAc (50 mL) and washed with brine (30 mL×2), concentrated to give the crude title compound (0.5 g, 83% yield) as a colorless oil. LC-MS (ESI) m/z 537.0 [M+Na]+. LC-MS RT=0.901 min; Method C.

Step 2: 1-((2R,3R,4S,5R)-4-(benzyloxy)-5-((benzyloxy)methyl)-3-hydroxy-5-methyltetrahydrofuran-2-yl)-5-chloropyrimidine-2,4(1H,3H)-dione. A mixture of (2R,3R,4S,5R)-4-(benzyloxy)-5-((benzyloxy)methyl)-2-(5-chloro-2,4-dioxo-3,4-dihydropyrimidin-1(2H)-yl)-5-methyltetrahydrofuran-3-yl acetate (0.5 g, 970.96 μmol) and NaOH (2 M, 1 mL) in MeOH (10 mL) was stirred at 20° C. for 0.2 hour. The reaction solution was concentrated and diluted with EtOAc (50 ml), washed with brine (30 mL×2), then concentrated to afford the crude title compound (0.4 g, 87% yield) as white solid used for next step directly.

Step 3: O-((2R,3R,4S,5R)-4-(benzyloxy)-5-((benzyloxy)methyl)-2-(5-chloro-2,4-dioxo-3,4-dihydropyrimidin-1(2H)-yl)-5-methyltetrahydrofuran-3-yl) O-phenyl carbonothioate. To a solution of 1-((2R,3R,4S,5R)-4-(benzyloxy)-5-((benzyloxy)methyl)-3-hydroxy-5-methyltetrahydrofuran-2-yl)-5-chloropyrimidine-2,4(1H,3H)-dione (0.2 g, 422.91 μmol) and DMAP (155 mg, 1.27 mmol) in MeCN (20 mL) was added O-phenyl chlorothionoformate (109 mg, 634.36 μmol), then stirred for 1 hour at 20° C. Then the reaction mixture was diluted with EtOAc (50 mL) and washed with brine (30 mL×2), concentrated to give the crude product. The residue was purified by column chromatography (DCM/MeOH=100/1 to 100/5) to afford the title compound (0.23 g, 89% yield) as a colorless solid.

Step 4: 1-((2R,4S,5R)-4-(benzyloxy)-5-((benzyloxy)methyl)-5-methyltetrahydrofuran-2-yl)-5-chloropyrimidine-2,4(1H,3H)-dione. A mixture of O-((2R,3R,4S,5R)-4-(benzyloxy)-5-((benzyloxy)methyl)-2-(5-chloro-2,4-dioxo-3,4-dihydropyrimidin-1(2H)-yl)-5-methyltetrahydrofuran-3-yl) O-phenyl carbonothioate (0.33 g, 541 umol), TTMSS (673.6 mg, 2.71 mmol) and AIBN (44 mg, 270 umol) in toluene (15 mL) was stirred at 110° C. for 2 hours. After cooling to 20° C., the reaction mixture was concentrated to give the crude product. The residue was purified by column chromatography (DCM: MeOH=100/1 to 20/1) to afford the title compound (0.11 g, 44% yield) as a colorless oil. LC-MS (ESI) m/z 479.0 [M+Na]+. LC-MS RT=0.892 min; Method C.

Step 5: 5-chloro-1-((2R,4S,5R)-4-hydroxy-5-(hydroxymethyl)-5-methyltetrahydrofuran-2-yl)pyrimidine-2,4(1H,3H)-dione (Cpd. No. 120). A mixture of 1-((2R,4S,5R)-4-(benzyloxy)-5-((benzyloxy)methyl)-5-methyltetrahydrofuran-2-yl)-5-chloropyrimidine-2,4(1H,3H)-dione (50 mg, 109.43 μmol) and PdCl₂ (9.7 mg, 54.71 μmol) in MeOH (10 mL) was stirred at 26° C. for 20 min under H₂ (15 Psi). After that, the reaction solution was filtered and the filtrate was concentrated to give the crude product. The residue was purified by pre-HPLC (acetonitrile 0-27%/FA-MeCN in water) to afford Cpd. No. 120 (13.9 mg, 46% yield) as a white solid. ¹H NMR (400 MHz, MeOD-d₄): δ 8.45 (s, 1H), 6.17-6.14 (m, 1H), 4.41-4.39 (m, 1H), 3.64-3.57 (m, 2H), 2.42-2.34 (m, 2H), 1.16 (s, 3H).

### Example 39

### Synthesis of 1-((2R,4S,5R)-4-hydroxy-5-(hydroxymethyl)-5-methyltetrahydrofuran-2-yl)-5-methylpyrimidine-2,4(1H,3H)-dione (Cpd. No. 122)

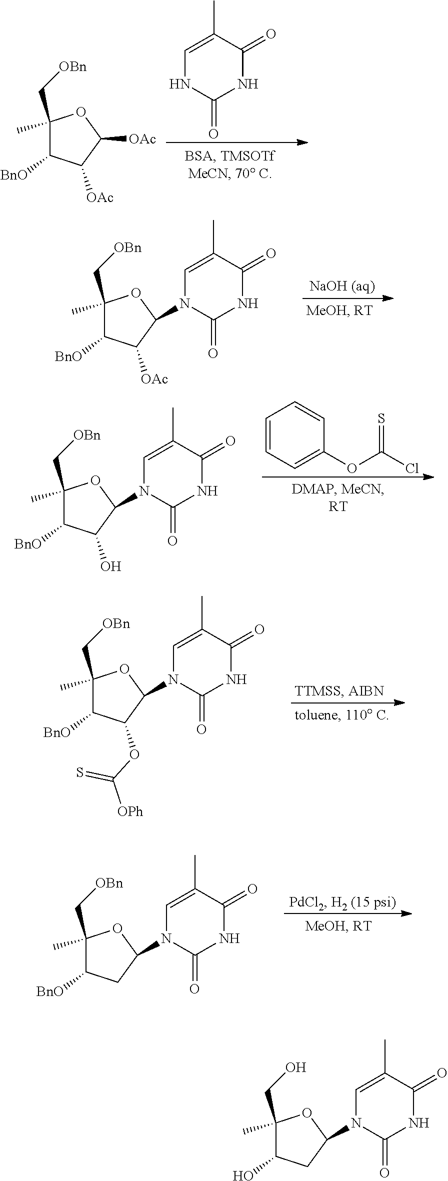

Step 1: (2R,3R,4S,5R)-4-(benzyloxy)-5-((benzyloxy)methyl)-5-methyl-2-(5-methyl-2,4-dioxo-3,4-dihydropyrimidin-1(2H)-yl)tetrahydrofuran-3-yl acetate. To a mixture of 5-methyl-1H-pyrimidine-2,4-dione (0.2 g, 1.59 mmol) in MeCN (15 mL) was added NO-bis(trimethylsilyl)acetamide (811 mg, 4.76 mmol) in one portion at 25° C. under N₂. Then the mixture was stirred at 70° C. for 1 hour. After cooling to 25° C., TMSOTf (519 mg, 2.33 mmol) and (2S,3R,4S,5R)-4-(benzyloxy)-5-((benzyloxy)methyl)-5-methyltetrahydrofuran-2,3-diyl diacetate (0.5 g, 1.17 mmol) were added at 25° C. The resulting mixture was stirred at 25° C. for 16 hours. Then the reaction was quenched by addition of 10 wt % of aqueous citric acid solution (10 mL) and then extracted by EtOAc (20 mL). The organic layer was washed with brine (15 mL), dried over Na₂SO₄, filtered and concentrated to give the title compound (0.5 g, 87% yield) as yellow oil. LC-MS (ESI) m/z 517.1 [M+Na]+. LC-MS RT=0.876 min; Method C.

Step 2: 1-((2R,3R,4S,5R)-4-(benzyloxy)-5-((benzyloxy)methyl)-3-hydroxy-5-methyltetrahydrofuran-2-yl)-5-methylpyrimidine-2,4(1H,3H)-dione. To a mixture of (2R,3R,4S,5R)-4-(benzyloxy)-5-((benzyloxy)methyl)-5-methyl-2-(5-methyl-2,4-dioxo-3,4-dihydropyrimidin-1(2H)-yl)tetrahydrofuran-3-yl acetate (0.5 g, 1.01 mmol) in MeOH (15 mL) was added NaOH (2 M, 2 mL) aqueous solution in one portion at 25° C. The mixture was stirred at 25° C. for 30 min. The reaction mixture was concentrated directly to give the title compound (0.45 g, 98% yield) as a white solid.

Step 3: O-((2R,3R,4S,5R)-4-(benzyloxy)-5-((benzyloxy)methyl)-5-methyl-2-(5-methyl-2,4-dioxo-3,4-dihydropyrimidin-1(2H)-yl)tetrahydrofuran-3-yl) O-phenyl carbonothioate. To a mixture of 1-((2R,3R,4S,5R)-4-(benzyloxy)-5-((benzyloxy)methyl)-3-hydroxy-5-methyltetrahydrofuran-2-yl)-5-methylpyrimidine-2,4(1H,3H)-dione (0.35 g, 773.48 μmol) in MeCN (15 mL) was added DMAP (283.5 mg, 2.32 mmol), then O-phenyl chlorothionoformate (200 mg, 1.16 mmol) was added dropwise at 25° C. under N₂. After stirring at 25° C. for 1 hour, the mixture was concentrated directly. The residue was purified by silica gel chromatography (column height: 250 mm, diameter. 100 mm, 100-200 mesh silica gel, DCM/MeOH=99/1) to give the title compound (0.4 g, 88% yield) as a light yellow solid. LC-MS (ESI) m/z 611.1 [M+Na]+. LC-MS RT=0.981 min; Method C.

Step 4: 1-((2R,4S,5R)-4-(benzyloxy)-5-((benzyloxy)methyl)-5-methyltetrahydrofuran-2-yl)-5-methylpyrimidine-2,4(1H,3H)-dione. To a mixture of O-((2R,3R,4S,5R)-4-(benzyloxy)-5-((benzyloxy)methyl)-5-methyl-2-(5-methyl-2,4-dioxo-3,4-dihydropyrimidin-1(2H)-yl)tetrahydrofuran-3-yl) O-phenyl carbonothioate (0.2 g, 339.75 μmol) in toluene (12 mL) was added AIBN (28 mg, 169.87 μmol) and TTMSS (422 mg, 1.70 mmol) in one portion at 25° C. under N₂. The mixture was heated to 110° C. and stirred for 2 hours. Then the reaction mixture was concentrated directly. The residue was purified by prep-TLC (DCM/MeOH=99/1) to give the title compound (100 mg, 67% yield) as colorless oil. LC-MS (ESI) m/z 437.0 [M+Na]+. LC-MS RT=0.886 min; Method C.

Step 5: 1-((2R,4S,5R)-4-hydroxy-5-(hydroxymethyl)-5-methyltetrahydrofuran-2-yl)-5-methylpyrimidine-2,4(1H,3H)-dione (Cpd. No. 122). To a mixture of 1-((2R,4S,5R)-4-(benzyloxy)-5-((benzyloxy)methyl)-5-methyltetrahydrofuran-2-yl)-5-methylpyrimidine-2,4(1H,3H)-dione (0.08 g, 183.28 μmol) in MeOH (2 mL) was added PdCl₂ (3 mg) at 25° C. under H₂ (15 psi). The mixture was stirred at 25° C. for 20 min. After that, the mixture was filtered and concentrated directly. The residue was purified by prep-HPLC (FA) to furnish Cpd. No. 122 (10.2 mg, 22% yield) as a white solid. ¹H NMR (400 MHz, CD₃OD) δ 7.90 (s, 1H), 6.21 (t, J=6.4 Hz, 1H), 4.40 (t, J=6.0 Hz, 1H), 3.66-3.51 (m, 2H), 2.34 (t, J=6.4 Hz, 2H), 1.88 (s, 3H), 1.17 (s, 3H).

### Example 40

### Synthesis of 4-amino-5-chloro-1-((2R,4S,5R)-4-hydroxy-5-(hydroxymethyl)-5-methyltetrahydrofuran-2-yl)pyrimidin-2(1H)-one (Cpd. No. 131)

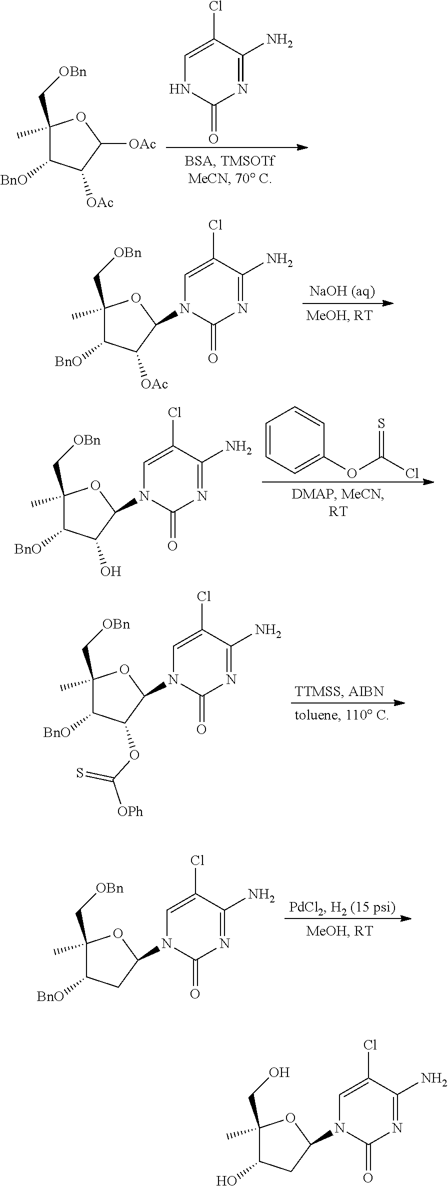

Step 1: (2R,3R,4S,5R)-2-(4-amino-5-chloro-2-oxopyrimidin-1(2H)-yl)-4-(benzyloxy)-5-((benzyloxy)methyl)-5-methyltetrahydrofuran-3-yl acetate. To a mixture of 4-amino-5-chloro-1H-pyrimidin-2-one (0.21 g, 1.44 mmol) in MeCN (15 mL) was added N,O-bis(trimethylsilyl)acetamide (881 mg, 4.33 mmol) in one portion at 25° C. under N₂. Then the mixture was stirred at 70° C. for 1 hour. After cooling to 25° C., TMSOTf (389 mg, 1.75 mmol) and (2S,3R,4S,5R)-4-(benzyloxy)-5-((benzyloxy)methyl)-5-methyltetrahydrofuran-2,3-diyl diacetate (0.5 g, 1.17 mmol) were added dropwise at 25° C. The resulting mixture was stirred at 25° C. for 16 hours. Then the reaction was quenched by addition of 10 wt % of aqueous citric acid solution (10 mL) and then extracted by EtOAc (20 mL). The organic layer was washed with brine (15 mL), dried over Na₂SO₄, filtered and concentrated. The residue was purified by column chromatography on silica gel (column height: 250 mm, diameter: 100 mm, 100-200 mesh silica gel) eluting with 2% MeOH in DCM to give the title compound (0.59 g, 98% yield) as yellow oil. LC-MS (ESI) m/z 236.1 [M+Na]+. LC-MS RT=0.846 min; Method C.

Step 2: 4-amino-1-((2R,3R,4S,5R)-4-(benzyloxy)-5-((benzyloxy)methyl)-3-hydroxy-5-methyltetrahydrofuran-2-yl)-5-chloropyrimidin-2(1H)-one. To a mixture of (2R,3R,4S,5R)-2-(4-amino-5-chloro-2-oxopyrimidin-1(2H)-yl)-4-(benzyloxy)-5-((benzyloxy)methyl)-5-methyltetrahydrofuran-3-yl acetate (0.55 g, 1.07 mmol) in MeOH (15 mL) was added NaOH (2 M, 2 mL) aqueous solution in one portion at 25° C. The mixture was stirred at 25° C. for 30 min. Then the reaction mixture was concentrated. The residue was purified by column chromatography on silica gel (column height: 250 mm, diameter: 100 mm, 100-200 mesh silica gel) eluting with 2% MeOH in DCM to give the title compound (0.5 g, 99% yield) as a white solid.

Step 3: O-((2R,3R,4S,5R)-2-(4-amino-5-chloro-2-oxopyrimidin-1(2H)-yl)-4-(benzyloxy)-5-((benzyloxy)methyl)-5-methyltetrahydrofuran-3-yl) O-phenyl carbonothioate. To a mixture of 4-amino-1-((2R,3R,4S,5R)-4-(benzyloxy)-5-((benzyloxy)methyl)-3-hydroxy-5-methyltetrahydrofuran-2-yl)-5-chloropyrimidin-2(1H)-one (0.5 g, 1.06 mmol) in MeCN (15 mL) was added DMAP (388 mg, 3.18 mmol), then O-phenyl chloromethanethioate (274 mg, 1.59 mmol) was added dropwise at 25° C. under N₂. After stirring at 25° C. for 1 hour, the mixture was concentrated directly. The residue was purified by column chromatography on silica gel (column height: 250 mm, diameter: 100 mm, 100-200 mesh silica gel) eluting with 1% MeOH in DCM) to afford the title compound (0.5 g, 78% yield) as a light yellow solid. ¹H NMR (400 MHz, DMSO-d₆) δ 8.22 (s, 1H), 7.42-7.28 (m, 13H), 7.07-7.01 (m, 2H), 6.33 (d, J=3.2 Hz, 1H), 6.02-5.96 (m, 1H), 5.58 (s, 1H), 4.75 (d, J=11.6 Hz, 1H), 4.58-4.52 (m, 1H), 4.50-4.40 (m, 3H), 3.64 (d, J=10.4 Hz, 1H), 3.38 (d, J=10.4 Hz, 1H), 1.31 (s, 3H).

Step 4: 4-amino-1-((2R,4S,5R)-4-(benzyloxy)-5-((benzyloxy)methyl)-5-methyltetrahydrofuran-2-yl)-5-chloropyrimidin-2(1H)-one. To a mixture of O-((2R,3R,4S,5R)-2-(4-amino-5-chloro-2-oxopyrimidin-1(2H)-yl)-4-(benzyloxy)-5-((benzyloxy)methyl)-5-methyltetrahydrofuran-3-yl) O-phenyl carbonothioate (0.5 g, 822.23 μmol) in toluene (12 mL) was added AIBN (68 mg, 411.11 μmol) and TTMSS (1.02 g, 4.11 mmol) in one portion at 25° C. under N₂. The mixture was heated to 110° C. and stirred for 2 hours. Then the reaction mixture was concentrated directly. The residue was purified by prep-TLC (DCM/MeOH=20/1) to give the title compound (80 mg, 21% yield) as colorless oil.

Step 5: 4-amino-5-chloro-1-((2R,4S,5R)-4-hydroxy-5-(hydroxymethyl)-5-methyltetrahydrofuran-2-yl)pyrimidin-2(1H)-one (Cpd. No. 131). To a mixture of 4-amino-1-((2R,4S,5R)-4-(benzyloxy)-5-((benzyloxy)methyl)-5-methyltetrahydrofuran-2-yl)-5-chloropyrimidin-2(1H)-one (0.02 g, 43.87 μmol) in MeOH (2 mL) was added PdCl₂ (778 ug) at 25° C. under H₂ (15 psi). The mixture was stirred at 25° C. for 0.3 hour. After that, the mixture was filtered and concentrated directly. The residue was purified by pre-HPLC (acetonitrile 0-27/0.225% FA in water) to afford Cpd. No. 131 (2.6 mg, 7% yield) as a white solid. ¹H NMR (400 MHz, CD₃OD) δ 8.50 (s, 1H), 6.15-6.05 (m, 1H), 4.36 (t, J=6.4 Hz. 1H), 3.68-3.51 (m. 2H), 2.55-2.41 (m, 1H), 2.35-2.20 (m, 1H), 1.17 (s, 3H).

### Example 41

### Synthesis of ((((3R,5R)-5-(6-amino-9H-purin-9-yl)tetrahydrofuran-3-yl)oxy)methyl)phosphonic Acid (Cpd. No. 113)

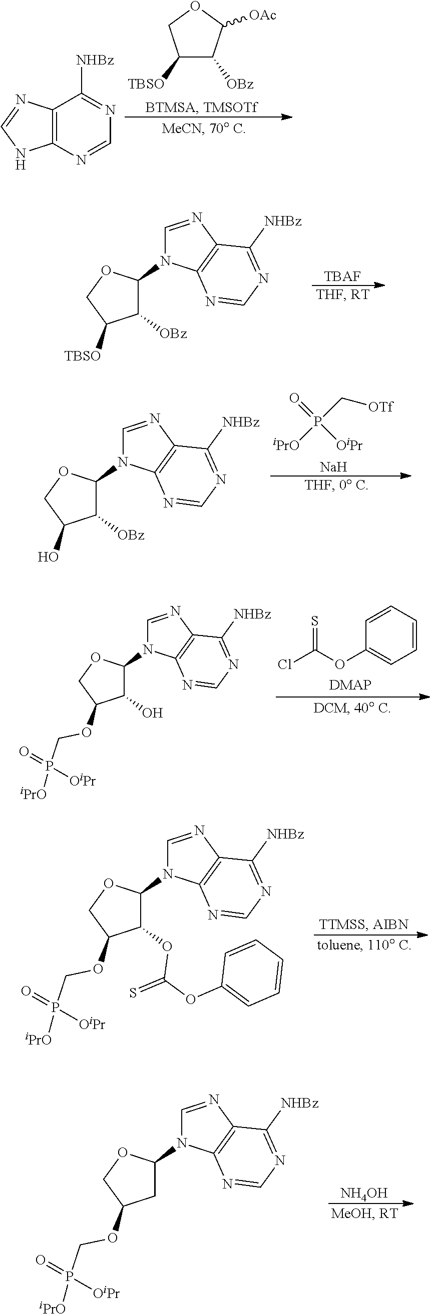

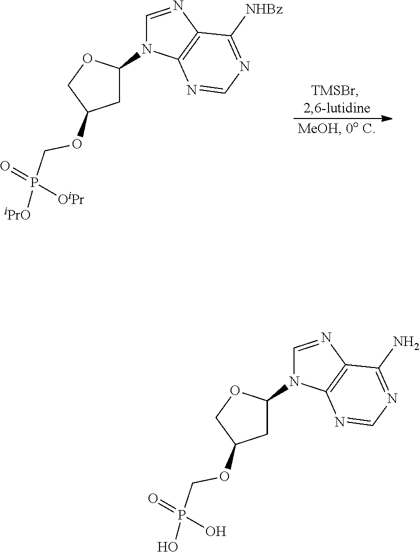

Step 1: (2R,3R,4S)-2-(6-benzamido-9H-purin-9-yl)-4-((tert-butyldimethylsilyl)oxy)tetrahydrofuran-3-yl benzoate. A mixture of N-(9H-purin-6-yl)benzamide (1.0 g, 4.2 mmol) in acetonitrile (20 mL) was degassed with N₂ for three times. Then N,O-bis(trimethylsilyl)acetamide (2.2 g, 10.5 mmol) was added. The resulting mixture was stirred at 70° C. for 1 h and then cooled to room temperature. After that, trimethylsilyl trifluoromethanesulfonate (873.0 mg, 3.9 mmol) was added and then (3R,4S)-2-acetoxy-4-((tert-butyldimethylsilyl)oxy)tetrahydrofuran-3-yl benzoate (1.3 g, 3.4 mmol) in acetonitrile (10 mL) was added. The reaction mixture was stirred at 75° C. for 16 h. After cooling to room temperature, the reaction mixture was diluted with water (30 mL), extracted with EtOAc (40 mL), washed with brine (30 mL), dried over Na₂SO₄, filtered, and concentrated. The residue was purified by silica gel chromatography eluting with 0-30% ethyl acetate in petroleum ether to afford the title product (0.8 g, 42% yield) as a white solid. ¹H NMR (400 MHz, CDCl₃) δ 8.83 (s, 1H), 8.49 (s, 1H), 8.08-7.93 (m, 5H), 7.56-7.50 (m, 5H), 6.53 (s, 1H), 5.64 (s, 1H), 4.59-4.52 (m, 1H), 4.50-4.43 (m, 2H), 0.88 (s, 9H), 0.17 (s, 3H), 0.08 (s, 3H).

Step 2: (2R,3R,4S)-2-(6-benzamido-9H-purin-9-yl)-4-hydroxytetrahydrofuran-3-yl benzoate. To a solution of (2R,3R,4S)-2-(6-benzamido-9H-purin-9-yl)-4-((tert-butyldimethylsilyl)oxy)tetrahydrofuran-3-yl benzoate (0.8 g, 1.4 mmol) in THF (10 mL) was added TBAF (1 M, 1.4 mL) in THF at 0° C. The reaction mixture was stirred at 25° C. for 1 h. Then the reaction mixture was diluted with water (10 mL), extracted with EtOAc (20 mL), washed with brine (20 mL×2), dried over Na₂SO₄ and concentrated. The residue was purified by silica gel chromatography eluting with 0-3% MeOH in DCM to afford the title product (0.35 g, 55% yield) as a white solid. ¹H NMR (400 MHz, CDCl₃) δ 9.06 (s, 1H), 8.85 (s, 1H), 8.18 (s, 1H), 8.06 (t, J=8.4 Hz, 4H), 7.71-7.63 (m, 2H), 7.58-7.46 (m, 4H), 6.90 (d, J=10.8 Hz, 1H), 6.02 (s, 1H), 5.62 (s, 1H), 4.73-4.66 (m, 1H), 4.43-4.35 (m, 1H), 4.32-4.25 (m, 1H).

Step 3: diisopropyl ((((3S,4R,5R)-5-(6-benzamido-9H-purin-9-yl)-4-hydroxytetrahydrofuran-3-yl)oxy)methyl)phosphonate. A solution of (2R,3R,4S)-2-(6-benzamido-9H-purin-9-yl)-4-hydroxytetrahydrofuran-3-yl benzoate (700.0 mg, 1.6 mmol) and diisopropoxyphosphorylmethyl trifluoromethanesulfonate (516.0 mg, 1.6 mmol) in THF (15 mL) was degassed with N₂ for three times and cooled to 0° C. NaH (189.0 mg, 4.7 mmol, 60% purity) was added in several batches and the reaction mixture was stirred at 0° C. for 1 h. Then the reaction mixture was quenched with saturated NH₄Cl aqueous (10 mL), extracted with EtOAc (20 mL). The organic layer was washed with brine (20 mL), dried over Na₂SO₄, filtered and concentrated. The residue was purified by silica gel chromatography eluting with 0-3% MeOH in DCM to afford the title product (350 mg, 43% yield) as a white solid. ¹H NMR (400 MHz, CDCl₃) 9.00 (s, 1H), 8.80 (s, 1H), 8.23 (s, 1H), 8.06-8.00 (m, 2H), 7.66-7.60 (m, 1H), 7.57-7.50 (m, 2H), 6.03 (d, J=4.4 Hz, 1H), 4.91 (t, J=4.0 Hz, 1H), 4.81-4.71 (m, 2H), 4.40-4.27 (m, 3H), 4.03-3.96 (m, 1H), 3.85-3.76 (m, 1H), 1.33 (d, J=6.0 Hz, 12H).

Step 4: O-((2R,3R,4S)-2-(6-benzamido-9H-purin-9-yl)-4-((diisopropoxyphosphoryl)methoxy)tetrahydrofuran-3-yl)O-phenyl carbonothioate. A solution of diisopropyl ((((3S,4R,5R)-5-(6-benzamido-9H-purin-9-yl)-4-hydroxytetrahydrofuran-3-yl)oxy)methyl)phosphonate (350.0 mg, 673 μmol) in MeCN (10 mL) was added DMAP (82.0 mg, 674 μmol) and O-phenyl chlorothionoformate (151.0 mg, 876 μmol). The reaction mixture was stirred at 25° C. for 16 h. Then the reaction mixture was diluted with water (10 mL), extracted with EtOAc (20 ml), washed with brine (20 mL), dried with Na₂SO₄, filtered and concentrated to afford the crude title compound (180.0 mg, 41% yield) as a yellow solid.

Step 5: diisopropyl ((((3R,5R)-5-(6-benzamido-9H-purin-9-yl)tetrahydrofuran-3-yl)oxy)methyl)phosphonate. A solution of O-((2R,3R,4S)-2-(6-benzamido-9H-purin-9-yl)-4-((diisopropoxyphosphoryl)methoxy)tetrahydrofuran-3-yl) O-phenyl carbonothioate (180.0 mg, 274 μmol) in toluene (6 mL) was purged with N₂ for 5 min. AIBN (36.0 mg, 219.0 μmol) and TTMSS (341.0 mg, 1.37 mmol) was added. The reaction mixture was stirred at 110° C. for 2 h. After cooling to room temperature, the reaction mixture was concentrated. The residue was purified by silica gel chromatography eluting with 0-3% MeOH in DCM to afford the title compound (90 mg, 65% yield) as a white solid. LC-MS (ESI) m/z 504.0 [M+H]+. LC-MS RT=0.754 min; Method C.

Step 6: diisopropyl ((((3R,5R)-5-(6-amino-9H-purin-9-yl)tetrahydrofuran-3-yl)oxy)methyl)phosphonate. To a solution of diisopropyl ((((3R,5R)-5-(6-benzamido-9H-purin-9-yl)tetrahydrofuran-3-yl)oxy)methyl)phosphonate (90.0 mg, 179 μmol) in MeOH (2 mL) was added NH₄OH (1 mL, 28% purity). The resulting reaction mixture was stirred at 25° C. for 16 h. Then the reaction mixture was concentrated. The residue was purified by silica gel chromatography eluting with 0-3% MeOH in DCM to afford the title compound (50 mg, 70% yield) as a white solid. LC-MS (ESI) m/z 400.2 [M+H]+. LC-MS RT=1.615 min; Method F.

Step 7: ((((3R,5R)-5-(6-amino-9H-purin-9-yl)tetrahydrofuran-3-yl)oxy)methyl)phosphonic acid (Cpd. No. 113). To a solution of diisopropyl ((((3R,5R)-5-(6-amino-9H-purin-9-yl)tetrahydrofuran-3-yl)oxy)methyl)phosphonate (50.0 mg, 125 μmol) in MeCN (3 mL) was added 2,6-lutidine (107.0 mg, 1.00 mmol) at 25° C. The reaction mixture was cooled to 0° C. and TMSBr (153.0 mg, 1.00 mmol) was added. The reaction mixture was stirred at 0° C. for 2 h. Then the reaction was quenched with 1 M TEAB solution (1 mL) and concentrated. The residue was purified by pre-HPLC (acetonitrile 0-15%/FA in water) to afford Cpd. No. 113 (2.5 mg, 7% yield) as a white solid. ¹H NMR (400 MHz, D₂O) δ 8.49 (s, 1H), 8.22 (s, 1H), 6.40 (d, J=6.4 Hz, 1H), 4.47-4.40 (m, 1H), 4.33 (d, J=10.0 Hz, 1H), 4.11-4.02 (m, 1H), 3.62-3.56 (m, 2H), 2.80-2.56 (m, 2H). LC-MS (ESI) m/z 315.9 [M+H]+. LC-MS RT=0.193 min; Method C.

### Example 42

### Synthesis of 5-(aminomethyl)-1-((2R,4S,5R)-4-hydroxy-5-(hydroxymethyl)-5-methyltetrahydrofuran-2-yl)pyrimidine-2,4(1H,3H)-dione (Cpd. No. 140)

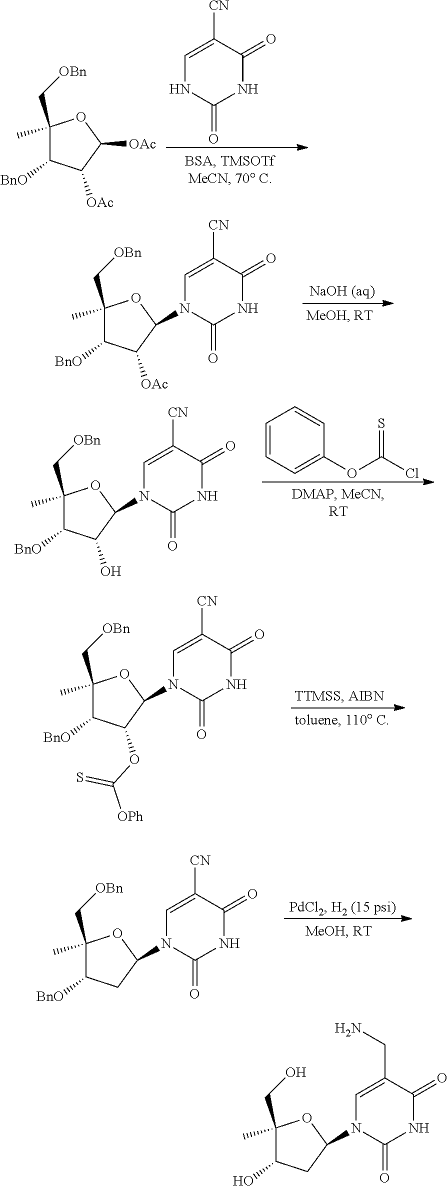

Step 1: (2R,3R,4S,5R)-4-(benzyloxy)-5-((benzyloxy)methyl)-2-(5-cyano-2,4-dioxo-3,4-dihydropyrimidin-1(2H)-yl)-5-methyltetrahydrofuran-3-yl acetate. A mixture of 2,4-dioxo-1H-pyrimidine-5-carbonitrile (0.25 g, 1.82 mmol) and BTMSA (932 mg, 5.47 mmol) in MeCN (10 mL) was stirred at 70° C. for 1 hour, then the reaction solution was cooled to room temperature. A solution of (2S,3R,4S,5R)-4-(benzyloxy)-5-((benzyloxy)methyl)-5-methyltetrahydrofuran-2,3-diyl diacetate (0.5 g, 1.17 mmol) in MeCN (10 mL) and TMSOTf (520 mg, 2.34 mmol) were added, then the mixture was stirred at 30° C. for 16 hours. After that, the reaction solution was diluted with EtOAc (50 mL) and washed with brine (30 mL×2), the organic layer was concentrated to give the crude title compound (0.55 g, 93% yield) as a colorless oil. LC-MS (ESI) m/z 528.0 [M+Na]+. LC-MS RT=0.888 min; Method C.

Step 2: 1-((2R,3R,4S,5R)-4-(benzyloxy)-5-((benzyloxy)methyl)-3-hydroxy-5-methyltetrahydrofuran-2-yl)-2,4-dioxo-1,2,3,4-tetrahydropyrimidine-5-carbonitrile. A mixture of (2R,3R,4S,5R)-4-(benzyloxy)-5-((benzyloxy)methyl)-2-(5-cyano-2,4-dioxo-3,4-dihydropyrimidin-1(2H)-yl)-5-methyltetrahydrofuran-3-yl acetate (0.59 g, 1.17 mmol) and NaOH (2 M, 1.20 mL) in MeOH (10 mL) was stirred at 20° C. for 0.2 hour. The reaction solution was concentrated and diluted with EtOAc (50 mL), washed with brine (30 mL×2), then concentrated to afford the crude title compound (0.5 g, 92% yield) as a white solid which was directly used for next step.

Step 3: O-((2R,3R,4S,5R)-4-(benzyloxy)-5-((benzyloxy)methyl)-2-(5-cyano-2,4-dioxo-3,4-dihydropyrimidin-1(2H)-yl)-5-methyltetrahydrofuran-3-yl)O-phenyl carbonothioate. To a solution of 1-((2R,3R,4S,5R)-4-(benzyloxy)-5-((benzyloxy)methyl)-3-hydroxy-5-methyltetrahydrofuran-2-yl)-2,4-dioxo-1,2,3,4-tetrahydropyrimidine-5-carbonitrile (0.5 g, 1.08 mmol) and DMAP (395.39 mg, 3.24 mmol) in MeCN (20 mL) was added O-phenyl chlorothionoformate (279 mg, 1.62 mmol), then stirred for 1 hour at 20° C. The reaction solution was diluted with EtOAc (50 mL) and washed with brine (30 mL×2), then the organic layer was concentrated to give the crude product. The residue was purified by column chromatography (DCM/MeOH=100/1 to 100/5) to afford the title compound (0.47 g, 73% yield) as a colorless solid.

Step 4: 1-((2R,4S,5R)-4-(benzyloxy)-5-((benzyloxy)methyl)-5-methyltetrahydrofuran-2-yl)-2,4-dioxo-1,2,3,4-tetrahydropyrimidine-5-carbonitrile. A mixture of 1-[(2R,3R,4S,5R)-4-benzyloxy-5-(benzyloxymethyl)-5-methyl-3-phenoxycarbothioyloxy-tetrahydrofuran-2-yl]-2,4-dioxo-pyrimidine-5-carbonitrile (0.47 g, 783.79 umol), TTMSS (974.48 mg, 3.92 mmol) and AIBN (64.35 mg, 391.89 umol) in toluene (15 mL) was stirred at 110° C. for 2 hours. The reaction solution was concentrated to give the crude product. The residue was purified by column chromatography (DCM:MeOH=100/1 to 20/1) to afford the title compound (0.09 g, 26% yield) as a colorless oil. LC-MS (ESI) m/z 470.1 [M+Na]+. LC-MS RT=0.882 min, Method C.

Step 5: 5-(aminomethyl)-1-((2R,4S,5R)-4-hydroxy-5-(hydroxymethyl)-5-methyltetrahydrofuran-2-yl)pyrimidine-2,4(1H,3H)-dione (Cpd. No. 140). A mixture of 1-((2R,4S,5R)-4-(benzyloxy)-5-((benzyloxy)methyl)-5-methyltetrahydrofuran-2-yl)-2,4-dioxo-1,2,3,4-tetrahydropyrimidine-5-carbonitrile (0.05 g, 111.74 umol) and PdCl₂ (9.91 mg, 55.87 umol) in MeOH (10 mL) was stirred at 26° C. under hydrogen (15 psi) for 20 min. The reaction solution was filtrated and concentrated to give the crude product. The residue was purified by prep-HPLC (acetonitrile 0-28%/FA-MeCN in water) to afford Cpd. No. 140 (13.08 mg, 43% yield) as a white solid. ¹H NMR (400 MHz, MeOD-d₄): δ 8.29 (s, 1H), 6.19-6.16 (m, 1H), 4.39-4.36 (m, 1H), 3.81-3.80 (m, 2H), 3.62-3.60 (m, 2H), 2.42-2.32 (m, 2H), 1.19 (s, 3H). LC-MS (ESI) m/z 293.8 [M+Na]+. LC-MS RT=0.168 min; Method C.

### Example 43

### Synthesis of 4-amino-5-fluoro-1-((2R,5S)-5-(hydroxymethyl)-5-methyltetrahydrofuran-2-yl)pyrimidin-2(1H)-one (Cpd. No. 219)

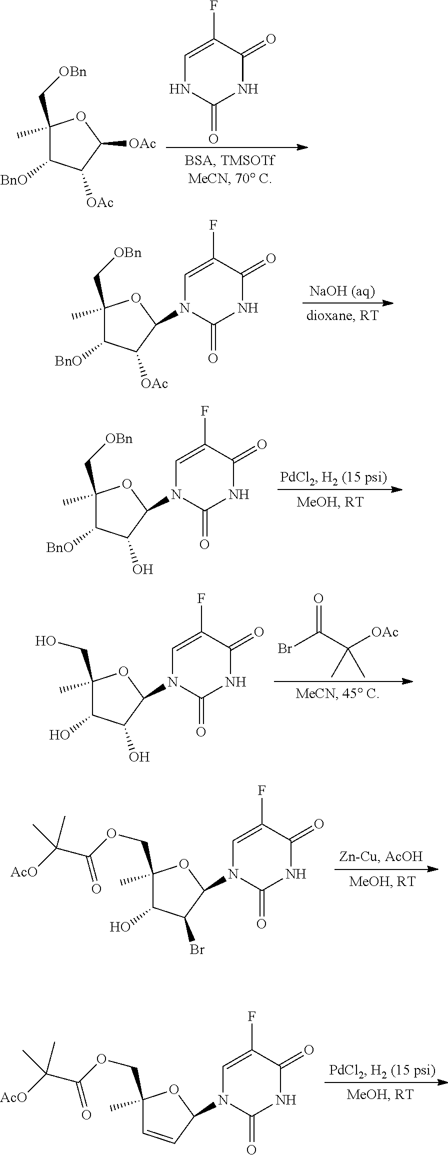

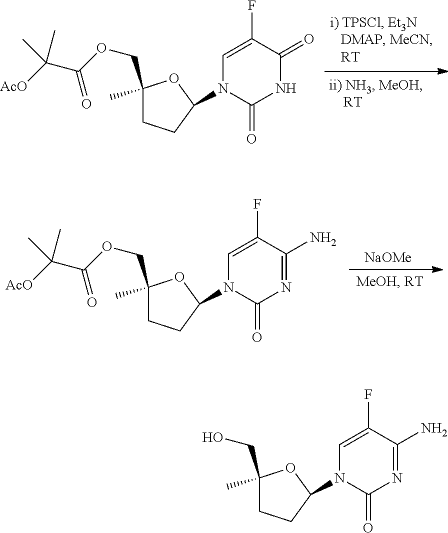 [SMILES: CC(=O)OC(C)(C)C(=O)OC[C@]1(C)CC[C@H](n2cc(F)c(=O)[nH]c2=O)O1 ; CC(=O)OC(C)(C)C(=O)OC[C@]1(C)CC[C@H](n2cc(F)c(N)nc2=O)O1 ; C[C@@]1(CO)CC[C@H](n2cc(F)c(N)nc2=O)O1 ; "NaOMe"]

Step 1: (2R,3R,4S,5R)-4-(benzyloxy)-5-((benzyloxy)methyl)-2-(5-fluoro-2,4-dioxo-3,4-dihydropyrimidin-1(2H)-yl)-5-methyltetrahydrofuran-3-yl acetate. Toa solution of 5-fluoro-1H-pyrimidine-2,4-dione (0.9 g, 7.3 mmol) in MeCN (8 mL) was added A O-bis(trimethylsilyl)acetamide (5.4 mL, 21.9 mmol). The mixture was stirred at 70° C. for 1 h under nitrogen atmosphere. After that, the reaction mixture was cooled to room temperature. Trimethylsilyl trifluoromethanesulfonate (1.4 mL, 7.9 mmol) and (2S,3R,4S,5R)-4-(benzyloxy)-5-((benzyloxy)methyl)-5-methyltetrahydrofuran-2,3-diyl diacetate (2 g, 4.7 mmol) in MeCN (20 mL) were added. The resulting reaction mixture was stirred at 25° C. for 16 h under nitrogen atmosphere. The reaction was quenched with water (20 mL), extracted with EtOAc (50×2 mL), washed with brine (20 mL), dried over Na₂SO₄, filtered and concentrated to give the crude product. The residue was purified by silica gel chromatography (solvent gradient: 0-3% MeOH in dichloromethane) to afford the title product (2.3 g, 99% yield) as a white solid. LC-MS (ESI) m/z 499.3 [M+H]+. LC-MS RT=0.955 min; Method C.

Step 2: 1-((2R,3R,4S,5R)-4-(benzyloxy)-5-((benzyloxy)methyl)-3-hydroxy-5-methyltetrahydrofuran-2-yl)-5-fluoropyrimidine-2,4(1H,3H)-dione. To a solution of (2R,3R,4S,5R)-4-(benzyloxy)-5-((benzyloxy)methyl)-2-(5-fluoro-2,4-dioxo-3,4-dihydropyrimidin-1(2H)-yl)-5-methyltetrahydrofuran-3-yl acetate (2.3 g, 4.6 mmol) in THF (25 mL) was added aqueous NaOH (2 M, 6.9 mL). The mixture was stirred at 25° C. for 1 h. Then the reaction was quenched with water (15 mL), extracted with EtOAc (50 mL), washed with brine (10 mL), dried over Na₂SO₄, filtered and concentrated to give the crude product. It was purified by silica gel chromatography (100-200 mesh silica gel, eluting 0-50% ethyl acetate in petroleum ether) to afford the title product (2.0 g, 98.8% yield). LC-MS (ESI) m/z 479.1 [M+Na]+. LC-MS RT=0.898 min; Method C.

Step 3: 1-((2R,3R,4S,5R)-3,4-dihydroxy-5-(hydroxymethyl)-5-methyltetrahydrofuran-2-yl)-5-fluoropyrimidine-2,4(1H,3H)-dione. A mixture of 1-((2R,3R,4S,5R)-4-(benzyloxy)-5-((benzyloxy)methyl)-3-hydroxy-5-methyltetrahydrofuran-2-yl)-5-fluoropyrimidine-2,4(1H,3H)-dione (1 g, 2.2 mmol) and PdCl₂ (388.5 mg, 2.2 mmol) in MeOH (10 mL) was degassed and purged with H₂ for 3 times, and then the mixture was stirred at 25° C. for 1 h under H₂ atmosphere (15 psi). After that, the reaction mixture was filtrated and concentrated to give the crude. It was purified by silica gel chromatography (100-200 mesh silica gel, eluting 0-50% ethyl acetate in petroleum ether) to afford the title compound (561.7 mg, 93% yield) as colorless solid.

Step 4: ((2R,3R,4S,5R)-4-bromo-5-(5-fluoro-2,4-dioxo-3,4-dihydropyrimidin-1(2H)-yl)-3-hydroxy-2-methyltetrahydrofuran-2-yl)methyl 2-acetoxy-2-methylpropanoate. To a solution of 1-[(2R,3R,4S,5R)-3,4-dihydroxy-5-(hydroxymethyl)-5-methyl-tetrahydrofuran-2-yl]-5-fluoro-pyrimidine-2,4-dione (200 mg, 724.1 umol) in MeCN (4 mL) was added (2-bromo-1,1-dimethyl-2-oxo-ethyl) acetate (1.6 g, 7.2 mmol) at room temperature. The reaction mixture was stirred at 50° C. for 16 h. Then the reaction was quenched with water (10 mL), extracted with EtOAc (20 mL), washed with brine (20 mL), dried over Na₂SO₄, filtered and concentrated to give the crude product. The residue was purified by silica gel chromatography (100-200 mesh silica gel, eluting 0-25% ethyl acetate in petroleum ether) to afford the title compound (262.5 mg, 95% yield) as a colorless oil. LC-MS (ESI) m/z 510 [M+H]+. LC-MS RT=0.753 min; Method C.

Step 5: ((2S,5R)-5-(5-fluoro-2,4-dioxo-3,4-dihydropyrimidin-1(2H)-yl)-2-methyl-2,5-dihydrofuran-2-yl)methyl 2-acetoxy-2-methylpropanoate. A mixture of ((2R,3R,4S,5R)-4-bromo-5-(5-fluoro-2,4-dioxo-3,4-dihydropyrimidin-1(2H)-yl)-3-hydroxy-2-methyltetrahydrofuran-2-yl)methyl 2-acetoxy-2-methylpropanoate (40 mg, 78.5 umol), AcOH (44.9 uL, 785.4 umol) and Zn—Cu (30 mg, 232.7 umol) in MeOH (4 mL) was degassed and purged with N₂ for 3 times, and then the mixture was stirred at 25° C. for 16 h under N₂ atmosphere. Then the reaction was quenched with water (5 mL), extracted with EtOAc (10 mL), washed with brine (10 mL), dried over Na₂SO₄, filtered and concentrated to give the crude product which was purified by silica gel chromatography (solvent gradient: 0-3% MeOH in dichloromethane) to afford the title product (25 mg, 85% yield). LC-MS (ESI) m/z 393.1 [M+Na]+. LC-MS RT=0.773 min; Method C.

Step 6: ((2S,5R)-5-(5-fluoro-2,4-dioxo-3,4-dihydropyrimidin-1(2H)-yl)-2-methyltetrahydrofuran-2-yl)methyl 2-acetoxy-2-methylpropanoate. To a solution of ((2S,5R)-5-(5-fluoro-2,4-dioxo-3,4-dihydropyrimidin-1(2H)-yl)-2-methyl-2,5-dihydrofuran-2-yl)methyl 2-acetoxy-2-methylpropanoate (25 mg, 67.5 umol) in MeOH (4 mL) was added PdCl₂ (12 mg, 67.5 umol). The mixture was stirred at 25° C. for 1 h under H₂ (15 psi). The reaction mixture was filtrated and concentrated to give the crude product. It was purified by silica gel chromatography (100-200 mesh silica gel, eluting 0-25% ethyl acetate in petroleum ether) to afford the title compound (24 mg, 95% yield) as colorless oil. LC-MS (ESI) m/z 395.1 [M+Na]+. LC-MS RT=0.768 min; Method C.

Step 7: ((2S,5R)-5-(4-amino-5-fluoro-2-oxopyrimidin-1(2H)-yl)-2-methyltetrahydrofuran-2-yl)methyl 2-acetoxy-2-methylpropanoate. To a solution of ((2S,5R)-5-(5-fluoro-2,4-dioxo-3,4-dihydropyrimidin-1(2H)-yl)-2-methyltetrahydrofuran-2-yl)methyl 2-acetoxy-2-methylpropanoate (50.0 mg, 134.0 μmol), Et₃N (60.0 μL, 402.8 μmol) and DMAP (16.0 mg, 134.0 μmol) in MeCN (3 mL) was added 2,4,6-triisopropylbenzenesulfonyl chloride (82.0 mg, 268.6 mol). The reaction mixture was stirred at 25° C. for 16 h. A solution of NH₃ in MeOH (7 M, 1 mL) was added. The reaction mixture was stirred at 25° C. for 16 h. The reaction mixture was quenched with water (10 mL), extracted with EtOAc (20 mL), washed with brine (20 mL), dried over Na₂SO₄, filtered, concentrated to give the crude product which was purified by silica gel chromatography (solvent gradient: 0-3% MeOH in Dichloromethane) to afford the title product (30.0 mg, 80% yield) as a yellow solid. LC-MS (ESI) m/z 394.1 [M+Na]+. LC-MS RT=0.654 min; Method C.

Step 8: 4-amino-5-fluoro-1-((2R,5S)-5-(hydroxymethyl)-5-methyltetrahydrofuran-2-yl)pyrimidin-2(1H)-one (Cpd. No. 219). To a solution of ((2S,5R)-5-(4-amino-5-fluoro-2-oxopyrimidin-1(2H)-yl)-2-methyltetrahydrofuran-2-yl)methyl 2-acetoxy-2-methylpropanoate (30.0 mg, 80.8 μmol) in MeOH (2 mL) was added NaOMe (5.0 mg, 80.8 μmol). The reaction mixture was stirred at 25° C. for 16 h. The mixture was concentrated to give the residue which was purified prep-HPLC (acetonitrile 0-30%/NH₃·H₂O+NH₄HCO₃ in water) to afford Cpd. No. 219 (9.4 mg, 48% yield) as a white solid. ¹H NMR (400 MHz, MeOD-d₄) δ 8.48 (d, J=7.2 Hz, 1H), 6.07-6.01 (m, 1H), 3.73-3.65 (m, 1H), 3.60-3.53 (m, 1H), 2.60-2.52 (m, 1H), 2.20-2.11 (m, 1H), 2.08-2.00 (m, 1H), 1.75-1.66 (m, 1H), 1.20 (s, 3H). LC-MS (ESI) m/z 487.2 [2M+H]+. LC-MS RT=1.040 min; Method D.

### Example 44

The analytical characterization for representative Compounds of the Disclosure is provided in Table 5.

TABLE 5

| | | | LC RT | LC % | |
|---|---|---|---|---|---|
| Cpd. | Exact | Observed | (min.), | Purity, | |
| No. | Mass | Mass | Method | λ | ¹H NMR data 400 MHz |
| 1 | 280.12 | (M + Na) | 0.91, E | 99%, | (DMSO-d6) δ 8.22 (s, 1H), 7.99 (s, |
| | | 303 | | 254 | 1H), 6.26-6.23 (m, 1H), 4.41-4.38 |
| | | | | nm | (m, 1H), 3.52-3.33 (m, 2H), 2.83- |
| | | | | | 2.76 (m, 1H), 2.30-2.24 (m, 1H), |
| | | | | | 1.68-1.53 (m, 2H), 0.87 (t, J = 7.2 |
| | | | | | Hz, 3H) |
| 2 | 334.02 | (M − H) | 0.44, D | 100%, | (DMSO-d6) δ 8.47 (s, 1H), 6.03 (t, J = |
| | | 333 | | 254 | 6.4 Hz, 1H), 5.27-5.18 (m, 1H), |
| | | | | nm | 5.14 (d, J = 4.8 Hz, 1H), 4.33-4.21 |
| | | | | | (m, 1H), 3.56-3.49 (m, 1H), 3.45- |
| | | | | | 3.40 (m, 1H), 2.31-2.24 (m, 1H), |
| | | | | | 2.22-2.13 (m, 1H), 1.64-1.53 (m, |
| | | | | | 1H), 1.52-1.41 (m, 1H), 0.85 (t, J = |
| | | | | | 7.6 Hz, 3H) |
| 3 | 307.28 | (M − H) | 0.98, E | 67%, | (DMSO-d6) δ 8.27 (s, 1H), 7.53 (s, |
| | | 306 | | 220 | 1H), 6.27 (t, J = 6.4 Hz, 1H), 5.73 (d, |
| | | | | nm | J = 8.8 Hz, 1H), 5.21 (t, J = 6.4 Hz, |
| | | | | | 1H), 4.38-4.30 (m, 1H), 3.48-3.40 |
| | | | | | (m, 3H), 2.79-2.71 (m, 1H), 2.68- |
| | | | | | 2.62 (m, 1H), 2.53 (s, 3H). |
| 4 | 332.16 | (M − H) | 1.90, C | 99%, | (DMSO-d6) δ 7.95 (s, 1H), 7.37 (s, |
| | | 331 | | 254 | 1H), 6.17 (t, J = 6.4 Hz, 1H), 6.03- |
| | | | | nm | 5.90 (m, 1H), 5.81 (s, 2H), 5.43 (s, |
| | | | | | 1H), 5.37-5.31 (m, 1H), 5.22 (s, 1H), |
| | | | | | 5.20-5.15 (m, 1H), 4.58 (t, J = 6.4 |
| | | | | | Hz, 1H), 3.55-3.47 (m, 1H), 3.45- |
| | | | | | 3.38 (m, 1H), 2.57-2.52 (m, 1H), |
| | | | | | 2.22-2.14 (m, 1H), 0.69-0.62 (m, |
| | | | | | 2H), 0.62-0.55 (m, 2H). |
| 5 | 335.14 | (M − H) | 0.67, C | 99%, | (DMSO-d6) δ 8.54 (br s, 1H), 8.37 (s, |
| | | 334 | | 254 | 1H), 6.28-6.20 (m, 1H), 6.00-5.90 |
| | | | | nm | (m, 1H), 5.42-5.36 (m, 1H), 5.30 (br |
| | | | | | d, J = 4.8 Hz, 1H), 5.26-5.18 (m, |
| | | | | | 1H), 5.10 (br t, J = 5.6 Hz, 1H), 4.71- |
| | | | | | 4.59 (m, 1H), 3.55-3.45 (m, 2H), |
| | | | | | 2.93 (br s, 1H), 2.61-2.70 (m, 1H), |
| | | | | | 2.31-2.22 (m, 1H), 0.79-0.60 (m, 4H). |
| 6 | 284.10 | (M + Na) | 0.33, D | 100%, | (DMSO-d6) δ 11.28 (s, 1H), 7.77 (s, |
| | | 307 | | 254 | 1H), 6.17-6.07 (m, 1H), 5.97-5.85 |
| | | | | nm | (m, 1H), 5.44 (t, J = 4.8 Hz, 1H), 5.39- |
| | | | | | 5.31 (m, 1H), 5.28 (d, J = 5.2 Hz, |
| | | | | | 1H), 5.23-5.15 (m, 1H), 4.54-4.46 |
| | | | | | (m, 1H), 3.65-3.55 (m, 1H), 3.59 (s, |
| | | | | | 3H), 3.43-3.36 (m, 1H), 2.24-2.15 |
| | | | | | (m, 1H), 2.13-2.04 (m, 1H). |
| 7 | 279.09 | (M − H) | 0.67, A | 71%, | (CD3OD) δ 8.63 (s, 1H), 6.28 (dd, J = |
| | | 278 | | 254 | 7.5, 1.3 Hz, 1H), 6.24-6.03 (m, 1H), |
| | | | | nm | 5.54-5.41 (m, 1H), 5.37 (dd, J = |
| | | | | | 11.2, 1.7 Hz, 1H), 4.37 (d, J = 4.8 Hz, |
| | | | | | 1H), 3.51-3.43 (m, 2H), 2.91 (ddd, J = |
| | | | | | 14.9, 7.5, 5.7 Hz, 1H), 1.94 (d, J = |
| | | | | | 41.4 Hz, 2H). |
| 8 | 271.08 | (M + Na) | 0.78, D | 100%, | (DMSO-d6) δ 8.32 (d, J = 7.6 Hz, |
| | | 294 | | 254 | 1H), 7.70 (s, 1H), 7.46 (s, 1H), 6.00- |
| | | | | nm | 5.97 (m, 1H), 5.93-5.85 (m, 1H), |
| | | | | | 5.38-5.31 (m, 2H), 5.23-5.18 (m, |
| | | | | | 2H), 4.40 (t, J = 7.6 Hz, 1H), 3.60- |
| | | | | | 3.56 (m, 1H), 3.40-3.37 (m, 1H), |
| | | | | | 2.13-2.04 (m, 1H) |
| 9 | 293.11 | (M − H) | 0.43, A | 94%, | (CD3OD) δ 8.54-7.73 (m, 1H), 6.54- |
| | | 292 | | 254 | 6.16 (m, 1H), 6.04-5.86 (m, 1H), |
| | | | | nm | 5.51 (ddd, J = 17.3, 11.3, 2.0 Hz, 1H), |
| | | | | | 5.37-5.22 (m, 1H), 4.79-4.53 (m, |
| | | | | | 1H), 3.76-3.46 (m, 2H), 2.73-2.19 |
| | | | | | (m, 2H) |
| 11 | 252.09 | (M − H) | 0.13, A | 90%, | (CD3OD) δ 8.72 (d, J = 27.5 Hz), |
| | | 251 | | 278 | 8.47 (s), 6.24 (ddd, J = 27.6, 7.1, 3.3 |
| | | | | nm | Hz), 4.57 (ddd, J = 11.4, 7.7, 5.7 Hz), |
| | | | | | 3.90-3.46 (m), 3.10 (d, J = 22.2 Hz), |
| | | | | | 3.00-2.96 (m), 2.91 (dt, J = 13.9, 6.9 |
| | | | | | Hz), 2.83 (d, J = 0.7 Hz), 2.79-2.49 |
| | | | | | (m) |
| 17 | 272.18 | (M + Na) | 0.16, D | 100%, | (DMSO-d6) δ 8.26 (d, J = 7.2 Hz, |
| | | 295 | | 254 | 1H), 6.06-6.04 (m, 1H), 5.90-5.80 |
| | | | | nm | (m, 1H), 5.48 (s, 1H), 5.35-5.30 (m, |
| | | | | | 2H), 5.20-5.17 (m, 1H), 4.42-4.31 |
| | | | | | (m, 1H), 3.68-3.33 (m, 2H), 2.21- |
| | | | | | 2.04 (m, 2H) |
| 18 | 274.10 | (M + Na) | 0.15, D | 100%, | (DMSO-d6) δ 8.00 (d, J = 7.2 Hz, |
| | | 297 | | 254 | 1H), 6.07 (t, J = 6.8 Hz, 1H), 5.10- |
| | | | | nm | 5.08 (m, 2H), 4.26 (t, J = 6.0 Hz, 1H), |
| | | | | | 3.50-3.30 (m, 2H), 2.18-2.08 (m, |
| | | | | | 2H), 1.59-1.45 (m, 2H), 0.85 (t, J = |
| | | | | | 7.6 Hz, 3H) |
| 37 | 257.25 | (M + Na) | 0.39, C | 98.0%, | (DMSO-d₆): δ 11.35 (s, 1H), 7.57 (s, |
| | | 274.1 | | 254 | 1H), 6.88-6.87 (m, 1H), 6.36-6.34 |
| | | | | nm | (m, 1H), 6.06-6.04 (m, 1H), 5.47 (t, |
| | | | | | J = 5.6 Hz, 1H), 3.71-3.68 (m, 1H), |
| | | | | | 3.66 (s, 1H), 3.61-3.57 (m, 1H). |
| 62 | 268.11 | (M − H) | 1.09, A | 100%, | (CD₃OD) 9.01 (s, 1H), 7.23 (t, J = 5.7 |
| | | 267 | | 254 | Hz, 1H), 6.99 (dd, J = 17.3, 10.9 Hz, |
| | | | | nm | 1H). 6.51 (dd, J = 17.3, 2.0 Hz, 1H), |
| | | | | | 6.30 (dd, J = 10.9, 1.2 Hz, 1H), 5.63 |
| | | | | | (t, J = 7.4 Hz, 1H), 4.65 (q_{AB}, J = 11.9, |
| | | | | | 5.0 Hz, 2H), 3.30 (dd, J = 7.4, 5.8 Hz, |
| | | | | | 2H), 2.93 (s, 3H) |
| 64 | 332.00 | (M − H) | 1.31, A | 96.3%, | (CD₃OD) δ 8.67 (s, 1H), 6.12 (t, J = |
| | | 331 | | 254 | 5.3 Hz, 1H), 5.92 (dd, J = 17.3, 10.9 |
| | | | | nm | Hz, 1H), 5.46 (dd, J = 17.3, 1.9 Hz, |
| | | | | | 1H), 5.28 (dd, J = 10.9, 1.9 Hz, 1H), |
| | | | | | 4.55 (t, J = 7.6 Hz, 1H), 3.69 (d, J = |
| | | | | | 12.0 Hz, 1H), 3.53 (d, J = 12.0 Hz, |
| | | | | | 1H), 2.27 (dd, J = 7.7, 5.5 Hz, 2H) |
| 66 | 284.10 | (M − H) | | 98.4%, | (CD₃OD) δ 8.94 (s, 1H), 8.80 (s, 1H), |
| | | 283 | | 254 | 7.53-7.16 (m, 1H), 7.02 (ddd, J = |
| | | | | nm | 59.1, 17.4, 11.0 Hz, 1H), 6.62-6.42 |
| | | | | | (m, 1H), 6.42-6.22 (m, 1H), 5.69- |
| | | | | | 5.54 (m, 1H), 5.41 (dt, J = 3.6, 1.8 Hz, |
| | | | | | 1H), 4.77-4.72 (m, 2H), 4.60-4.41 |
| | | | | | (m, 2H), 3.91 (ddd, J = 9.2, 6.3, 3.3 |
| | | | | | Hz, 1H), 3.28 (dd, J = 7.9, 5.6 Hz, |
| | | | | | 1H) |
| 68 | 286.12 | (M − H) | 1.11, A | 96%, | (CD₃OD) δ 7.82 (d, J = 22.7 Hz, 1H), |
| | | 285 | | 254 | 6.38-6.07 (m, 1H), 4.55-4.39 (m, |
| | | | | nm | 0.5H), 4.33-4.10 (m, 0.5H), 3.64- |
| | | | | | 3.37 (m, 2H), 2.90 (m, 0.5H), 2.33 |
| | | | | | (td, J = 7.0, 6.0 Hz, 1H), 1.95-1.75 |
| | | | | | (m, 1H), 1.62 (dd, J = 41.8, 7.6 Hz, |
| | | | | | 1H), 1.54 (m, 0.5), 0.96 (dt, J = 11.9, |
| | | | | | 7.6 Hz, 3H) |
| 69 | 279.09 | (M − H) | 0.67, A | 84%, | (CD₃OD) δ 9.13 (d, J = 4.5 Hz, 1H), |
| | | 278 | | 254 | 6.21-5.98 (m, 1H), 5.92 (dd, J = |
| | | | | nm | 17.3, 11.0 Hz, 1H), 5.46 (dd, J = 17.3, |
| | | | | | 1.9 Hz, 1H), 5.29 (dd, J = 11.0, 1.9 |
| | | | | | Hz, 1H), 4.60-4.45 (m, 1H), 3.72 |
| | | | | | (dd, J = 11.9, 6.9 Hz, 1H), 3.65-3.54 |
| | | | | | (m, 1H), 2.48-2.22 (m, 2H) |
| 70 | 333.03 | (M − H) | 1.21, A | 99%, | (CD₃OD) δ 8.55 (s, 1H), 6.11-6.03 |
| | | 332 | | 254 | (m, 1H), 4.47-4.34 (m, 1H), 4.12- |
| | | | | nm | 4.00 (m, 1H), 3.70 (d, J = 11.7 Hz, |
| | | | | | 1H), 3.61-3.44 (m, 1H), 2.87 |
| | | | | | (ddd, J = 14.8, 7.7, 5.8 Hz, 1H), 2.43 |
| | | | | | (ddd, J = 26.1, 18.8, 13.1 Hz, 1H), |
| | | | | | 2.23 (ddd, J = 13.8, 6.9, 5.5 Hz, 1H), |
| | | | | | 1.86 (dd, J = 15.3, 7.5 Hz, 1H), 1.68 |
| | | | | | (t, J = 7.6 Hz, 1H), 0.96 (tt, J = 22.6, |
| | | | | | 11.3 Hz, 3H) |
| 71 | 270.02 | (M − H) | 1.22, A | 99%, | (CD3OD) δ 7.85 (d, J = 1.2 Hz, 1H), |
| | | 269 | | 254 | 6.17 (dd, J = 8.1, 2.9 Hz, 1H), 4.23 |
| | | | | nm | (dd, J = 6.1, 1.5 Hz, 1H), 3.45 (q, J = |
| | | | | | 11.8 Hz, 2H), 2.89 (tdd, J = 14.4, 8.1, |
| | | | | | 6.1 Hz, 1H), 2.04-1.88 (m, 1H), 1.80 |
| | | | | | (dt, J = 14.0, 6.9 Hz, 1H), 1.58-1.43 |
| | | | | | (m, 2H), 0.97 (t, J = 7.6 Hz, 3H). |
| 72 | 382.13 | (M − H) | 0.79, A | 100%, | (DMSO-d6) δ 8.26 (s, 2H), 8.14 (s, |
| | | 381 | | 254 | 1H), 6.36 (s, 2H), 6.10 (t, J = 6.1 Hz, |
| | | | | nm | 1H), 5.52 (s, 2H), 4.38 (t, J = 6.7 Hz, |
| | | | | | 1H), 3.56-3.42 (m, 3H), 2.70 (dd, J = |
| | | | | | 12.9, 6.6 Hz, 1H), 2.53-2.47 (m, 1H). |
| 73 | 334.02 | (M − H) | 0.645, | 99%, | (DMSO-d6): δ 11.99 (br s, 1H), 8.44 |
| | | 333 | C | 254 | (s, 1H), 6.87 (s, 1H), 6.47-6.40 (m, |
| | | | | nm | 1H), 6.11 (d, J = 5.6 Hz, 1H), 5.58- |
| | | | | | 5.50 (m, 1H), 3.74 (s, 1H), 3.72-3.66 |
| | | | | | (m, 1H), 3.62-3.55 (m, 1H). |
| 74 | 251.21 | (M − H) | 0.38, C | 100%, | (DMSO-d6) δ 8.38 (br s, 1H), 7.92 (d, |
| | | 250 | | 254 | J = 7.2 Hz, 1H), 7.85 (br s, 1H), 7.61 |
| | | | | nm | (br s, 1H), 6.92 (s, 1H), 6.33-6.26 |
| | | | | | (m, 1H), 6.03 (d, J = 5.6 Hz, 1H), 3.76- |
| | | | | | 3.56 (m, 3H). |
| 75 | 307.11 | (M − H) | 0.964, | 95%, | (DMSO-d6): δ 8.24 (s, 1H), 7.51 (s, |
| | | 307 | E | 220 | 1H), 6.37-6.23 (m, 1H), 5.46 (d, J = |
| | | | | nm | 6.0 Hz, 1H), 4.98 (t, J = 6.0 Hz, 1H), |
| | | | | | 4.60-4.47 (m, 1H), 3.61-3.53 (m, |
| | | | | | 1H), 3.48-3.39 (m, 2H), 2.82-2.69 |
| | | | | | (m, 1H), 2.53 (s, 3H), 2.40-2.34 (m, |
| | | | | | 1H) |
| 76 | 251.24 | (M + H) | 0.212, | 95.8%, | (DMSO-d6) δ 8.37 (s, 2H), 7.80 (d, J = |
| | | 252 | C | 254 | 7.2 Hz, 1H), 7.23-7.07 (m, 2H), |
| | | | | nm | 6.20-6.10 (m, 1H), 5.73 (d, J = 7.6 |
| | | | | | Hz, 1H), 4.24 (d, J = 4.8 Hz, 1H), 3.74 |
| | | | | | (d, J = 11.2 Hz, 1H), 3.63 (d, J = 11.2 |
| | | | | | Hz, 1H), 3.51 (s, 1H), 2.78-2.69 (m, |
| | | | | | 1H), 1.85-1.77 (m, 1H). |
| 77 | 251.09 | (M + H) | 0.21, D | 94%, | (DMSO-d6): δ 7.95 (s, 1H), 7.37 (s, |
| | | 252 | | 220 | 1H), 6.17 (t, J = 6.4 Hz, 1H), 6.03- |
| | | | | nm | 5.90 (m, 1H), 5.81 (s, 2H), 5.43 (s, |
| | | | | | 1H), 5.37-5.31 (m, 1H), 5.22 (s, 1H), |
| | | | | | 5.20-5.15 (m, 1H), 4.58 (t, J = 6.4 |
| | | | | | Hz, 1H), 3.55-3.47 (m, 1H), 3.45- |
| | | | | | 3.38 (m, 1H), 2.57-2.52 (m, 1H), |
| | | | | | 2.22-2.14 (m, 1H), 0.69-0.62 (m, |
| | | | | | 2H), 0.62-0.55 (m, 2H) |
| 79 | 291.21 | (M + Na) | 0.20, C | 87%, | (DMSO-d6): δ 10.84 (br s, 1H), 8.24 |
| | | 314 | | 254 | (s, 1H), 6.50-6.41 (m, 1H), 6.15 (s, |
| | | | | nm | 2H), 5.52 (d, J = 5.2 Hz, 1H), 5.36 (t, |
| | | | | | J = 5.6 Hz, 1H), 4.53-4.41 (m, 1H), |
| | | | | | 3.72-3.53 (m, 2H), 3.51 (s, 1H), 2.46- |
| | | | | | 2.34 (m, 2H). |
| 80 | 321.29 | (M − H) | 0.24, A | 96%, | (DMSO-d6) δ = 8.56 (s, 1H), 8.29 (s, |
| | | 274 | | 254 | 1H), 8.16 (s, 1H), 6.86 (s, 2H), 6.41 (t, |
| | | | | nm | J = 5.8, 1H), 6.00-5.15 (br d, 2H), |
| | | | | | 4.49 (t, J = 7.3, 1H), 3.60 (m, 3H), 2.44 |
| | | | | | (m, 1H), 2.42 (m, 1H) |
| 81 | 276.21 | (M − H) | 0.26, A | 99%, | (DMSO-d6) δ = 12.39 (s, 1H), 8.51 |
| | | 275 | | 254 | (s, 0.6H), 8.45 (s, 0.3H), 8.03 (d, |
| | | | | nm | J = 4.0, 0.1H), 7.96 (d, J = 3.5, 1H), 6.64- |
| | | | | | 6.50 (m, 1H), 6.27 (dd, J = 7.0, 4.9, |
| | | | | | 0.8H), 5.80-5.09 (m, 1H), 4.52 (s, |
| | | | | | 0.1H), 4.45 (t, J = 7.3, 0.7H), 4.32 (t, |
| | | | | | J = 5.3, 0.4H), 3.68-3.43 (m, 3H), |
| | | | | | 3.42-3.24 (m, 1H), 2.82 (dt, J = 13.6, |
| | | | | | 6.9, 0.3H), 2.50 (dd, J = 7.8, 5.5, 0.4H), |
| | | | | | 2.44-2.36 (m, 0.4H), 2.28 (dt, |
| | | | | | J = 13.9, 4.2, 0.4H) |
| 91 | 331.02 | (M − H) | 0.89, A | 95%, | (CD3OD) δ 8.29 (s, 1H), 6.25 (dd, J = |
| | | 330 | | 254 | 7.5, 2.0 Hz, 1H), 6.13 (dd, J = 17.5, |
| | | | | nm | 11.2 Hz, 1H), 5.44 (dd, J = 17.5, 1.8 |
| | | | | | Hz, 1H), 5.34 (dd, J = 11.2, 1.8 Hz, |
| | | | | | 1H), 4.52-4.38 (m, 1H), 3.45 (dd, J = |
| | | | | | 28.0, 11.8 Hz, 2H), 2.89 (ddd, J = |
| | | | | | 14.4, 7.5, 5.9 Hz, 1H), 2.05-1.88 (m, |
| | | | | | 1H) |
| 82 | 330.12 | (M + H) | 0.68, A | 100%, | (DMSO-d6) δ 8.23 (s, 1H), 8.07 (s, |
| | | 331 | | 254 | 1H), 6.53 (d, J = 2.7 Hz, 1H), 6.03 (t, |
| | | | | nm | J = 6.2 Hz, 1H), 5.65 (s, 2H), 4.38 (t, |
| | | | | | J = 6.8 Hz, 1H), 3.56-3.43 (m, 3H), |
| | | | | | 2.93-2.82 (m, 1H), 2.68 (dt, J = 12.8, |
| | | | | | 6.4 Hz, 1H), 2.45-2.37 (m, 1H), 0.75- |
| | | | | | 0.62 (m, 2H), 0.59-0.40 (m, 2H) |
| 92 | 316.03 | (M + Na) | 0.64, C | 100%, | (DMSO-d6): δ 11.87 (br s, 1H), 8.21 |
| | | 315 | | 254 | (s, 1H), 7.42-6.95 (m, 1H), 6.87 (s, |
| | | | | nm | 1H), 6.45-6.37 (m, 1H), 6.20-6.00 |
| | | | | | (m, 1H), 5.51 (t, J = 5.6 Hz, 1H), 3.72 |
| | | | | | (s, 1H), 3.71-3.65 (m, 1H), 3.62- |
| | | | | | 3.53 (m, 1H) |
| 83 | 330.12 | (M + H) | 0.25, B | 100%, | (DMSO-d6) δ 8.22 (s, 1H), 8.14 (s, |
| | | 331 | | 254 | 1H), 6.52 (s, 1H), 6.16 (t, J = 6.3 Hz, |
| | | | | nm | 1H), 5.67 (s, 2H), 4.39 (t, J = 6.4 Hz, |
| | | | | | 1H), 3.62 (s, 1H), 3.59-3.50 (m, |
| | | | | | 2H), 2.88-2.77 (m, 1H), 2.47-2.39 |
| | | | | | (m, 1H), 2.37-2.26 (m, 1H), 0.76- |
| | | | | | 0.62 (m, 2H), 0.50 (m, 2H) |
| 84 | 379.3 | (M − H) | 1.36, A | 90%, | (CD3OD) δ 7.99 (d, J = 26.3 Hz, 1H), |
| | | 332 | | 254 | 6.28 (dd, J = 7.2, 4.6 Hz, 1H), 4.53- |
| | | | | nm | 4.42 (m, 2H), 3.68 (q, J = 12.1 |
| | | | | | Hz, 1H), 3.19-3.10 (m, 1H), 3.00- |
| | | | | | 2.85 (m, 2H), 2.84 (d, J = 0.6 Hz, 1H), |
| | | | | | 2.49 (dt, J = 13.8, 4.9 Hz, 1H), 0.90- |
| | | | | | 0.81 (m, 2H), 0.66-0.57 (m, 2H). |
| 85 | 333.09 | (M + H) | 2.21, A | 96%, | (CD3OD) δ 8.12 (d, J = 78.6 Hz, 1H), |
| | | 334 | | 254 | 6.33 (dd, J = 7.1, 4.9 Hz, 1H), 4.71 (t, |
| | | | | nm | J = 6.9 Hz, 1H), 3.79 (dd, J = 35.3, |
| | | | | | 12.2 Hz, 2H), 3.08 (s, 1H), 2.87 (d, J = |
| | | | | | 28.2 Hz, 1H), 2.79-2.45 (m, 2H), |
| | | | | | 0.90-0.76 (m, 2H), 0.73-0.45 (m, |
| | | | | | 2H). |
| 86 | 315.09 | (M + H) | 1.40, A | 100%, | (CD3OD) δ 9.11-8.96 (m, 1H), 7.21 |
| | | 316 | | 254 | (dd, J = 6.9, 5.5 Hz, 1H), 5.48 (t, J = |
| | | | | nm | 6.5 Hz, 1H), 4.58 (dd, J = 40.2, 12.2 |
| | | | | | Hz, 2H), 3.76-3.63 (m, 1H), 3.59 (d, |
| | | | | | J = 19.3 Hz, 1H), 3.36 (d, J = 6.4 Hz, |
| | | | | | 1H), 2.77 (s, 1H), 1.64 (td, J = 6.9, 5.1 |
| | | | | | Hz, 2H), 1.49-1.33 (m, 2H) |
| 87 | 293.11 | (M − H) | 0.30, A | 95%, | (CD3OD) δ 8.17 (d, J = 99.1 Hz, 1H), |
| | | 292 | | 254 | 6.28 (dd, J = 7.9, 3.6 Hz, 1H), 6.04 |
| | | | | nm | (ddd, J = 17.3, 13.6, 11.1 Hz, 1H), |
| | | | | | 5.37-5.08 (m, 2H), 4.47 (dd, J = 6.5, |
| | | | | | 2.9 Hz, 1H), 3.58-3.37 (m, 2H), 3.04- |
| | | | | | 2.84 (m, 1H), 2.37-2.23 (m, 1H) |
| 89 | 253.11 | (M − H) | 0.18, A | 100%, | (CD3OD) δ 7.99 (d, J = 7.5 Hz, 1H), |
| | | 252 | | 254 | 6.28 (dd, J = 7.6, 2.9 Hz, 1H), 6.09 |
| | | | | nm | (dd, J = 17.5, 11.2 Hz, 1H), 5.85 (d, J = |
| | | | | | 7.5 Hz, 1H), 5.35 (ddd, J = 14.3, |
| | | | | | 13.0, 1.8 Hz, 3H), 4.37 (dd, J = 6.0, |
| | | | | | 2.1 Hz, 1H), 3.46 (dd, J = 29.0, 11.7 |
| | | | | | Hz, 2H), 2.86 (ddd, J = 13.8, 7.5, 6.1 |
| | | | | | Hz, 1H), 1.97 (ddd, J = 8.0, 7.1, 5.4 |
| | | | | | Hz, 1H) |
| 90 | 332.15 | (M − H) | 0.87, A | 100%, | (CD3OD) δ 8.70 (s, 1H), 6.06 (dd, J = |
| | | 331.1 | | 254 | 6.9, 3.0 Hz, 1H), 6.02-5.82 (m, 1H), |
| | | | | nm | 5.46 (dd, J = 17.3, 1.8 Hz, 1H), 5.36- |
| | | | | | 5.19 (m, 1H), 4.59-4.43 (m, 1H), |
| | | | | | 4.44-4.36 (m, 1H), 3.70 (dd, J = |
| | | | | | 12.0, 5.7 Hz, 1H), 3.55 (d, J = 12.0 |
| | | | | | Hz, 1H), 2.38-2.24 (m, 1H), 2.18 |
| | | | | | (ddd, J = 13.4, 7.1, 3.0 Hz, 1H). |
| 93 | 307.11 | (M + H) | 0.56, C | 99%, | (DMSO-d6) δ 8.34 (br d, J = 4.4 Hz, |
| | | 306 | | 254 | 1H), 8.30 (s, 1H), 6.28-6.22 (m, 1H), |
| | | | | nm | 5.58 (br d, J = 5.6 Hz, 1H), 5.30 (br s, |
| | | | | | 1H), 4.60-4.54 (m, 1H), 3.68-3.62 |
| | | | | | (m, 1H), 3.59-3.53 (m, 1H), 3.51 (s, |
| | | | | | 1H), 2.92 (d, J = 4.4 Hz, 3H), 2.74- |
| | | | | | 2.65 (m, 1H), 2.47-2.39 (m, 1H). |
| 94 | 317.15 | (M − H) | 0.93, C | 99%, | (DMSO-d6) δ 8.37 (s, 1H), 8.24 (s, |
| | | 316 | | 254 | 1H), 7.96 (s, 1H), 6.40-6.30 (m, 1H), |
| | | | | nm | 6.03-5.91 (m, 1H), 5.44-5.32 (m, |
| | | | | | 2H), 5.28 (d, J = 5.2 Hz, 1H), 5.23- |
| | | | | | 5.16 (m, 1H), 4.68-4.61 (m, 1H), |
| | | | | | 3.56-3.41 (m, 2H), 2.66-2.61 (m, |
| | | | | | 1H), 2.31-2.21 (m, 1H), 0.76-0.68 |
| | | | | | (m, 2H), 0.65-0.55 (m, 2H). |
| 96 | 319.16 | (M − H) | 0.96, C | 63%, | (DMSO-d6) δ 8.34 (s, 1H), 8.23 (s, |
| | | 318 | | 220 | 1H), 7.94 (s, 1H), 6.35-6.27 (m, 1H), |
| | | | | nm | 5.25-5.12 (m, 2H), 4.46-4.36 (m, |
| | | | | | 1H), 3.60-3.49 (m, 1H), 3.46-3.39 |
| | | | | | (m, 1H), 2.94-2.84 (m, 1H), 2.31- |
| | | | | | 2.22 (m, 1H), 1.72-1.51 (m, 2H), |
| | | | | | 0.88 (t, J = 7.5 Hz, 3H), 0.75-0.68 |
| | | | | | (m, 2H), 0.64-0.56 (m, 2H) |
| 97 | 334.18 | (M − H) | 1.0, C | 43%, | (DMSO-d6) δ 7.91 (s, 1H), 7.34 (s, |
| | | 333 | | 220 | 1H), 6.21-6.10 (m, 1H), 5.80 (s, 2H), |
| | | | | nm | 5.17-5.26 (m, 1H), 5.10 (d, J = 4.4 |
| | | | | | Hz, 1H), 4.43-4.30 (m, 1H), 3.52- |
| | | | | | 3.45 (m, 1H), 3.44-3.39 (m, 1H), |
| | | | | | 2.81-2.69 (m, 1H), 2.23-2.13 (m, |
| | | | | | 1H), 1.69-1.49 (m, 2H), 0.86 (t, J = |
| | | | | | 7.6 Hz, 3H), 0.70-0.63 (m, 2H), 0.61- |
| | | | | | 0.54 (m, 2H) |
| 98 | 281.10 | (M − H) | 0.2, D | 100%, | (DMSO-d6) δ 8.91 (s, 1H), 5.94 (t, J = |
| | | 280 | | 254 | 6.0 Hz, 1H), 5.32-5.25 (m, 1H), 5.17- |
| | | | | nm | 5.12 (m, 1H), 4.31-4.23 (m, 1H), |
| | | | | | 3.60-3.52 (m, 1H), 3.45-3.39 (m, |
| | | | | | 1H), 2.37-2.22 (m, 2H), 1.65-1.55 |
| | | | | | (m, 1H), 1.54-1.39 (m, 1H), 0.86 (t, J = |
| | | | | | 7.6 Hz, 3H) |
| 99 | 337.16 | (M − H) | 1.23, C | 100%, | (DMSO-d6) δ 8.53 (br s, 1H), 8.33 (s, |
| | | 336 | | 254 | 1H), 6.20 (t, J = 6.8 Hz, 1H), 5.17 (d, |
| | | | | nm | J = 4.8 Hz, 1H), 4.92 (t, J = 5.2 Hz, |
| | | | | | 1H), 4.46-4.36 (m, 1H), 3.53-3.40 |
| | | | | | (m, 2H), 2.93 (br s, 1H), 2.84-2.76 |
| | | | | | (m, 1H), 2.36-2.23 (m, 1H), 1.70- |
| | | | | | 1.49 (m, 2H), 0.92-0.83 (m, 3H), |
| | | | | | 0.75-0.61 (m, 4H) |
| 100 | 278.1 | (M + H) | 0.19, C | 97%, | (DMSO-d6) δ 8.26 (s, 1H), 8.00 (s, |
| | | 279 | | 254 | 1H), 6.29-6.26 (m, 1H), 5.98-5.92 |
| | | | | nm | (m, 1H), 5.40-5.35 (m, 1H), 5.24- |
| | | | | | 5.18 (m, 1H), 4.62 (t, J = 6.4 Hz, 1H) |
| | | | | | 3.52-3.33 (m, 2H), 2.62-2.57 (m, |
| | | | | | 1H), 2.26-2.21 (m, 1H) |
| 101 | 305.12 | (M + H) | 0.82 | 82%, | (DMSO-d6) δ 8.39 (s, 1H), 6.37- |
| | | 306 | | 220 | 6.25 (m, 1H), 6.18 (s, 2H), 5.54 (d, J = |
| | | | | nm | 5.6 Hz, 1H), 5.36 (t, J = 6.0 Hz, 1H), |
| | | | | | 4.51-4.39 (m, 1H), 3.97 (s, 3H), 3.68- |
| | | | | | 3.63 (m, 1H), 3.61-3.54 (m, 1H), |
| | | | | | 3.52 (s, 1H), 2.46-2.38 (m, 2H) |
| 102 | 307.12 | (M + H) | 0.90 | 96%, | (CD3OD) δ 8.61 (s, 1H), 6.46-6.39 |
| | | 308 | | 220 | (m, 1H), 6.06-6.95 (m, 1H), 5.62- |
| | | | | nm | 5.51 (m, 1H), 5.38-5.31 (m, 1H), |
| | | | | | 4.65 (t, J = 7.6 Hz, 1H), 4.08 (s, 3H), |
| | | | | | 3.76-3.67 (m, 1H), 3.61-3.56 (m, |
| | | | | | 1H), 2.53-2.39 (m, 2H) |
| 103 | 286.12 | (M + H) | 0.26, D | 99%, | (CD3OD) δ 7.87 (s, 1H), 6.25 (t, J = |
| | | 287 | | 254 | 6.4 Hz, 1H), 4.55-4.47 (m, 1H), 3.76- |
| | | | | nm | 3.72 (m, 1H), 3.71 (s, 3H), 3.63- |
| | | | | | 3.58 (m, 1H), 2.45-2.27 (m, 2H), |
| | | | | | 1.79-1.66 (m, 1H), 1.64-1.52 (m, |
| | | | | | 1H), 0.97 (t, J = 7.6 Hz, 3H) |
| 105 | 334.12 | (2M + H) | 0.89, E | 97%, | (CD3OD): δ 8.56 (s, 1H), 6.09 (t, J = |
| | | 669 | | 220 | 6.0 Hz, 1H), 4.44 (t, J = 6.4 Hz, 1H), |
| | | | | nm | 3.73 (d, J = 12.0 Hz, 1H), 3.58 (d, |
| | | | | | J = 12.0 Hz, 1H), 2.56-2.42 (m, 1H), |
| | | | | | 2.34-2.19 (m, 1H), 1.80-1.66 (m, |
| | | | | | 1H), 1.65-1.54 (m, 1H), 0.97 (t, J = |
| | | | | | 7.6 Hz, 3H) |
| 108 | 256.11 | (M + Na) | 0.88, D | 99%, | (DMSO-d6) δ 7.91 (d, J = 8.0 Hz, |
| | | 279 | | 220 | 1H), 6.10-5.90 (m, 2H), 5.61 (d, J = |
| | | | | nm | 8.0 Hz, 1H), 4.27-4.24 (m, 1H), 3.44- |
| | | | | | 3.40 (m, 2H), 2.21-2.14 (m, 2H), |
| | | | | | 1.61-1.48 (m, 2H), 0.85 (t, J = 7.6 |
| | | | | | Hz, 3H). |

### Example 33

### Human LINE-1 Retrotransposition Assay

Representative Compounds of the Disclosure were tested for inhibition of retrotransposition activity of human LINE-1 in HeLa cells according to the following procedure.

HeLa cervical cancer cells were cultivated at 37° C. in a humidified 5% CO₂ incubator in Dulbecco's Modified Eagle's Medium (DMEM)—high glucose, with 4500 mg/L glucose, L-glutamine, sodium pyruvate and sodium bicarbonate (Sigma), supplemented with 10% of heat inactivated fetal bovine serum (Thermo Fisher).

Assays were performed using reporter plasmid pYX017 as described (Xie, et al., 2011) with several modifications. The reporter assay was performed in 96-well white optical bottom plates. HeLa cells were seeded in wells 24 h prior to transfection and compound treatment so that cells were approximately 30% confluent on the day of transfection. Different cell plating densities were tested and a density of 2×10³ cells was determined to be optimal.

Compounds were resuspended in DMSO. Serial dilutions (1:3) were prepared in DMSO. Medium containing different concentrations of the compounds were prepared by adding 2 μl of the compound dilution to 1 ml of the culture medium. The final concentration of DMSO in the medium was 0.2%.

FuGENE® HD transfection reagent (Promega, E2311, Lot 382574 and Lot 397842) was used to transfect the plasmids into the cells. The transfection reagent: DNA mixture was prepared in OpiMEM (Thermo Fisher) according to manufacturer's instructions. Different ratios of transfection reagent to DNA were tested and a ratio of 3:1 was determined to be optimal. Culture medium was removed from the cells and discarded. The transfection reagent: DNA mixture (5 μl) was mixed with the compound containing medium (100 μl/well) and this was added onto the cells of each well. Cells were incubated at 37° C./5% CO₂ for different incubation time. A 72 h incubation time was determined to be optimal.

Luciferase reporter activity was quantified with the Dual-Luciferase® Reporter Assay System (Promega) according to manufacturer's instructions for multiwell plates except that cells were lysed directly on the multiwell plate with 30 μl of the passive lysis buffer (PLB) for 20 min at room temperature, with gentle shaking to ensure complete cell lysis.

Firefly and Renilla luciferase signals were measured using a SpectraMax i3× Multi-Mode Microplate Reader. Integration times of 100 ms and 10 ms were used to measure the Firefly and Renilla signals respectively. Relative L1 activity is calculated as Firefly/Renilla*1000 or Firefly/Renilla*10,000. Dose response inhibition data were fit to a four parameter logistic equation using non-linear regression (using Graphpad Prism 8), to determine IC₅₀ values for each inhibitor. The results are provided in Table 6.

TABLE 6
Human LINE-1 Activity Inhibition

| Cpd. No. | LINE-1 IC₅₀ (μM) |
|---|---|
| 1 | 0.062 |
| 2 | 6.900 |
| 3 | 0.041 |
| 4 | 0.401 |
| 5 | 0.469 |
| 6 | >50 |
| 7 | 0.890 |
| 8 | 0.009 |
| 9 | 0.013 |
| 11 | >50 |
| 12 | |
| 13 | 370 |
| 14 | 50.000 |
| 17 | 2.349 |
| 18 | 3.747 |
| 19 | >50 |
| 20 | 7.206 |
| 21 | >50 |
| 22 | 20.060 |
| 23 | >50 |
| 24 | >50 |
| 25 | >50 |
| 26 | >50 |
| 27 | >50 |
| 28 | >12.5 |
| 29 | >50 |
| 30 | 15.970 |
| 31 | >50 |
| 32 | 0.840 |
| 33 | >12.5 |
| 34 | >50 |
| 35 | >50 |
| 36 | >12.5 |
| 37 | 0.067 |
| 38 | >50 |
| 39 | >50 |
| 40 | >50 |
| 41 | >50 |
| 42 | >50 |
| 43 | 0.876 |
| 44 | 2.200 |
| 45 | 3.811 |
| 46 | >50 |
| 47 | >25 |
| 48 | >50 |
| 49 | 0.052 |
| 50 | 25.510 |
| 51 | 5.075 |
| 52 | >50 |
| 53 | >50 |
| 55 | >50 |
| 55 | 2.014 |
| 56 | >50 |
| 57 | >50 |
| 58 | >50 |
| 59 | 20.940 |
| 60 | 5.650 |
| 61 | 7.429 |
| 62 | 3.669 |
| 63 | >12.5 |
| 64 | 9.934 |
| 65 | 21.920 |
| 66 | 0.738 |
| 67 | 0.004 |
| 68 | 0.721 |
| 69 | 6.204 |
| 70 | 0.432 |
| 71 | 1.597 |
| 72 | 23.370 |
| 73 | >50 |
| 74 | >50 |
| 75 | >50 |
| 76 | >6.25 |
| 77 | >6.25 |
| 79 | 17.000 |
| 80 | 3.700 |
| 81 | 4.400 |
| 82 | 10.040 |
| 83 | 0.429 |
| 84 | 14.000 |
| 85 | 0.124 |
| 86 | 3.400 |
| 87 | 0.173 |
| 89 | 1.285 |
| 90 | 0.658 |
| 81 | 7.449 |
| 82 | >50 |
| 93 | 14.970 |
| 94 | >50 |
| 95 | >50 |
| 96 | 6.997 |
| 97 | 0.946 |
| 98 | >25 |
| 99 | 5.533 |
| 100 | 1.700 |
| 101 | 0.259 |
| 102 | 1.775 |
| 103 | >12.5 |
| 104 | 4.896 |
| 105 | 0.150 |
| 106 | 24.000 |
| 107 | 7.300 |
| 125 | 0.11 |
| 139 | 3165 |
| 154 | >40 |
| 163 | 14 |
| 164 | >50 |
| 166 | 417 |
| 182 | 293 |
| 189 | 9172 |
| 190 | ~50 |
| 191 | 56 |
| 192 | 122 |
| 193 | 2815 |
| 194 | 1943 |
| 195 | >50 |
| 196 | >50 |
| 198 | >50 |
| 200 | 414 |

Having now fully described the compounds, methods, kits, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

1-23. (canceled)

24. A compound selected from the group consisting of: 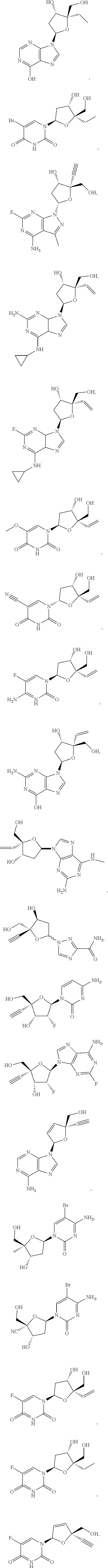 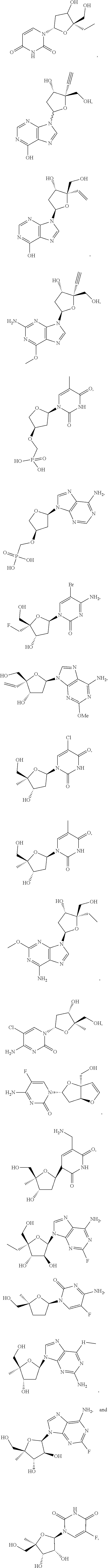
or a pharmaceutically acceptable salt or solvate thereof, or a tautomer thereof.

25. The compound of claim 24, wherein the compound is selected from the group consisting of: 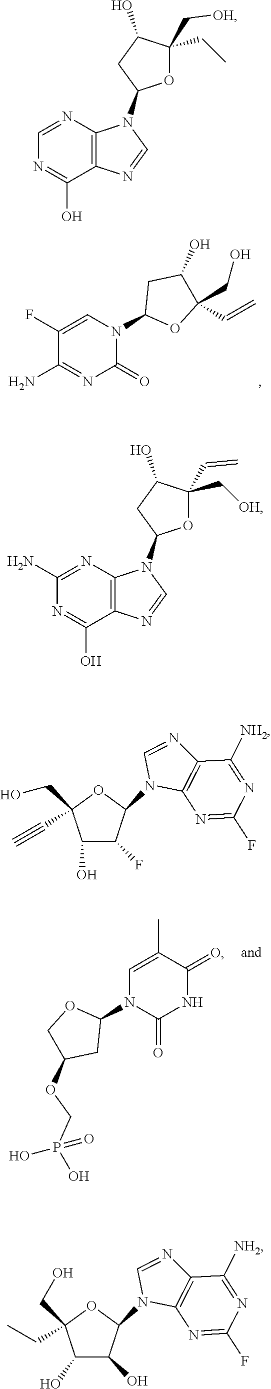
or a pharmaceutically acceptable salt or solvate thereof, or a tautomer thereof.

26. A pharmaceutical composition comprising the compound of claim 24, or a pharmaceutically acceptable salt or solvate thereof, or a tautomer thereof, and one or more pharmaceutically acceptable excipients.

27. A method of treating a disease, disorder, or condition in a subject in need thereof, and/or treating a symptom of the disease, disorder, or condition, the method comprising administering a therapeutically effective amount of the compound of claim 24, or a pharmaceutically acceptable salt or solvate thereof, or a tautomer thereof, to the subject, wherein the disease, disorder, or condition is a neurodegenerative disease, an autoimmune disease, an age-associated disease, autism spectrum disorder, cardiovascular dysfunction, hearing loss, hematopoietic stem cell function, pulmonary fibrosis, schizophrenia, vision loss, progressive supra nuclear palsy, amyotrophic lateral sclerosis, Aicardi-Goutières syndrome, ataxia-telangiectasia, age-related macular degeneration, systemic lupus erythematosus, IFN-associated autoimmune disease, Fanconi Anemia, idiopathic pulmonary fibrosis, or cardiovascular disease.

28. The method of claim 27, wherein the disease, disorder, or condition is a neurodegenerative disease.

29. The method of claim 28, wherein the neurodegenerative disease is Alzheimer's disease, amyotrophic lateral sclerosis, Parkinson's disease, dementia with Lewy Bodies, multi systems atrophy, Huntington's disease, frontotemporal dementia (FTD), frontotemporal lobar degeneration, mild cognitive impairment, corticobasal degeneration, progressive supra nuclear palsy, Rett Syndrome, peripheral degenerative disease, or Aicardi-Goutières syndrome.

30. The method of claim 27, wherein the disease, disorder, or condition is an autoimmune disease.

31. The method of claim 30, wherein the autoimmune disease is lupus, rheumatoid arthritis, Sjogrens syndrome, or multiple sclerosis.

32. The method of claim 27, wherein the disease, disorder, or condition is an age-associated disease.

33. The method of claim 32, wherein the age-associated disease is Alzheimer's disease, Parkinson's disease, atherosclerosis, osteoarthritis, osteoporosis, rheumatoid arthritis, macular degeneration, peripheral degenerative disease, or skin aging.

34. The method of claim 27, wherein the disease, disorder, or condition is autism spectrum disorder, cardiovascular dysfunction, hearing loss, hematopoietic stem cell function, pulmonary fibrosis, schizophrenia, or vision loss.

35. The method of claim 27, wherein the disease, disorder, or condition is progressive supra nuclear palsy.

36. The method of claim 27, wherein the disease, disorder, or condition is amyotrophic lateral sclerosis.

37. The method of claim 27, wherein the disease, disorder, or condition is Aicardi-Goutières syndrome.

38. The method of claim 27, wherein the disease, disorder, or condition is ataxia-telangiectasia, age-related macular degeneration, systemic lupus erythematosus, IFN-associated autoimmune disease, Fanconi Anemia, idiopathic pulmonary fibrosis, or cardiovascular disease.

39. The method of claim 27 for treating the disease, disorder, or condition in the subject.

40. The method of claim 27 for treating the symptom of a disease, disorder, or condition in the subject.

41. The method of claim 27, wherein the compound inhibits human LINE-1 retrotransposition activity with a half maximal inhibitory concentration of 1 μM or less in an in vitro HeLa cell-based dual-luciferase assay.

42. The method of claim 27, further comprising administering one or more optional therapeutic agents to the subject.

43. A kit for carrying out the method of claim 27, the kit comprising the compound, or a pharmaceutically acceptable salt or solvate thereof, or a stereoisomer thereof, or a tautomer thereof, and instructions for administering the compound to the subject.
